(19) United States
(12) Patent Application Publication
(10) Pub. No.: US 2010/0171417 A1
(43) Pub. Date: Jul. 8, 2010

(54) CHARGE TRANSPORT MATERIAL AND ORGANIC ELECTROLUMINESCENCE DEVICE

(75) Inventors: Tetsu KITAMURA, Ashigarakami-gun (JP); Satoshi SANO, Minami-Ashigara-shi (JP); Toshihiro ISE, Ashigarakami-gun (JP); Saki TAKADA, Ashigarakami-gun (JP)

Correspondence Address: SUGHRUE-265550, 2100 PENNSYLVANIA AVE. NW, WASHINGTON, DC, 20037-3213, US

(73) Assignee: FUJIFILM CORPORATION, Tokyo (JP)

(21) Appl. No.: 12/683,011

(22) Filed: Jan. 6, 2010

(30) Foreign Application Priority Data
Jan. 6, 2009 (JP) 2009-001161

(51) Int. Cl. H01J 1/62; C07D 401/10

(52) U.S. Cl. 313/504; 546/102

(57) ABSTRACT

Provided is a charge transport material represented by the following general formula (1) or (2).

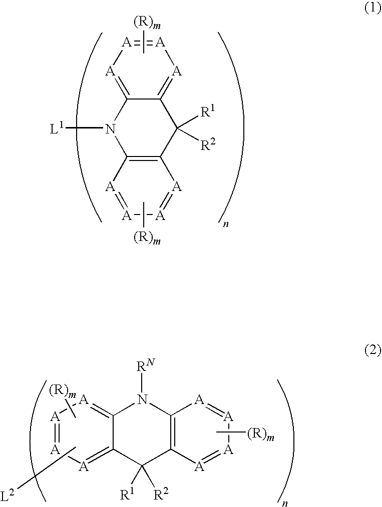

In the foregoing general formulae, each of L¹ and L² independently represents a connecting group; each of R and R^{N} independently represents a substituent; each of R¹ and R² independently represents a substituent, provided that R¹ and R² do not represent an aryl group at the same time; each A independently represents a carbon atom or a nitrogen atom; n represents an integer of 2 or more and not more than 10; and each m independently represents an integer.

### BACKGROUND OF THE INVENTION

1. Field of the Invention

The present invention relates to a charge transport material and an organic electroluminescence device.

2. Description of the Related Art

An organic electroluminescence device (hereinafter also referred to as “organic EL device”) is being actively researched and developed because light emission with high brightness is obtained by low-voltage driving. The organic electroluminescence device has an organic layer between a pair of electrodes, and an electron injected from a cathode and a hole injected from an anode are recombined with each other in the organic layer, thereby utilizing energy of a generated exciton for light emission.

In recent years, by using a phosphorescent material, realization of high efficiency of a device is being advanced. There are disclosed inventions regarding a phosphorescene device using an indium complex, a platinum complex or the like as the phosphorescent material (see, for example, U.S. Pat. No. 6,303,238 and WO 00/57676). However, a device which is capable of making both high efficiency and high durability compatible with each other has not been developed yet.

As one of causes for the fact that both high efficiency and high durability cannot be made compatible with each other in the phosphorescence device, there is exemplified the matter that host materials having high chemical stability and carrier injection and transport properties and large lowest excited triplet energy (T₁ energy) are restricted. When T₁ of the host material is smaller than T₁ of the phosphorescent material, the light emission is quenched, and hence, the host material is required to have larger T₁ than the phosphorescent material. Also, even in the case where T₁ of the host material is larger than T₁ of the phosphorescent material, when a difference in T₁ between the both is small, reverse energy transport from the phosphorescent material to the host material partially occurs, and hence, a lowering in the efficiency or a lowering in the durability may be caused. Accordingly, a host material having sufficiently large T₁ and high chemical stability and carrier injection and transport properties is demanded.

There is disclosed an invention regarding an organic electroluminescence device using a material composed of the following tetraphenylsilane compound as a host material capable of forming a light emitting layer together with a phosphorescent material (see, for example, US 2004/0209116). Though this material has large T₁, it is low in charge injection and transport capability. Therefore, a driving voltage of the device is high, and driving durability of the device is low. Thus, improvements are required.

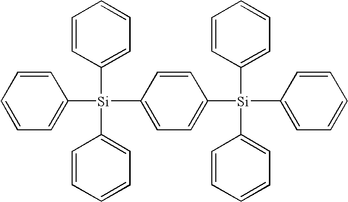

Also, WO 07/110228 discloses an organic EL device using a material composed of the following acridan structure in which a connection moiety thereof is a diarylmethylene.

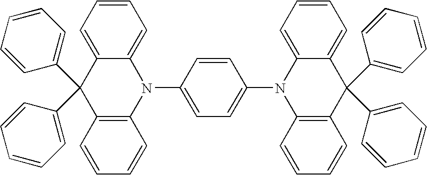

According to studies made by the present inventor, the foregoing material involves such problems as low chemical stability and insufficient driving durability.

Also, JP-A-2002-231453 discloses an organic EL device using a material composed of the following phenoxazine or phenothiazine structure.

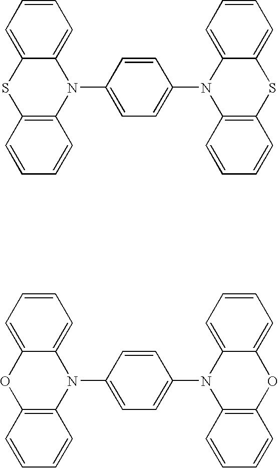

However, since the foregoing material is small in T₁, when it is used together with a phosphorescent material (in particular, a blue phosphorescent material with a short light emission wavelength), the light emission of the phosphorescent material is quenched. Thus, there are involved problems that the efficiency is largely lowered and that the driving durability is insufficient.

### SUMMARY OF THE INVENTION

An object of the invention is to provide a charge transport material having high chemical stability and large T₁. Another object of the invention is to provide an organic EL device having high efficiency, low driving voltage and high driving durability using the instant charge transport material.

### Means for Solving the Problems

The invention is as follows.
[1] A compound represented by the following general formula (1-1) or (2-1).

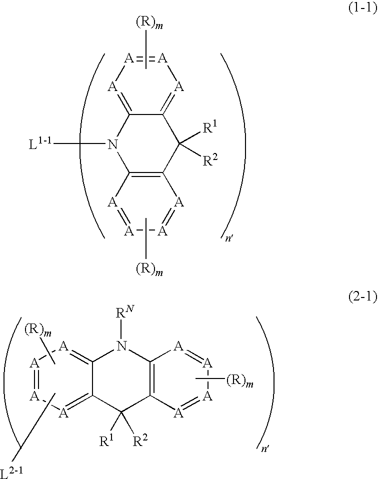

In the foregoing general formulae, each of R and R^{N} independently represents a substituent; each of R¹ and R² independently represents a substituent, provided that R¹ and R² do not represent an aryl group at the same time; each A independently represents a carbon atom or a nitrogen atom; each of L¹⁻¹ and L²⁻¹ independently represents phenylene or biphenylene; n′ represents an integer of 2 or more and not more than 10; and each m independently represents an integer.
[2] A charge transport material represented by the following general formula (1) or (2).

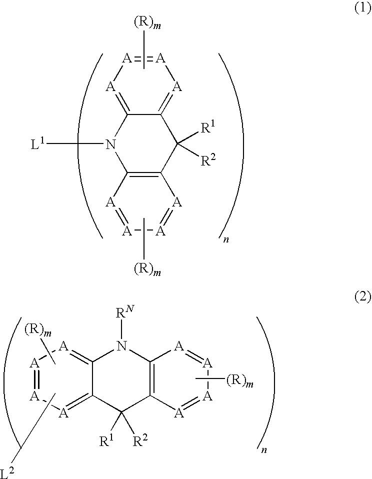

In the foregoing general formulae, each of L¹ and L² independently represents a connecting group; each of R and R^{N} independently represents a substituent; each of R¹ and R² independently represents a substituent, provided that R¹ and R² do not represent an aryl group at the same time; each A independently represents a carbon atom or a nitrogen atom; n represents an integer of 2 or more and not more than 10; and each m independently represents an integer.
[3] The charge transport material as set forth in [2], wherein the general formula (1) or (2) is represented by the following general formula (1-1) or (2-1).

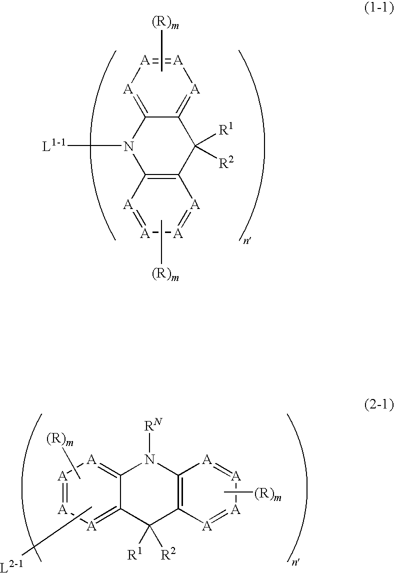

In the foregoing general formulae, each of R and R^{N} independently represents a substituent; each of R¹ and R² independently represents a substituent, provided that R¹ and R² do not represent an aryl group at the same time; each A independently represents a carbon atom or a nitrogen atom; each of L¹⁻¹ and L²⁻¹ independently represents phenylene or biphenylene; n′ represents an integer of 2 or more and not more than 10; and each m independently represents an integer.
[4] The charge transport material as set forth in [2], wherein the general formula (1) is represented by the following general formula (3).

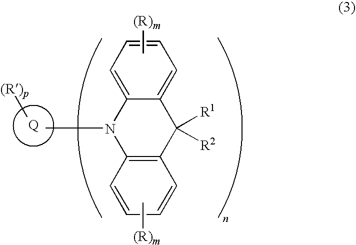

In the foregoing general formula, each of R and R′ independently represents a substituent; each of R¹ and R² independently represents a substituent, provided that R¹ and R² do not represent an aryl group at the same time; Q represents a 5-membered ring or a 6-membered ring; n represents 2 or 3; and each of m and p represents an integer.
[⁵] The charge transport material as set forth in [4], wherein the general formula (3) is represented by the following general formula (4).

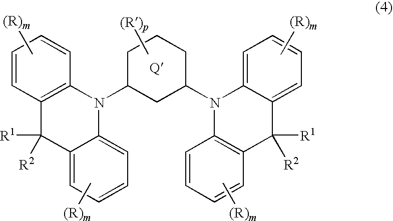

In the foregoing general formula, each of R and R′ independently represents a substituent; each of R¹ and R² independently represents a substituent, provided that R¹ and R² do not represent an aryl group at the same time; Q′ represents an aromatic 6-membered ring; and each of m and p represents an integer.
[6] The charge transport material as set forth in [5], wherein R¹ in the general formula (4) is a methyl group.
[7] The charge transport material as set forth in any one of [2] to [6], which has an excited triplet level (T₁) in a thin film state of 3.0 eV or more and not more than 3.5 eV.
[8] A composition comprising the compound as set forth in [1] or the charge transport material as set forth in any one of [2] to [6].
[9] A thin film comprising the compound as set forth in [1] or the charge transport material as set forth in any one of [2] to [6].
[10] An organic electroluminescence device comprising at least one organic layer including a light emitting layer containing a light emitting material between a cathode and an anode, wherein the at least one organic layer contains the compound as set forth in [1] or the charge transport material as set forth in any one of [2] to [7].
[11] The organic electroluminescence device as set forth in [10], wherein the light emitting layer contains a phosphorescent material.

[12] The organic electroluminescence device as set forth in [11], wherein the phosphorescent material is an Ir complex or a Pt complex.
[13] The organic electroluminescence device as set forth in [12], wherein the phosphorescent material is a Pt complex including a tridentate or more multidentate ligand.
[14] The organic electroluminescence device as set forth in [13], wherein the phosphorescent material is a Pt complex represented by the following general formula (C-2).

### General Formula (C-2)

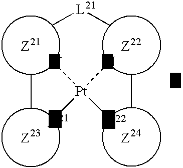

In the foregoing general formula, L²¹ represents a single bond or a divalent connecting group; each of A²¹ and A²² independently represents C or N; each of Z²¹ and Z²² independently represents a nitrogen-containing aromatic heterocyclic ring; and each of Z²³ and Z²⁴ independently represents a benzene ring or an aromatic heterocyclic ring.
[15] The organic electroluminescence device as set forth in [14], wherein the phosphorescent material is a Pt complex represented by the following general formula (5).

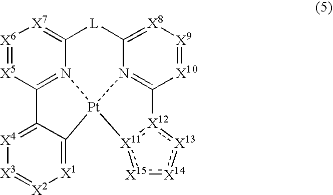

In the foregoing general formula, each of X¹, X², X³ and X⁴ independently represents a carbon atom or a nitrogen atom, provided that at least one of X¹, X², X³ and X⁴ represents a nitrogen atom; each of X⁵, X⁶, X⁷, X⁸, X⁹ and X¹⁰ independently represents a carbon atom or a nitrogen atom; each of X¹¹ and X¹² independently represents a carbon atom or a nitrogen atom; each of X¹³, X¹⁴ and X¹⁵ independently represents a carbon atom, a nitrogen atom, an oxygen atom or a sulfur atom; the number of nitrogen atoms contained in the 5-membered ring structure formed by X¹¹, X¹², X¹³, X¹⁴ and X¹⁵ is not more than 2; and L represents a single bond or a divalent connecting group.
[16] The organic electroluminescence device as set forth in [12], wherein the phosphorescent material is an Ir complex represented by the following general formula (T-1).

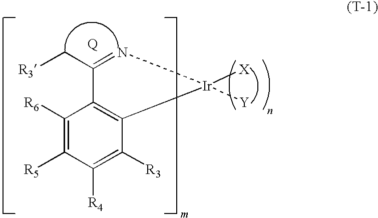

In the general formula (T-1), R₃′ represents an alkyl group, a heteroalkyl group, an aryl group or a heteroaryl group and may further have a substituent Z; R₅ represents an aryl group or a heteroaryl group and may be further substituted with a non-aromatic group; the ring Q represents an aromatic heterocyclic ring or a condensed aromatic heterocyclic ring each having at least one nitrogen atom, which is coordinated on Ir, and may be further substituted with a non-aromatic group; each of R₃, R₄ and R₆ represents a hydrogen atom, an alkyl group, an alkenyl group, an alkynyl group, —CN, a perfluoroalkyl group, a trifluorovinyl group, —CO₂R, —C(O)R, —NR₂, —NO₂, —OR, a halogen atom, an aryl group or a heteroaryl group and may further have a substituent Z; R₃ and R₄ may be bonded to each other to form a condensed 4-membered to 7-membered ring, the condensed 4-membered to 7-membered ring is a cycloalkyl, a cycloheteroalkyl, an aryl or a heteroaryl, and the condensed 4-membered to 7-membered ring may further have a substituent Z; R₃′ and R₆ may be connected to each other via a connecting group selected among —CR₂—CR₂—, —CR═CR—, —CR₂—, —O—, —NR—, —O—CR₂—, —NR—CR₂— and —N═CR— to form a ring; and each R independently represents a hydrogen atom, an alkyl group, an alkenyl group, an alkynyl group, a heteroalkyl group, an aryl group or a heteroaryl group and may further have a substituent Z; each Z independently represents a halogen atom, —R′, —OR′, —N(R′)₂, —SR′, —C(O)R′, —C(O)OR′, —C(O)N(R′)₂, —CN, —NO₂, —SO₂, —SOR′, —SO₂R′ or —SO₃R′; each R′ independently represents a hydrogen atom, an alkyl group, a perhaloalkyl group, an alkenyl group, an alkynyl group, a heteroalkyl group, an aryl group or a heteroaryl group; (X—Y) represents an auxiliary ligand; and m represents an integer of from 1 to 3 and n represents an integer of from 0 to 2, provided that m+n is 3.
[17] The organic electroluminescence device as set forth in any one of [11] to [16], wherein the phosphorescent material has a maximum light emission wavelength of not more than 500 nm.
[18] A light emission apparatus comprising the organic electroluminescence device as set forth in any one of [10] to [17].
[19] A display apparatus comprising the organic electroluminescence device as set forth in any one of [10] to [17].
[20] An illumination apparatus comprising the organic electroluminescence device as set forth in any one of [10] to [17].

### BRIEF DESCRIPTION OF THE DRAWINGS

The features of the invention will appear more fully upon consideration of the exemplary embodiments of the inventions, which are schematically set forth in the drawings, in which:

FIG. 1 is a schematic cross-sectional view illustrating an exemplary embodiment of a layer constitution of an organic electroluminescent device of the invention;

FIG. 2 is a schematic cross-sectional view illustrating an exemplary embodiment of a light emitting apparatus of the invention; and

FIG. 3 is a schematic cross-sectional view illustrating an exemplary embodiment of an illumination apparatus of the invention.

### DETAILED DESCRIPTION OF THE INVENTION

The charge transport material of the invention includes a specified structure expressed below (hereinafter also referred to as “specified site”) and is represented by the general formula (1) or (2).

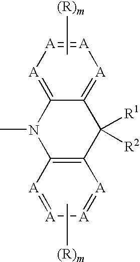

Also, the organic electroluminescence device of the invention is an organic electroluminescence device comprising a cathode and an anode having therebetween at least one organic layer including a light emitting layer containing a light emitting material, wherein the organic layer contains a charge transport material represented by the following general formula (1) or (2) (hereinafter also referred to as “compound of the invention”).

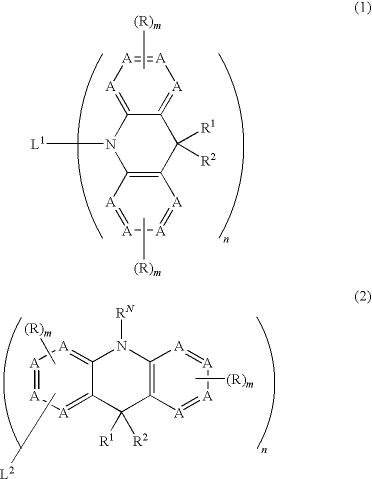

In the foregoing general formulae, each of L¹ and L² independently represents a connecting group; each of R and R^{N} independently represents a substituent; each of R¹ and R² independently represents a substituent, provided that R¹ and R² do not represent an aryl group at the same time; each A independently represents a carbon atom or a nitrogen atom; n represents an integer of 2 or more and not more than 10; and each m independently represents an integer.

That is, the organic electroluminescence device of the invention has at least one light emitting layer as the organic layer. Also, as the organic layer other than light emitting layer, a hole injection layer, a hole transport layer, an electron blocking layer, an exciton blocking layer, a hole blocking layer, an electron transport layer, an electron injection layer, a protective layer and so on may be properly disposed, and each of these layers may also be provided with a function of other layer. Also, each of the layers may be constituted of plural layers.

Though the organic electroluminescence device of the invention may be an organic electroluminescence device utilizing light emission (fluorescence) from an excited singlet or an organic electroluminescence device utilizing light emission (phosphorescence) from an excited triplet, an organic electroluminescence device utilizing phosphorescence is more preferable from the viewpoint of luminous efficiency.

It is preferable that the organic electroluminescence device of the invention is constituted of at least one light emitting material and at least one host material. The “host material” as referred to herein is a material other than the light emitting material among materials constituting the light emitting layer and means a material having at least one function among a function of dispersing the light emitting material therein to hold it in the layer, a function of accepting a hole from an anode, a hole transport layer or the like, a function of accepting an electron from a cathode, an electron transport layer or the like, a function of transporting a hole and/or an electron, a function of providing a site of recombination of a hole and an electron, a function of transporting the energy of an exciton generated through recombination into the light emitting material and a function of transporting a hole and/or an electron into the light emitting material.

The compound of the invention may be contained in any of the organic layers or may be contained in plural layers. However, the compound of the invention is preferably contained in a hole injection layer, a hole transport layer, an electron blocking layer, a light emitting layer, a hole blocking layer, an electron transport layer or an electron injection layer; more preferably contained in a light emitting layer, an electron blocking layer, a hole transport layer or a hole injection layer; further preferably contained in a light emitting layer; and most preferably contained as a host material in a light emitting layer. In the case where the compound of the invention is contained as a host material in a light emitting layer, a content of the compound of the invention in the light emitting layer is preferably from 10 to 99.9% by mass, more preferably from 30 to 99% by mass, and further preferably from 50 to 99% by mass. Also, in the case where the compound of the invention is contained in a hole injection layer, a hole transport layer, an electron blocking layer, a hole blocking layer, an electron transport layer or an electron injection layer, a content of the compound of the invention in each of the layers is preferably from 30 to 100% by mass, more preferably from 50 to 100% by mass, and most preferably from 70 to 100% by mass.

The compound represented by the general formula (1) or (2) is hereunder described.

Since the compound represented by the general formula (1) or (2) is high in chemical stability and carrier transport properties and has a structure with large T₁, it can be used as the electron transport material of the invention.

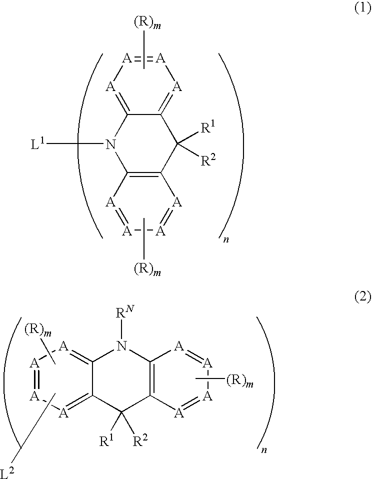

In the foregoing general formulae, each of L¹ and L² independently represents a connecting group; each of R and R^{N} independently represents a substituent; each of R¹ and R² independently represents a substituent, provided that R¹ and R² do not represent an aryl group at the same time; each A independently represents a carbon atom or a nitrogen atom; n represents an integer of 2 or more and not more than 10; and each m independently represents an integer.

The molecular aggregation state in a film, the ionization potential energy (Ip), the T₁ energy and so on can be controlled by the kind of a substituent in the general formula (1) or (2), selection of L₁ and L₂ and selection of Q and Q′ as described below. Though Ip varies depending upon an application, from the viewpoints of chemical stability and hole transport properties of the compound, Ip is preferably from 5.0 to 7.0, more preferably from 5.3 to 6.5, and especially preferably from 5.5 to 6.2; and the T₁ energy is preferably from 2.5 to 4.0 eV, more preferably from 2.7 to 3.7 eV, and especially preferably from 3.0 to 3.5 eV.

In the invention, Ip means a value measured by the photoelectron spectroscopy in air of a thin film of the material (for example, measured by using AC-2, manufactured by Riken Keiki Co., Ltd.). The T₁ energy can be determined from a short-wavelength end which is obtained by measuring a phosphorescent emission spectrum of a thin film of the material. For example, the material is subjected to thin-film deposition in a thickness of about 50 nm on a rinsed quartz glass substrate by means of vacuum vapor deposition, and a phosphorescent emission spectrum of the thin film is measured at a temperature of liquid nitrogen by using a Hitachi's fluorescence spectrophotometer F-7000 (manufactured by Hitachi High Technologies Corporation). The T₁ energy can be determined by reducing a build-up wavelength on the short-wavelength side of the obtained emission spectrum into an energy unit.

Examples of the substituent include those selected from the following group A of substituents.

### (Group A of Substituents)

Examples of the group A of substituents include an alkyl group (preferably an alkyl group having from 1 to 30 carbon atoms, more preferably an alkyl group having from 1 to 20 carbon atoms, and especially preferably an alkyl group having from 1 to 10 carbon atoms; for example, methyl, ethyl, isopropyl, tert-butyl, n-octyl, n-decyl, n-hexadecyl, cyclopropyl, cyclopentyl, cyclohexyl, etc.); an alkenyl group (preferably an alkenyl group having from 2 to 30 carbon atoms, more preferably an alkenyl group having from 2 to 20 carbon atoms, and especially preferably an alkenyl group having from 2 to 10 carbon atoms; for example, vinyl, allyl, 2-butenyl, 3-pentenyl, etc.); an alkynyl group (preferably an alkynyl group having from 2 to 30 carbon atoms, more preferably an alkynyl group having from 2 to 20 carbon atoms, and especially preferably an alkynyl group having from 2 to 10 carbon atoms; for example, propargyl, 3-pentynyl, etc.); an aryl group (preferably an aryl group having from 6 to 30 carbon atoms, more preferably an aryl group having from 6 to 20 carbon atoms, and especially preferably an aryl group having from 6 to 12 carbon atoms; for example, phenyl, p-methylphenyl, naphthyl, anthranyl, etc.); an amino group (preferably an amino group having from 0 to 30 carbon atoms, more preferably an amino group having from 0 to 20 carbon atoms, and especially preferably an amino group having from 0 to 10 carbon atoms; for example, amino, methylamino, dimethylamino, diethylamino, dibenzylamino, diphenylamino, ditolylamino, etc.); an alkoxy group (preferably an alkoxy group having from 1 to 30 carbon atoms, more preferably an alkoxy group having from 1 to 20 carbon atoms, and especially preferably an alkoxy group having from 1 to 10 carbon atoms; for example, methoxy, ethoxy, butoxy, 2-ethylhexyloxy, etc.); an aryloxy group (preferably an aryloxy group having from 6 to 30 carbon atoms, more preferably an aryloxy group having from 6 to 20 carbon atoms, and especially preferably an aryloxy group having from 6 to 12 carbon atoms; for example, phenyloxy, 1-naphthyloxy, 2-naphthyloxy, etc.); a heterocyclic oxy group (preferably a heterocyclic oxy group having from 1 to 30 carbon atoms, more preferably a heterocyclic oxy group having from 1 to 20 carbon atoms, and especially preferably a heterocyclic oxy group having from 1 to 12 carbon atoms; for example, pyridyloxy, pyrazyloxy, pyrimidyloxy, quinolyloxy, etc.); an acyl group (preferably an acyl group having from 2 to 30 carbon atoms, more preferably an acyl group having from 2 to 20 carbon atoms, and especially preferably an acyl group having from 2 to 12 carbon atoms; for example, acetyl, benzoyl, formyl, pivaloyl, etc.); an alkoxycarbonyl group (preferably an alkoxycarbonyl group having from 2 to 30 carbon atoms, more preferably an alkoxycarbonyl group having from 2 to 20 carbon atoms, and especially preferably an alkoxycarbonyl group having from 2 to 12 carbon atoms; for example, methoxycarbonyl, ethoxycarbonyl, etc.); an aryloxycarbonyl group (preferably an aryloxycarbonyl group having from 7 to 30 carbon atoms, more preferably an aryloxycarbonyl group having from 7 to 20 carbon atoms, and especially preferably an aryloxycarbonyl group having from 7 to 12 carbon atoms; for example, phenyloxycarbonyl, etc.); an acyloxy group (preferably an acyloxy group having from 2 to 30 carbon atoms, more preferably an acyloxy group having from 2 to 20 carbon atoms, and especially preferably an acyloxy group having from 2 to 10 carbon atoms; for example, acetoxy, benzoyloxy, etc.); an acylamino group (preferably an acylamino group having from 2 to 30 carbon atoms, more preferably an acylamino group having from 2 to 20 carbon atoms, and especially preferably an acylamino group having from 2 to 10 carbon atoms; for example, acetylamino, benzoylamino, etc.); an alkoxycarbonylamino group (preferably an alkoxycarbonylamino group having from 2 to 30 carbon atoms, more preferably an alkoxycarbonylamino group having from 2 to 20 carbon atoms, and especially preferably an alkoxycarbonylamino group having from 2 to 12 carbon atoms; for example, methoxycarbonylamino, etc.); an aryloxycarbonylamino group (preferably an aryloxycarbonylamino group having from 7 to 30 carbon atoms, more preferably an aryloxycarbonylamino group having from 7 to 20 carbon atoms, and especially preferably an aryloxycarbonylamino group having from 7 to 12 carbon atoms; for example, phenyloxycarbonylamino, etc.); a sulfonylamino group (preferably a sulfonylamino group having from 1 to 30 carbon atoms, more preferably a sulfonylamino group having from 1 to 20 carbon atoms, and especially preferably a sulfonylamino group having from 1 to 12 carbon atoms; for example, methanesulfonylamino, benzenesulfonylamino, etc.); a sulfamoyl group (preferably a sulfamoyl group having from 0 to 30 carbon atoms, more preferably a sulfamoyl group having from 0 to 20 carbon atoms, and especially preferably a sulfamoyl group having from 0 to 12 carbon atoms; for example, sulfamoyl, methylsulfamoyl, dimethylsulfamoyl, phenylsulfamoyl, etc.); a carbamoyl group (preferably a carbamoyl group having from 1 to 30 carbon atoms, more preferably a carbamoyl group having from 1 to 20 carbon atoms, and especially preferably a carbamoyl group having from 1 to 12 carbon atoms; for example, carbamoyl, methylcarbamoyl, diethylcarbamoyl, phenylcarbamoyl, etc.); an alkylthio group (preferably an alkylthio group having from 1 to 30 carbon atoms, more preferably an alkylthio group having from 1 to 20 carbon atoms, and especially preferably an alkylthio group having from 1 to 12 carbon atoms; for example, methylthio, ethylthio, etc.); an arylthio group (preferably an arylthio group having from 6 to 30 carbon atoms, more preferably an arylthio group having from 6 to 20 carbon atoms, and especially preferably an arylthio group having from 6 to 12 carbon atoms; for example, phenylthio, etc.); a heterocyclic thio group (preferably a heterocyclic thio group having from 1 to 30 carbon atoms, more preferably a heterocyclic thio group having from 1 to 20 carbon atoms, and especially preferably a heterocyclic thio group having from 1 to 12 carbon atoms; for example, pyridylthio, 2-benzimizolylthio, 2-benzoxazolylthio, 2-benzthiazolylthio, etc.); a sulfonyl group (preferably a sulfonyl group having from 1 to 30 carbon atoms, more preferably a sulfonyl group having from 1 to 20 carbon atoms, and especially preferably a sulfonyl group having from 1 to 12 carbon atoms; for example, mesyl, tosyl, etc.); a sulfinyl group (preferably a sulfinyl group having from 1 to 30 carbon atoms, more preferably a sulfinyl group having from 1 to 20 carbon atoms, and especially preferably a sulfinyl group having from 1 to 12 carbon atoms; for example, methanesulfinyl, benzenesulfinyl, etc.); a ureido group (preferably a ureido group having from 1 to 30 carbon atoms, more preferably a ureido group having from 1 to 20 carbon atoms, and especially preferably a ureido group having from 1 to 12 carbon atoms; for example, ureido, methylureido, phenylureido, etc.); a phosphoric amide group (preferably a phosphoric amide group having from 1 to 30 carbon atoms, more preferably a phosphoric amide group having from 1 to 20 carbon atoms, and especially preferably a phosphoric amide group having from 1 to 12 carbon atoms; for example, diethylphosphoric amide, phenylphosphoric amide, etc.); a hydroxyl group; a mercapto group; a halogen atom (for example, a fluorine atom, a chlorine atom, a bromine atom, an iodine atom, etc.); a cyano group; a sulfo group; a carboxyl group; a nitro group; a hydroxamic acid group; a sulfino group; a hydrazino group; an imino group; a heterocyclic group (including an aromatic heterocyclic group; preferably a heterocyclic group having from 1 to 30 carbon atoms, and more preferably a heterocyclic group having from 1 to 12 carbon atoms; examples of the hetero atom include a nitrogen atom, an oxygen atom, a sulfur atom, a phosphorus atom, a silicon atom, a selenium atom and a tellurium atom; and specific examples thereof include pyridyl, pyrazinyl, pyrimidyl, pyridazinyl, pyrrolyl, pyrazolyl, triazolyl, imidazolyl, oxazolyl, thiazolyl, isoxazolyl, isothiazolyl, quinolyl, furyl, thienyl, selenophenyl, tellurophenyl, piperidyl, piperidino, morpholino, pyrrolidyl, pyrrolidino, benzoxazolyl, benzimidazolyl, benzothiazolyl, carbazolyl, azepinyl, silolyl, etc.); a silyl group (preferably a silyl group having from 3 to 40 carbon atoms, more preferably a silyl group having from 3 to 30 carbon atoms, and especially preferably a silyl group having from 3 to 24 carbon atoms; for example, trimethylsilyl, triphenylsilyl, etc.); a silyloxy group (preferably a silyloxy group having from 3 to 40 carbon atoms, more preferably a silyloxy group having from 3 to 30 carbon atoms, and especially preferably a silyloxy group having from 3 to 24 carbon atoms; for example, trimethylsilyloxy, triphenylsilyloxy, etc.); and a phosphoryl group (for example, diphenylphosphoryl, dimethylphosphoryl, etc.). Each of these substituents may be further substituted. As the further substituent, the groups selected among those in the foregoing group A of substituents may be exemplified.

### (Group B of Substituents)

Examples of the group B of substituents include an alkyl group (preferably an alkyl group having from 1 to 30 carbon atoms, more preferably an alkyl group having from 1 to 20 carbon atoms, and especially preferably an alkyl group having from 1 to 10 carbon atoms; for example, methyl, ethyl, isopropyl, tert-butyl, n-octyl, n-decyl, n-hexadecyl, cyclopropyl, cyclopentyl, cyclohexyl, trifluoromethyl, pentafluoroethyl, etc.); an alkenyl group (preferably an alkenyl group having from 2 to 30 carbon atoms, more preferably an alkenyl group having from 2 to 20 carbon atoms, and especially preferably an alkenyl group having from 2 to 10 carbon atoms; for example, vinyl, allyl, 2-butenyl, 3-pentenyl, etc.); an alkynyl group (preferably an alkynyl group having from 2 to 30 carbon atoms, more preferably an alkynyl group having from 2 to 20 carbon atoms, and especially preferably an alkynyl group having from 2 to 10 carbon atoms; for example, propargyl, 3-pentynyl, etc.); an aryl group (preferably an aryl group having from 6 to 30 carbon atoms, more preferably an aryl group having from 6 to 20 carbon atoms, and especially preferably an aryl group having from 6 to 12 carbon atoms; for example, phenyl, p-methylphenyl, naphthyl, anthranyl, pentafluorophenyl, etc.); an acyl group (preferably an acyl group having from 1 to 30 carbon atoms, more preferably an acyl group having from 1 to 20 carbon atoms, and especially preferably an acyl group having from 1 to 12 carbon atoms; for example, acetyl, benzoyl, formyl, pivaloyl, etc.); an alkoxycarbonyl group (preferably an alkoxycarbonyl group having from 2 to 30 carbon atoms, more preferably an alkoxycarbonyl group having from 2 to 20 carbon atoms, and especially preferably an alkoxycarbonyl group having from 2 to 12 carbon atoms; for example, methoxycarbonyl, ethoxycarbonyl, etc.); an aryloxycarbonyl group (preferably an aryloxycarbonyl group having from 7 to 30 carbon atoms, more preferably an aryloxycarbonyl group having from 7 to 20 carbon atoms, and especially preferably an aryloxycarbonyl group having from 7 to 12 carbon atoms; for example, phenyloxycarbonyl, etc.); an acyloxy group (preferably an acyloxy group having from 2 to 30 carbon atoms, more preferably an acyloxy group having from 2 to 20 carbon atoms, and especially preferably an acyloxy group having from 2 to 10 carbon atoms; for example, acetoxy, benzoyloxy, etc.); a sulfamoyl group (preferably a sulfamoyl group having from 0 to 30 carbon atoms, more preferably a sulfamoyl group having from 0 to 20 carbon atoms, and especially preferably a sulfamoyl group having from 0 to 12 carbon atoms; for example, sulfamoyl, methylsulfamoyl, dimethylsulfamoyl, phenylsulfamoyl, etc.); a carbamoyl group (preferably a carbamoyl group having from 1 to 30 carbon atoms, more preferably a carbamoyl group having from 1 to 20 carbon atoms, and especially preferably a carbamoyl group having from 1 to 12 carbon atoms; for example, carbamoyl, methylcarbamoyl, diethylcarbamoyl, phenylcarbamoyl, etc.); and a heterocyclic group (preferably a heterocyclic group having from 1 to 30 carbon atoms, and more preferably a heterocyclic group having from 1 to 12 carbon atoms; examples of the hetero atom include a nitrogen atom, an oxygen atom and a sulfur atom; and specific examples thereof include imidazolyl, pyridyl, quinolyl, furyl, thienyl, piperidyl, morpholino, benzoxazolyl, benzimidazolyl, benzthiazolyl, carbazolyl, azepinyl group, etc.). Each of these substituents may be further substituted. As the further substituent, the groups selected among those in the foregoing group B of substituents may be exemplified.

The “carbon atom number” of the substituent such as the foregoing alkyl group as referred to in the invention is used in a sense such that inclusive of the case where the substituent such as the alkyl group may be substituted with other substituent, the carbon atom number of the instant other substituent is also included.

Each of R and R^{N} independently represents a substituent. Each of R¹ and R² independently represents a substituent. As the substituent represented by each of R, R^{N}, R¹ and R², each of the groups exemplified above for the group A of substituents is independently applicable.

From the viewpoints of chemical stability, carrier transport capability and T₁ energy of the compound of the invention, R is preferably an alkyl group having from 1 to 18 carbon atoms, an aryl group having from 6 to 18 carbon atoms, an amino group having from 2 to 12 carbon atoms, an alkoxy group having from 1 to 18 carbon atoms, an aryloxy group having from 6 to 18 carbon atoms, a heterocyclic oxy group having from 2 to 10 carbon atoms, an acyl group having from 1 to 18 carbon atoms, an acylamino group having from 1 to 18 carbon atoms, a sulfonylamino group having from 1 to 18 carbon atoms, a sulfamoyl group having from 2 to 18 carbon atoms, a carbamoyl group having from 2 to 18 carbon atoms, an alkylthio group having from 1 to 18 carbon atoms, a heterocyclic thio group having from 2 to 10 carbon atoms, a sulfonyl group having from 1 to 18 carbon atoms, a halogen atom, a cyano group, a nitro group, a heterocyclic group having from 2 to 10 carbon atoms, a silyl group having from 3 to 18 carbon atoms, a silyloxy group having from 3 to 18 carbon atoms or a phosphoryl group having from 1 to 18 carbon atoms; more preferably an alkyl group having from 1 to 18 carbon atoms, an aryl group having from 6 to 18 carbon atoms, an amino group having from 2 to 12 carbon atoms, a halogen atom, a cyano group, a nitro group, a heterocyclic group having from 2 to 10 carbon atoms, a silyl group having from 3 to 18 carbon atoms or a phosphoryl group having from 1 to 18 carbon atoms; and especially preferably an alkyl group having from 1 to 18 carbon atoms, a halogen atom, a cyano group or a silyl group having from 3 to 18 carbon atoms. R^{N} is preferably an alkyl group having from 1 to 18 carbon atoms, an aryl group having from 6 to 18 carbon atoms or a heterocyclic group having from 2 to 10 carbon atoms; more preferably an aryl group having from 6 to 18 carbon atoms or a heterocyclic group having from 2 to 10 carbon atoms; and especially preferably an aryl group having from 6 to 18 carbon atoms.

From the viewpoints of chemical stability, carrier transport capability and T₁ energy of the compound of the invention, each of R¹ and R² is preferably an alkyl group having from 1 to 6 carbon atoms, an aryl group having from 6 to 12 carbon atoms, a halogen atom or a cyano group; more preferably an alkyl group having from 1 to 4 carbon atoms, a halogen atom or a cyano group; especially preferably an alkyl group having from 1 to 4 carbon atoms; and most preferably a methyl group.

From the viewpoints of chemical stability, carrier transport capability and T₁ energy of the compound of the invention, as each of L¹ and L², a combination of connecting groups which are arbitrarily selected among an alkyl group having from 1 to 18 carbon atoms, an aryl group having from 6 to 24 carbon atoms, a heterocyclic group having from 2 to 10 carbon atoms, an amino group, a silyl group, a phosphoryl group, a carbonyl group, a sulfonyl group, an oxy group (—O—) and a thio group (—S—) can be preferably used.

Each of L¹ and L² is more preferably a connecting group which is arbitrarily selected among an alkyl group having from 1 to 18 carbon atoms, an aryl group having from 6 to 24 carbon atoms, a heterocyclic group having from 2 to 10 carbon atoms, a silyl group and a phosphoryl group.

Each of L¹ and L² is further preferably a connecting group in which two to four of the instant connecting groups are respectively connected to each other via a single bond, for example, a connecting group in which an amino group and a heterocyclic group having from 2 to 10 carbon atoms are arbitrarily combined, a connecting group in which an aryl group and a heterocyclic group are arbitrarily combined, a silyl group or a phosphoryl group; and even further preferably a connecting group in which an aryl group having from 6 to 24 carbon atoms, a heterocyclic group having from 2 to 10 carbon atoms and a silyl group are arbitrarily combined.

From the viewpoints of carrier injection and transport properties, n is preferably from 2 to 10, more preferably from 2 to 4, and especially preferably from 2 to 3.

m is preferably from 0 to 3, more preferably from 0 to 2, and especially preferably from 0 to 1.

Each A independently represents a carbon atom or a nitrogen atom. In the case where A represents a nitrogen atom, the nitrogen atom number is preferably from 1 to 6, more preferably from 1 to 4, and especially preferably from 1 to 2 in each of the general formulae (1) and (2).

R¹ and R² do not represent an aryl group at the same time. A structure in which both R¹ and R² represent an aryl group is of a tetraarylmethane structure and has low chemical stability. A reason for this resides in the fact that the chemical stability of a radical cation or a radical anion generated when the linkage is broken is so high that the linkage is easily broken.

From the viewpoints of chemical stability and carrier transport properties, the compound represented by the general formula (1) or (2) is more preferably a compound represented by the following general formula (1-1) or (2-1). Next, the compound represented by the general formula (1-1) or (2-1) is described.

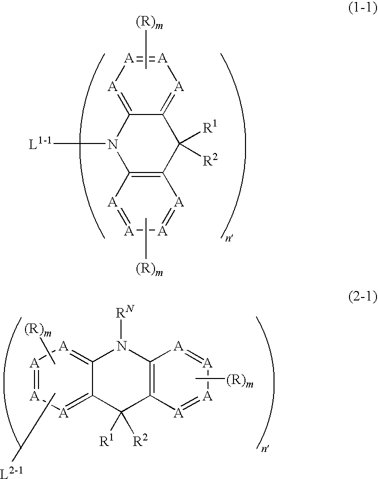

In the foregoing general formulae, each of R and R^{N} independently represents a substituent; each of R¹ and R² independently represents a substituent, provided that R¹ and R² do not represent an aryl group at the same time; each A independently represents a carbon atom or a nitrogen atom; each of L¹⁻¹ and L²⁻¹ independently represents phenylene or biphenylene; n′ represents an integer of 2 or more and not more than 10; and each m independently represents an integer.

The invention is also concerned with the compound represented by the general formula (1-1) or (2-1). The compound represented by the general formula (1-1) or (2-1) is useful as a charge transport compound.

The definitions of R, R^{N}, R¹, R² and m are synonymous with those in the general formula (1), respectively, and preferred ranges thereof are also the same.

Each n′ is independently preferably from 2 to 6, more preferably from 2 to 4, and especially preferably 2.

In the case where each of L¹⁻¹ and L²⁻¹ represents biphenylene, the compound represented by the general formula (1-1) or (2-1) can be represented by the following general formula (1-1)′ or (2-1)′.

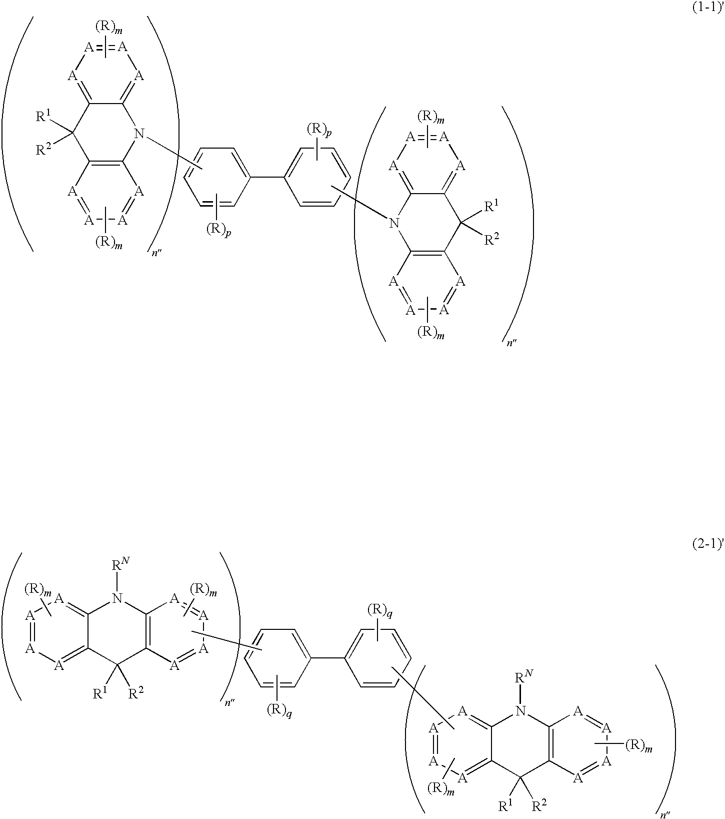

In the case where each of L¹⁻¹ and L²⁻¹ represents phenylene, the compound represented by the general formula (1-1) or (2-1) can be represented by the following general formula (1-1)″ or (2-1)″.

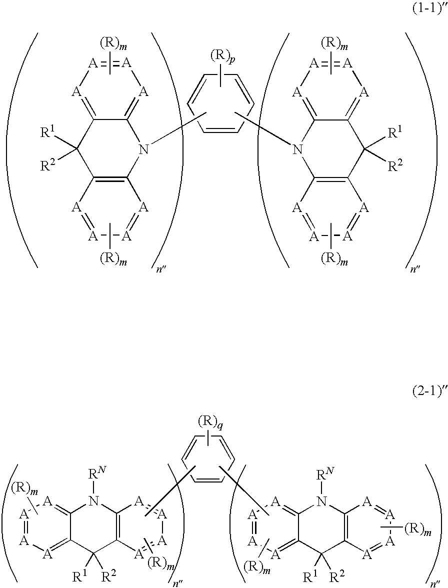

In the general formulae (1-1)′, (2-1)′, (1-1)″ and (2-1)″, each of R and R^{N} independently represents a substituent; each of R¹ and R² independently represents a substituent, provided that R¹ and R² do not represent an aryl group at the same time; each A independently represents a carbon atom or a nitrogen atom; each n″ independently represents an integer of 1 or more and not more than 5; and each of m, p and q independently represents an integer.

The definitions of R, R^{N}, R¹, R² and m are synonymous with those in the general formula (1), respectively, and preferred ranges thereof are also the same.

Each of p and q is preferably from 0 to 3, more preferably from 0 to 2, and especially preferably from 0 to 1.

Each n″ is independently preferably from 1 to 3, more preferably from 1 to 2, and especially preferably 1.

From the viewpoints of chemical stability and carrier transport properties, the compound represented by the general formula (1) is more preferably a compound represented by the following general formula (3). Next, the compound represented by the general formula (3) is described.

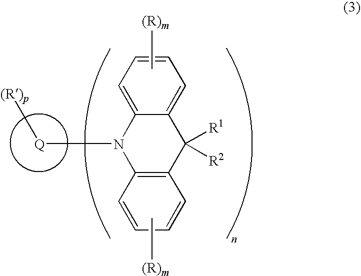

In the foregoing general formula, each of R and R′ independently represents a substituent; R¹ and R² do not represent an aryl group at the same time; Q represents a 5-membered ring or a 6-membered ring; n represents 2 or 3; and each of m and p represents an integer.

The definitions of R, R¹, R² and m are synonymous with those in the general formula (1), respectively, and preferred ranges thereof are also the same.

Examples of the substituent in R′ include those of the foregoing group A of substituents. From the viewpoints of chemical stability, carrier transport capability and T₁ energy of the compound of the invention, R′ is preferably an alkyl group having from 1 to 18 carbon atoms, an aryl group having from 6 to 18 carbon atoms, an amino group having from 2 to 12 carbon atoms, an alkoxy group having from 1 to 18 carbon atoms, an aryloxy group having from 6 to 18 carbon atoms, a heterocyclic oxy group having from 2 to 10 carbon atoms, an acyl group having from 1 to 18 carbon atoms, an acylamino group having from 1 to 18 carbon atoms, a sulfonylamino group having from 1 to 18 carbon atoms, a sulfamoyl group having from 2 to 18 carbon atoms, a carbamoyl group having from 2 to 18 carbon atoms, an alkylthio group having from 1 to 18 carbon atoms, a heterocyclic thio group having from 2 to 10 carbon atoms, a sulfonyl group having from 1 to 18 carbon atoms, a halogen atom, a cyano group, a nitro group, a heterocyclic group having from 2 to 10 carbon atoms, a silyl group having from 3 to 18 carbon atoms, a silyloxy group having from 3 to 18 carbon atoms or a phosphoryl group having from 1 to 18 carbon atoms; more preferably an alkyl group having from 1 to 18 carbon atoms, an aryl group having from 6 to 18 carbon atoms, an amino group having from 2 to 12 carbon atoms, a halogen atom, a cyano group, a nitro group, a heterocyclic group having from 2 to 10 carbon atoms, a silyl group having from 3 to 18 carbon atoms or a phosphoryl group having from 1 to 18 carbon atoms; and especially preferably an alkyl group having from 1 to 18 carbon atoms, a halogen atom, a cyano group or a silyl group having from 3 to 18 carbon atoms.

Also, from the viewpoints of chemical stability, carrier transport capability and T₁ energy of the compound of the invention, the 5-membered ring or 6-membered ring in Q is preferably a benzene ring, a pyridine ring, a pyrazine ring, a pyrimidine ring, a triazine ring, a thiophene ring, a furan ring, a pyrrole ring, a thiazole ring, an oxazole ring, a thiadiazole ring, an oxadiazole ring, a pyrazole ring, a triazole ring, a silole ring or a phosphole ring; more preferably a benzene ring, a pyridine ring, a pyrazine ring, a pyrimidine ring, a triazine ring, a thiophene ring, a furan ring, a pyrrole ring, a thiazole ring, an oxazole ring, a thiadiazole ring, an oxadiazole ring or a pyrazole ring; and especially preferably a benzene ring, a pyridine ring, a pyrazine ring, a pyrimidine ring or a triazine ring.

In the case where the connecting group Q is a 6-membered ring, as connecting places of plural specified sites, connection modes in which the respective specified sites are the o-position, m-position or p-position may be thought. However, in the case of connection at the p-position or m-position, strain of the molecule is small, and chemical stability is high. In the connection at the o-position or m-position, symmetry of the molecule is low, and crystallinity is low; and therefore, the film is hardly crystallized. From the foregoing reasons, as the connection mode of the specified site to the connecting group Q, connection at the m-position is preferable.

n is especially preferably 2.

p is preferably from 0 to 3, more preferably from 0 to 2, and especially preferably from 0 to 1.

From the viewpoints of chemical stability and carrier transport capability, the compound represented by the general formula (3) is more preferably a compound represented by the following general formula (4). Next, the compound represented by the general formula (4) is described.

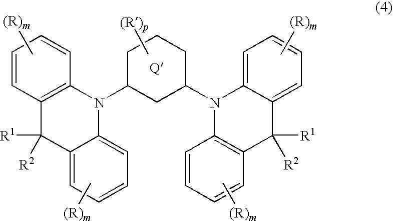

In the foregoing general formula, each of R and R′ independently represents a substituent; each of R¹ and R² independently represents a substituent, provided that R¹ and R² do not represent an aryl group at the same time; Q′ represents an aromatic 6-membered ring; and each of m and p represents an integer.

The definitions of R, R′, R¹, R², m and p are synonymous with those in the general formula (3), respectively, and preferred ranges thereof are also the same.

Also, from the viewpoints of chemical stability, carrier transport capability and T₁ energy of the compound of the invention, the 6-membered ring in Q is preferably a benzene ring, a pyridine ring, a pyrazine ring, a pyrimidine ring or a triazine ring; more preferably a benzene ring, a pyridine ring, a pyrazine ring or a triazine ring; and especially preferably a benzene ring.

In general, when a it-conjugated system is expanded in the molecule, the T₁ energy becomes small. Also, since the T₁ energy of the molecule is determined by a partial structure where the T₁ energy becomes the lowest in the molecule, when a portion where the π-conjugated system is wide is present even in one place in the molecule, the T₁ energy is determined in that portion. Accordingly, what a biaryl structure or a structure where aromatic rings are condensed is present in the molecule is not preferable because T₁ is in general small. However, in the case where the two aryl ring planes of the biaryl structure are twisted each other due to steric hindrance or the like, the biaryl structure may be present because the T₁ energy can keep a relatively large state.

From the standpoints of efficiency and durability of the device, it is preferable that the charge transport material of the invention has an excited triplet level (T₁) in a thin film state of 3.0 eV or more and not more than 3.5 eV.

A molecular weight of the compound of the invention is preferably from 300 to 1,200, more preferably from 400 to 1,000, and especially preferably from 450 to 800. When the molecular weight falls within this range, stability in a film state is excellent, and a high purity of the compound can be easily realized from the standpoints of solubility in a solvent and sublimation temperature. As an index of the stability in a film state, there is exemplified a glass transition temperature Tg. T g is preferably from 60 to 400° C., more preferably from 100 to 400° C., and especially preferably from 130 to 400° C.

Here, Tg can be confirmed by thermal measurement such as differential scanning calorimetry (DSC) and differential thermal analysis (DTA), X-ray diffraction (XRD), polarizing microscopic observation or the like. Also, when the purity of the compound of the invention is low, the compound serves as a trap of the charge transport, and therefore, it is preferable that the purity of the compound of the invention is as high as possible. The purity can be measured by, for example, high performance liquid chromatography (HPLC), and an area ratio when detected at an optical absorption intensity of 254 nm is preferably 95.0% or more, more preferably 97.0% or more, especially preferably 99.0% or more, and most preferably 99.5% or more.

As known by the carbazole based material disclosed in WO 2008/117889, a material obtained by substituting a part or all of hydrogen atoms of the material of the invention with a deuterium atom is also preferable and useful as the charge transport material.

Specific examples of the compound of the invention are described below, but it should not be construed that the invention is limited thereto.

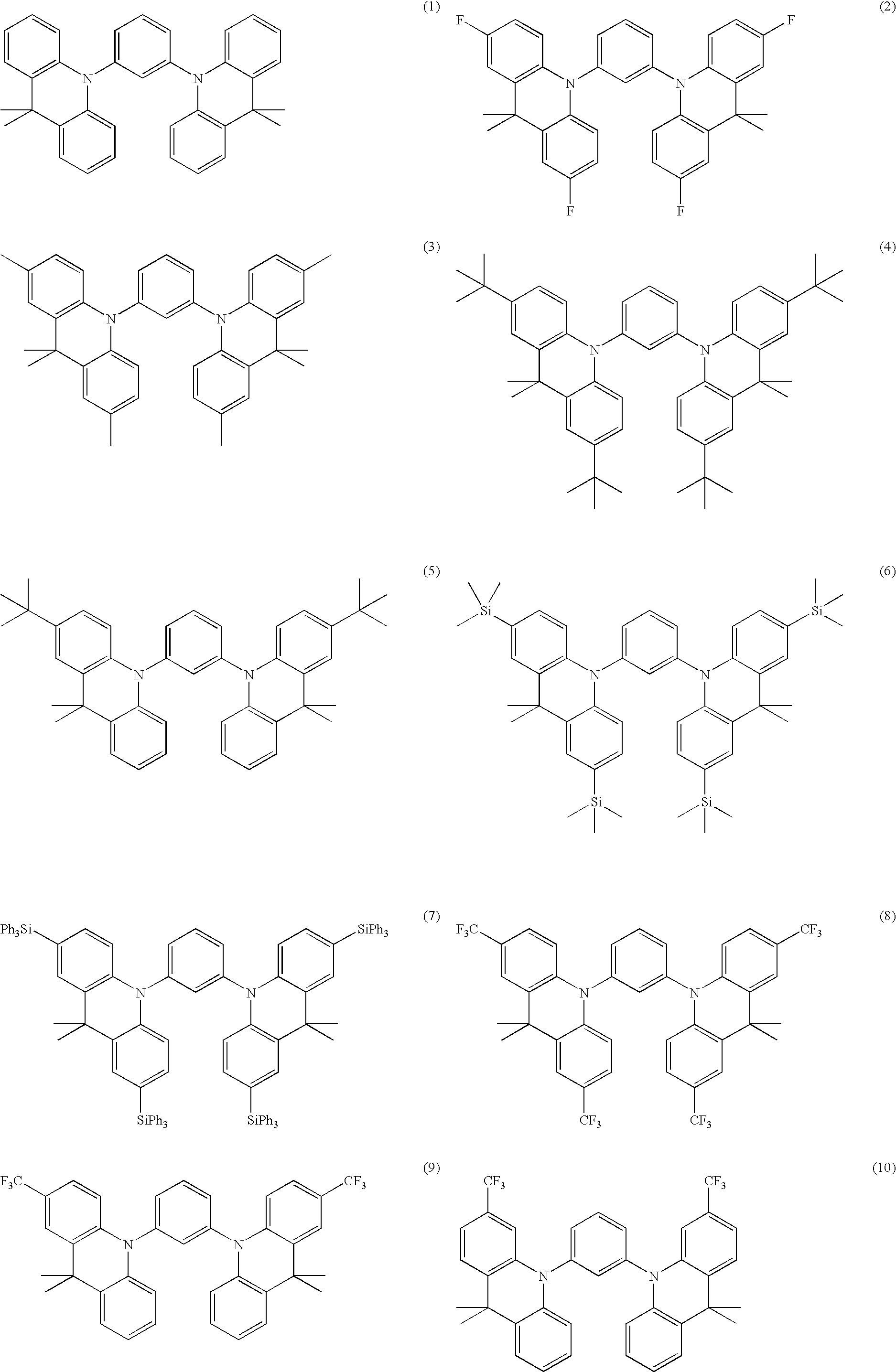

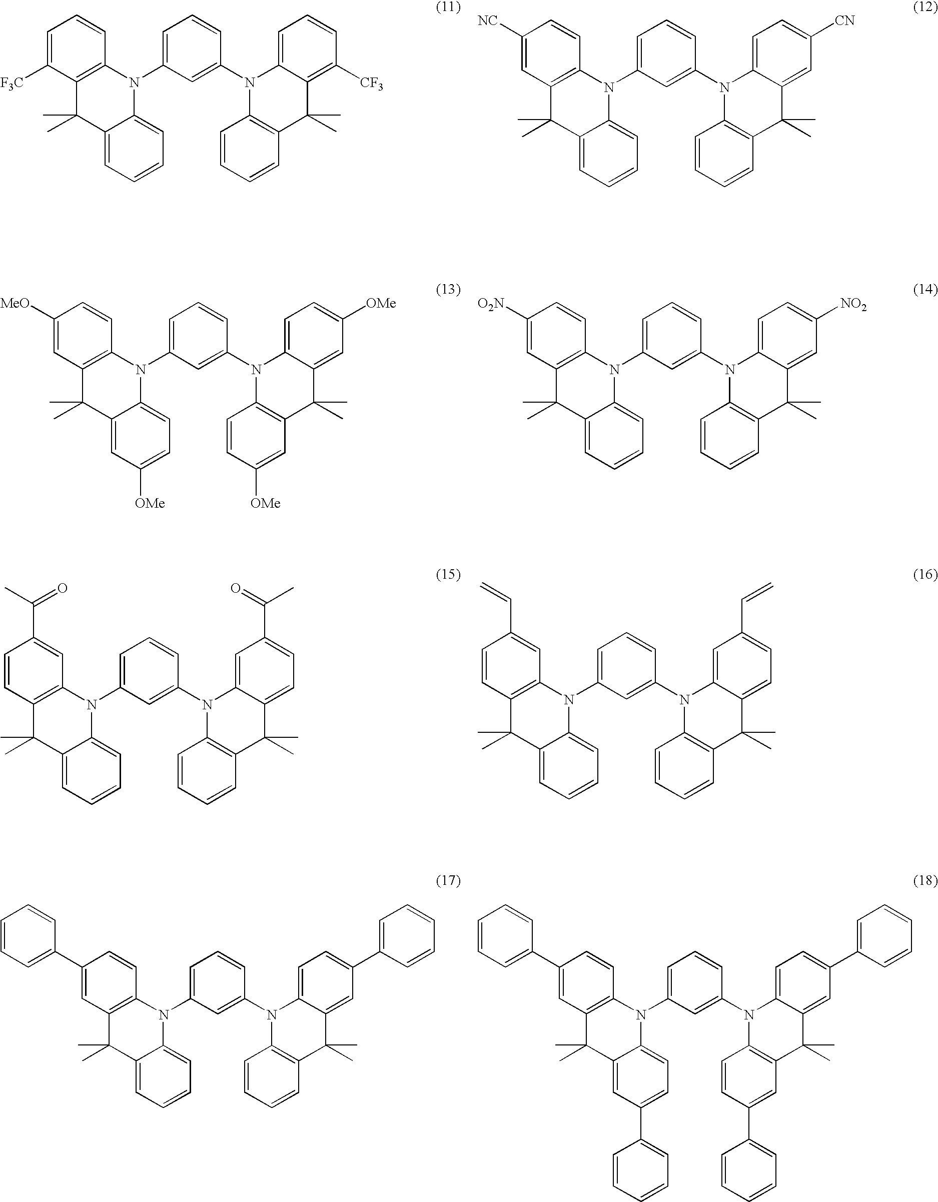

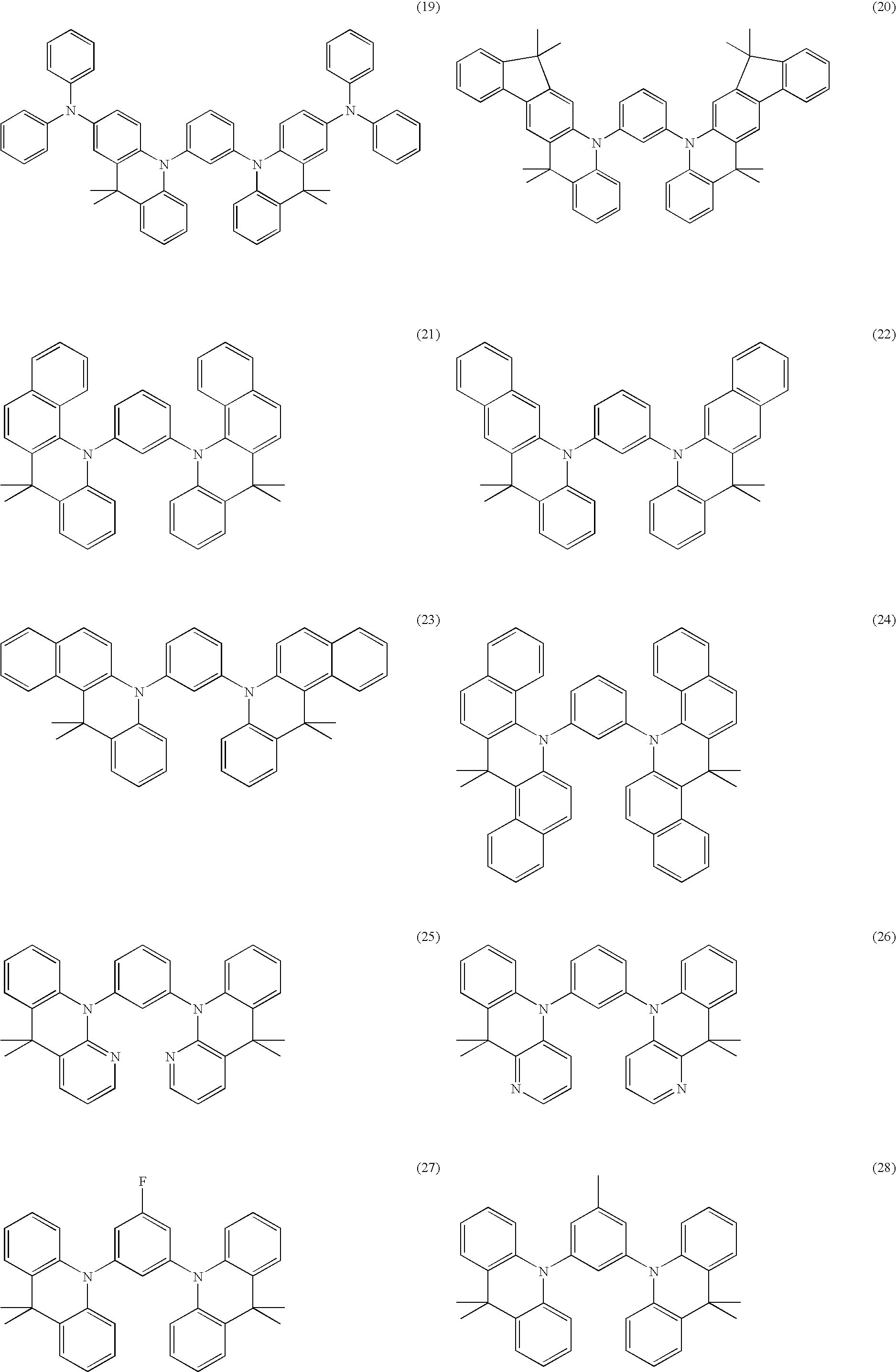

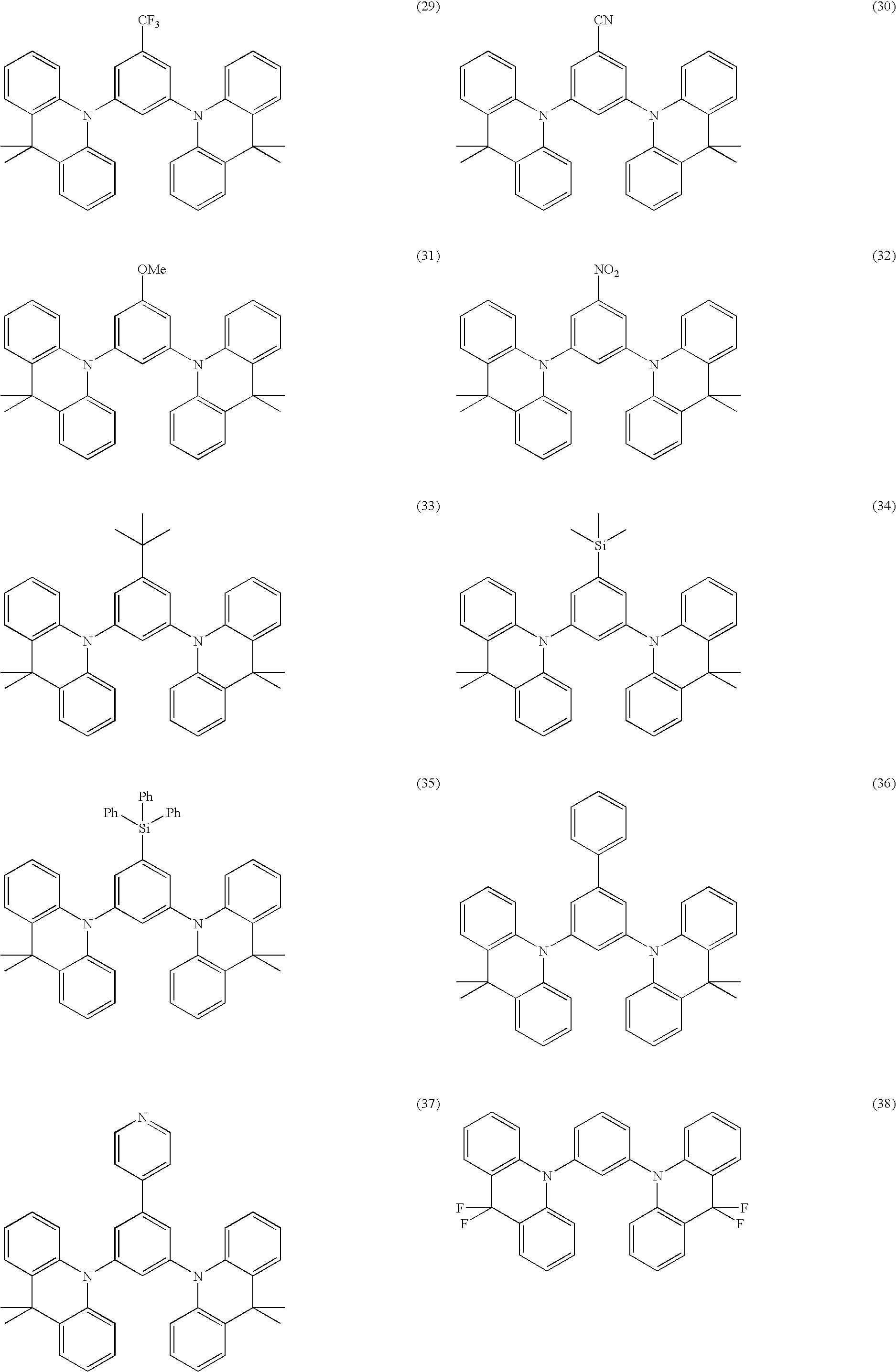

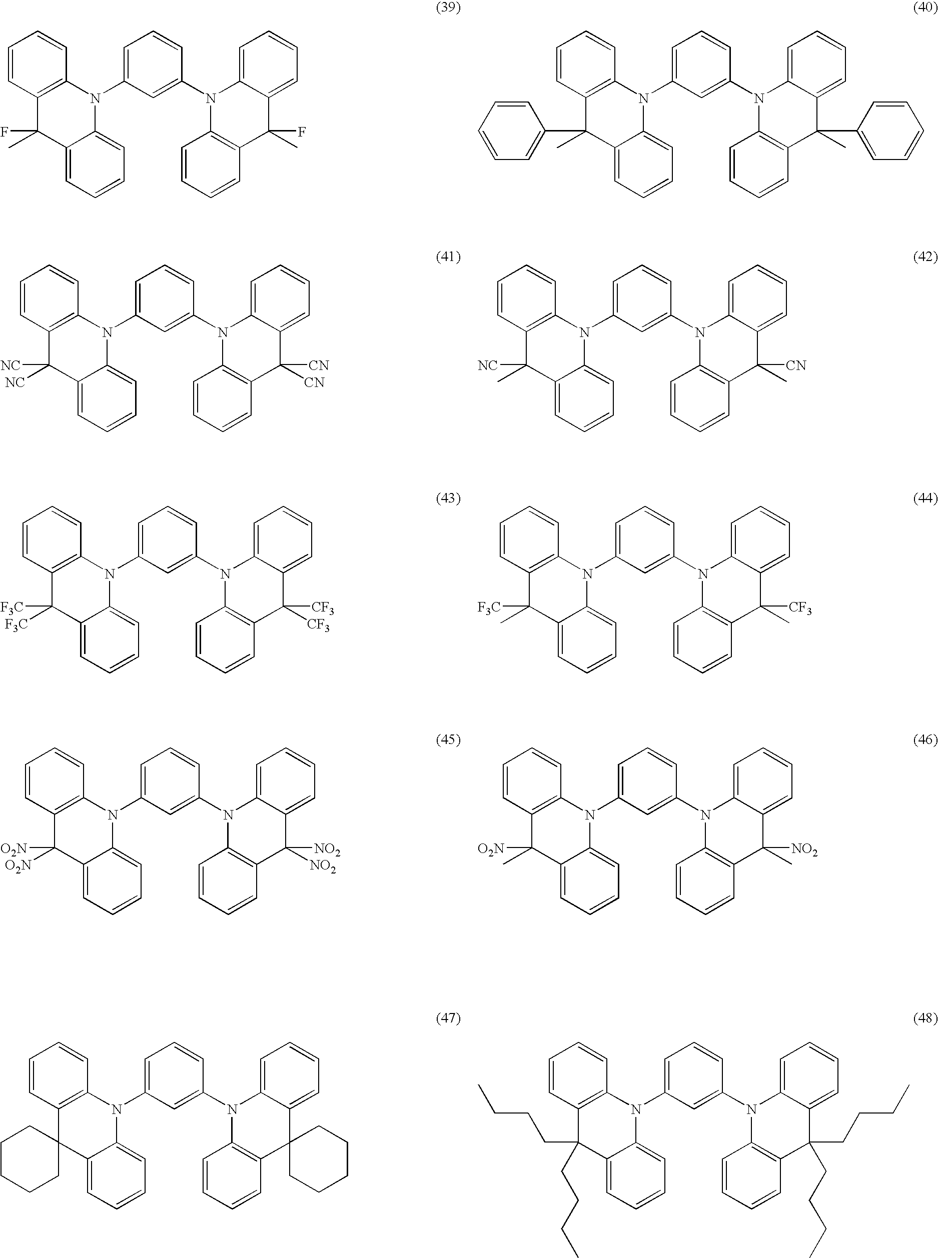

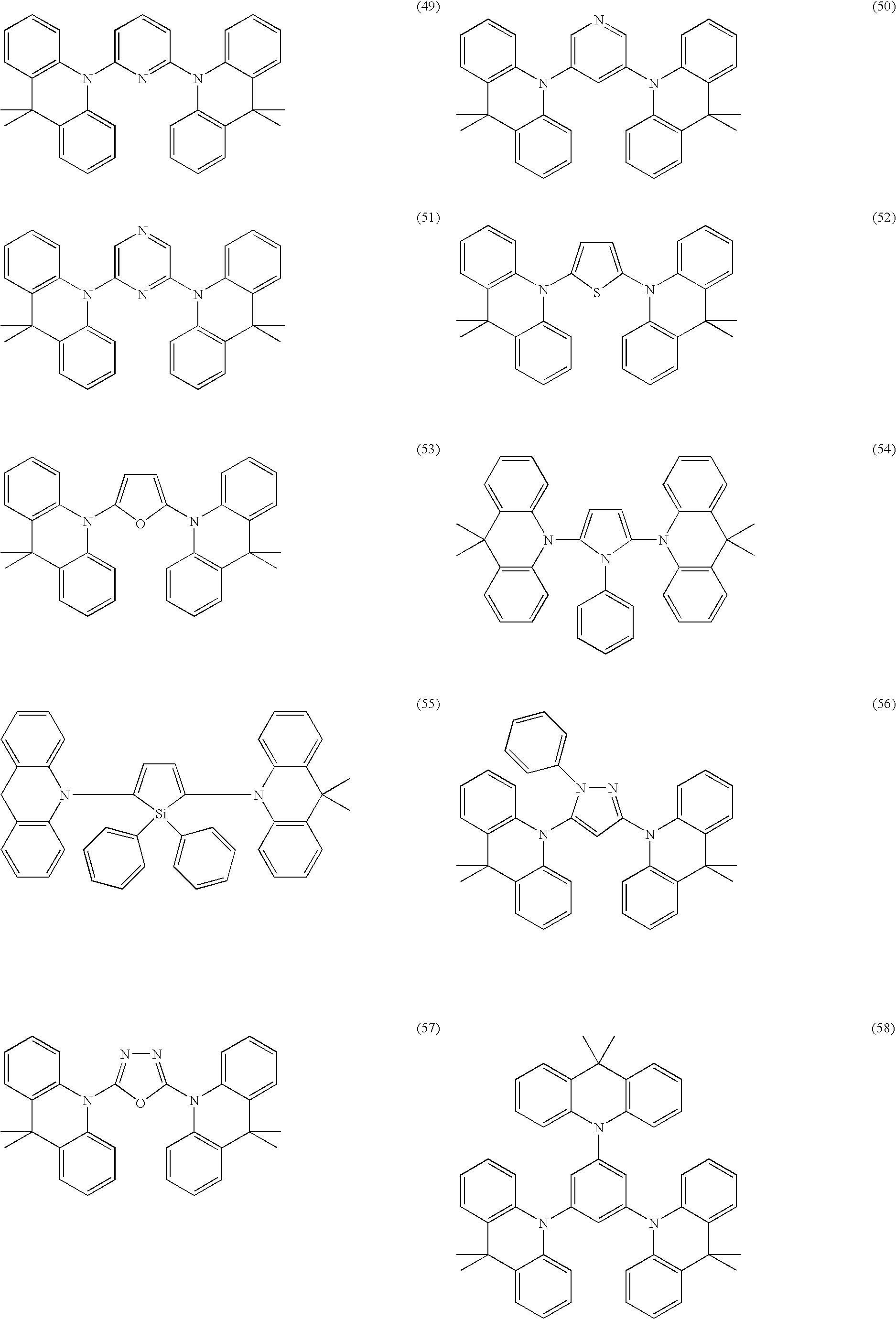

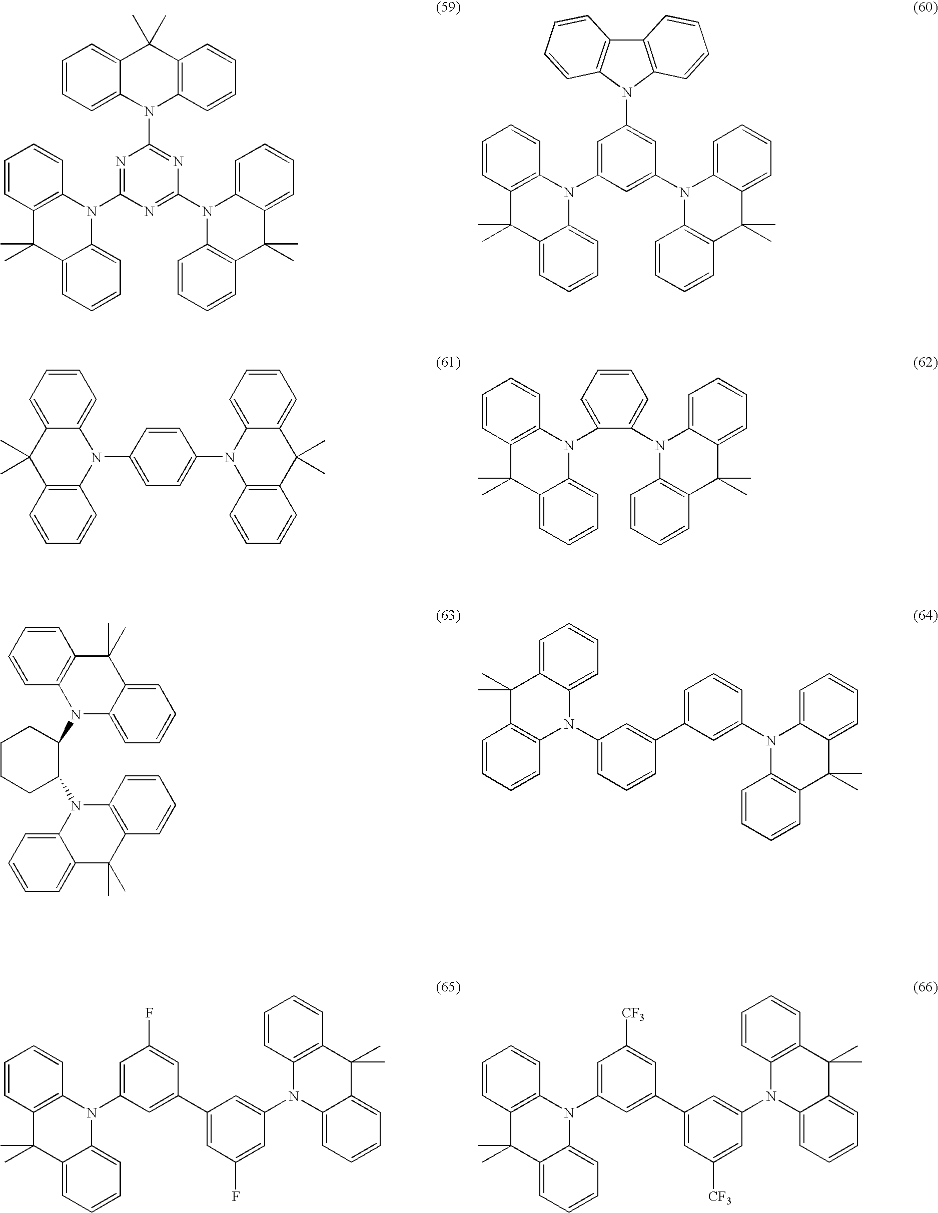

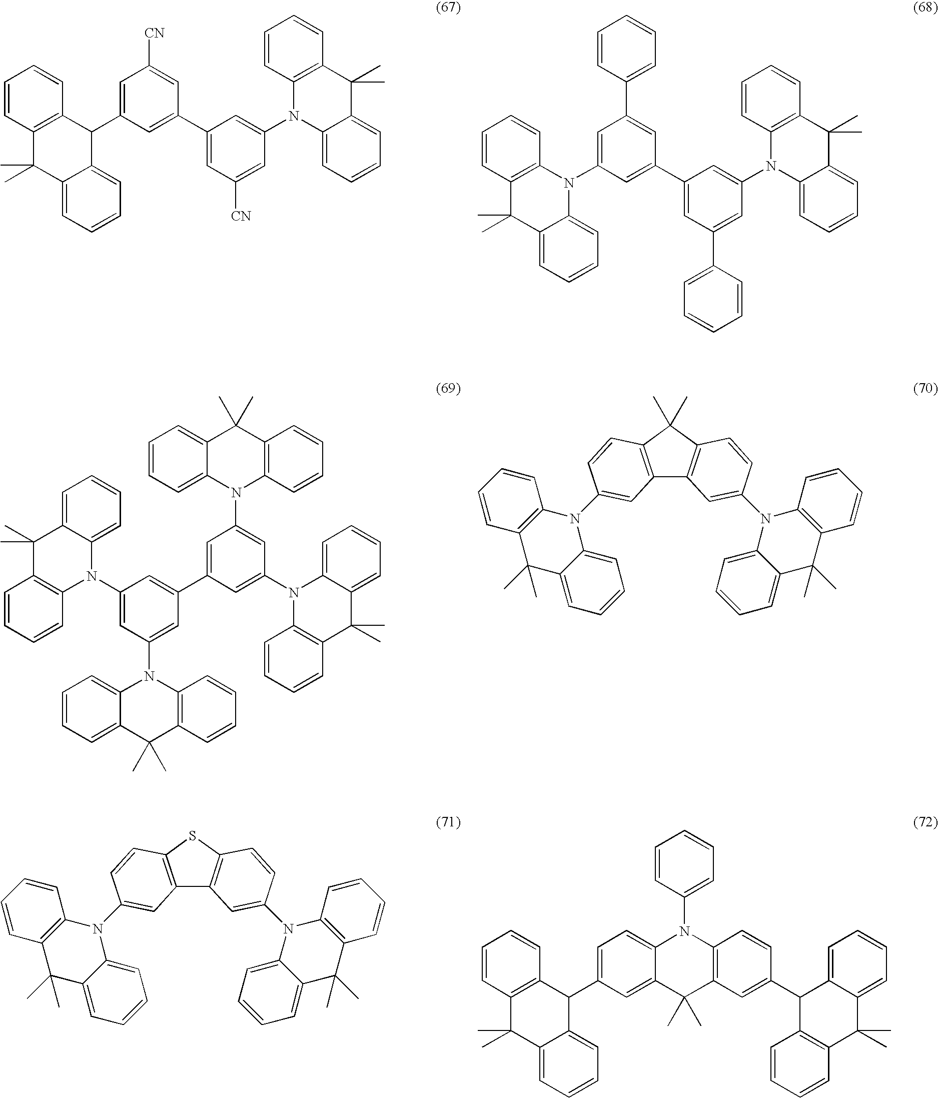

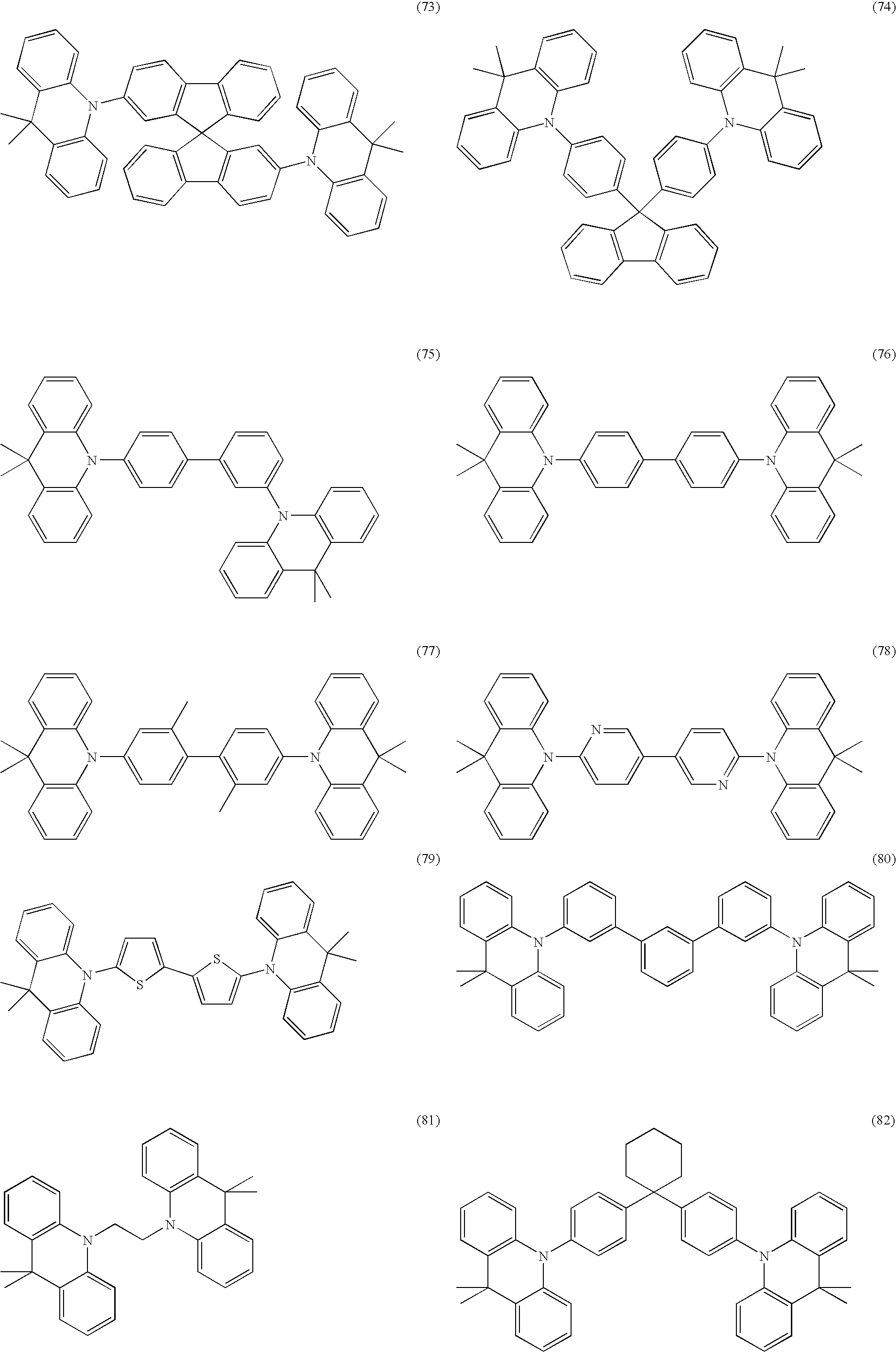

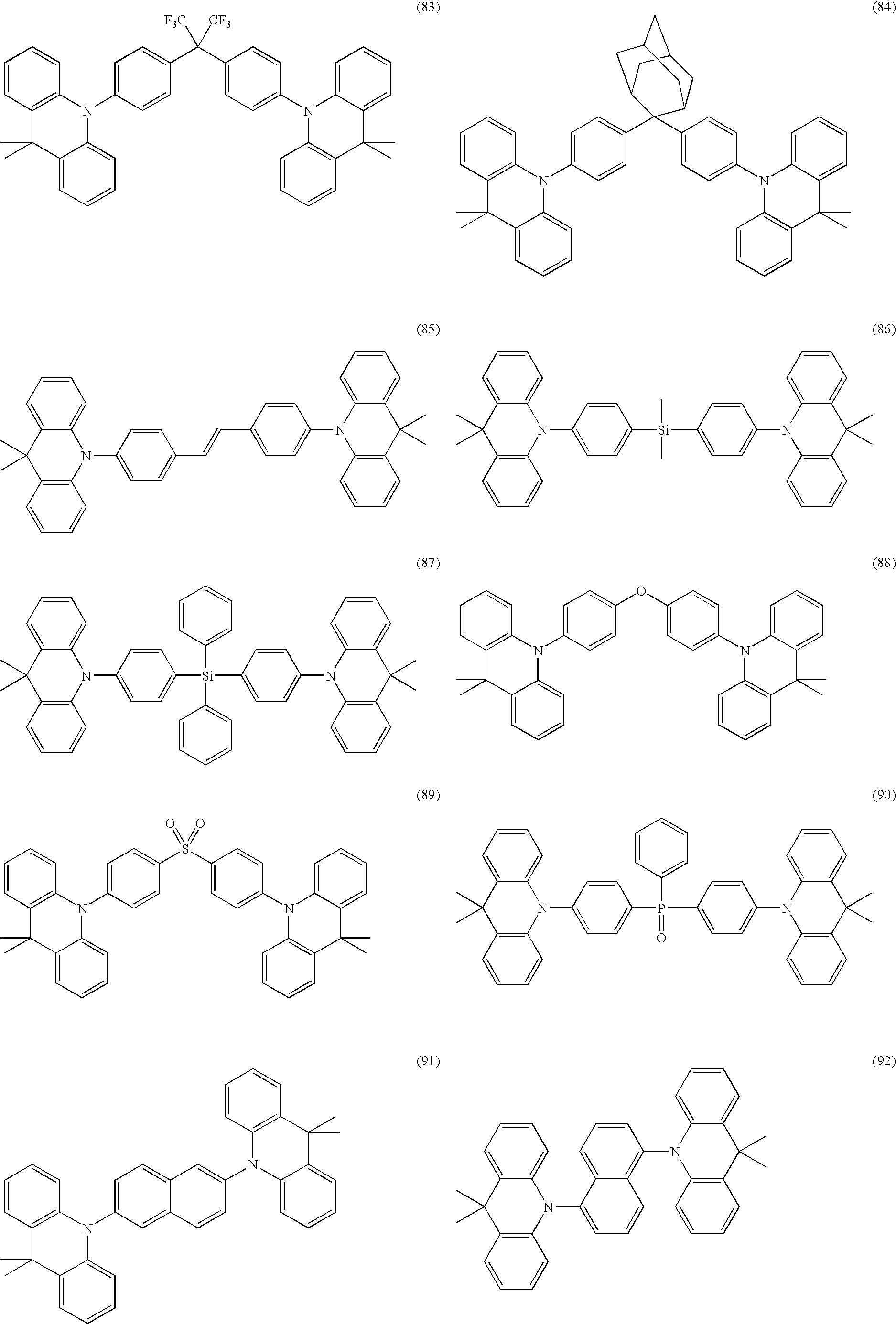

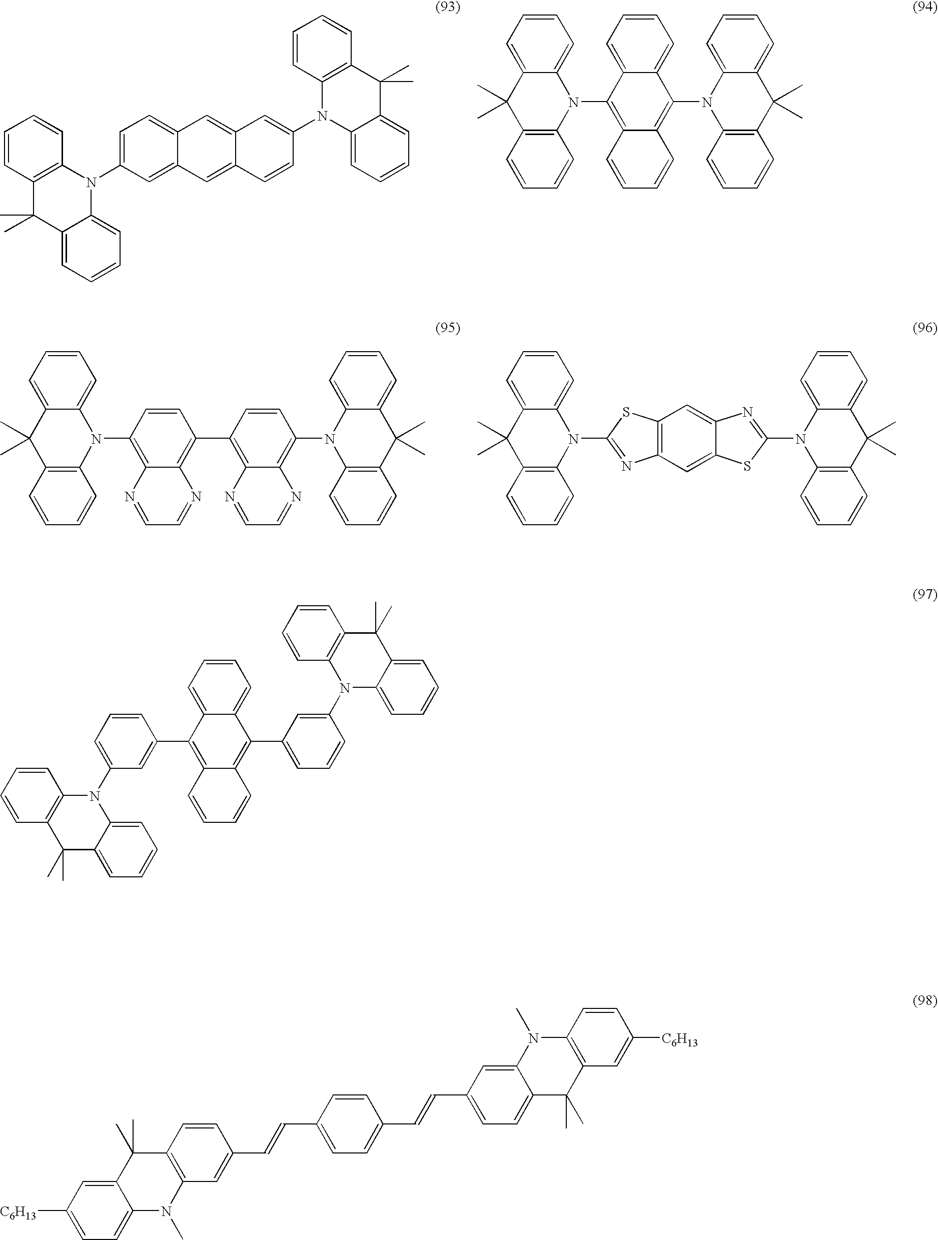

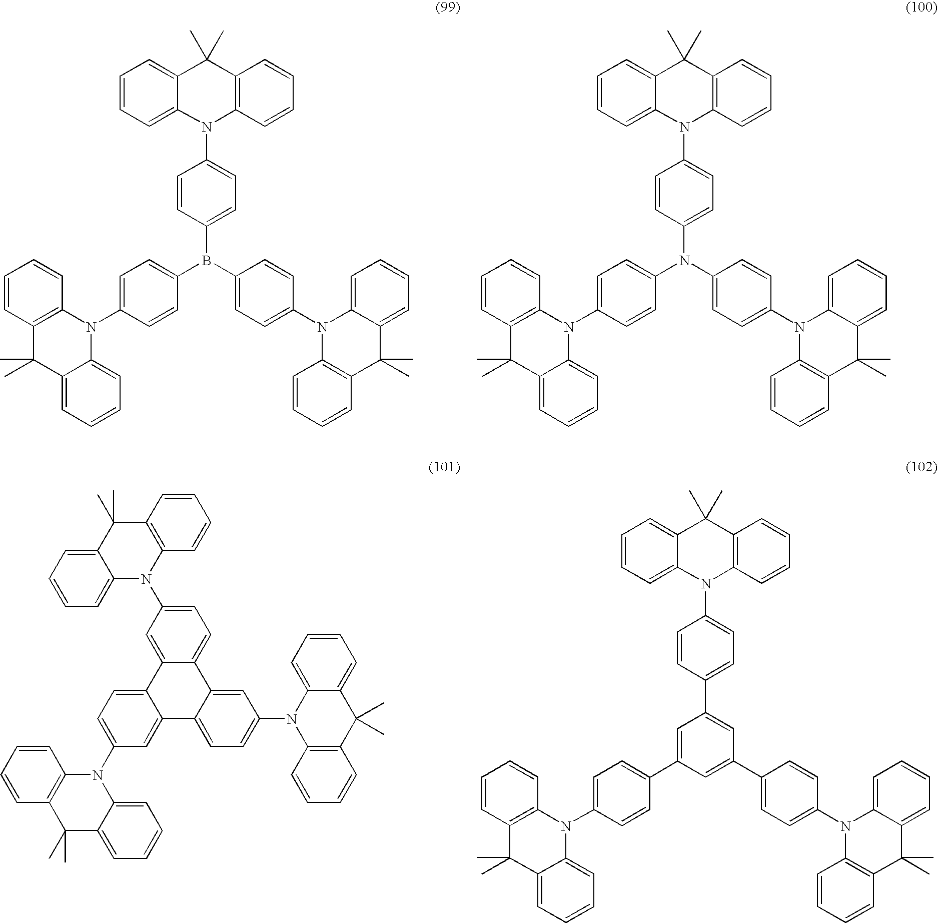

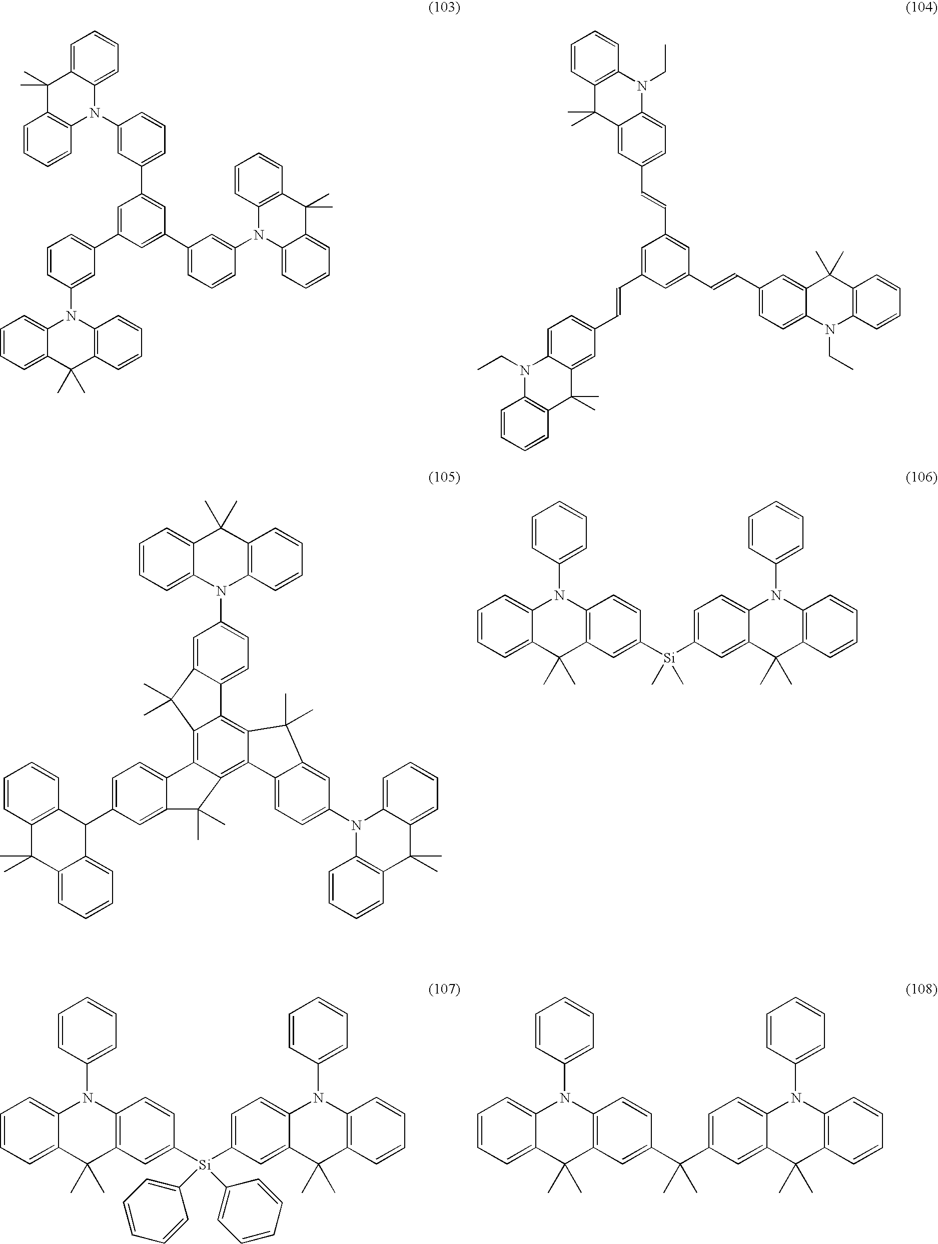

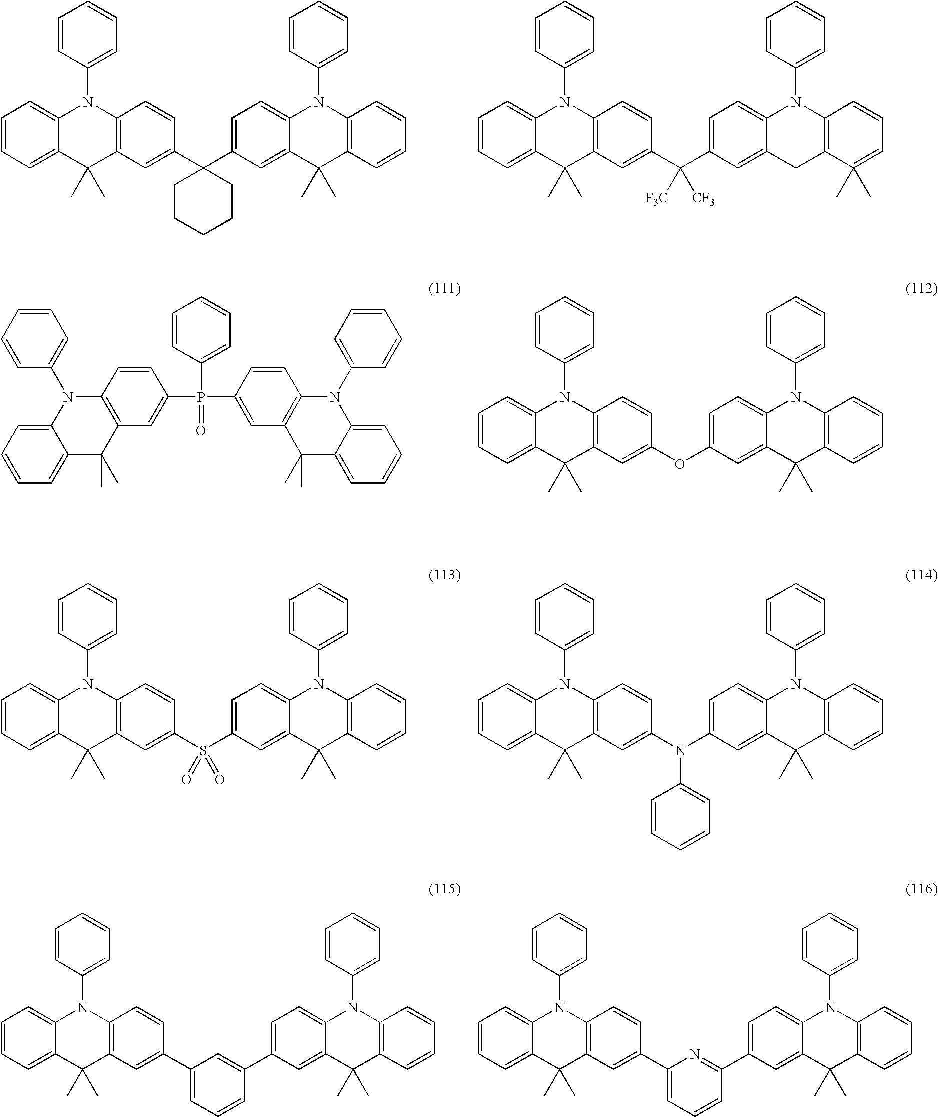

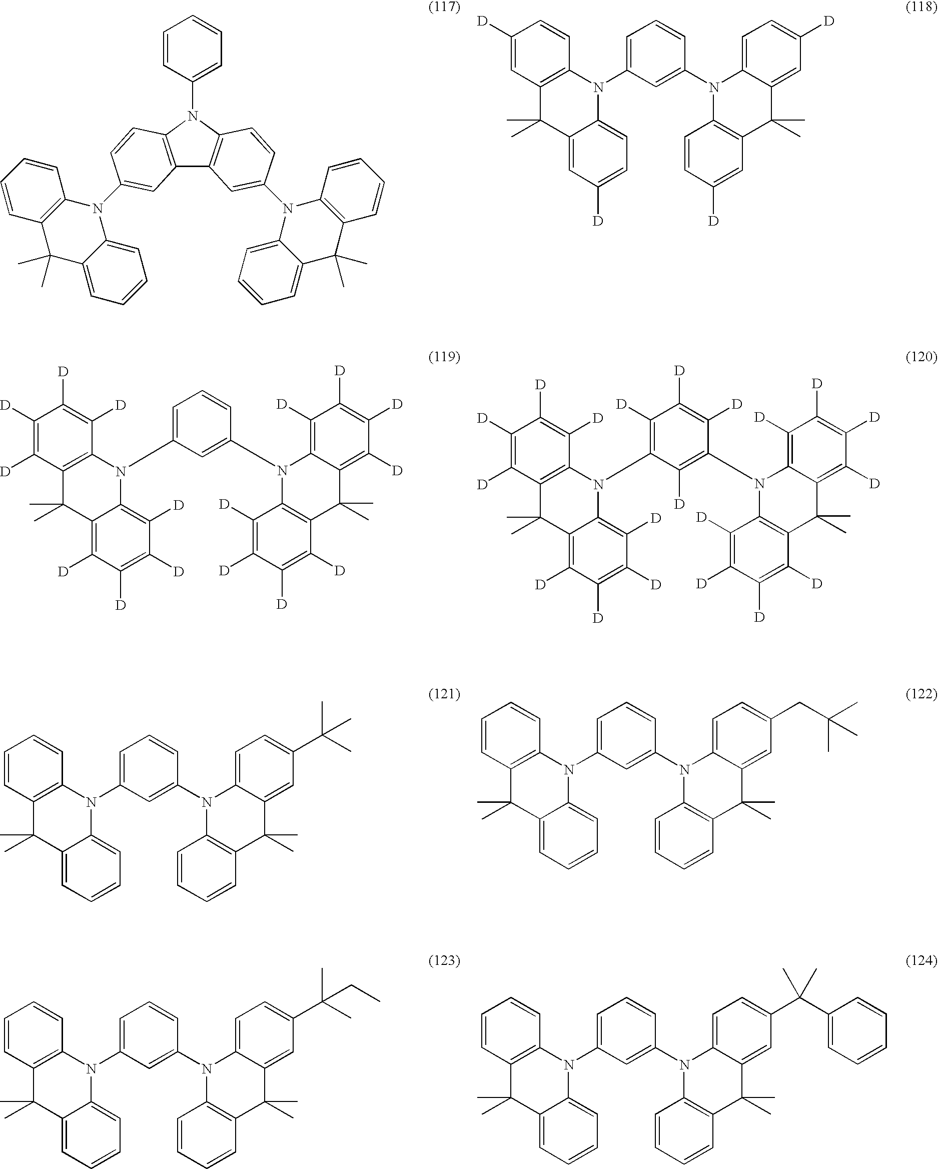

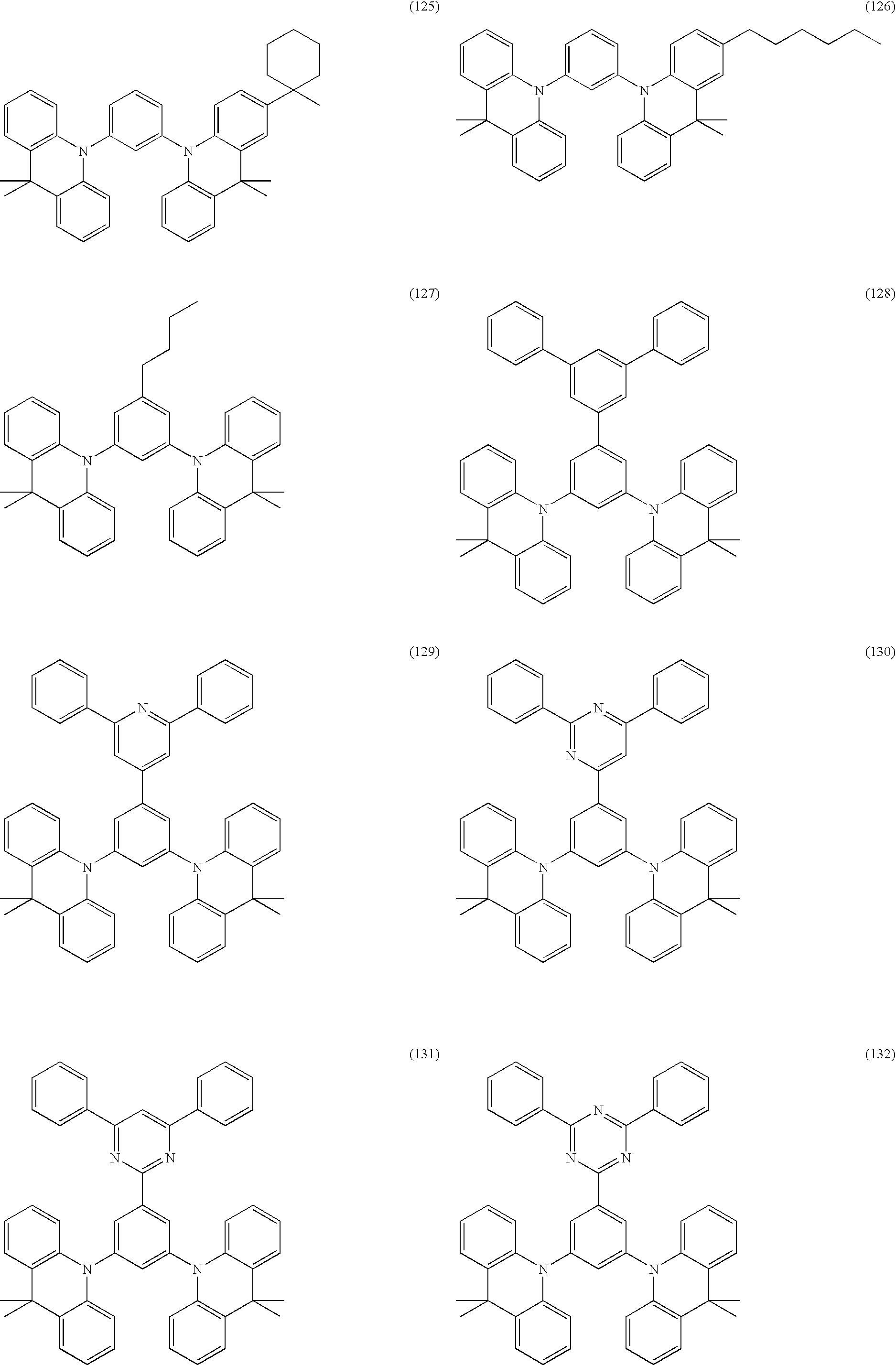

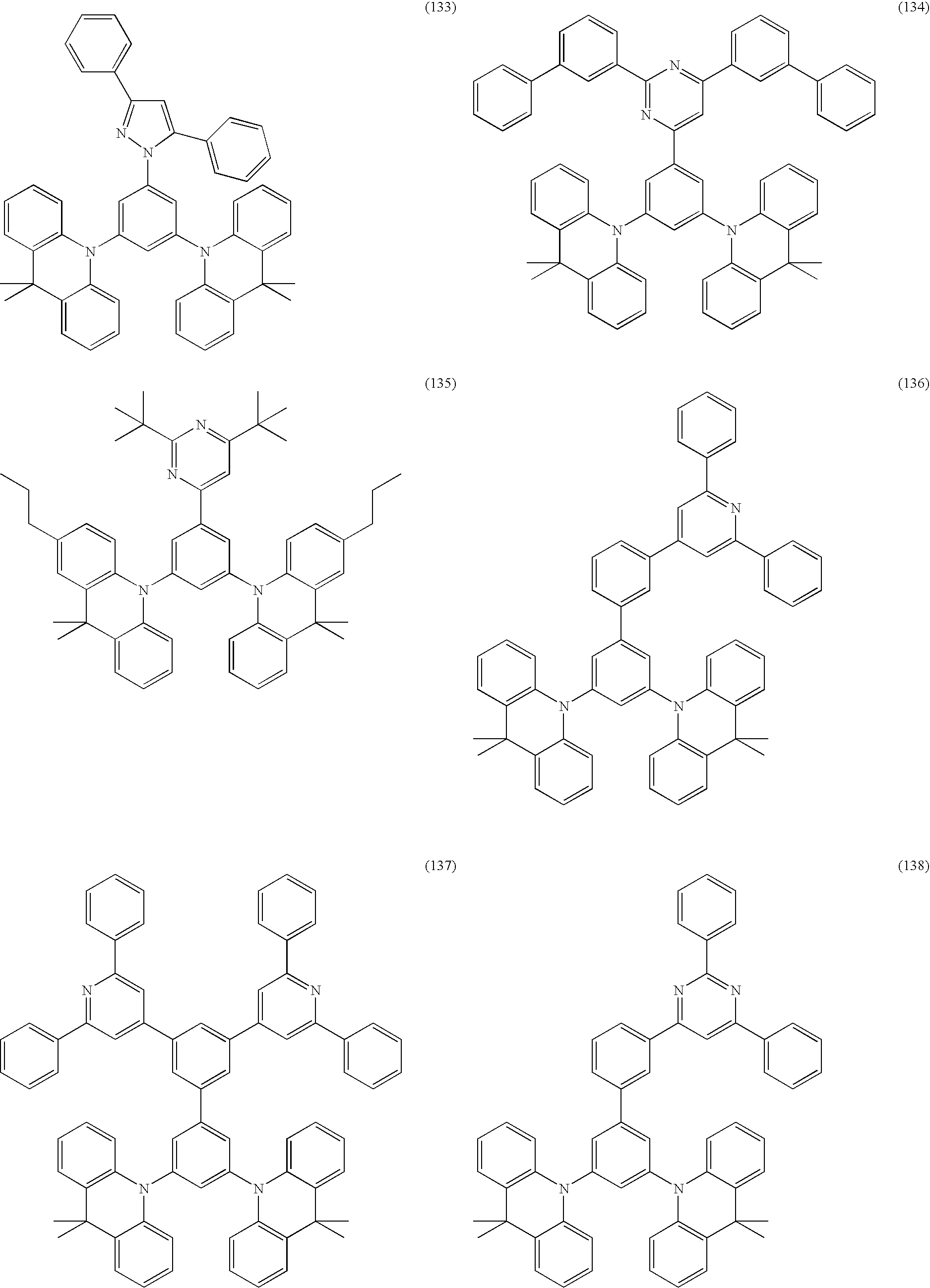

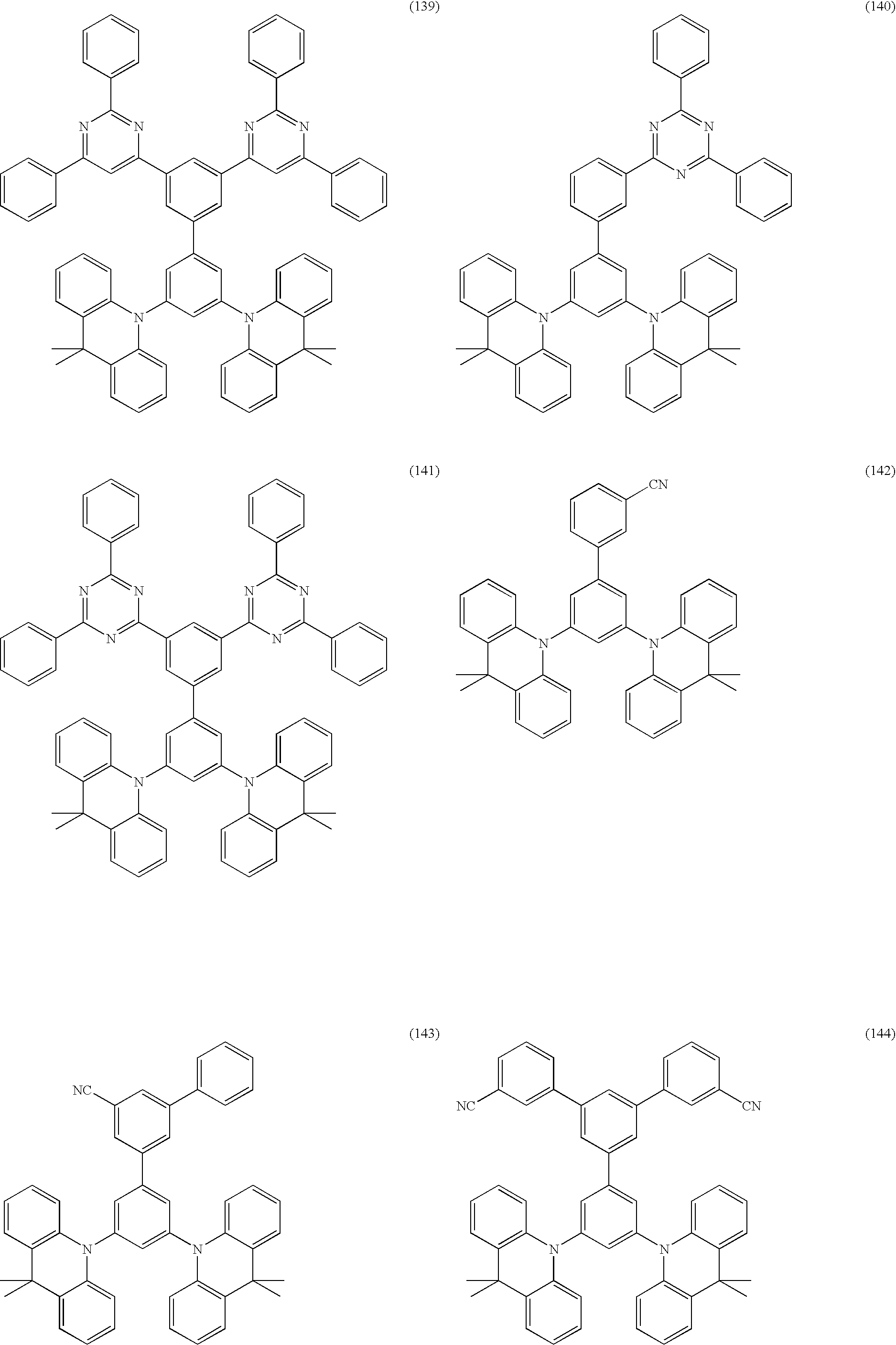

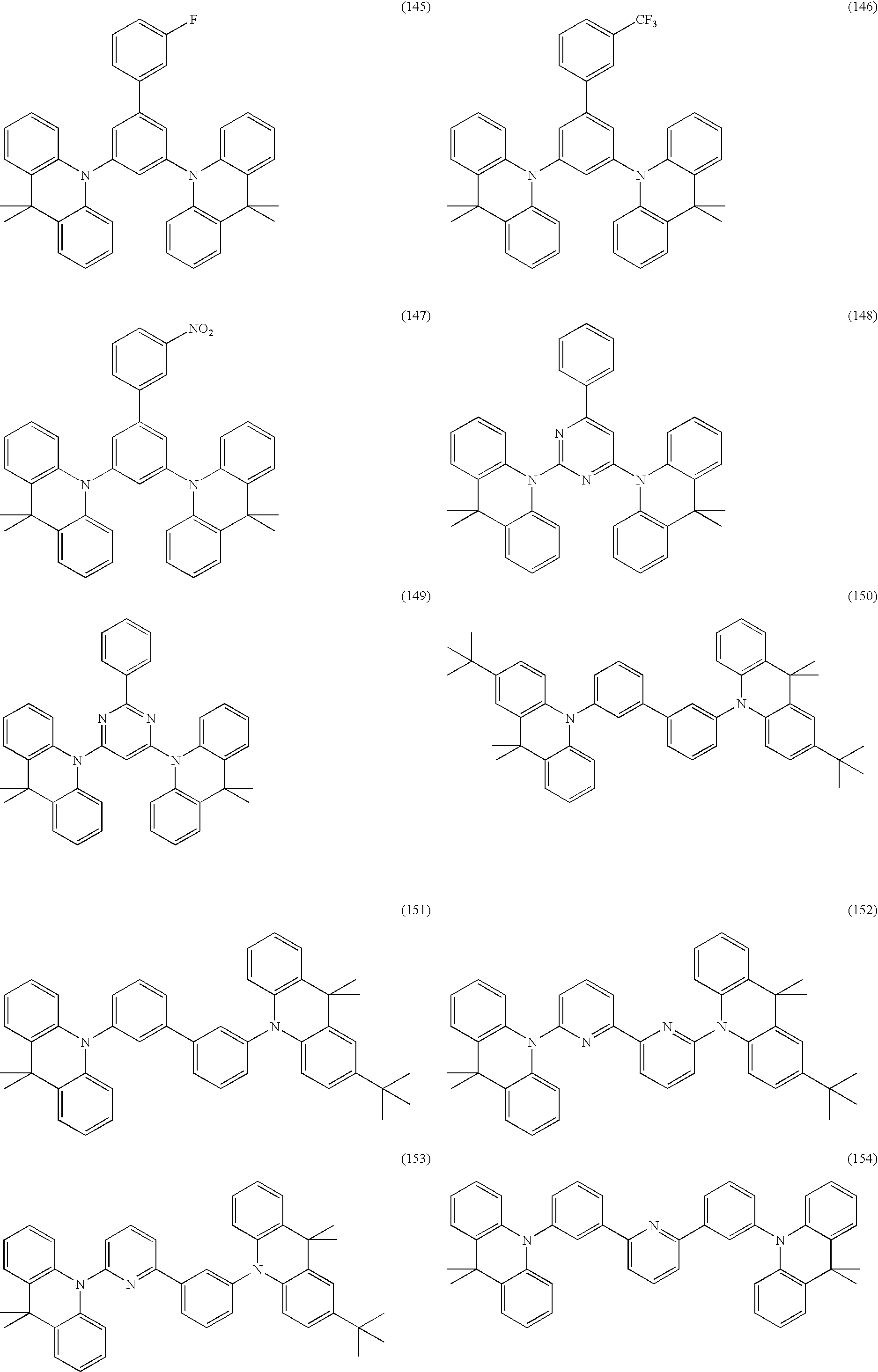

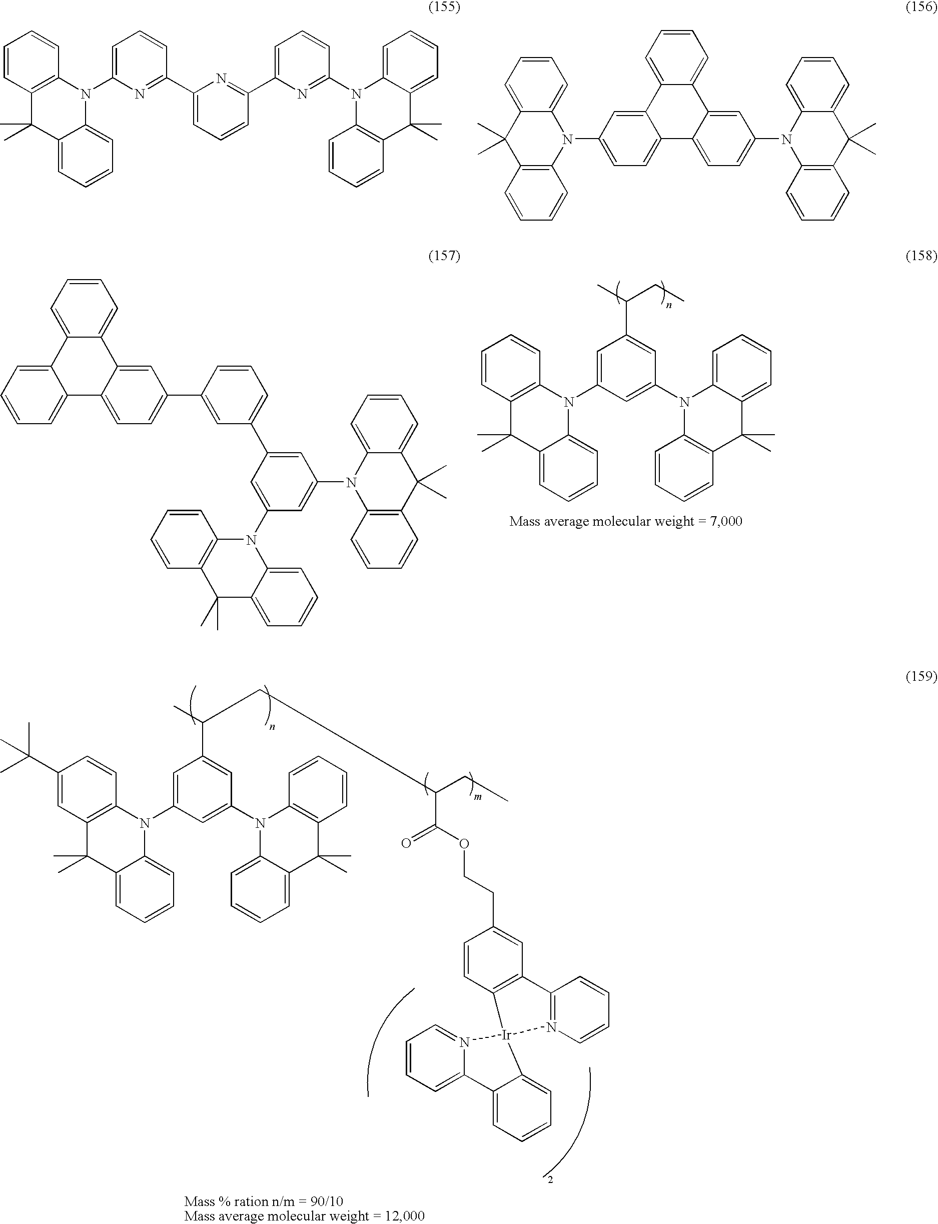

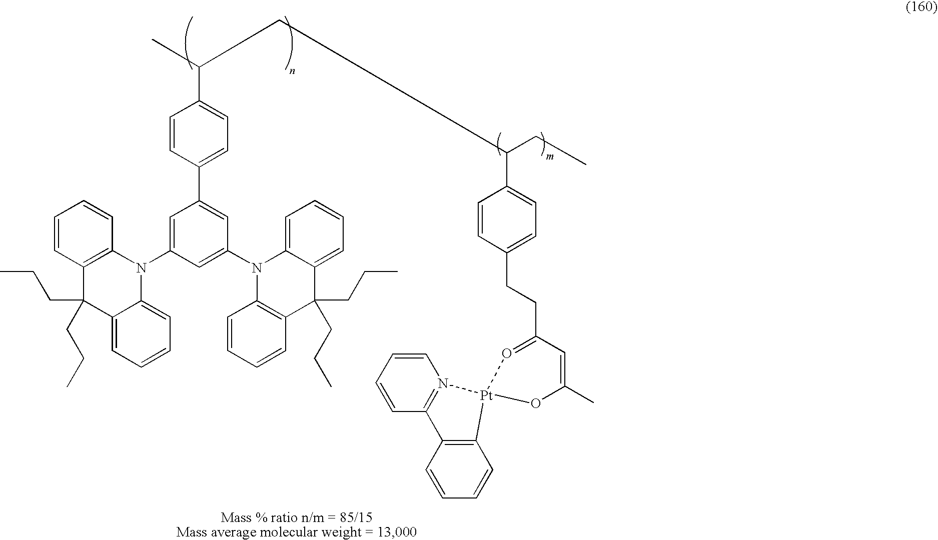

The compound of the invention can be synthesized through a combination of various known synthesis methods. The synthesis method is hereunder described.

Acridan derivatives (compounds wherein the N-position thereof is H) having various substituents can be synthesized while referring to the descriptions of documents inclusive of Chem. Soc., 1931, 2568; J. Am. Chem. Soc., 1936, 58, 1278; J. Am. Chem. Soc., 1938, 60, 1458; J. Chem. Soc. C, 1971, 2537; Angew. Chem. Int. Ed, 1991, 30, 1646; J. Mater. Chem., 2007, 17, 1209; and WO 2007/110228. The compound of the invention can be synthesized through coupling of an acridan derivative and an aryl halide by an N-arylating reaction, an aromatic nucleophilic displacement reaction or the like using a palladium catalyst (see, for example, Angew. Chem. Ind. Ed., 2003, 42, 5400). A representative synthesis route of the compound of the invention is shown below.

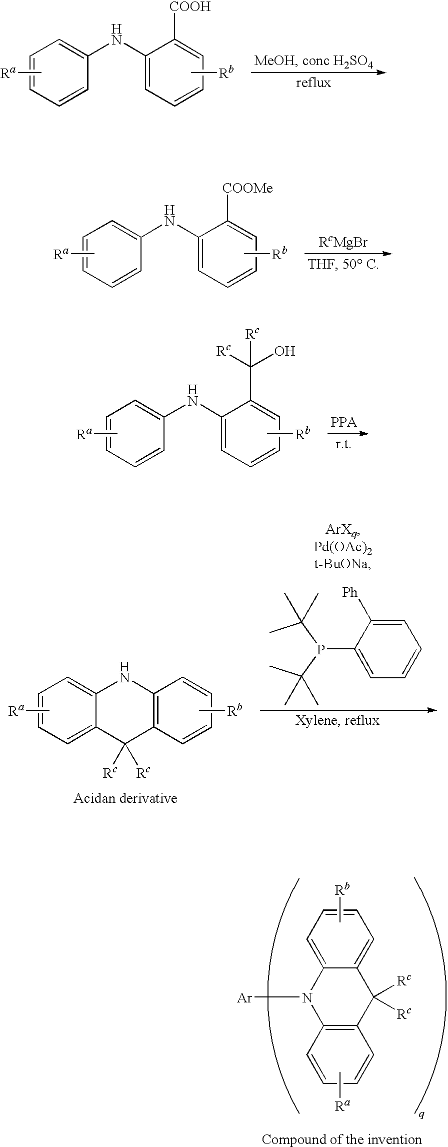

In the foregoing reaction scheme, each of R^{a}, R^{b} and R^{c} represents a substituent; Ar represents an aryl group; X represents a halogen atom; and q represents an integer.

The charge transport material of the invention is excellent in chemical stability and carrier transport properties and can be preferably used for various organic electronic devices. Any electronic device is useful, and examples thereof include organic electroluminescence devices, organic transistors, organic photoelectric conversion devices, gas sensors, organic rectifying devices, organic inverters and information recording devices. The organic photoelectric conversion device can be used for any of an optical sensor application (solid-state image device) or an energy conversion application (solar cell). Of these, organic electroluminescence devices, organic photoelectric conversion devices and organic transistors are preferable; organic electroluminescence devices and organic photoelectric conversion devices are more preferable; and organic electroluminescence devices are especially preferable.

### [Composition Containing the Compound Represented by the General Formula (1-1) or (2-1) or the Charge Transport Material Represented by the General Formula (1) or (2)]

The invention is also concerned with a composition containing the compound represented by the general formula (1-1) or (2-1) or the charge transport material represented by the general formula (1) or (2).

A content of the compound represented by the general formula (1-1) or (2-1) or the charge transport material represented by the general formula (1) or (2) in the composition of the invention is preferably from 50 to 95% by mass, and more preferably from 70 to 90% by mass.

Other component which may be contained in the composition of the invention may be an organic material or an inorganic material. As the organic material, materials which are exemplified later as a hole transport material, an electron transport material, a host material, a fluorescent material, a phosphorescent material and a hydrocarbon material can be applied. Of these, a host material and a hydrocarbon material are preferable; and a compound represented by the general formula (VI) is more preferable.

The organic layer of the organic electroluminescence device can be formed using the composition of the invention by a dry thin-film deposition method such as a vapor deposition method and a sputtering method, a transfer method, a printing method or the like.

### [Thin Film Containing the Compound Represented by the General Formula (1-1) or (2-1) or the Charge Transport Material Represented by the General Formula (1) or (2)]

The invention is also concerned with a thin film containing the compound represented by the general formula (1-1) or (2-1) or the charge transport material represented by the general formula (1) or (2). The thin film of the invention can be formed using the composition of the invention by a dry thin-film deposition method such as a vapor deposition method and a sputtering method, a transfer method, a printing method or the like. Though a thickness of the thin film may be arbitrary depending upon an application, it is preferably from 0.1 nm to 1 mm, more preferably from 0.5 nm to 1 μm, further preferably from 1 nm to 200 nm, and especially preferably from 1 nm to 100 nm.

Next, the organic electroluminescence device containing the compound of the invention is described.

### [Organic Electroluminescence Device]

It is preferable that the organic electroluminescence device of the invention has at least one organic layer between a light emitting layer and a cathode and contains the compound of the invention in the organic layer between the instant light emitting layer and the instant cathode.

The organic electroluminescence device of the invention is an organic electroluminescence device having at least one organic layer including a light emitting layer containing a light emitting material between a cathode and an anode, wherein the organic layer contains the compound represented by the foregoing general formula (1-1) or (2-1) or the charge transport material represented by the foregoing general formula (1) or (2) (hereinafter also referred to as “compound of the invention”). In view of natures of the luminescence device, it is preferable that at least one electrode of the anode and the cathode is transparent.

In the invention, as to a form of lamination of the organic layers, an embodiment in which a hole transport layer, a light emitting layer and an electron transport layer are laminated in this order from the anode side is preferable. Furthermore, a hole injection layer is provided between the hole transport layer and the anode, and/or an electron transporting interlayer is provided between the light emitting layer and the electron transport layer. Also, a hole transporting interlayer may be provided between the light emitting layer and the hole transport layer, and an electron injection layer may be similarly provided between the cathode and the electron transport layer.

Each of the layers may be divided into plural secondary layers.

Each of the layers constituting the organic layer can be suitably formed by any method, for example, a dry thin-film deposition method such as a vapor deposition method and a sputtering method, a transfer method, a printing method, a coating method, an inkjet method, a spraying method, etc.

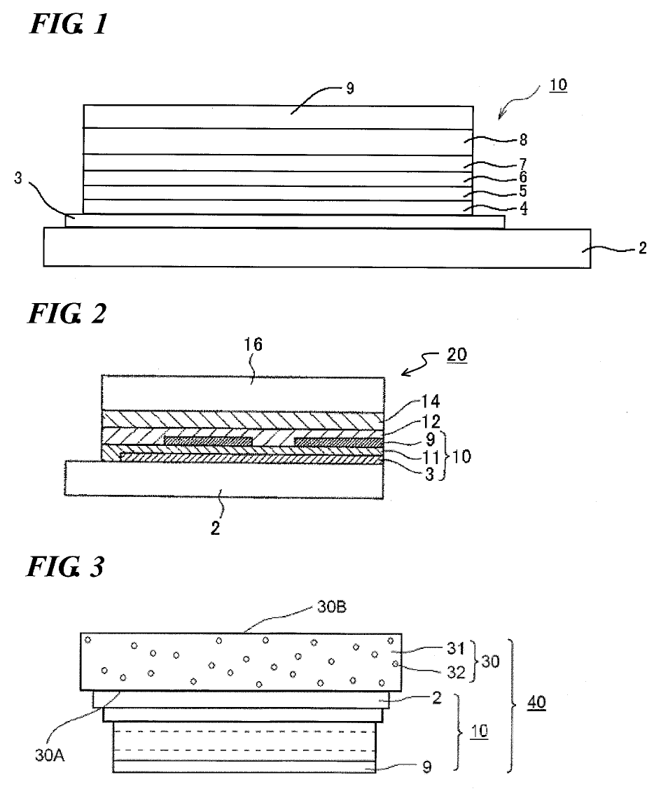

FIG. 1 illustrates an exemplary embodiment of a layer constitution of an organic electroluminescent device according to the invention. In an organic electroluminescent device 10 according to the invention shown in FIG. 1, a light emitting layer 6 is provided between an anode 3 and a cathode 9 above a supporting substrate 2. Specifically, a hole injection layer 4, a hole transport layer 5, the light emitting layer 6, a hole blocking layer 7 and an electron transport layer 8 are provided in this order between the anode 3 and the cathode 9.

Next, each of the elements constituting the luminescence device of the invention is described.

### (Substrate)

It is preferable that the substrate which is used in the invention is a substrate which does not scatter or decay light emitted from the organic layer.

### (Anode)

In general, the anode may have a function as an electrode for feeding a hole into the organic layer. The anode is not particularly limited with respect to its shape, structure and size and so on and may be properly selected among known electrode materials depending upon an application or purpose of the luminescence device. As described previously, the anode is usually provided as a transparent anode.

### (Cathode)

In general, the cathode may have a function as an electrode for injecting an electron into the organic layer. The cathode is not particularly limited with respect to its shape, structure and size and so on and may be properly selected among known electrode materials depending upon an application or purpose of the luminescence device.

### (Organic Layer)

The organic EL device of the invention has at least one organic layer including the light emitting layer. As described previously, examples of other organic layers than the light emitting layer include respective layers such as a hole transport layer, an electron transport layer, a charge blocking layer, a hole injection layer and an electron injection layer.

### (Light Emitting Layer)

The light emitting layer is a layer having functions such that at the time of impressing an electric field, it accepts a hole from the anode, the hole injection layer or the hole transport layer, accepts an electron from the cathode, the electron injection layer or the electron transport layer and provides a site of recombination of the hole and the electron, thereby achieving light emission.

The substrate, the anode, the cathode, the organic layer and the light emitting layer are described in detail in, for example, JP-A-2008-270736 and JP-A-2007-266458, and the matters disclosed in these patent documents can be applied to the invention.

### <Light Emitting Material>

As the light emitting material in the invention, any of a phosphorescent material or a fluorescent material can be used.

For the purposes of enhancing a color purity and broadening a light emission wavelength region, the light emitting layer in the invention can contain two or more kinds of light emitting materials. It is preferable that at least one kind of the light emitting materials is a phosphorescent material.

From the viewpoint of driving durability, it is preferable that the light emitting material in the invention is further satisfied with the relationships of (1.2 eV>ΔIp>0.2 eV) and/or (1.2 eV>{Ea>0.2 eV) relative to the host material. Here, ΔIp means a difference in the Ip value between the host material and the light emitting material; and ΔEa means a difference in the Ea value between the host material and the light emitting material.

It is preferable that at least one kind of the light emitting materials is a platinum complex or an iridium complex.

In the invention, the light emitting layer preferably includes a platinum complex material. The light emitting layer preferably includes a platinum complex material having a tridentate or more multidentate ligand and more preferably includes a platinum complex material having a tetradentate ligand. A maximum light emitting wavelength of the phosphorescent material is preferably not more than 500 nm.

The fluorescent material and the phosphorescent material are described in detail, in, for example, paragraphs [0100] to [0164] of JP-A-2008-270736 and paragraphs [0088] to [0090] of JP-A-2007-266458, and the matters disclosed in these patent documents can be applied to the invention.

The platinum complex is preferably a platinum complex represented by the following general formula (C-1).

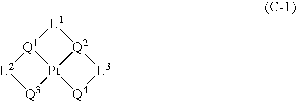

In the foregoing general formula, each of Q¹, Q², Q³ and Q⁴ independently represents a ligand which is coordinated on Pt; and each of L¹, L² and L³ independently represents a single bond or a divalent connecting group.

The genera formula (C-1) is described. Each of Q¹, Q², Q³ and Q⁴ independently represents a ligand which is coordinated on Pt. At that time, the bond of each of Q¹, Q², Q³ and Q⁴ to Pt may be any of a covalent bond, an ionic bond or a coordination bond. As an atom bonding to Pt in each of Q¹, Q², Q³ and Q⁴, a carbon atom, a nitrogen atom, an oxygen atom, a sulfur atom and a phosphorus atom are preferable. Among the atoms bonding to Pt in Q¹, Q², Q³ and Q⁴, it is preferable that at least one of them is a carbon atom; it is more preferable that two of them are a carbon atom; and it is especially preferable that two of them are a carbon atom, with other two being a nitrogen atom.

As Q¹, Q², Q³ and Q⁴ bonding to Pt with a carbon atom, any of an anionic ligand or a neutral ligand is useful. Examples of the anionic ligand include a vinyl ligand, an aromatic hydrocarbon ring ligand (for example, a benzene ligand, a naphthalene ligand, an anthracene ligand, a phenanthracene ligand, etc.) and a heterocyclic ring ligand (for example, a furan ligand, a thiophene ligand, a pyridine ligand, a pyrazine ligand, a pyrimidine ligand, a pyridazine ligand, a triazine ligand, a thiazole ligand, an oxazole ligand, a pyrrole ligand, an imidazole ligand, a pyrazole ligand, a triazole ligand and condensed ring materials including the same (for example, a quinoline ligand, a benzothiazole ligand, etc.)). Examples of the neutral ligand include a carbene ligand.

As Q¹, Q², Q³ and Q⁴ bonding to Pt with a nitrogen atom, any of a neutral ligand or an anionic ligand is useful. Examples of the neutral ligand include a nitrogen-containing aromatic heterocyclic ring ligand (for example, a pyridine ligand, a pyrazine ligand, a pyrimidine ligand, a pyridazine ligand, a triazine ligand, an imidazole ligand, a pyrazole ligand, a triazole ligand, an oxazole ligand, a thiazole ligand and condensed ring materials including the same (for example, a quinoline ligand, a benzimidazole ligand, etc.)), an amine ligand, a nitrile ligand and an imine ligand. Examples of the anionic ligand include an amino ligand, an imino ligand and a nitrogen-containing aromatic heterocyclic ring ligand (for example, a pyrrole ligand, an imidazole ligand, a triazole ligand and condensed ring materials including the same (for example, an indole ligand, a benzimidazole ligand, etc.)).

As Q¹, Q², Q³ and Q⁴ bonding to Pt with an oxygen atom, any of a neutral ligand or an anionic ligand is useful. Examples of the neutral ligand include an ether ligand, a ketone ligand, an ester ligand, an amide ligand and a nitrogen-containing heterocyclic ring ligand (for example, a furan ligand, an oxazole ligand and condensed ring materials including the same (for example, a benzoxazole ligand, etc.)). Examples of the anionic ligand include an alkoxy ligand, an aryloxy ligand, a heteroaryloxy ligand, an acyloxy ligand and a silyloxy ligand.

As Q¹, Q², Q³ and Q⁴ bonding to Pt with a sulfur atom, any of a neutral ligand or an anionic ligand is useful. Examples of the neutral ligand include a thioether ligand, a thioketone ligand, a thioester ligand, a thioamide ligand and a sulfur-containing heterocyclic ring ligand (for example, a thiophene ligand, a thiazole ligand and condensed ring materials (for example, a benzothiazole ligand, etc.)). Examples of the anionic ligand include an alkyl mercapto ligand, an aryl mercapto ligand and a heteroaryl mercapto ligand.

As Q¹, Q², Q³ and Q⁴ bonding to Pt with a phosphorus atom, any of a neutral ligand or an anionic ligand is useful. Examples of the neutral ligand include a phosphine ligand, a phosphoric ester ligand, a phosphorous ester ligand and a phosphorus-containing heterocyclic ring ligand (for example, a phosphinine ligand, etc.). Examples of the anionic ligand include a phosphino ligand, a phosphinyl ligand and a phosphoryl ligand.

The group represented by each of Q¹, Q², Q³ and Q⁴ may have a substituent. As the substituent, those exemplified above for the group A of substituents can be properly applied. Also, the substituents may be connected to each other (in the case where Q³ and Q⁴ are connected to each other, a Pt complex of a cyclic tetradentate ligand is formed).

The group represented by each of Q¹, Q², Q³ and Q⁴ is preferably an aromatic hydrocarbon ring ligand bonding to Pt with a carbon atom, an aromatic heterocyclic ring ligand bonding to Pt with a carbon atom, a nitrogen-containing aromatic heterocyclic ring ligand bonding to Pt with a nitrogen atom, an acyloxy ligand, an alkyloxy ligand, an aryloxy ligand, a heteroaryloxy ligand or a silyloxy ligand; more preferably an aromatic hydrocarbon ring ligand bonding to Pt with a carbon atom, an aromatic heterocyclic ring ligand bonding to Pt with a carbon atom, a nitrogen-containing aromatic heterocyclic ring ligand bonding to Pt with a nitrogen atom, an acyloxy ligand or an aryloxy ligand; and further preferably an aromatic hydrocarbon ring ligand bonding to Pt with a carbon atom, an aromatic heterocyclic ring ligand bonding to Pt with a carbon atom, a nitrogen-containing aromatic heterocyclic ring ligand bonding to Pt with a nitrogen atom or an acyloxy ligand.

Each of L¹, L² and L³ represents a single bond or a divalent connecting group. Examples of the divalent connecting group represented by each of L¹, L² and L³ include an alkylene group (for example, methylene, ethylene, propylene, etc.), an arylene group (for example, phenylene, naphthalenediyl, etc.), a heteroarylene group (for example, pyridinediyl, thiophenediyl, etc.), an imino group (—NR—) (for example, a phenylimino group, etc.), an oxy group (—O—), a thio group (—S—), a phosphinidene group (—PR—) (for example, a phenylphosphinidene group, etc.), a silylene group (—SiRR′—) (for example, a dimethylsilylene group, a diphenylsilylene group, etc.) and a combination thereof. These connecting groups may further have a substituent.

From the viewpoints of stability and light emission quantum yield of the complex, each of L¹, L² and L³ is preferably a single bond, an alkylene group, an arylene group, a heteroarylene group, an imino group, an oxy group, a thio group or a silylene group; more preferably a single bond, an alkylene group, an arylene group or an imino group; further preferably a single bond, an alkylene group or an arylene group; even further preferably a single bond, a methylene group or a phenylene group; even more preferably a single bond or a di-substituted methylene group; even still further preferably a single bond, a dimethylmethylene group, a diethylmethylene group, a diisobutylmethylene group, a dibenzylmethylene group, an ethylmethylmethylene group, a methylpropylmethylene group, an isobutylmethylmethylene group, a diphenylmethylene group, a methylphenylmethylene group, a cyclohexanediyl group, a cyclopentanediyl group, a fluorenediyl group or a fluoromethylmethylene group; and especially preferably a single bond, a dimethylmethylene group, a diphenylmethylene group or a cyclohexanediyl group.

The platinum complex represented by the general formula (C-1) is more preferably a platinum complex represented by the following general formula (C-2).

### General Formula (C-2)

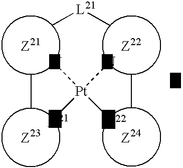

In the foregoing general formula, L²¹ represents a single bond or a divalent connecting group; each of A²¹ and A²² independently represents C or N; each of Z²¹ and Z²² independently represents a nitrogen-containing aromatic heterocyclic ring; and each of Z²³ and Z²⁴ independently represents a benzene ring or an aromatic heterocyclic ring.

The general formula (C-2) is described. L²¹ is synonymous with L¹ in the foregoing general formula (C-1), and preferred ranges thereof are also the same.

Each of A²¹ and A²² independently represents a carbon atom or a nitrogen atom. It is preferable that at least one of A²¹ and A²² is a carbon atom. From the viewpoint of stability of the complex and the viewpoint of light emission quantum yield of the complex, it is preferable that both A²¹ and A²² are a carbon atom.

Each of Z²¹ and Z²² independently represents a nitrogen-containing aromatic heterocyclic ring. Examples of the nitrogen-containing aromatic heterocyclic ring represented by each of Z²¹ and Z²² include a pyridine ring, a pyrimidine ring, a pyrazine ring, a triazine ring, an imidazole ring, a pyrazole ring, an oxazole ring, a thiazole ring, a triazole ring, an oxadiazole ring and a thiadiazole ring. From the viewpoints of stability, control of light emission wavelength and light emission quantum yield of the complex, the ring represented by each of Z²¹ and Z²² is preferably a pyridine ring, a pyrazine ring, an imidazole ring or a pyrazole ring; more preferably a pyridine ring, an imidazole ring or a pyrazole ring; further preferably a pyridine ring or a pyrazole ring; and especially preferably a pyridine ring.

The nitrogen-containing aromatic heterocyclic ring represented by each of Z²¹ and Z²² may have a substituent. As the substituent on the carbon atom, those exemplified above for the group A of substituents can be applied; and as the substituent on the nitrogen atom, those exemplified above for the group B of substituents can be applied. The substituent on the carbon atom is preferably an alkyl group, a polyfluoroalkyl group, an aryl group, an aromatic heterocyclic group, a dialkylamino group, a diarylamino group, an alkoxy group, a cyano group or a halogen atom. Though the substituent is properly selected for the purpose of controlling the light emission wavelength or potential, in the case of making the wavelength short, the substituent is preferably an electron donating group, a fluorine atom or an aromatic ring group, and for example, an alkyl group, a dialkylamino group, an alkoxy group, a fluorine atom, an aryl group, an aromatic heterocyclic group and so on are selected. Also, in the case of making the wavelength long, the substituent is preferably an electron withdrawing group, and for example, a cyano group, a polyfluoroalkyl group and so on are selected. The substituent on N is preferably an alkyl group, an aryl group or an aromatic heterocyclic group; and from the viewpoint of stability of the complex, an alkyl group and an aryl group are preferable. The substituents may be connected to each other to form a condensed ring. Examples of the ring to be formed include a benzene ring, a pyridine ring, a pyrazine ring, a pyridazine ring, a pyrimidine ring, an imidazole ring, an oxazole ring, a thiazole ring, a pyrazole ring, a thiophene ring and a furan ring.

Each of Z²³ and Z²⁴ independently represents a benzene ring or an aromatic heterocyclic ring. Examples of the nitrogen-containing aromatic heterocyclic ring represented by each of Z²³ and Z²⁴ include a pyridine ring, a pyrimidine ring, a pyrazine ring, a pyridazine ring, a triazine ring, an imidazole ring, a pyrazole ring, an oxazole ring, a thiazole ring, a triazole ring, an oxadiazole ring, a thiadiazole ring, a thiophene ring and a furan ring. From the viewpoints of stability, control of light emission wavelength and light emission quantum yield of the complex, the ring represented by each of Z²³ and Z²⁴ is preferably a benzene ring, a pyridine ring, a pyrazine ring, an imidazole ring, a pyrazole ring or a thiophene ring; more preferably a benzene ring, a pyridine ring or a pyrazole ring; and further preferably a benzene ring or a pyridine ring.

The benzene ring or the nitrogen-containing aromatic heterocyclic ring represented by each of Z²³ and Z²⁴ may have a substituent. As the substituent on the carbon atom, those exemplified above for the group A of substituents can be applied; and as the substituent on the nitrogen atom, those exemplified above for the group B of substituents can be applied. The substituent on the carbon atom is preferably an alkyl group, a polyfluoroalkyl group, an aryl group, an aromatic heterocyclic group, a dialkylamino group, a diarylamino group, an alkoxy group, a cyano group or a halogen atom. Though the substituent is properly selected for the purpose of controlling the light emission wavelength or potential, in the case of making the wavelength long, the substituent is preferably an electron donating group or an aromatic ring group, and for example, an alkyl group, a dialkylamino group, an alkoxy group, an aryl group, an aromatic heterocyclic group and so on are selected. Also, in the case of making the wavelength short, the substituent is preferably an electron withdrawing group, and for example, a fluorine group, a cyano group, a polyfluoroalkyl group and so on are selected. The substituent on N is preferably an alkyl group, an aryl group or an aromatic heterocyclic group; and from the viewpoint of stability of the complex, an alkyl group and an aryl group are preferable. The substituents may be connected to each other to form a condensed ring. Examples of the ring to be formed include a benzene ring, a pyridine ring, a pyrazine ring, a pyridazine ring, a pyrimidine ring, an imidazole ring, an oxazole ring, a thiazole ring, a pyrazole ring, a thiophene ring and a furan ring.

Of the platinum complexes represented by the general formula (C-2), one of more preferred embodiments is a platinum complex represented by the following general formula (C-4).

### General Formula (C-4)

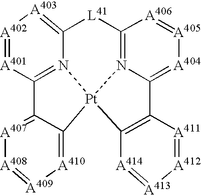

In the general formula (C-4), each of A⁴⁰¹ to A⁴¹⁴ independently represents C—R or N; R represents a hydrogen atom or a substituent; and L⁴¹ represents a single bond or a divalent connecting group.

The general formula (C-4) is described.

Each of A⁴⁰¹ to A⁴¹⁴ independently represents C—R or N; and R represents a hydrogen atom or a substituent. As the substituent represented by R, those exemplified above for the group A of substituents can be applied.

Each of A⁴⁰¹ to A⁴⁰⁶ is preferably C—R and Rs may be connected to each other to form a ring. When each of A⁴⁰¹ to A⁴⁰⁶ represents C—R, the Rs of A⁴⁰² and A⁴⁰⁵ are preferably a hydrogen atom, an alkyl group, an aryl group, an amino group, an alkoxy group, an aryloxy group, a fluorine group, or a cyano group, more preferably a hydrogen atom, an amino group, an alkoxy group, an aryloxy group, or a fluorine group, especially preferably a hydrogen atom or a fluorine group. The Rs of A⁴⁰¹, A⁴⁰³, A⁴⁰⁴, and A⁴⁰⁶ are preferably a hydrogen atom, an alkyl group, an aryl group, an amino group, an alkoxy group, an aryloxy group, a fluorine group, or a cyano group, more preferably a hydrogen atom, an amino group, an alkoxy group, an aryloxy group, or a fluorine group, especially preferably a hydrogen atom.

L⁴¹ is synonymous with L¹ in the foregoing general formula (C-1), and preferred ranges thereof is also the same.

As A^{407 to A}⁴¹⁴, in each of A⁴⁰⁷ to A⁴¹⁰ and A⁴¹¹ to A⁴¹⁴, the number of N (nitrogen atom) is preferably from 0 to 2, and more preferably from 0 to 1. In the case of shifting the light emission wavelength to the short wavelength side, it is preferable that any one of A⁴⁰⁸ and A⁴¹² is an N atom; and it is more preferable that both A⁴⁰⁸ and A⁴¹² are an N atom.

In the case where each of A⁴⁰⁷ to A⁴¹⁴ represents C—R, R in each of A⁴⁰⁸ and A⁴¹² is preferably a hydrogen atom, an alkyl group, a polyfluoroalkyl group, an aryl group, an amino group, an alkoxy group, an aryloxy group, a fluorine group or a cyano group; more preferably a hydrogen atom, a polyfluoroalkyl group, an alkyl group, an aryl group, a fluorine group or a cyano group; and especially preferably a hydrogen atom, a phenyl group, a polyfluoroalkyl group or a cyano group. R in each of A⁴⁰⁷, A⁴⁰⁹, A⁴¹¹ and A⁴¹³ is preferably a hydrogen atom, an alkyl group, a polyfluoroalkyl group, an aryl group, an amino group, an alkoxy group, an aryloxy group, a fluorine group or a cyano group; more preferably a hydrogen atom, a polyfluoroalkyl group, a fluorine group or a cyano group; and especially preferably a hydrogen atom, a phenyl group or a fluorine group. R in each of A⁴¹⁰ and A⁴¹⁴ is preferably a hydrogen atom or a fluorine group, and more preferably a hydrogen atom. In the case where any one of A⁴⁰⁷ to A⁴⁰⁹ and A⁴¹¹ to A⁴¹³ represents C—R, Rs may be connected to each other to form a ring.

Of the platinum complexes represented by the general formula (C-2), one of more preferred embodiments is a platinum complex represented by the following general formula (C-5).

### General Formula (C-5)

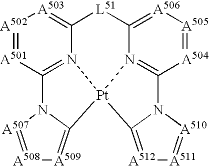

In the general formula (C-5), each of A⁵⁰¹ to A⁵¹² independently represents C—R or N; R represents a hydrogen atom or a substituent; and L⁵¹ represents a single bond or a divalent connecting group.

The general formula (C-5) is described. A⁵⁰¹ to A⁵⁰⁶ and L⁵¹ are synonymous with A⁴⁰¹ to A⁴⁰⁶ and L⁴¹ in the foregoing general formula (C-4), respectively, and preferred ranges thereof are also the same.

Each of A⁵⁰⁷, A⁵⁰⁸ and , and A⁵¹⁰, A⁵¹¹ and A⁵¹² independently represents C—R or N; and R represents a hydrogen atom or a substituent. As the substituent represented by R, those exemplified above for the group A of substituents can be applied.

When each of A⁵⁰⁷, A⁵⁰⁸ and A⁵⁰⁹, and A⁵¹⁰, A⁵¹¹ and A⁵¹² represents C—R, R is preferably a hydrogen atom, an alkyl group, a polyfluoroalkyl group, an aryl group, an aromatic heterocyclic group, a dialkylamino group, a diarylamino group, an alkyloxy group, a cyano group, or a halogen atom, more preferably a hydrogen atom, an alkyl group, a polyfluoroalkyl group, an aryl group, a dialkylamino group, a cyano group, or a fluorine atom, still more preferably a hydrogen atom, an alkyl group, a trifluoromethyl group, or a fluorine atom. If possible, the substituents may be connected to each other to form a condensed ring structure. Preferably at least one of A⁵⁰⁷, A⁵⁰⁸ and A⁵⁰⁹, and A⁵¹⁰, A⁵¹¹ and A⁵¹² represents N and especially preferably A⁵⁰⁷ or A⁵¹⁰ represents N.

Of the platinum complexes represented by the general formula (C-1), another more preferred embodiment is a platinum complex represented by the following general formula (C-6).

### General Formula (C-6)

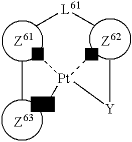

In the foregoing general formula, L⁶¹ represents a single bond or a divalent connecting group; A⁶¹ represents C or N; each of Z⁶¹ and Z⁶² independently represents a nitrogen-containing aromatic heterocyclic ring; Z⁶³ represents a benzene ring or an aromatic heterocyclic ring; and Y represents an anionic non-cyclic ligand bonding to Pt.

The general formula (C-6) is described. L⁶¹ is synonymous with L¹ in the foregoing general formula (C-1), and preferred ranges thereof are also the same.

A⁶¹ represents C or N. From the viewpoint of stability of the complex and the viewpoint of light emission quantum yield of the complex, A⁶¹ is preferably C.

Z⁶¹ and Z⁶² are synonymous with Z²¹ and Z²² in the foregoing general formula (C-2), respectively, and preferred ranges thereof are also the same. Z⁶³ is synonymous with Z²³ in the foregoing general formula (C-2), and preferred ranges thereof are also the same.

Y is an anionic non-cyclic ligand bonding to Pt. The non-cyclic ligand as referred to herein is one in which an atom bonding to Pt does not form a ring in a ligand state. The atom bonding to Pt in Y is preferably a carbon atom, a nitrogen atom, an oxygen atom or a sulfur atom; more preferably a nitrogen atom or an oxygen atom; and most preferably an oxygen atom. Examples of Y bonding to Pt with a carbon atom include a vinyl ligand. Examples of Y bonding to Pt with a nitrogen atom include an amino ligand and an imino ligand. Examples of Y bonding to Pt with an oxygen atom include an alkoxy ligand, an aryloxy ligand, a heteroaryloxy ligand, an acyloxy ligand, a silyloxy ligand, a carboxyl ligand, a phosphate ligand or a sulfonate ligand. Examples of Y bonding to Pt with a sulfur atom include an alkyl mercapto ligand, an aryl mercapto ligand, a heteroaryl mercapto ligand and a thiocarboxylate ligand.

The ligand represented by Y may have a substituent. As the substituent, those exemplified above for the group A of substituents can be properly applied. Also, the substituents may be connected to each other.

The ligand represented by Y is preferably a ligand bonding to Pt with an oxygen atom; more preferably an acyloxy ligand, an alkyloxy ligand, an aryloxy ligand, a heteroaryloxy ligand or a silyloxy ligand; and further preferably an acyloxy ligand.

Of the platinum complexes represented by the general formula (C-6), one of more preferred embodiments is a platinum complex represented by the following general formula (C-7).

### General Formula (C-7)

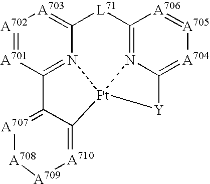

In the foregoing general formula, each of A⁷⁰¹ to A⁷¹⁰ independently represents C—R or N; R represents a hydrogen atom or a substituent; L⁷¹ represents a single bond or a divalent connecting group; Y represents an anionic non-cyclic ligand bonding to Pt.

The general formula (C-7) is described. L⁷¹ is synonymous with L⁶¹ in the foregoing general formula (C-6), and preferred ranges thereof are also the same. A⁷⁰¹ to A⁷¹⁰ are synonymous with A⁴⁰¹ to A⁴¹⁰ in the foregoing general formula (C-4), respectively, and preferred ranges thereof are also the same. Y is synonymous with Y in the general formula (C-6), and preferred ranges thereof are also the same.

Specific examples of the platinum complex represented by the general formula (C-1) include compounds disclosed in paragraphs [0143] to [0152], [0157] to and [0162] to [0168] of JP-A-2005-310733; compounds disclosed in paragraphs to [0083] of JP-A-2006-256999; compounds disclosed in paragraphs [0065] to [0090] of JP-A-2006-93542; compounds disclosed in paragraphs [0063] to [0071] of JP-A-2007-73891; compounds disclosed in paragraphs [0079] to [0083] of JP-A-2007-324309; compounds disclosed in paragraphs [0065] to [0090] of JP-A-2006-93542; compounds disclosed in paragraphs [0055] to [0071] of JP-A-2007-96255; and compounds disclosed in paragraphs [0043] to [0046] of JP-A-2006-313796. Besides, the following platinum complexes can be exemplified.

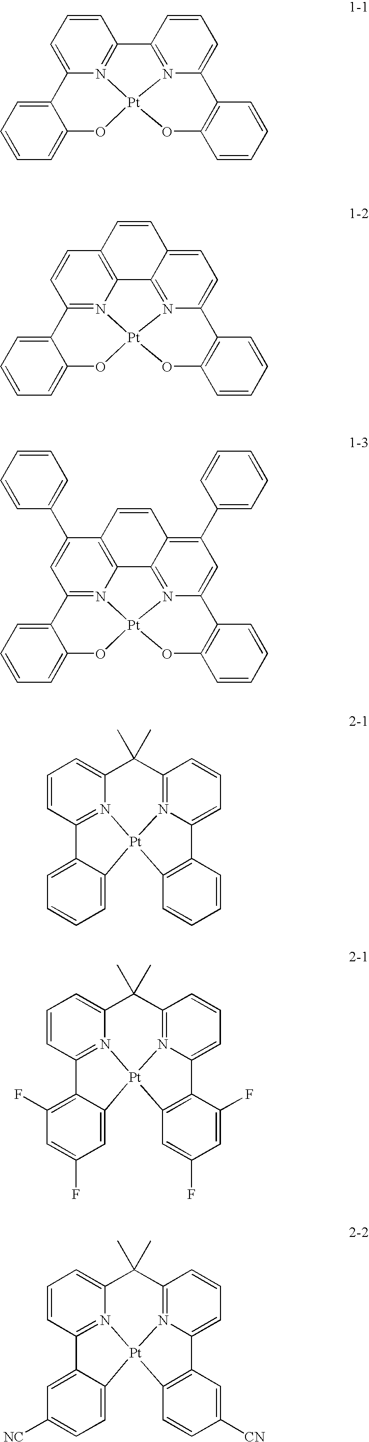

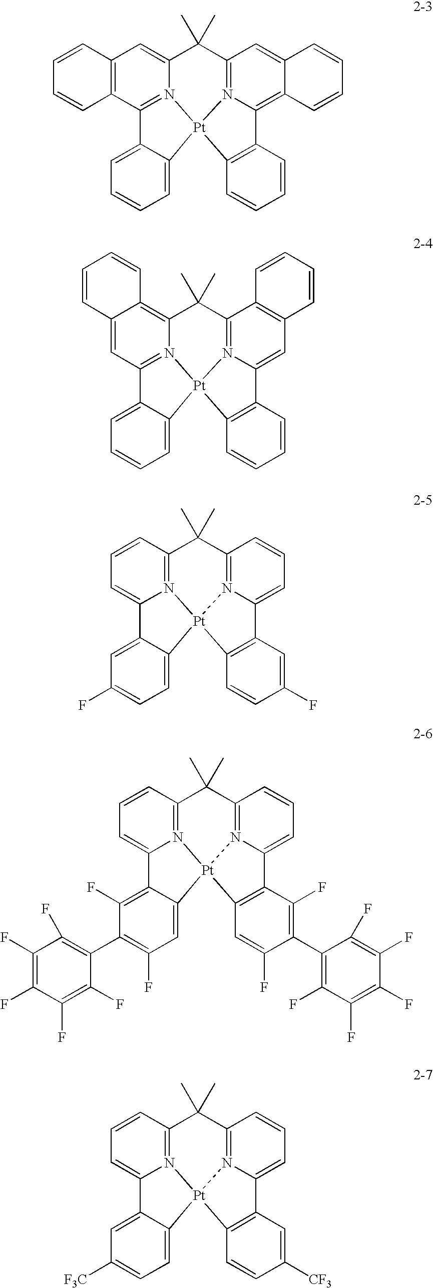

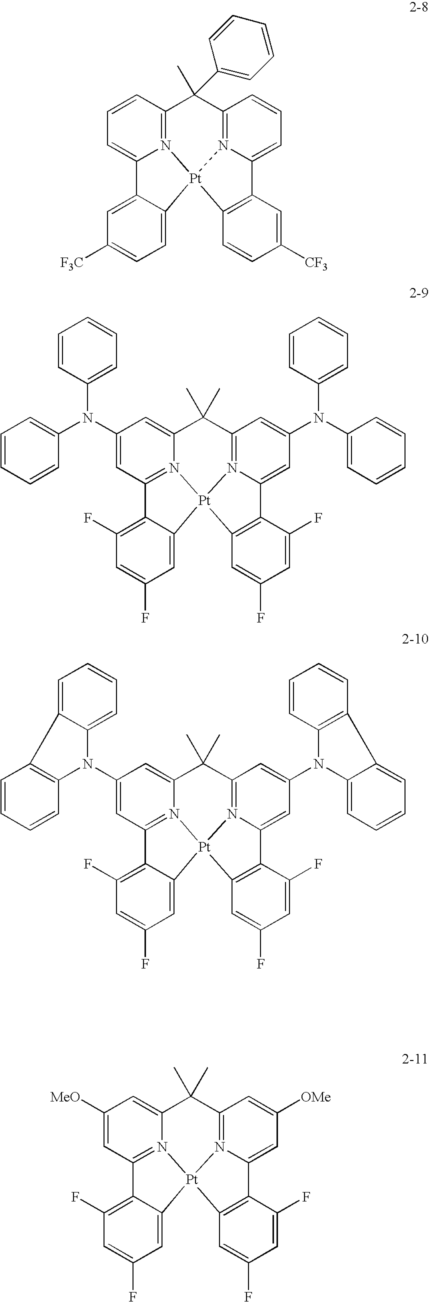

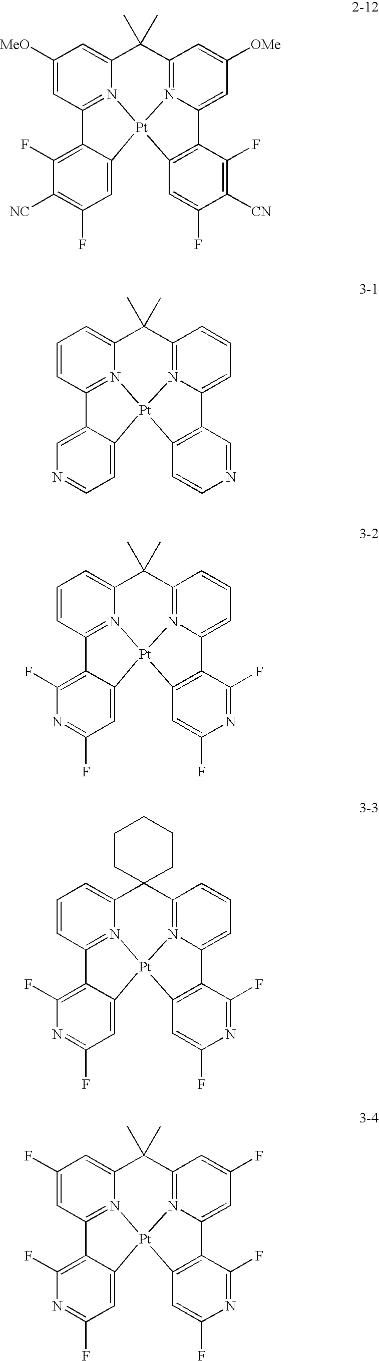

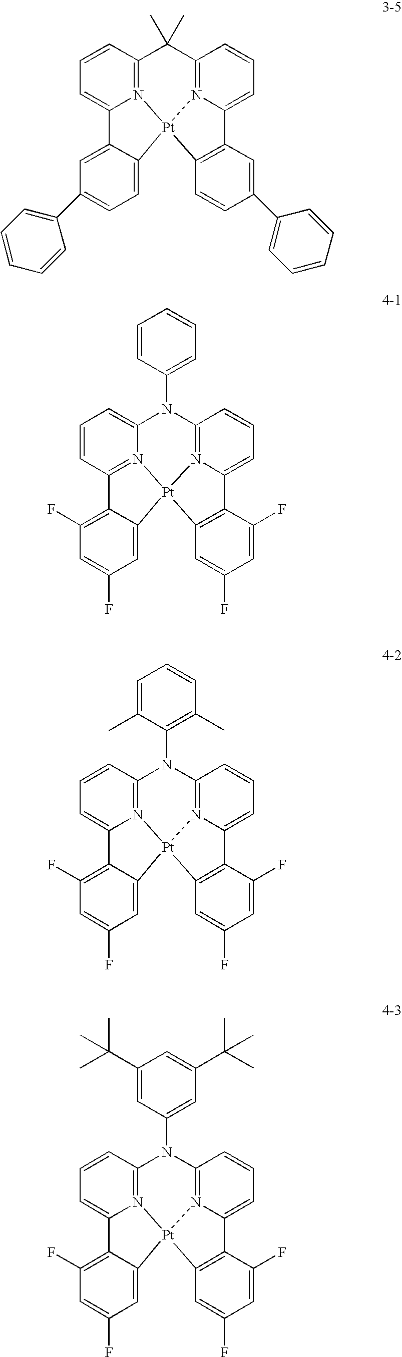

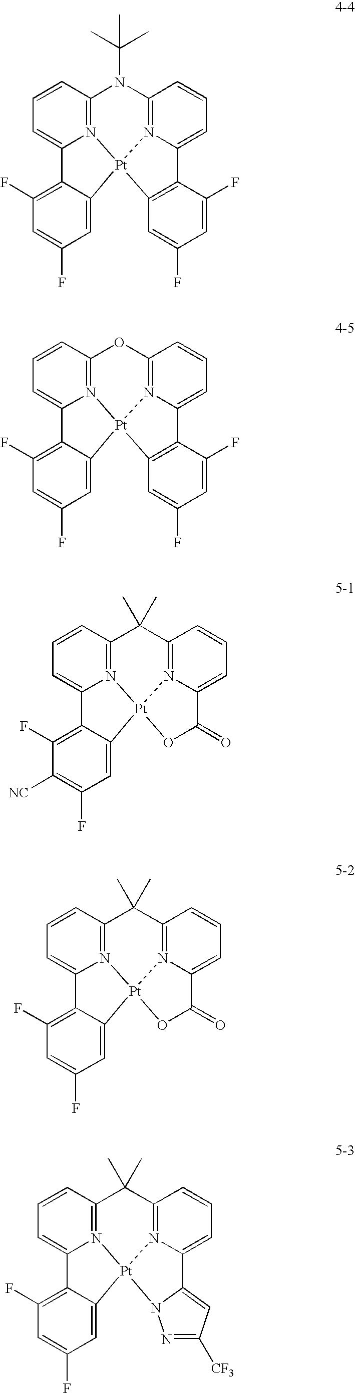

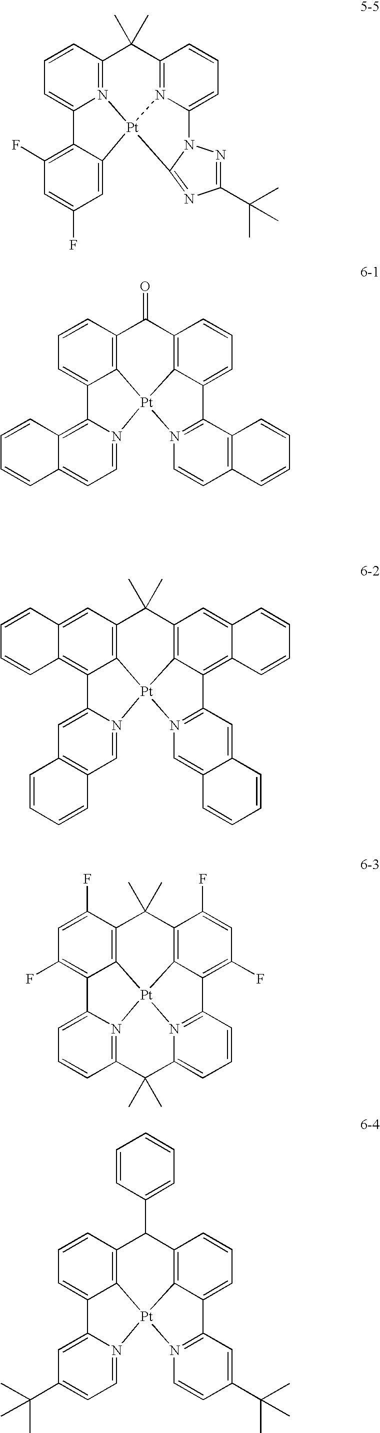

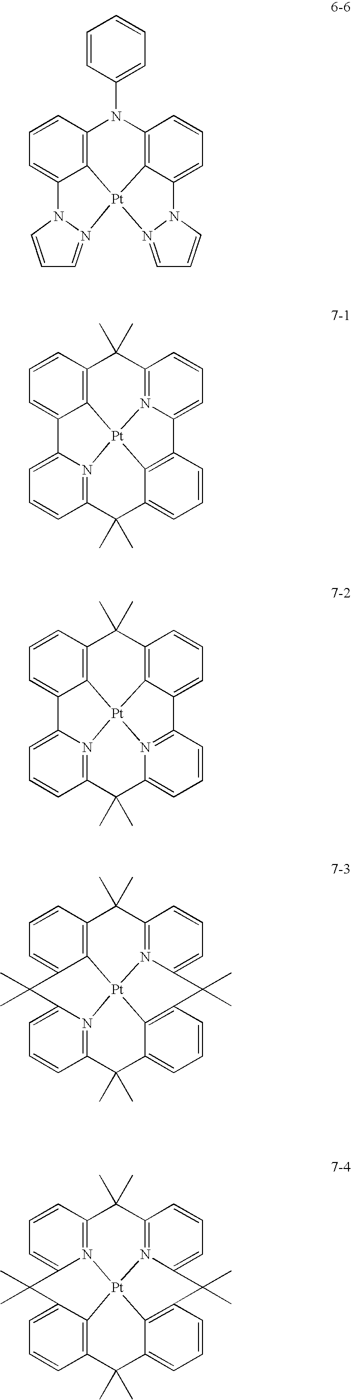

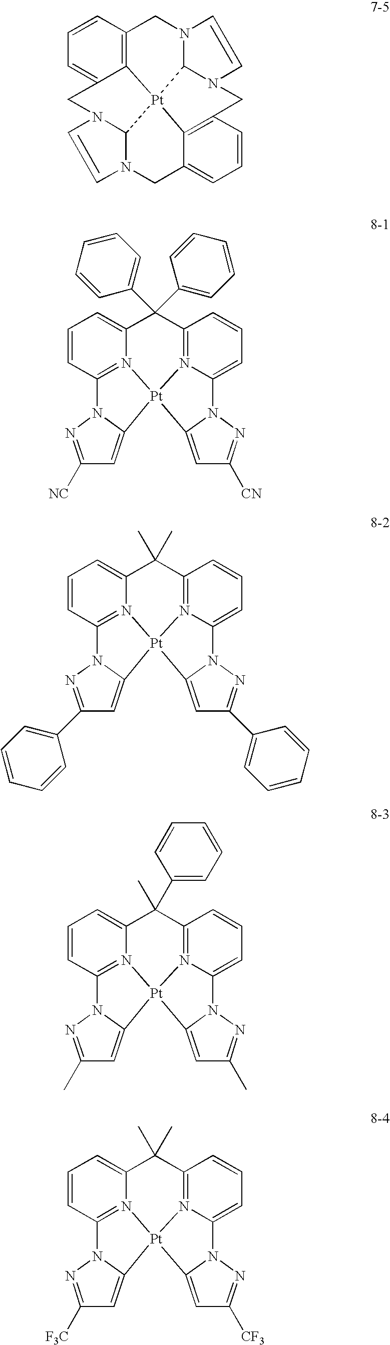

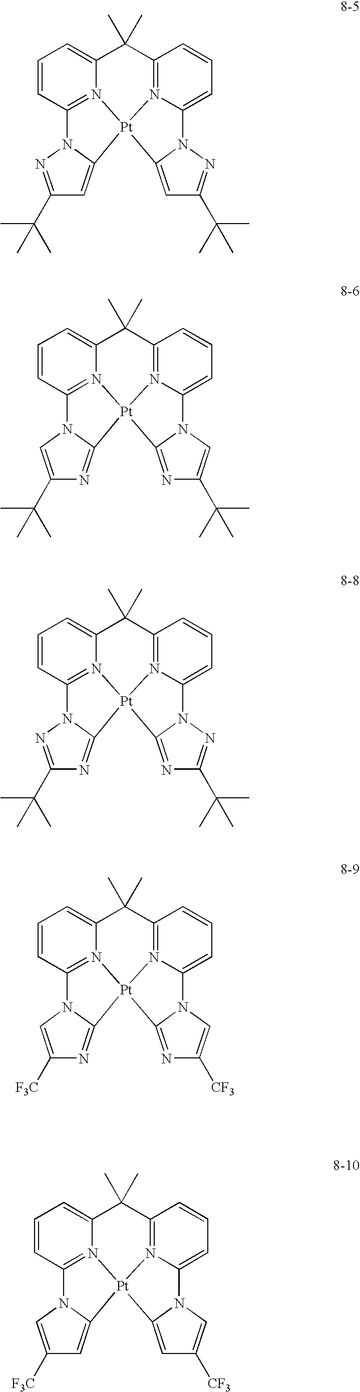

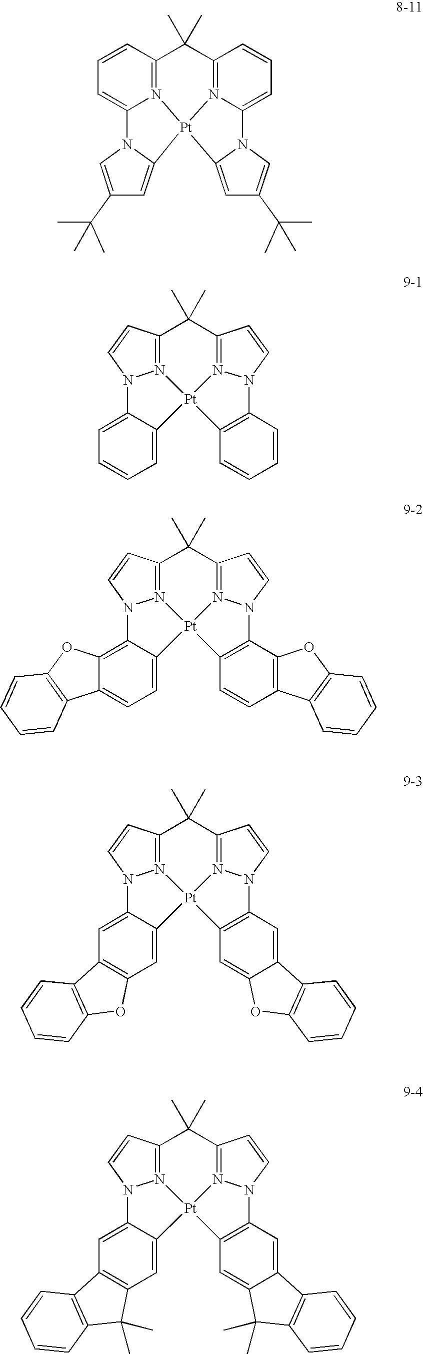

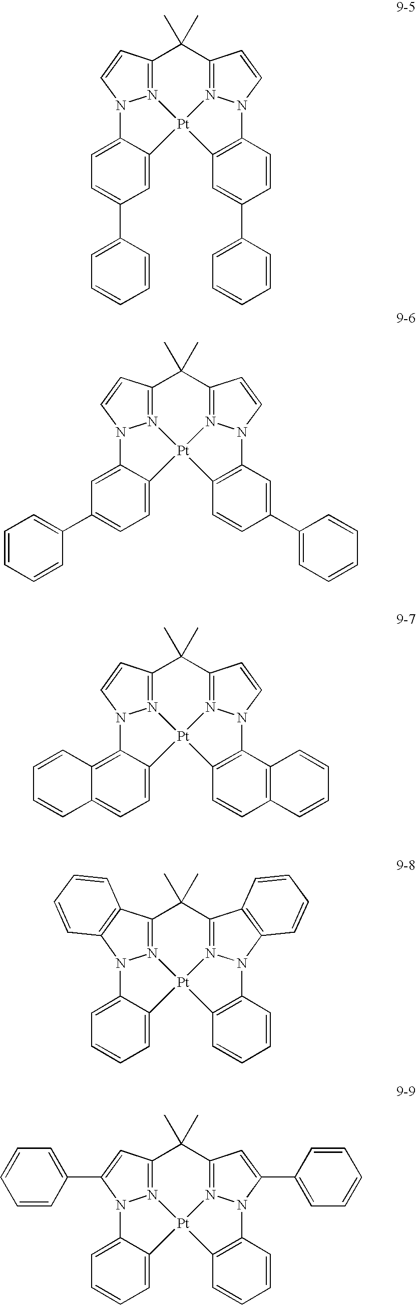

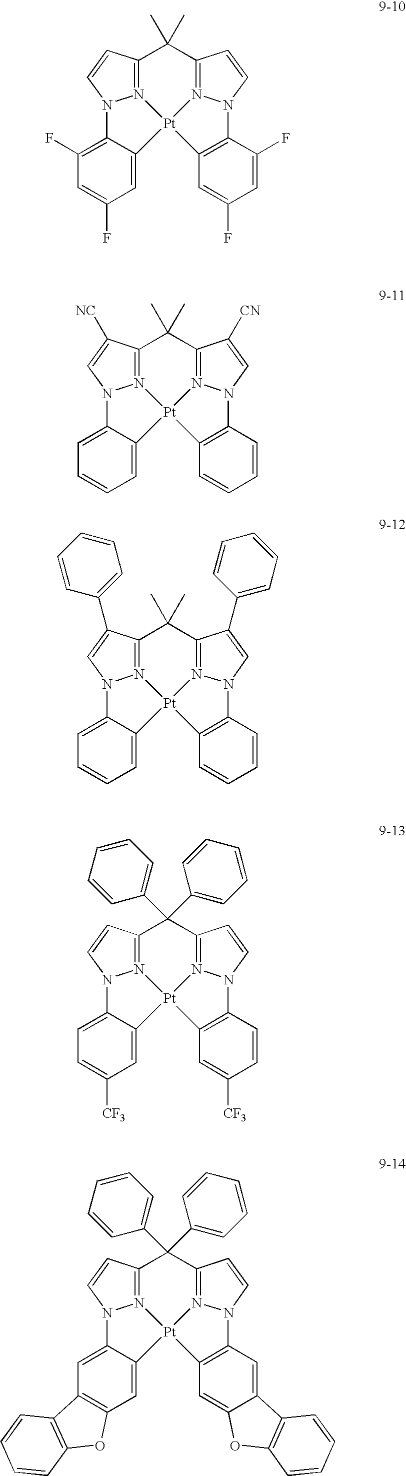

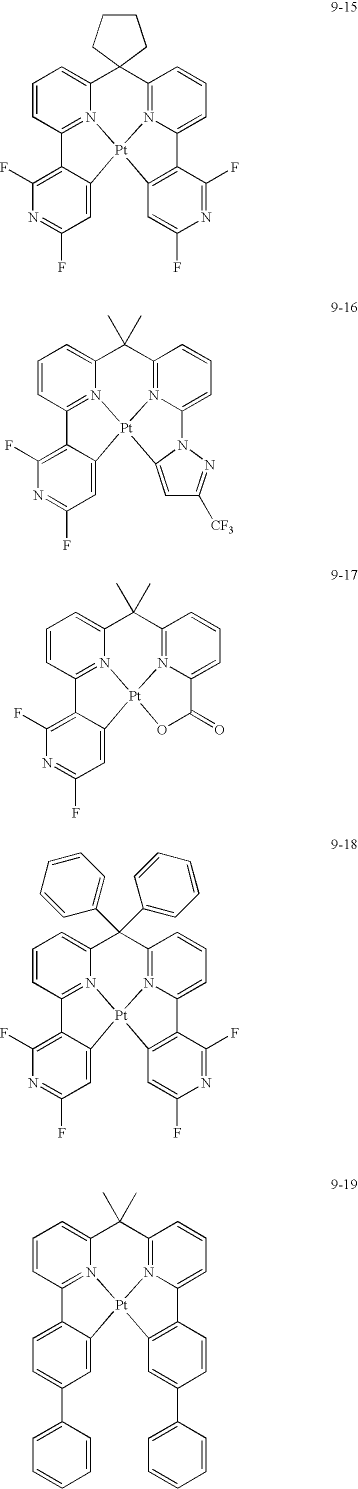

The platinum complex compound represented by the general formula (C-1) can be synthesized by various techniques, for example, a method described at page 789, left-hand column, line 53 to right-hand column, line 7, a method described at page 790, left-hand column lines 18 to 38, a method described page 790, right-hand, lines 19 to 30 and a combination thereof in Journal of Organic Chemistry, 53, 786 (1988), G. R. Newkome, et al.; a method described at page 2752, lines 26 to 35 in Chemische Berichte, 113, 2749 (1980), H. Lexy, et al.; and the like.

For example, the platinum complex compound represented by the general formula (C-1) can be obtained by treating a ligand or a dissociation material thereof and a metal compound in the absence or presence of a solvent (for example, a halogen based solvent, an alcohol based solvent, an ether based solvent, an ester based solvent, a ketone based solvent, a nitrile based solvent, an amide based solvent, a sulfone based solvent, a sulfoxide based solvent, water, etc.) and in the absence or presence of a base (various inorganic or organic bases, for example, sodium methoxide, t-butoxy potassium, triethylamine, potassium carbonate, etc.) at room temperature or a lower temperature or by heating (in addition to usual heating, a technique for achieving heating by microwaves is also effective).

A content of the compound represented by the general formula (C-1) in the light emitting layer of the invention is preferably from 1 to 30% by mass, a more preferably from 3 to 25% by mass, and further preferably from 5 to 20% by mass in the light emitting layer.

The foregoing platinum complex material is preferably a platinum complex material represented by the following general formula (5).

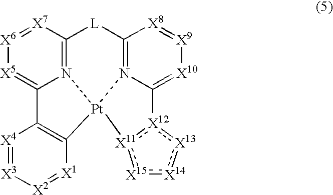

In the general formula (5), each of X¹, X², X³ and X⁴ independently represents a carbon atom or a nitrogen atom, provided that at least one of X¹, X², X³ and X⁴ represents a nitrogen atom; each of X⁵, X⁶, X⁷, X⁸, X⁹ and X¹⁰ independently represents a carbon atom or a nitrogen atom; each of X¹¹ and X¹² independently represents a carbon atom or a nitrogen atom; each of X¹³, X¹⁴ and X¹⁵ independently represents a carbon atom, a nitrogen atom, an oxygen atom or a sulfur atom; the number of nitrogen atoms contained in the 5-membered ring structure formed by X¹¹, X¹², X¹³, X¹⁴ and X¹⁵ is not more than 2; and L represents a single bond or a divalent connecting group.

Each of X¹, X², X³ and X⁴ independently represents a carbon atom or a nitrogen atom. In the case where each of X¹, X², X³ and X⁴ can be further substituted, each of them may independently have a substituent. In the case where each of X¹, X², X³ and X⁴ has a substituent, as the substituent, those exemplified above for the group A of substituents can be exemplified. Preferred examples of the substituent include an alkyl group, a perfluoroalkyl group, an aryl group, an aromatic heterocyclic group, a dialkylamino group, a diarylamino group, an alkyloxy group, a cyano group and a halogen atom. Of these, an alkyl group, a perfluoroalkyl group, an aryl group, a dialkylamino group, a cyano group and a fluorine atom are more preferable; and an alkyl group, a trifluoromethyl group and a fluorine atom are further preferable. Also, if possible, the substituents may be connected to each other to form a condensed ring structure.

At least one of X¹, X², X³ and X⁴ represents a nitrogen atom. The number of nitrogen atoms is preferably from 1 to 2, and more preferably 1.

Though the position of the nitrogen atom may be any of X¹, X², X³ and X⁴, it is preferable that X² or X³ is a nitrogen atom; and it is more preferable that X³ is a nitrogen atom.

In the general formula (5), examples of the 6-membered ring formed from two carbon atoms, X¹, X², X³ and X⁴ include a pyridine ring, a pyrazine ring, a pyrimidine ring, a pyridazine ring and a triazine ring. Of these, a pyridine ring, a pyrazine ring, a pyrimidine ring and a pyridazine ring are more preferable; and a pyridine ring is especially preferable. What the 6-membered ring formed from X¹, X², X³ and X⁴ is a pyridine ring, a pyrazine ring, a pyrimidine ring or a pyridazine ring (especially preferably a pyridine ring) is advantageous because the acidity of the hydrogen atom existing at a position at which a metal-carbon bond is formed is enhanced as compared with the case of a benzene ring, and thus, a metal complex is more easily formed.

Each of X⁵, X⁶, X⁷, X⁸, X⁹ and X¹⁰ independently represents a carbon atom or a nitrogen atom. Each of X⁵, X⁶, X⁷, X⁸, X⁹ and X¹⁰ is preferably a carbon atom.

In the case where each of X⁵, X⁶, X⁷, X⁸, X⁹ and X¹⁰ can be further substituted, each of them may independently have a substituent. In the case where each of X⁵, X⁶, X⁷, X⁸, X⁹ and X¹⁰ has a substituent, as the substituent, those exemplified above for the group A of substituents can be exemplified. Preferred examples of the substituent include an alkyl group, a perfluoroalkyl group, an aryl group, an aromatic heterocyclic group, a dialkylamino group, a diarylamino group, an alkyloxy group, a cyano group and a halogen atom. Of these, an alkyl group, a perfluoroalkyl group, an aryl group, a dialkylamino group, a cyano group and a fluorine atom are more preferable; and an alkyl group, a dialkylamino group, a trifluoromethyl group and a fluorine atom are further preferable. Also, if possible, the substituents may be connected to each other to form a condensed ring structure.

Each of X¹¹ and X¹² independently represents a carbon atom or a nitrogen atom. It is preferable that either one of X¹¹ or X¹² is a carbon atom, with the other being a nitrogen atom.

Each of X¹³, X¹⁴ and X¹⁵ independently represents a carbon atom, a nitrogen atom, an oxygen atom or a sulfur atom. Of these, a carbon atom and a nitrogen atom are preferable.

The number of nitrogen atoms contained in the 5-membered ring structure formed by X¹¹, X¹², X¹³, X¹⁴ is not more than 2 (0, 1 or 2), preferably 1 or 2, and further preferably 2.

In the case where each of X¹¹, X¹², X¹³, X¹⁴ and X¹⁵ can be further substituted, each of them may independently have a substituent. In the case where each of X¹¹, X¹², X¹³, X¹⁴ and X¹⁵ has a substituent, as the substituent, those exemplified above for the group A of substituents can be exemplified. Preferred examples of the substituent include an alkyl group, a perfluoroalkyl group, an aryl group, an aromatic heterocyclic group, a dialkylamino group, a diarylamino group, an alkyloxy group, a cyano group and a halogen atom. Of these, an alkyl group, a perfluoroalkyl group, an aryl group, a dialkylamino group, a cyano group and a fluorine atom are more preferable; and an alkyl group, a cyano group, a trifluoromethyl group and a fluorine atom are further preferable. Also, if possible, the substituents may be connected to each other to form a condensed ring structure.

The bond in the 5-membered ring structure formed by X¹¹, X¹², X¹³, X¹⁴ and X¹⁵ may be any combination of a single bond and a double bond. Examples of the 5-membered ring formed by X¹¹, X¹², X¹³X¹⁴ and X¹⁵ include a pyrrole ring, a pyrazole ring, an imidazole ring, a furan ring and a thiophene ring. Of these, a pyrrole ring, a pyrazole ring and an imidazole ring are more preferable; and a pyrrole ring and a pyrazole ring are further preferable. What the 5-membered ring formed by X¹¹, X¹², X¹³, X¹⁴ and X¹⁵ is a pyrrole ring, a pyrazole ring or an imidazole ring (further preferably a pyrrole ring or a pyrazole ring) is advantages because stability of the metal complex is enhanced.

L represents a single bond or a divalent connecting group. Examples of the divalent connecting group represented by L include an alkylene group (for example, methylene, ethylene, propylene, etc.), an arylene group (for example, phenylene, naphthalenediyl, etc.), a heteroarylene group (for example, pyridinediyl, thiophenediyl, etc.), an imino group (—NR—) (for example, a phenylimino group, etc.), an oxy group (—O—), a thio group (—S—), a phosphinidene group (—PR—) (for example, a phenylphosphinidene group, etc.), a silylene group (—SiRR′—) (for example, a dimethylsilylene group, a diphenylsilylene group, etc.) and a combination thereof These connecting groups may further have a substituent. In the case where such a connecting group has a substituent, as the substituent, those exemplified above for the group A of substituents can be exemplified.

L is preferably a single bond, an alkylene group, an arylene group, a heteroarylene group, an imino group, an oxy group, a thio group or a silylene group; more preferably a single bond, an alkylene group, an arylene group or an imino group; further preferably a single bond, an alkylene group or an arylene group; even further preferably a single bond, a methylene group or a phenylene group; even more preferably a single bond or a di-substituted methylene group; even still further preferably a single bond, a dimethylmethylene group, a diethylmethylene group, a diisobutylmethylene group, a dibenzylmethylene group, an ethylmethylmethylene group, a methylpropylmethylene group, an isobutylmethylmethylene group, a diphenylmethylene group, a methylphenylmethylene group, a cyclohexanediyl group, a cyclopentanediyl group, a fluorenediyl group or a fluoromethylmethylene group; especially preferably a single bond, a dimethylmethylene group, a diphenylmethylene group or a cyclohexanediyl group; and most preferably a dimethylmethylene group or a diphenylmethylene group.

Specific examples of the divalent connecting group are given below, but it should not be construed that the invention is limited thereto.

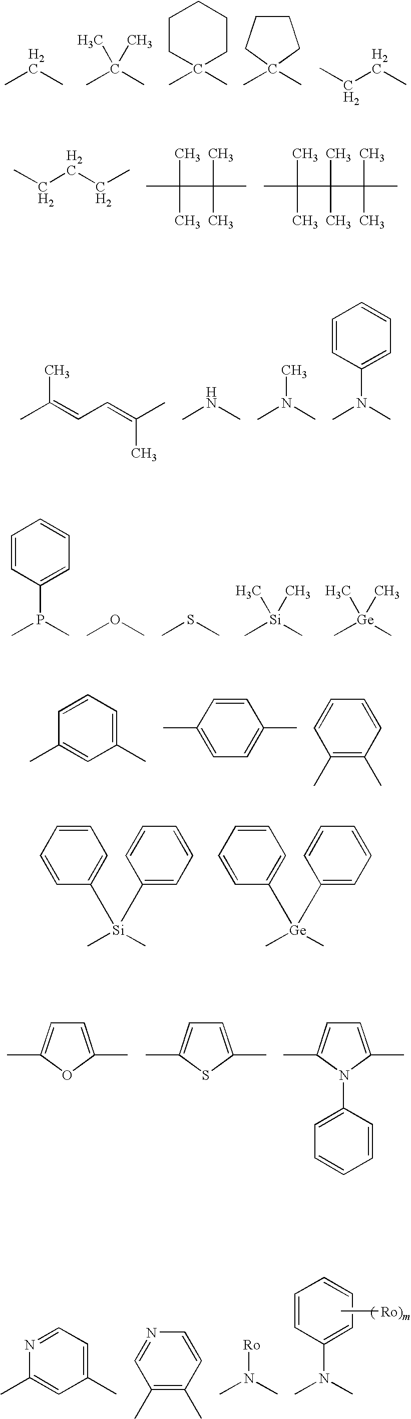

In the foregoing general formulae, Ro represents a substituent selected from the foregoing group A of substituents. Ro is preferably an alkyl group, and more preferably an alkyl group having from 1 to 6 carbon atoms. m represents an integer of from 1 to 5. m is preferably from 2 to 5, and more preferably from 2 to 3.

The platinum complex represented by the general formula (5) is preferably a platinum complex represented by the following general formula (6).

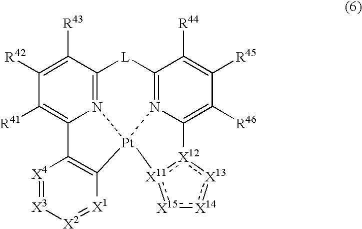

In the foregoing general formula, each of X¹, X², X³ and X⁴ independently represents a carbon atom or a nitrogen atom, provided that at least one of X¹, X², X³ and X⁴ represents a nitrogen atom; each of R⁴¹, R⁴², R⁴³, R⁴⁴, R⁴⁵ and R⁴⁶ independently represents a hydrogen atom or a substituent; each of X¹¹ and X¹² independently represents a carbon atom or a nitrogen atom; each of X¹³, X¹⁴ and X¹⁵ independently represents a carbon atom, a nitrogen atom, an oxygen atom or a sulfur atom; the number of nitrogen atoms contained in the 5-membered ring structure formed by X¹¹, X¹², X¹³, X¹⁴ and X¹⁵ is not more than 2; and L represents a single bond or a divalent connecting group.

In the general formula (6), X¹, X², X³, X⁴, X¹¹, X¹², X¹³, X¹⁴, X¹⁵ and L are synonymous with X¹, X², X³, X⁴, X¹¹, X¹², X¹³, X¹⁴, X¹⁵ and L in the foregoing general formula (5), respectively, and preferred ranges thereof are also the same.

In the general formula (6), each of R⁴¹, R⁴², R⁴³, R⁴⁴, R⁴⁵ and R⁴⁶ independently represents a hydrogen atom or a substituent. The substituent represented by each of R⁴¹, R⁴², R⁴³, R⁴⁴, R⁴⁵ and R⁴⁶ synonymous with the group A of substituents. If possible, R⁴¹, R⁴², R⁴³, R⁴⁴, R⁴⁵ and R⁴⁶ may be bonded to each other to form a ring.

Each of R⁴¹ and R⁴⁶ is preferably a hydrogen atom, an alkyl group, an aryl group, an amino group, an alkoxy group, an aryloxy group, an acyl group, an alkoxycarbonyl group, an alkylthio group, a sulfonyl group, a hydroxyl group, a halogen atom, a cyano group, a nitro group or a heterocyclic group; more preferably a hydrogen atom, an alkyl group, an aryl group, a halogen atom, a cyano group or a heterocyclic group; further preferably a hydrogen atom, a methyl group, a t-butyl group, a trifluoromethyl group, a phenyl group, a fluorine atom, a cyano group or a pyridyl group; even further preferably a hydrogen atom, a methyl group or a fluorine atom; and especially preferably a hydrogen atom.

R⁴³ and R⁴⁴ are preferably synonymous with R⁴¹ and R⁴⁶ with respect to the preferred ranges thereof.

Each of R⁴² and R⁴⁵ is preferably a hydrogen atom, an alkyl group, an aryl group, an amino group, an alkoxy group, an aryloxy group, an alkylthio group, an arylthio group, a halogen atom, a cyano group or a heterocyclic group; more preferably a hydrogen atom, an alkyl group, an aryl group, an amino group, an alkoxy group, an aryloxy group, a halogen atom or a heterocyclic group; further preferably a hydrogen atom, an alkyl group, an amino group, an alkoxy group, a halogen atom or a heterocyclic group; even further preferably a hydrogen atom, a methyl group, a t-butyl group, a dialkylamino group, a diphenylamino group, a methoxy group, a phenoxy group, a fluorine atom, an imidazolyl group, a pyrrolyl group or a carbazolyl group; especially preferably a hydrogen atom, a fluorine atom or a methyl group; and most preferably a hydrogen atom.

One of preferred embodiments of the platinum complex represented by the general formula (6) is a platinum complex represented by the following general formula (6a-1).

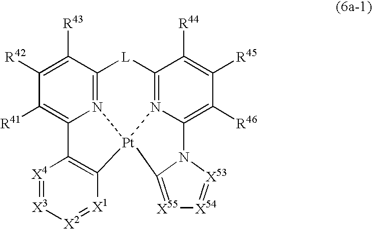

In the foregoing general formula, each of X¹, X², X³ and X⁴ independently represents a carbon atom or a nitrogen atom, provided that at least one of X¹, X², X³ and X⁴ represents a nitrogen atom; each of R⁴¹, R⁴², R⁴³, R⁴⁴, R⁴⁵ and R⁴⁶ independently represents a hydrogen atom or a substituent; each of X⁵³, X⁵⁴ and X⁵⁵ independently represents a carbon atom or a nitrogen atom; the number of nitrogen atoms included in the 5-membered ring structure containing X⁵³, X⁵⁴ and X⁵⁵ is 1 or 2; and L represents a single bond or a divalent connecting group.

In the general formula (6a-1), X¹, X², X³, X⁴, R⁴¹, R⁴², R⁴³, R⁴⁴, R⁴⁵, R⁴⁶ and L are synonymous with X¹, X², X³, X⁴, R⁴¹, R⁴², R⁴³, R⁴⁴, R⁴⁵, R⁴⁶ and L in the foregoing general formula (6), respectively, and preferred ranges thereof are also the same.

Each of X⁵³, X⁵⁴ and X⁵⁵ independently represents a carbon atom or a nitrogen atom. In the case where each of X⁵³, X⁵⁴ and X⁵⁵ can be further substituted, each of them may independently have a substituent. In the case where each of X⁵³, X⁵⁴ and X⁵⁵ has a substituent, as the substituent, those exemplified above for the group A of substituents can be exemplified. Preferred examples of the substituent include an alkyl group, a perfluoroalkyl group, an aryl group, an aromatic heterocyclic group, a dialkylamino group, a diarylamino group, an alkyloxy group, a cyano group and a halogen atom. Of these, an alkyl group, a perfluoroalkyl group, an aryl group, a dialkylamino group, a cyano group and a fluorine atom are more preferable; and an alkyl group, a trifluoromethyl group and a fluorine atom are further preferable. Also, if possible, the substituents may be connected to each other to form a condensed ring structure.

In the general formula (6a-1), the number of nitrogen atoms contained in the 5-membered ring structure which is formed by a carbon atom, a nitrogen atom, X⁵³, X⁵⁴ and X⁵⁵ is 1 or 2, and preferably 2.

Examples of the 5-membered ring which is formed by a carbon atom, a nitrogen atom, X⁵³, X⁵⁴ and X⁵⁵ include a pyrrole ring, a pyrazole ring and an imidazole ring. Of these, a pyrrole ring and a pyrazole ring are more preferable; and a pyrazole ring is the most preferable.

The platinum complex represented by the general formula (6a-1) is preferably a platinum complex represented by the following general formula (6a-2).

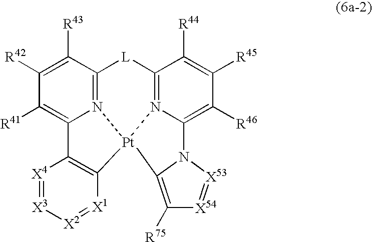

In the foregoing general formula, each of X¹, X², X³ and X⁴ independently represents a carbon atom or a nitrogen atom, provided that at least one of X¹, X², X³ and X⁴ represents a nitrogen atom; each of R⁴¹, R⁴², R⁴³, R⁴⁴, R⁴⁵ and R⁴⁶ independently represents a hydrogen atom or a substituent; each of X⁵³ and X⁵⁴ independently represents a carbon atom or a nitrogen atom; the number of nitrogen atoms contained in the 5-membered ring structure containing X⁵³ and X⁵⁴ is 1 or 2; R⁷⁵ represents a hydrogen atom or a substituent; and L represents a single bond or a divalent connecting group.

In the general formula (6a-2), X¹, X², X³, X⁴, X⁵³, X⁵⁴, R⁴¹, R⁴², R⁴³, R⁴⁴, R⁴⁵, R⁴⁶ and L are synonymous with X¹, X², X³, X⁴, X⁵³, X⁵⁴, R⁴¹, R⁴², R⁴³, R⁴⁴, R⁴⁵, R⁴⁶ and L in the foregoing general formula (6a-1), respectively, and preferred ranges thereof are also the same.

R⁷⁵ represents a hydrogen atom or a substituent. As the substituent, those exemplified above for the group A of substituents can be exemplified. R⁷⁵ is preferably a hydrogen atom, an alkyl group, a perfluoroalkyl group, an aryl group, an aromatic heterocyclic group, a dialkylamino group, a diarylamino group, an alkyloxy group, a cyano group or a halogen atom; more preferably a hydrogen atom, an alkyl group, a perfluoroalkyl group, an aryl group, a dialkylamino group, a cyano group or a fluorine atom; further preferably a hydrogen atom, an alkyl group, a trifluoromethyl group, a cyano group or a fluorine atom; and most preferably a cyano group, a fluorine atom or a hydrogen atom. Also, if possible, R⁷⁵ may be connected to the substituent of X⁵⁴ or X⁵³ to form a condensed ring structure.

The platinum complex represented by the general formula (6a-2) is preferably a platinum complex represented by the following general formula (6a-3).

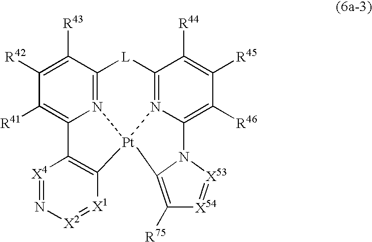

In the foregoing general formula, each of X¹, X² and X⁴ independently represents a carbon atom or a nitrogen atom; each of R⁴¹, R⁴², R⁴³, R⁴⁴, R⁴⁵ and R⁴⁶ independently represents a hydrogen atom or a substituent; each of X⁵³ and X⁵⁴ independently represents a carbon atom or a nitrogen atom; the number of nitrogen atoms contained in the 5-membered ring structure containing X⁵³ and X⁵⁴ is 1 or 2; R⁷⁵ represents a hydrogen atom or a substituent; and L represents a single bond or a divalent connecting group.

In the general formula (6a-3), X¹, X², X⁴, X⁵³, X⁵⁴, R⁴¹, R⁴², R⁴³, R⁴⁴, R⁴⁵, R⁴⁶, R⁷⁵ and L are synonymous with X¹, X², X⁴, X⁵³, X⁵⁴, R⁴¹, R⁴², R⁴³, R⁴⁴, R⁴⁵, R⁴⁶, R⁷⁵ and L in the foregoing general formula (6a-2), respectively, and preferred ranges thereof are also the same.

In the general formula (6a-3), the number of nitrogen atoms contained in the 6-membered ring structure which is formed by X¹, X², a nitrogen atom, X⁴, a carbon atom and a carbon atom is preferably 1 or more and not more than 3, more preferably 1 or 2, and further preferably 1. Specific examples of the 6-membered ring include a pyridine ring, a pyrazine ring, a pyrimidine ring, a pyridazine ring and a triazine ring. Of these, a pyridine ring, a pyrazine ring, a pyrimidine ring and a pyridazine ring are preferable; a pyridine ring, a pyrazine ring and a pyrimidine ring are more preferable; and a pyridine ring is especially preferable.

The platinum complex represented by the general formula (6a-3) is preferably a platinum complex represented by the following general formula (6a-4). This platinum complex represented by the general formula (6a-4) is a novel compound.

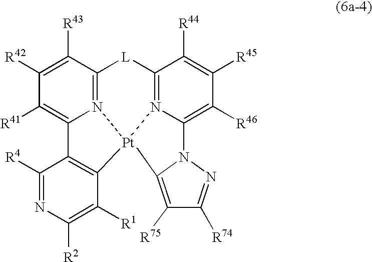

In the foregoing general formula, each of R¹, R², R⁴, R⁴¹, R⁴², R⁴³, R⁴⁵, R⁴⁶, R⁷⁴ and R⁷⁵ independently represents a hydrogen atom or a substituent; and L represents a single bond or a divalent connecting group.

In the general formula (6a-4), R⁴¹, R⁴², R⁴³, R⁴⁴, R⁴⁵, R⁴⁶, R⁷⁵ and L are synonymous with R⁴¹, R⁴², R⁴³, R⁴⁴, R⁴⁵, R⁴⁶, R⁷⁵ and L in the foregoing general formula (6a-3), respectively, and preferred ranges thereof are also the same.

Each of R¹, R², R⁴ and R⁷⁴ independently represents a hydrogen atom or a substituent. As the substituent, those exemplified above for the group A of substituents can be exemplified. Also, if possible, in R⁴ and R⁴¹, and. R¹ and R², the substituents may be connected to each other to form a condensed ring structure. The substituents of R¹ and R⁷⁵ may be connected to each other to form a cyclic structure as the whole of the ligand.

R¹ is preferably a hydrogen atom, an alkyl group, an aryl group, an amino group, an alkoxy group, an aryloxy group, an acyl group, an alkoxycarbonyl group, an alkylthio group, a sulfonyl group, a hydroxyl group, a halogen atom, a cyano group, a nitro group or a heterocyclic group; more preferably a hydrogen atom, an alkyl group, an aryl group, an alkoxy group, an aryloxy group, an alkylthio group, a halogen atom or a cyano group; further preferably a hydrogen atom, an alkyl group, a perfluoroalkyl group, an aryl group, a halogen atom or a cyano group; even further preferably a hydrogen atom, a methyl group, a trifluoromethyl group or a cyano group; and especially preferably a hydrogen atom, a trifluoromethyl group, a fluorine atom or a cyano group.

Each of R² and R⁴ is preferably a hydrogen atom, a halogen atom, a fluorine atom-substituted phenyl group, a fluorine-substituted alkoxy group, a perfluoroalkyl group, a cyano group, a nitro group or an aryloxy group; more preferably a hydrogen atom, a fluorine atom, a fluorine atom-substituted phenyl group, a trifluoromethoxy group, a trifluoromethyl group, a cyano group or a phenoxy group; further preferably a hydrogen atom, a fluorine atom, a perfluorophenyl group, a trifluoromethyl group, a cyano group or an electron withdrawing substituent-substituted phenoxy group; especially preferably a hydrogen atom or a fluorine atom; and most preferably a fluorine atom.

R⁷⁴ is preferably a hydrogen atom, an alkyl group, an aryl group, an amino group, an alkoxy group, an aryloxy group, an acyl group, an alkoxycarbonyl group, an alkylthio group, a sulfonyl group, a hydroxyl group, a halogen atom, a cyano group, a nitro group or a heterocyclic group; more preferably a hydrogen atom, an alkyl group, an aryl group, an alkoxy group, an aryloxy group, an alkylthio group, a halogen atom or a cyano group; further preferably a hydrogen atom, an alkyl group, a perfluoroalkyl group, an aryl group, a halogen atom or a cyano group; even further preferably a hydrogen atom, a methyl group, a trifluoromethyl group or a cyano group; especially preferably a hydrogen atom, a trifluoromethyl group, a fluorine atom or a cyano group; and most preferably a trifluoromethyl group or a cyano group.

The platinum complex represented by the general formula (6a-4) can be used as, in addition to various materials to be used for organic EL devices, light emitting materials which can be suitably used in the fields including display devices, displays, backlights, electro-photographs, illumination light sources, recording light sources, exposure light sources, read light sources, markers, signboards, interiors and so on; medical applications; fluorescent brighteners; photographic materials, UV absorbing materials, laser coloring matters, materials for recording media, pigments for inkjet, dyes for color filters, color conversion filters, analysis applications, materials for solar cells, materials for organic thin film transistors and so on.

Next, a compound represented by the following general formula (6a-4′) is described. The compound represented by the general formula (6a-4′) is a novel compound which can be a ligand of the platinum complex represented by the foregoing general formula (6a-4).

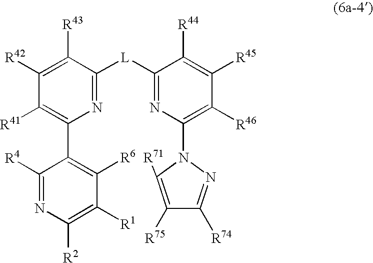

In the foregoing general formula, each of R¹, R², R⁴, R⁶, R⁴¹, R⁴², R⁴³, R⁴⁴, R⁴⁵, R⁴⁶, R⁷¹, R⁷⁴ and R⁷⁵ independently represents a hydrogen atom or a substituent; and L represents a single bond or a divalent connecting group.

In the general formula (6a-4′) R¹, R², R⁴, R⁴¹, R⁴², R⁴³, R⁴⁴, R⁴⁵, R⁴⁶, R⁷⁴, R⁷⁵ and L are synonymous with R¹, R², R⁴, R⁴¹, R⁴², R⁴³, R⁴⁴, R⁴⁵, R⁴⁶, R⁷⁴, R⁷⁵ and L in the foregoing general formula (6a-4), respectively, and preferred ranges thereof are also the same. Each of R⁶ and R⁷¹ independently represents a hydrogen atom a substituent. As the substituent, those exemplified above for the group A of substituents can be exemplified. Each of R⁶ and R⁷¹ is preferably a halogen atom or a hydrogen atom, and more preferably a hydrogen atom.

The compound represented by the general formula (6a-4′) can be utilized as, in addition to the ligand of the metal complex represented by the foregoing general formula, fluorescent materials, charge transport materials, intermediates of drugs, pesticides, etc. and so on.

Another preferred embodiment of the platinum complex represented by the general formula (6) is a platinum complex represented by the following general formula (6b-1).

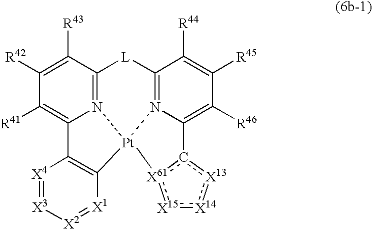

In the foregoing general formula, each of X¹, X², X³ and X⁴ independently represents a carbon atom or a nitrogen atom, provided that at least one of X¹, X², X³ and X⁴ represents a nitrogen atom; each of R⁴¹, R⁴², R⁴³, R⁴⁴, R⁴⁵ and R⁴⁶ independently represents a hydrogen atom or a substituent; X⁶¹ represents a carbon atom or a nitrogen atom; each of X¹³, X¹⁴ and X¹⁵ independently represents a carbon atom, a nitrogen atom, an oxygen atom or a sulfur atom; the number of nitrogen atoms contained in a 5-membered ring structure represented by X⁶¹, a carbon atom, X¹³, X¹⁴ and X¹⁵ is not more than 2; and L represents a single bond or a divalent connecting group.

In the general formula (6b-1), X¹, X², X³, X⁴, X¹³, X¹⁴, X¹⁵, R⁴¹, R⁴², R⁴³, R⁴⁴, R⁴⁵, R⁴⁶ and L are synonymous with X¹, X², X³, X⁴, X¹³, X¹⁴, X¹⁵, R⁴¹, R⁴², R⁴³, R⁴⁴, R⁴⁵, R⁴⁶ and L in the general formula (6), respectively, and preferred ranges thereof are also the same.

X⁶¹ represents a carbon atom or a nitrogen atom, and preferably a nitrogen atom.

In the general formula (6b-1), the number of nitrogen atoms contained in the 5-membered ring structure formed by X⁶¹, a carbon atom, X¹³, X¹⁴ and X¹⁵ is 0, 1 or 2, preferably 1 or 2, and more preferably 2.

The bond in the 5-membered ring structure formed by X⁶¹, a carbon atom, X¹³, X¹⁴ and X¹⁵ may be any combination of a single bond and a double bond. Examples of the 5-membered ring formed by X⁶¹, a carbon atom, X¹³, X¹⁴ and X¹⁵ include a pyrrole ring, a pyrazole ring, an imidazole ring, a furan ring and a thiophene ring. Of these, a pyrrole ring, a pyrazole ring and an imidazole ring are more preferable; and a pyrazole ring is further preferable.

The platinum complex represented by the general formula (6b-1) is preferably a platinum complex represented by the following general formula (6b-2).

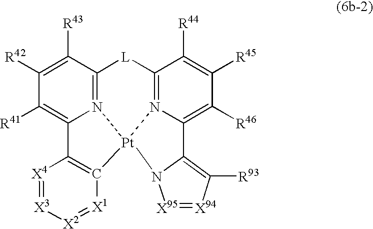

In the foregoing general formula, each of X¹, X², X³ and X⁴ independently represents a carbon atom or a nitrogen atom, provided that at least one of X¹, X², X³ and X⁴ represents a nitrogen atom; each of R⁴¹, R⁴², R⁴³, R⁴⁴, R⁴⁵ and R⁴⁶ independently represents a hydrogen atom or a substituent; each of X⁹⁴ and X⁹⁵ independently represents a carbon atom or a nitrogen atom; at least one of X⁹⁴ and X⁹⁵ represents a carbon atom; R⁹³ represents a hydrogen atom or a substituent; and L represents a single bond or a divalent connecting group.

In the general formula (6b-2), X¹, X², X³, X⁴, R⁴¹, R⁴², R⁴³, R⁴⁴, R⁴⁵, R⁴⁶ and L are synonymous with X¹, X², X³, X⁴, R⁴¹, R⁴², R⁴³, R⁴⁴, R⁴⁵, R⁴⁶ and L in the general formula (6b-1), respectively, and preferred ranges thereof are also the same.

Each of X⁹⁴ and X⁹⁵ independently represents a carbon atom or a nitrogen atom, provided that either one of X⁹⁴ or X⁹⁵ represents a carbon atom. It is preferable that X⁹⁴ represents a carbon atom, whereas X⁹⁵ represents a nitrogen atom.

In the case where each of X⁹⁴ and X⁹⁵ can be further substituted, each of X⁹⁴ and X⁹⁵ may independently have a substituent. In the case where each of X⁹⁴ and X⁹⁵ has a substituent, examples of the substituent include those represented by the foregoing group A of substituents. As the substituent, an alkyl group, a perfluoroalkyl group, an aryl group, an aromatic heterocyclic group, a dialkylamino group, a diarylamino group, an alkyloxy group, a cyano group and a halogen atom are preferable; an alkyl group, a perfluoroalkyl group, an aryl group, a dialkylamino group, a cyano group and a fluorine atom are more preferable; and an alkyl group, a trifluoromethyl group and a fluorine atom are further preferable. Also, if possible, the substituents may be connected to each other to form a condensed ring structure.

In the general formula (6b-2), examples of the 5-membered ring formed by a nitrogen atom, a carbon atom, a carbon atom, X⁹⁴ and X⁹⁵ include a pyrrole ring, a pyrazole ring and an imidazole ring. Of these, a pyrazole ring and an imidazole ring are more preferable; and a pyrazole ring is further preferable.

R⁹³ represents a hydrogen atom or a substituent. Examples of the substituent include those represented by the foregoing group A of substituents. R⁹³ is preferably a hydrogen atom, an alkyl group, a perfluoroalkyl group, an aryl group, an aromatic heterocyclic group, a dialkylamino group, a diarylamino group, an alkyloxy group, a cyano group or a halogen atom; more preferably a hydrogen atom, an alkyl group, a perfluoroalkyl group, an aryl group, a dialkylamino group, a cyano group or a fluorine atom; further preferably a hydrogen atom, an alkyl group, a trifluoromethyl group or a fluorine atom; and most preferably a fluorine atom or a hydrogen atom. Also, if possible, the substituents of X⁹⁴ and X⁹⁵ may be connected to each other to form a condensed ring structure.

The platinum complex represented by the general formula (6b-2) is preferably a platinum complex represented by the general formula (6b-3).

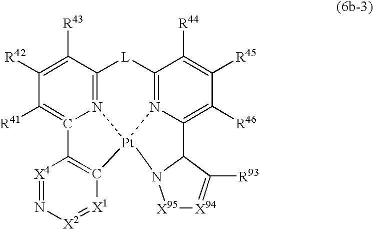

In the foregoing general formula, each of X¹, X² and X⁴ independently represents a carbon atom or a nitrogen atom, provided that at least one of X¹, X² and X⁴ represents a nitrogen atom; each of R⁴¹, R⁴², R⁴³, R⁴⁴, R⁴⁵ and R⁴⁶ independently represents a hydrogen atom or a substituent; each of X⁹⁴ and X⁹⁵ independently represents a carbon atom or a nitrogen atom; at least one of X⁹⁴ and X⁹⁵ represents a carbon atom; R⁹³ represents a hydrogen atom or a substituent; and L represents a single bond or a divalent connecting group.

In the general formula (6b-3), X¹, X², X⁴, X⁹⁴, X⁹⁵, R⁴¹, R⁴², R⁴³, R⁴⁴, R⁴⁵, R⁴⁶, R⁹³ and L are synonymous with X¹, X², X⁴, X⁹⁴, X⁹⁵, R⁴¹, R⁴², R⁴³, R⁴⁴, R⁴⁵, R⁴⁶, R⁹³ and L in the general formula (6b-2), respectively, and preferred ranges thereof are also the same.

In the general formula (6b-3), the number of nitrogen atoms contained in the 6-membered ring structure formed by X¹, X², a nitrogen atom, X⁴, a carbon atom and a carbon atom is preferably 1 or more and not more than 3, more preferably 1 or 2, and further preferably 1. Specific examples of the 6-membered ring include a pyridine ring, a pyrazine ring, a pyrimidine ring, a pyridazine group and a triazine ring. Of these, a pyridine ring, a pyrazine ring, a pyrimidine ring and a pyridazine ring are more preferable; a pyridine ring, a pyrazine ring and a pyrimidine ring are further preferable; and a pyridine ring is especially preferable.

The foregoing metal complex with a specified structure may be a low-molecular weight compound, or may be a high-molecular weight compound in which a residue structure is connected to a polymer principal chain (preferably a high-molecular weight compound having a mass average molecular weight of from 1,000 to 5,000,000, more preferably a high-molecular weight compound having a mass average molecular weight of from 5,000 to 2,000,000, and further preferably a high-molecular weight compound having a mass average molecular weight of from 10,000 to 1,000,000) or a high-molecular weight compound having a structure of the foregoing metal complex with a specified structure in a principal chain thereof (preferably a high-molecular weight compound having a mass average molecular weight of from 1,000 to 5,000,000, more preferably a high-molecular weight compound having a mass average molecular weight of from 5,000 to 2,000,000, and further preferably a high-molecular weight compound having a mass average molecular weight of from 10,000 to 1,000,000). The metal complex with a specified structure is preferably a low-molecular weight compound.

In the case of a high-molecular weight compound, the high-molecular weight compound may be a homopolymer or may be a copolymer with other polymer. In the case of a copolymer, the copolymer may be a random copolymer or may be a block copolymer. Furthermore, in the case of a copolymer, a compound having a light emitting function and/or a compound having a charge transport function may be present within the polymer.

Preferred specific examples of the metal complex represented by the general formula (5) are given below, but it should not be construed that the invention is limited thereto.

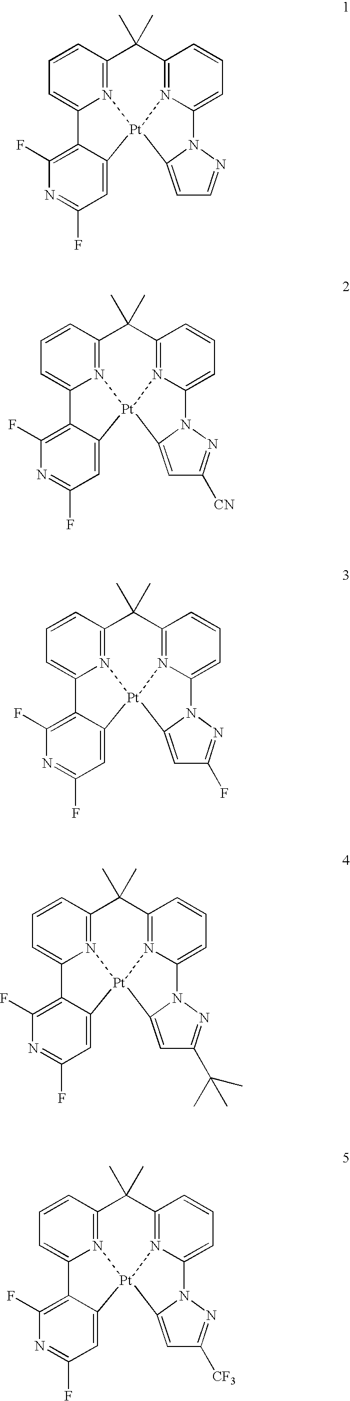

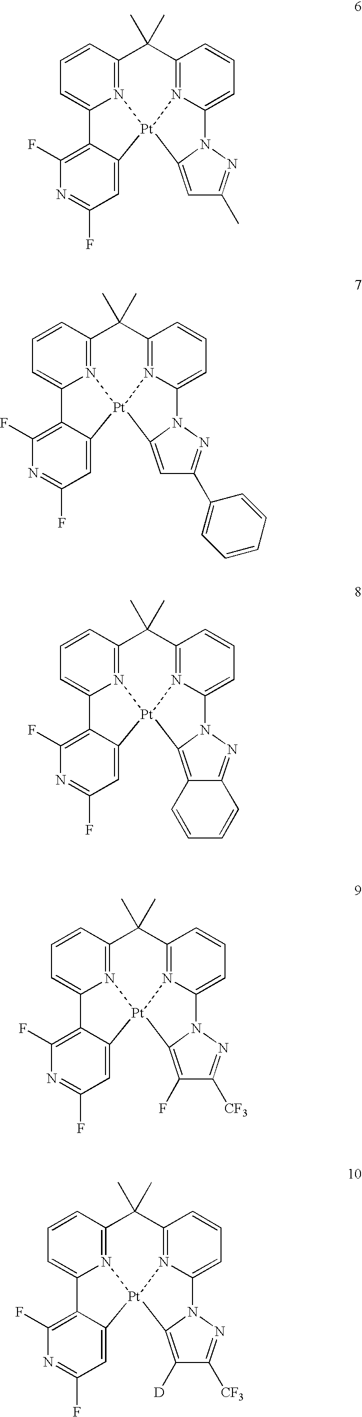

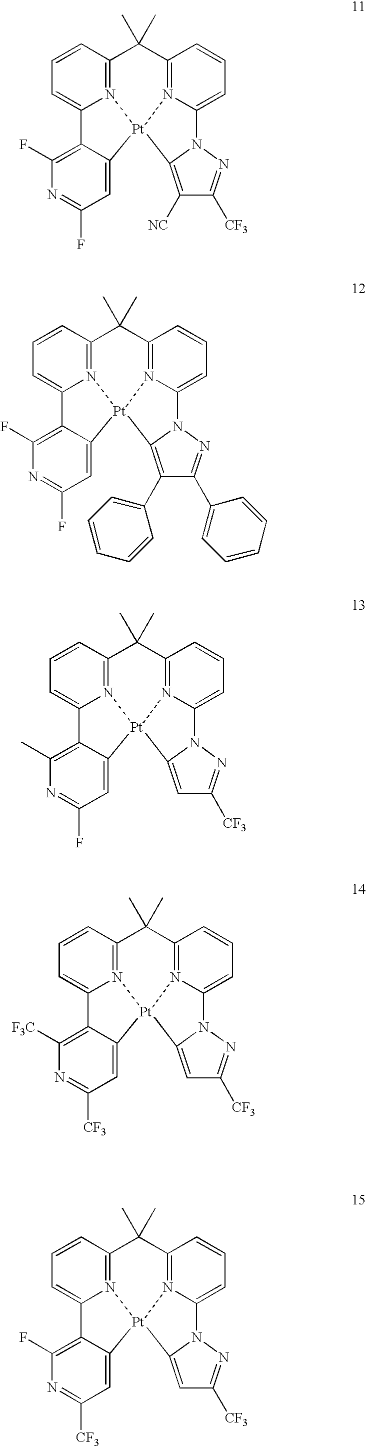

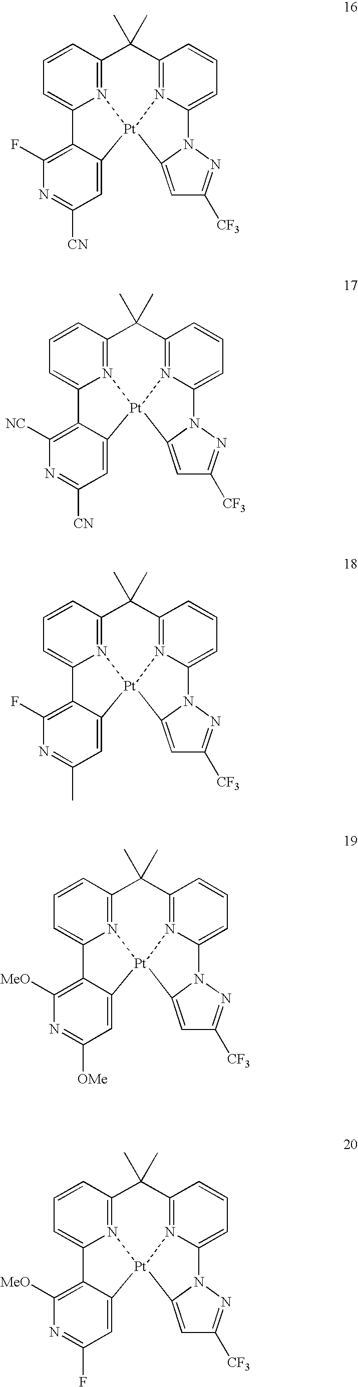

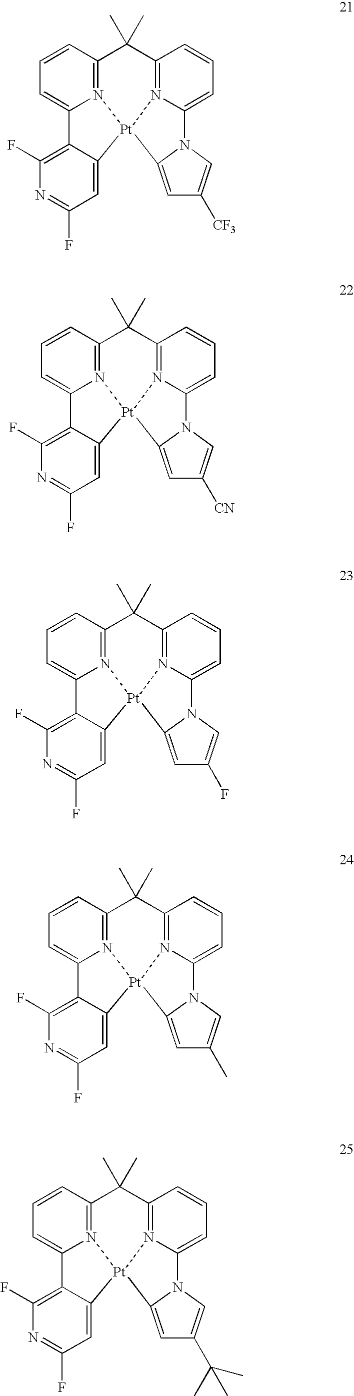

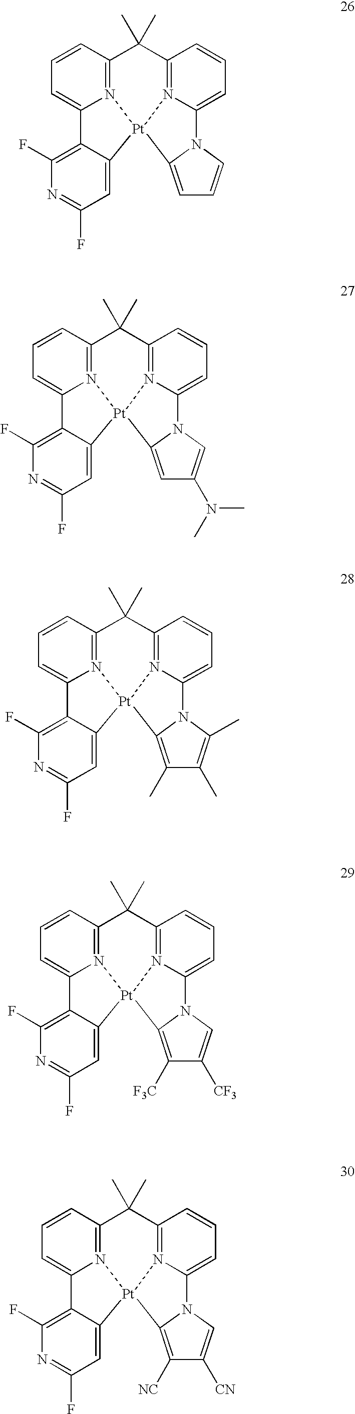

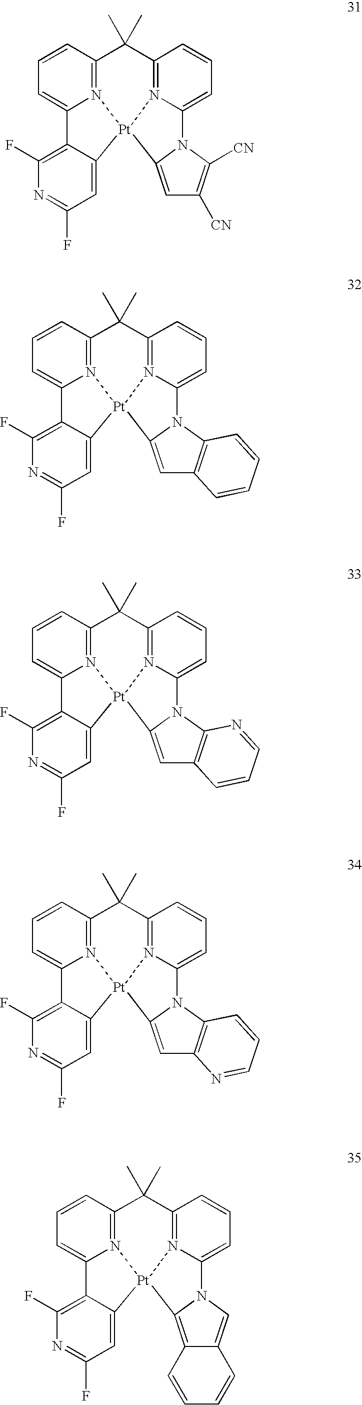

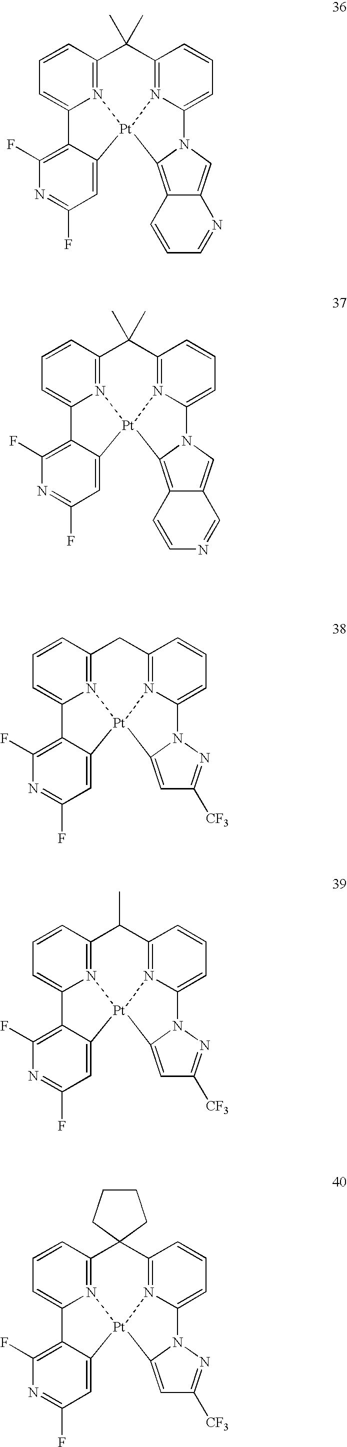

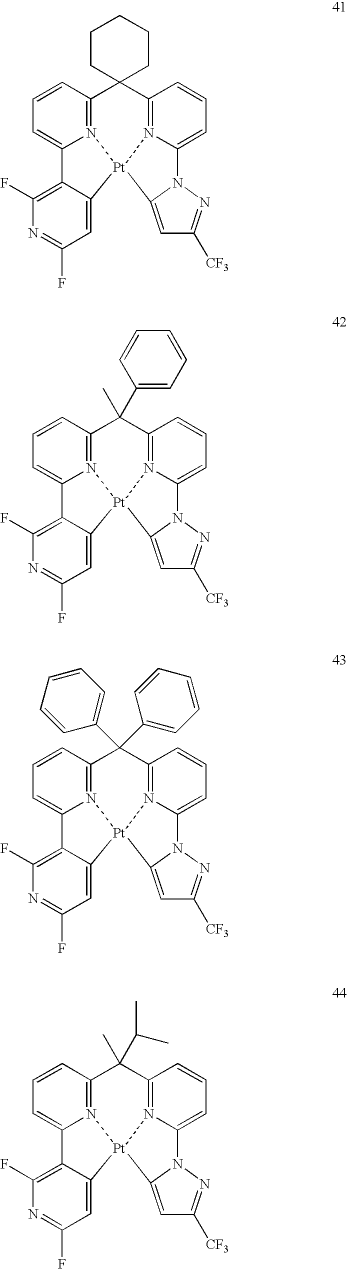

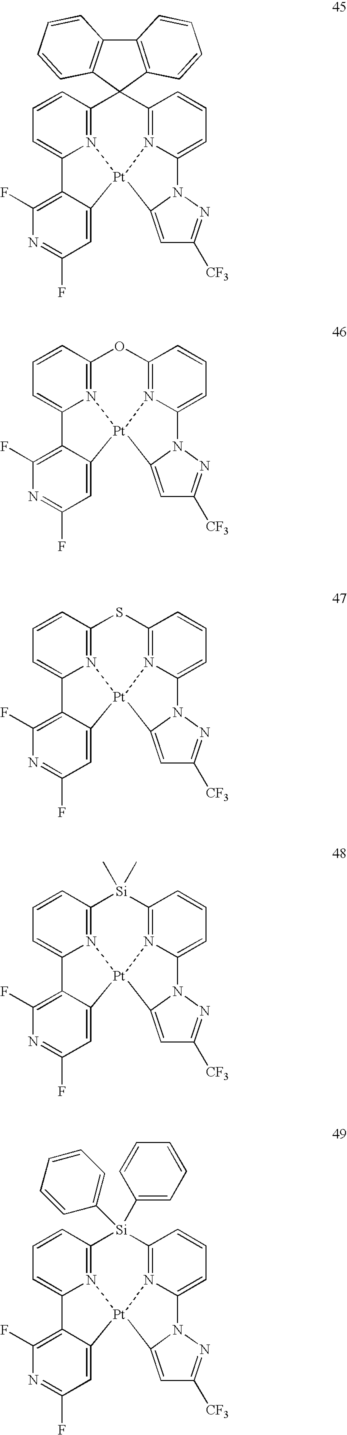

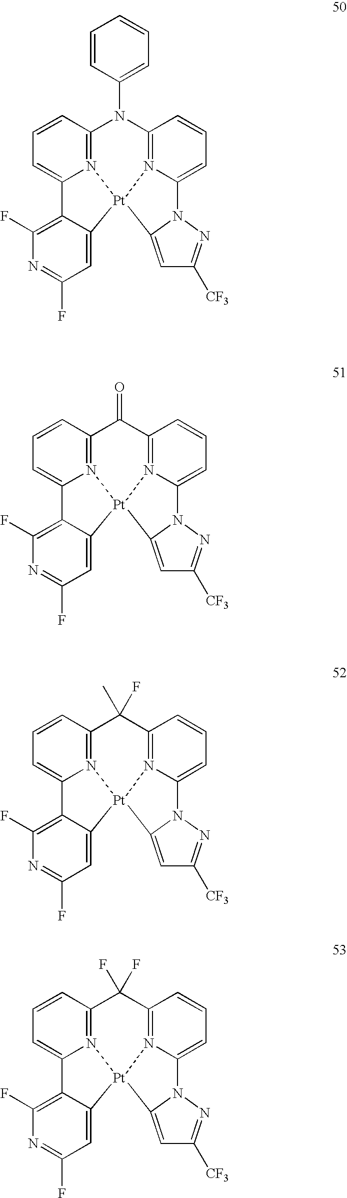

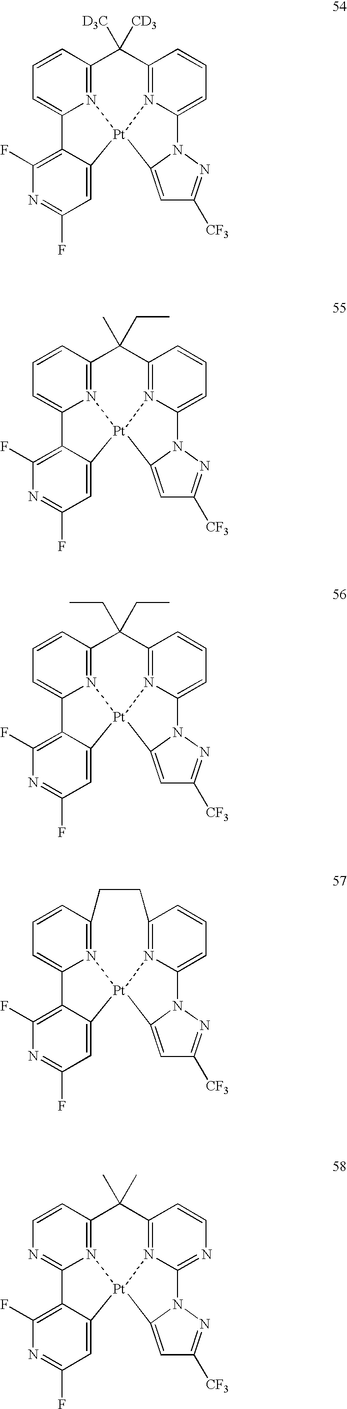

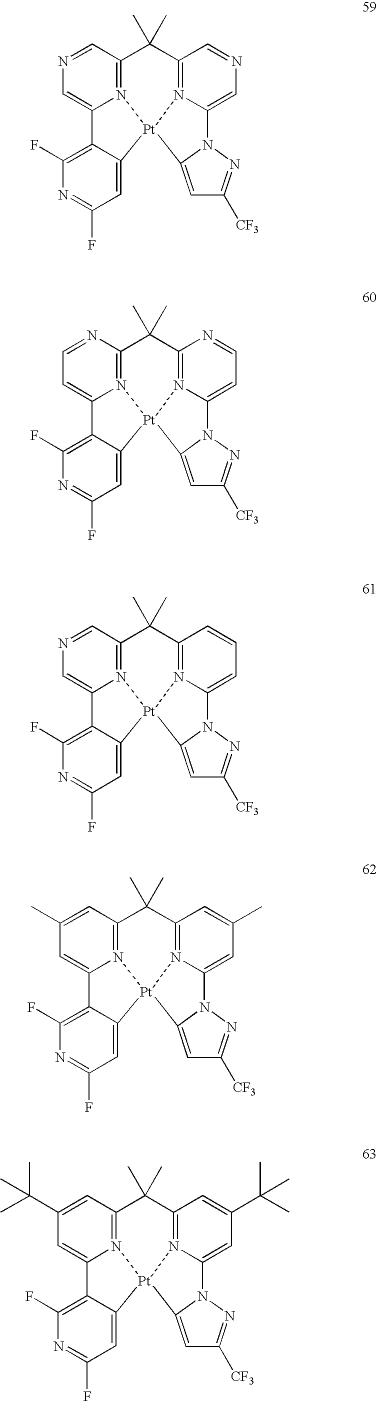

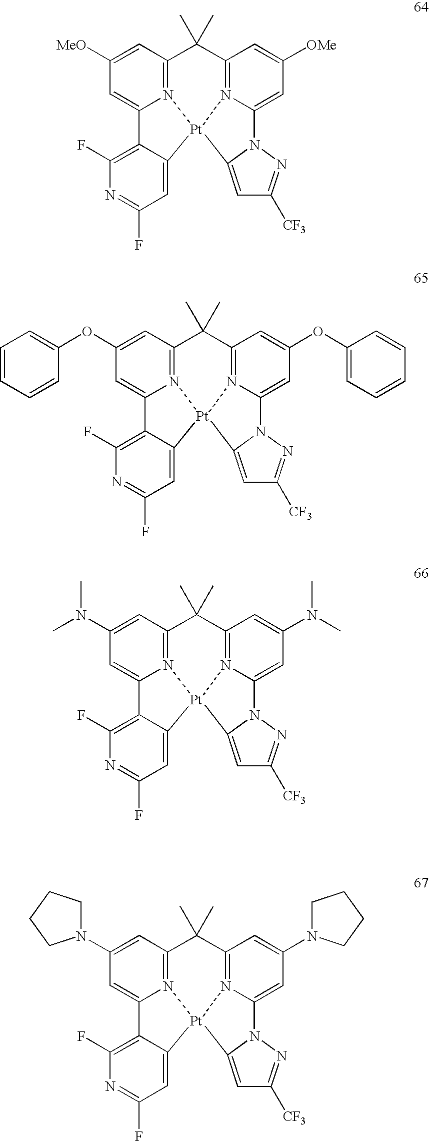

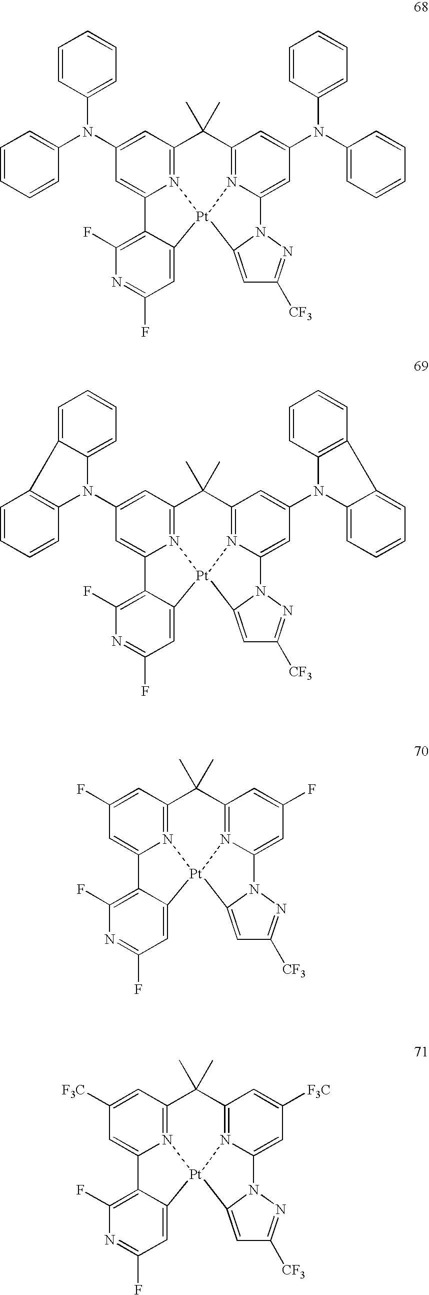

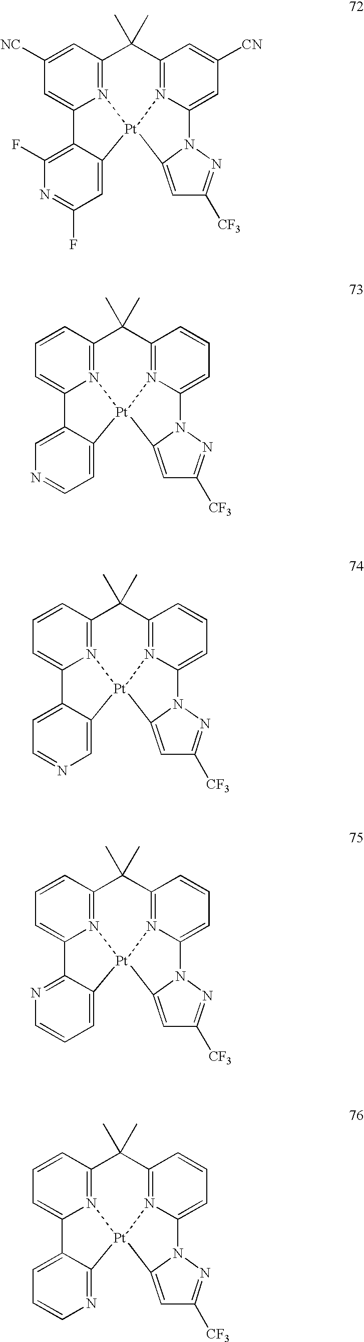

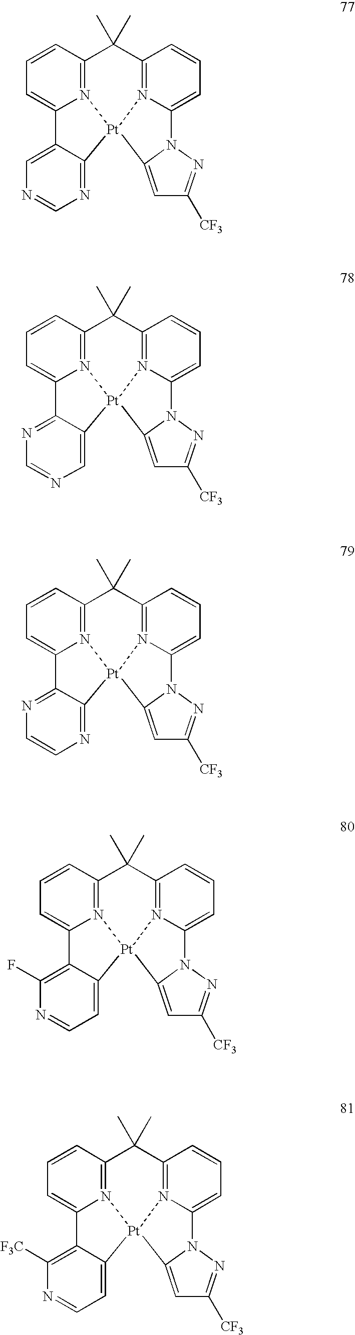

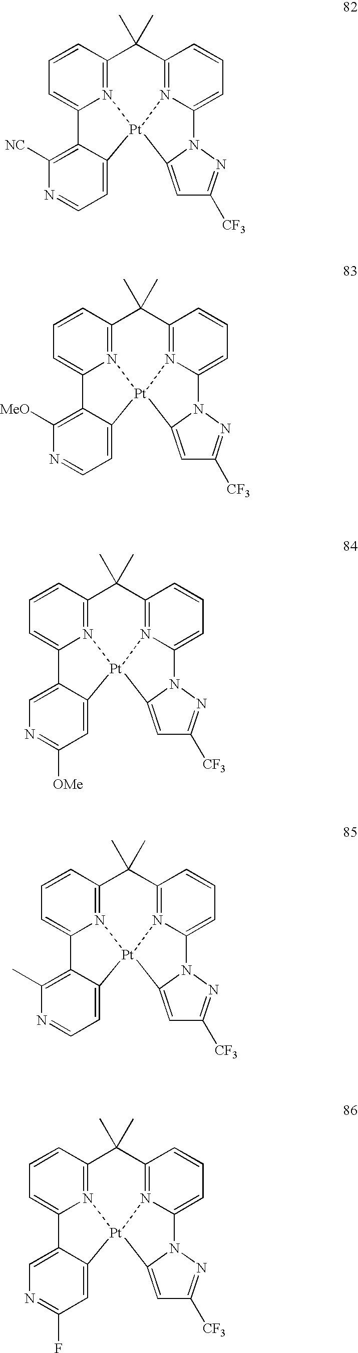

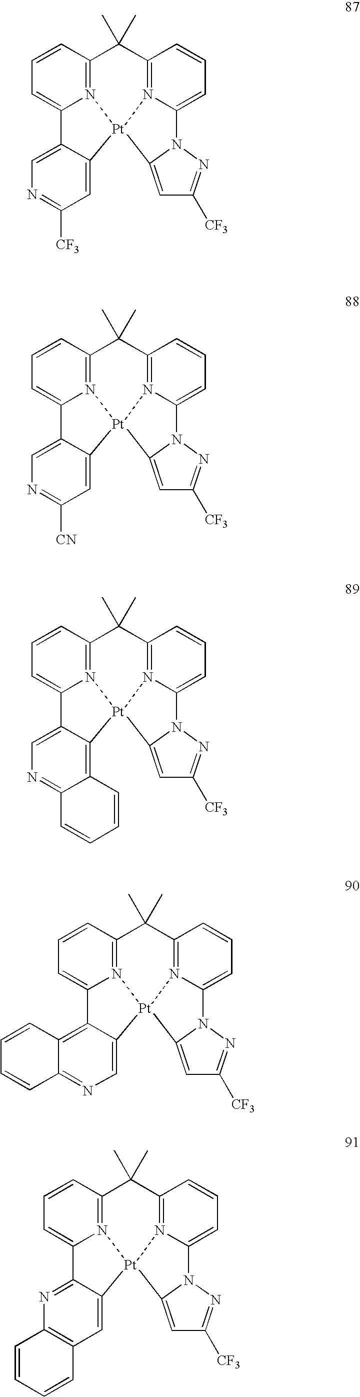

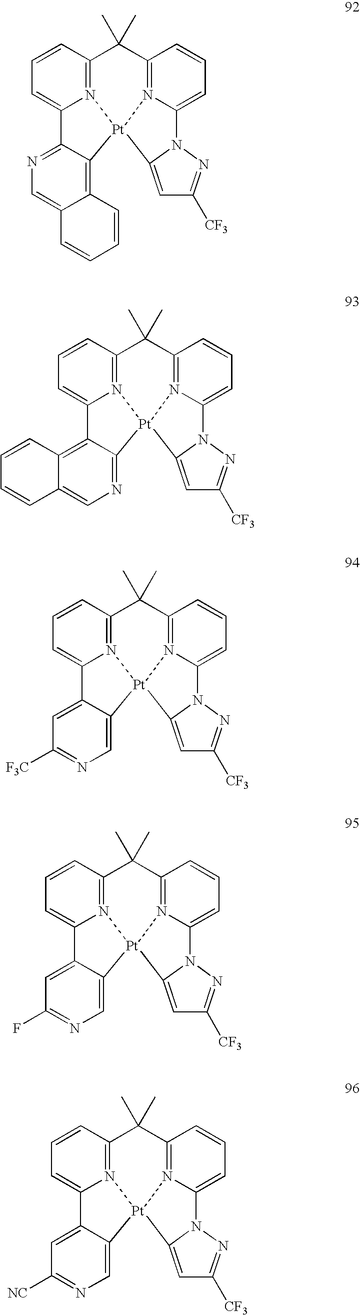

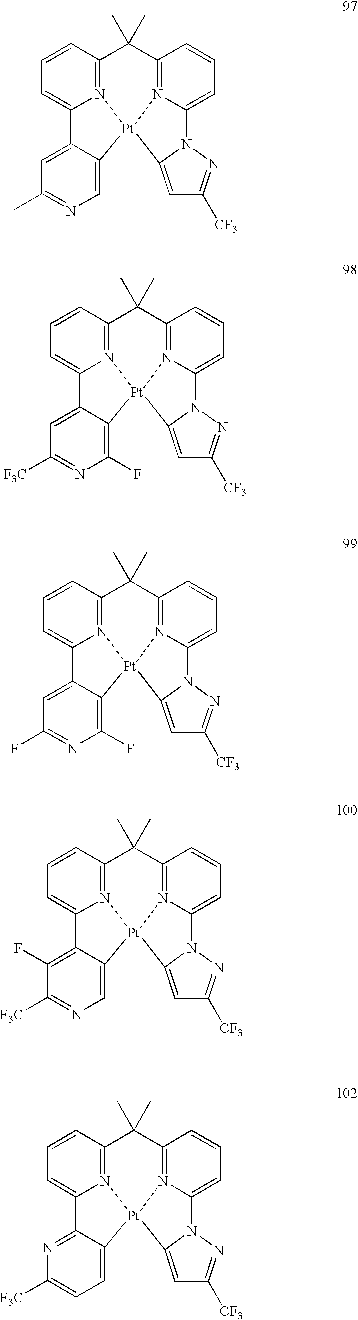

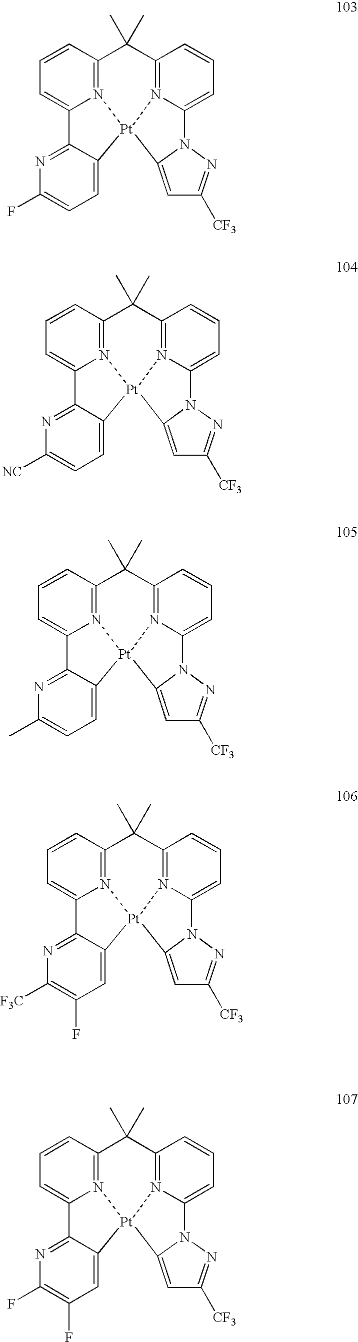

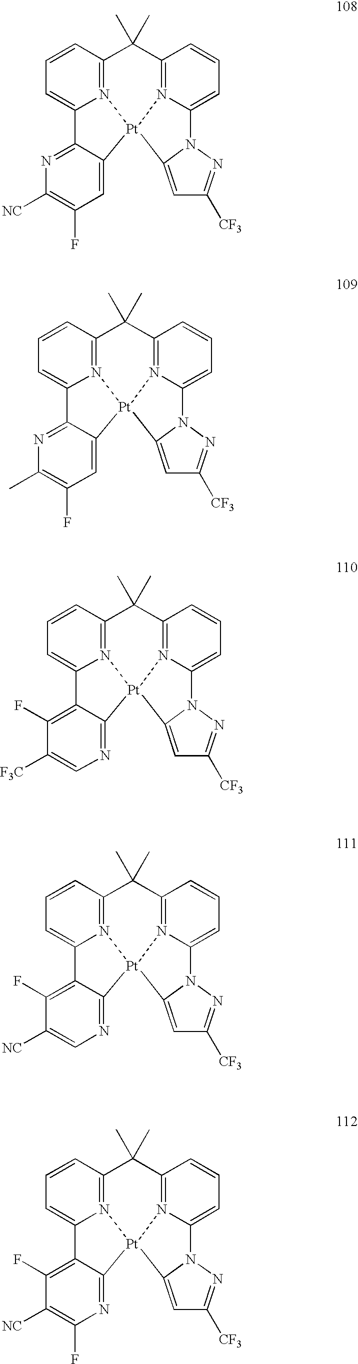

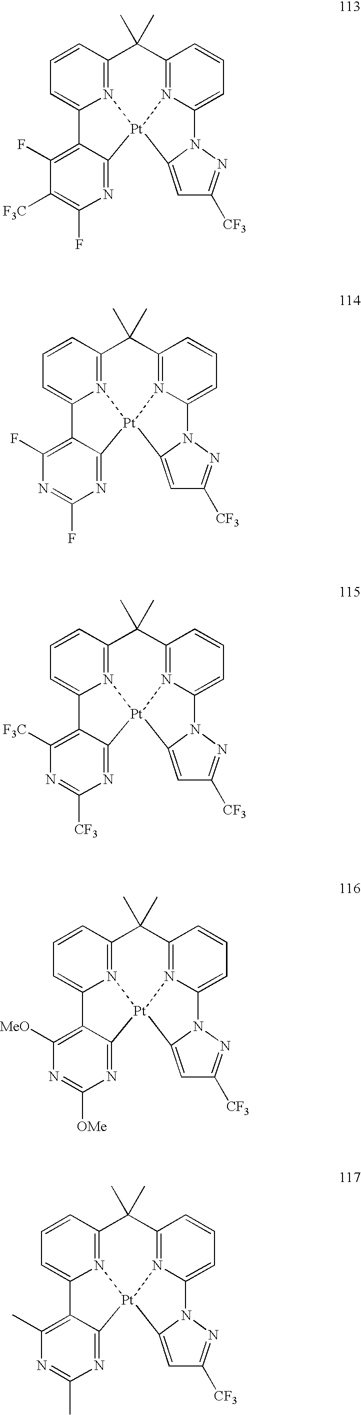

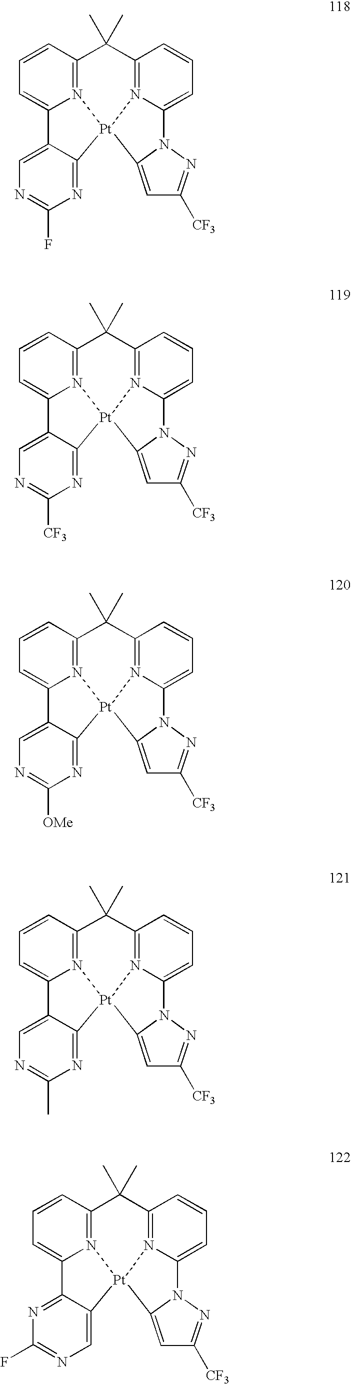

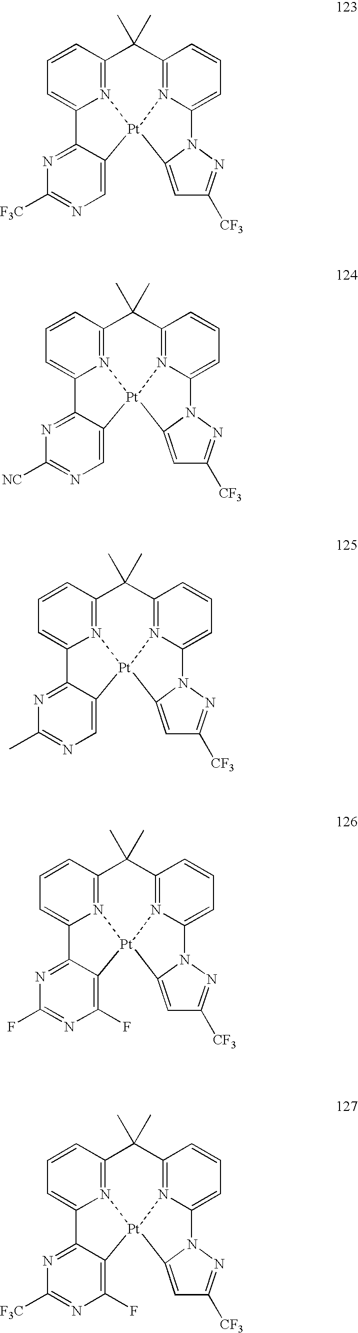

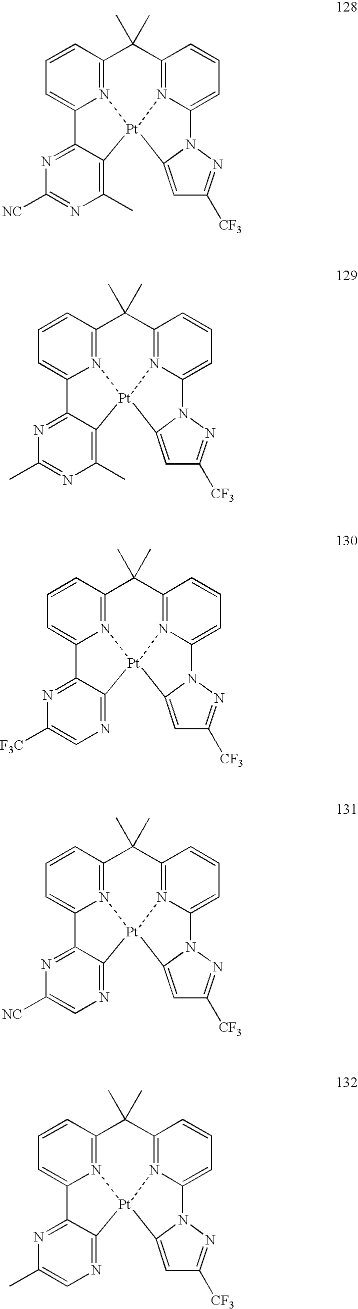

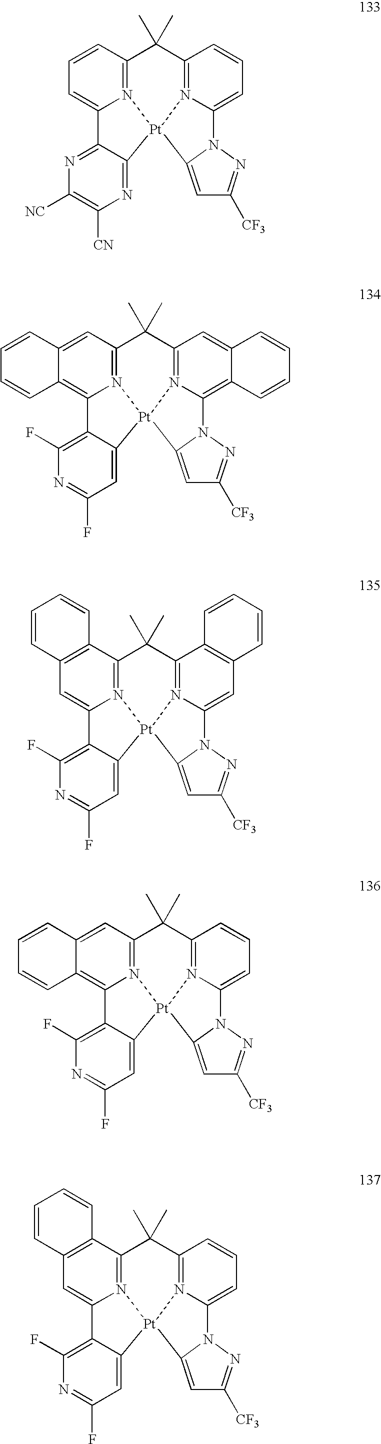

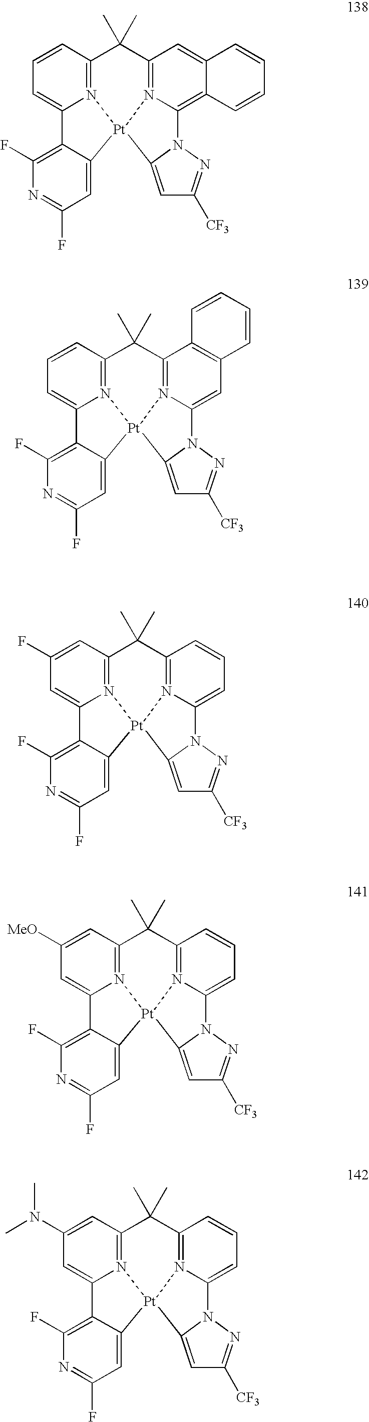

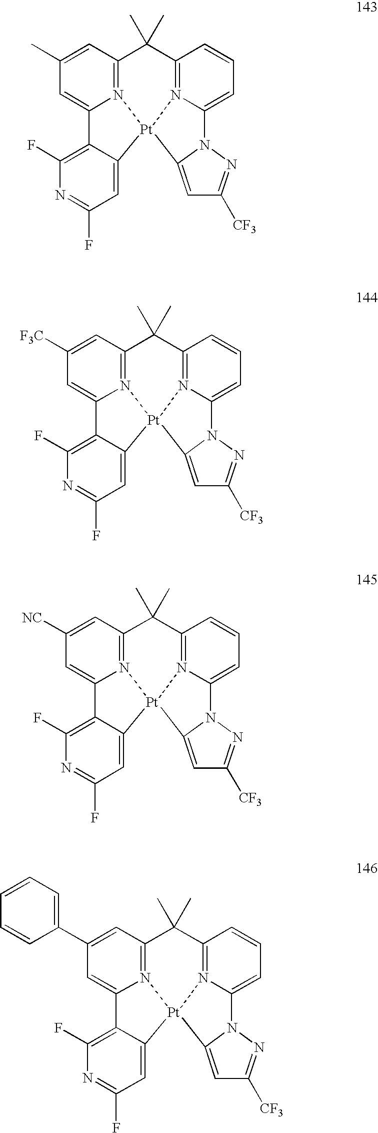

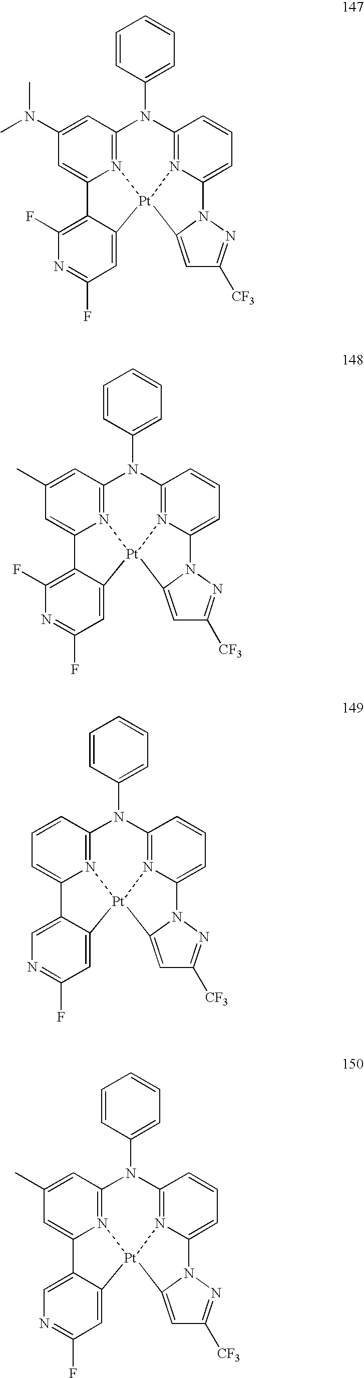

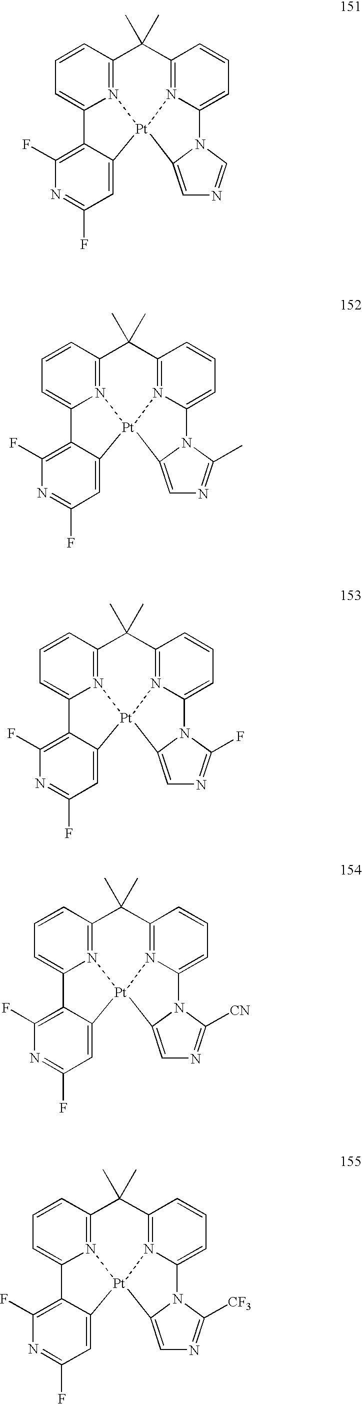

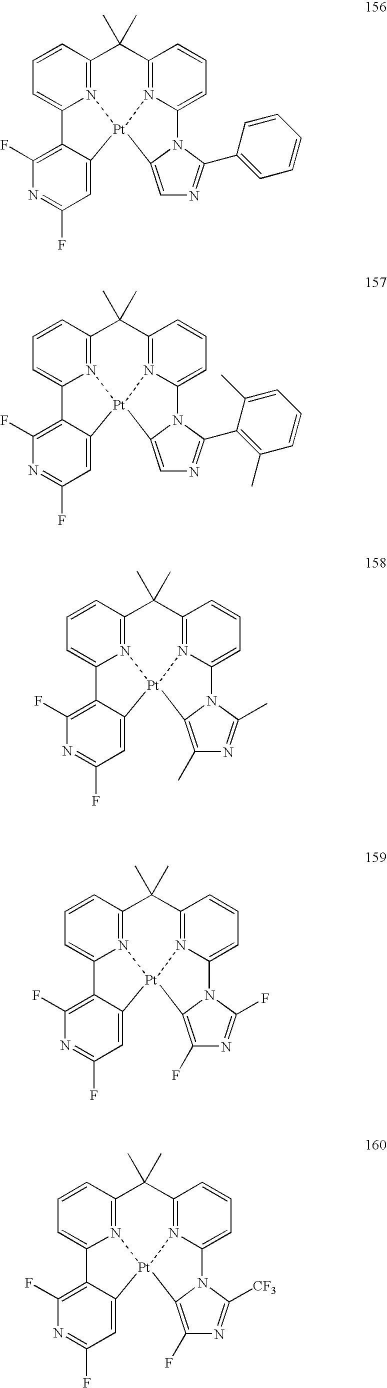

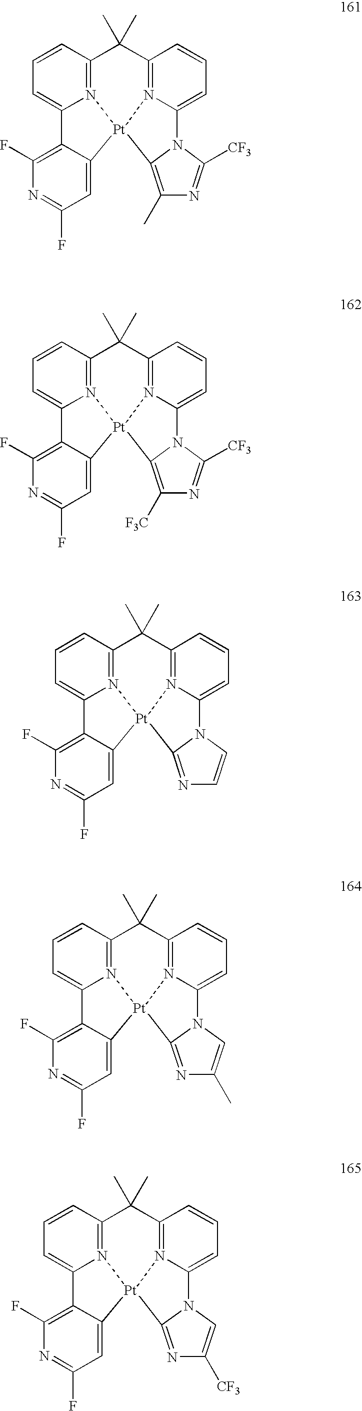

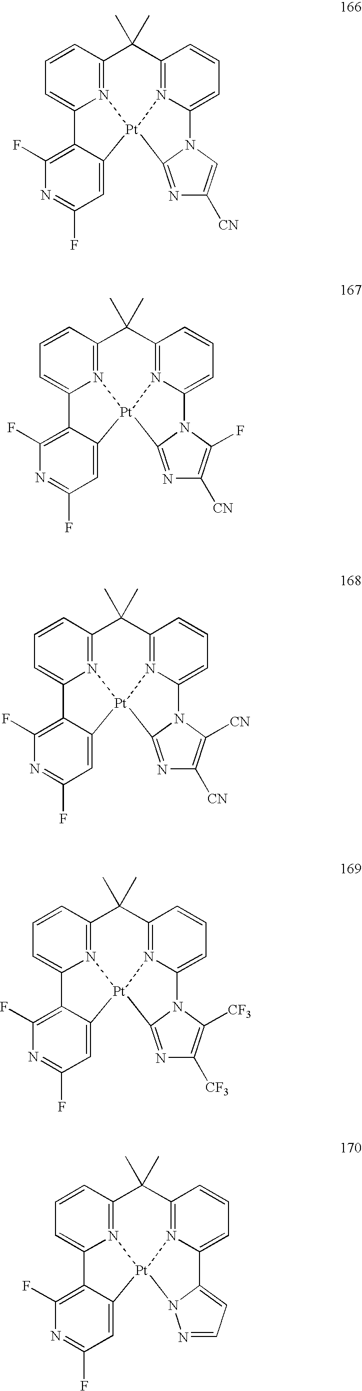

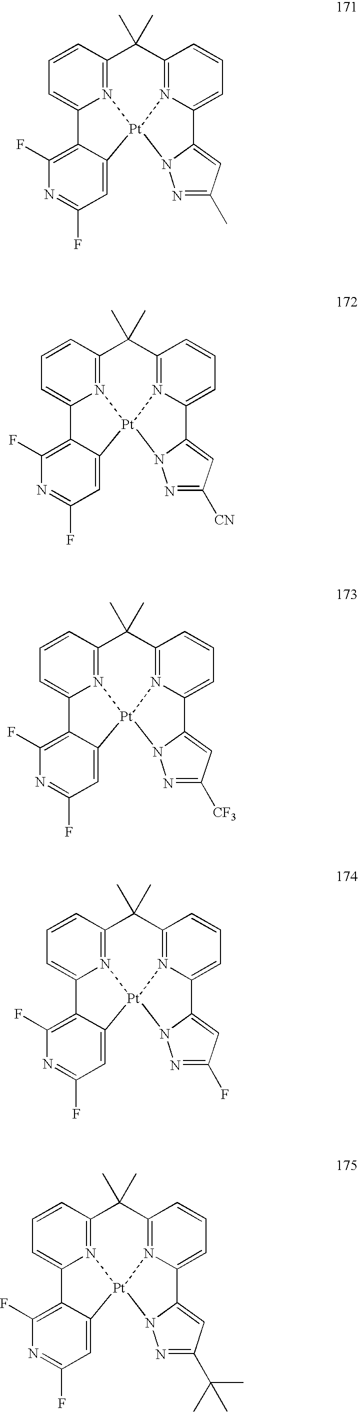

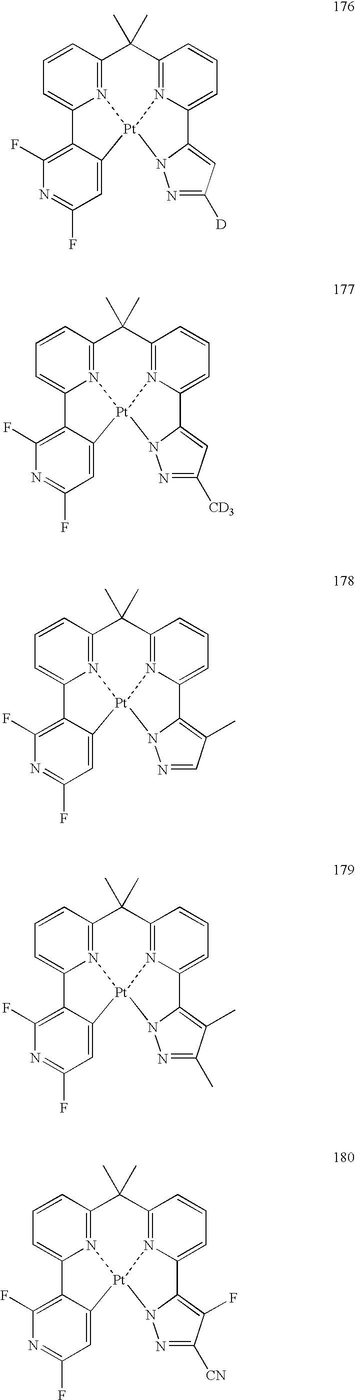

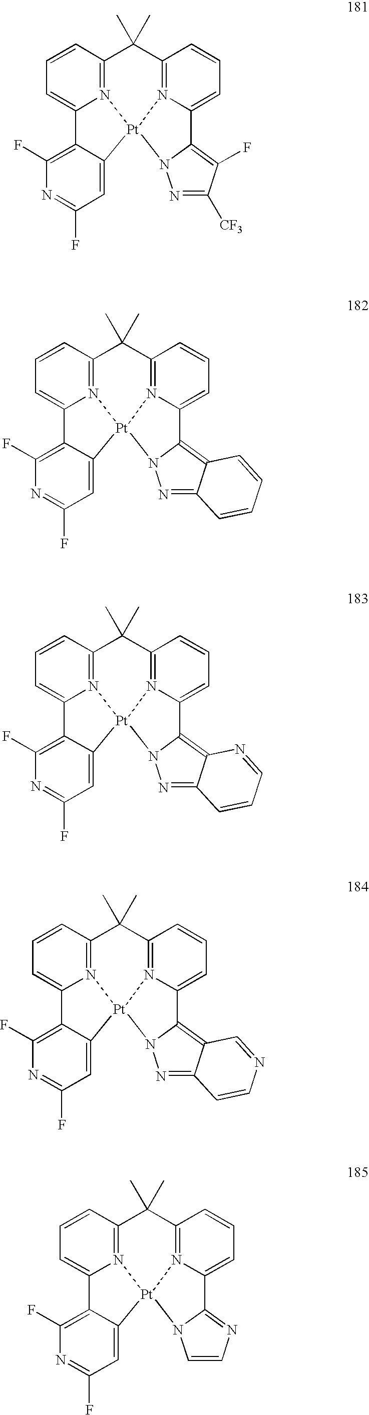

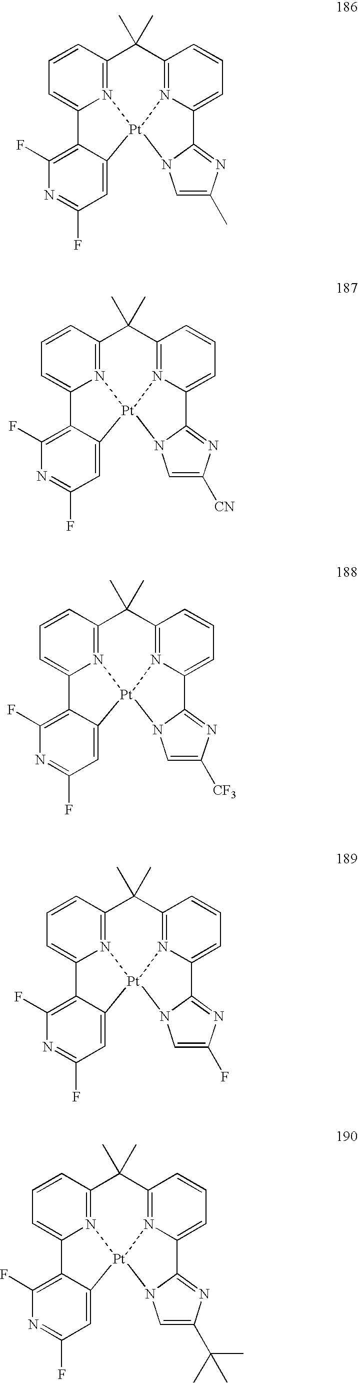

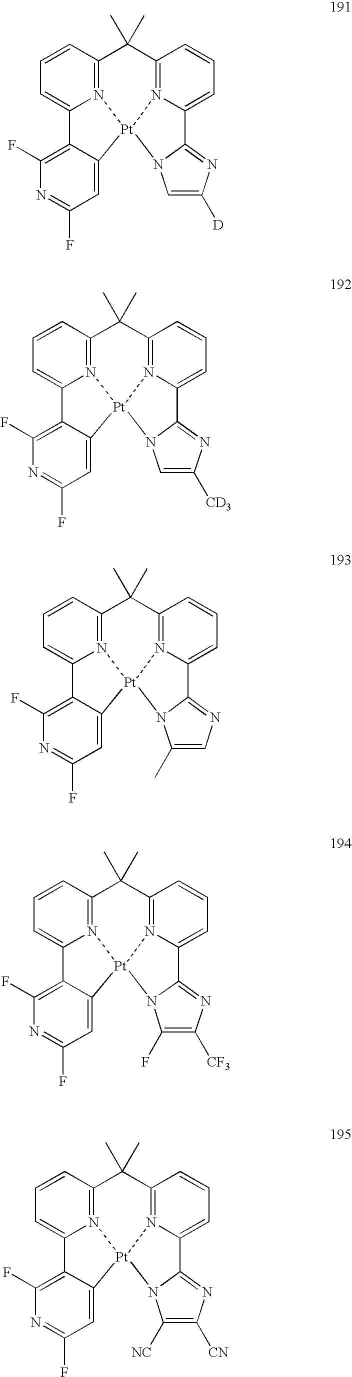

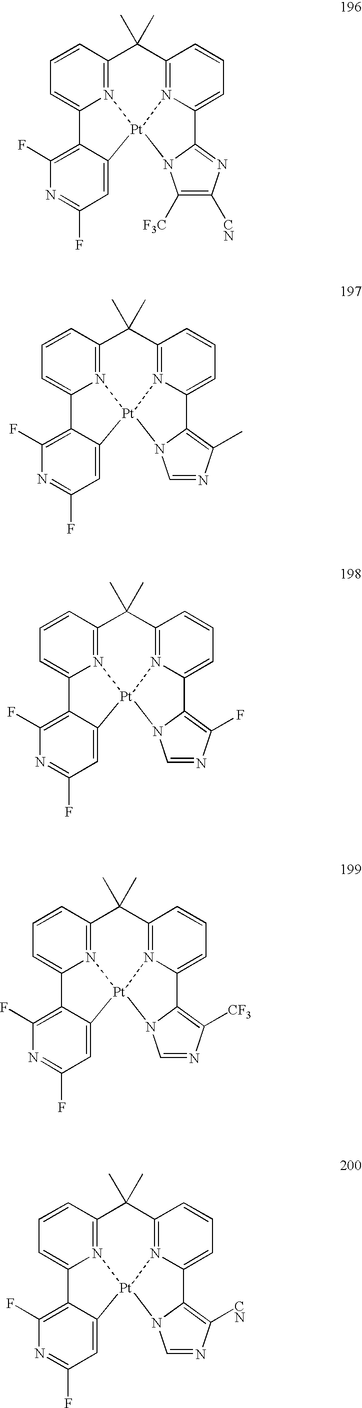

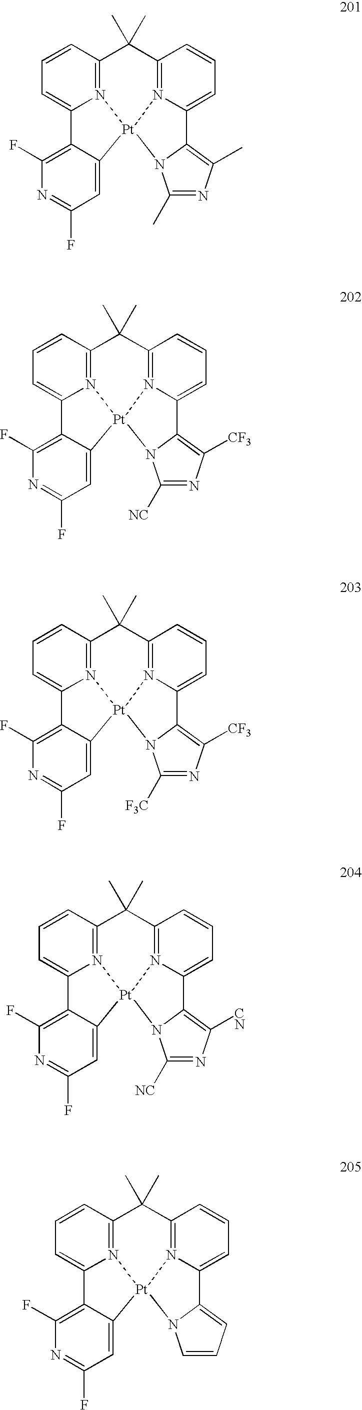

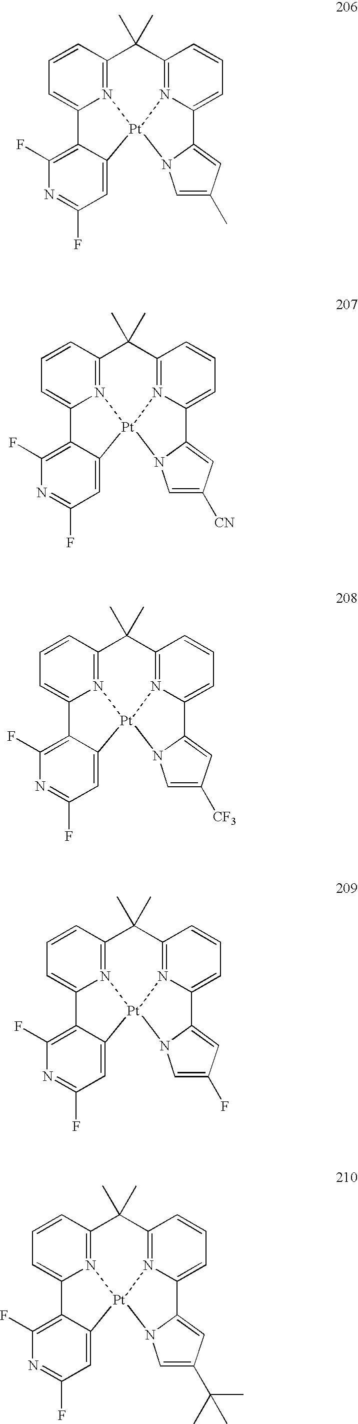

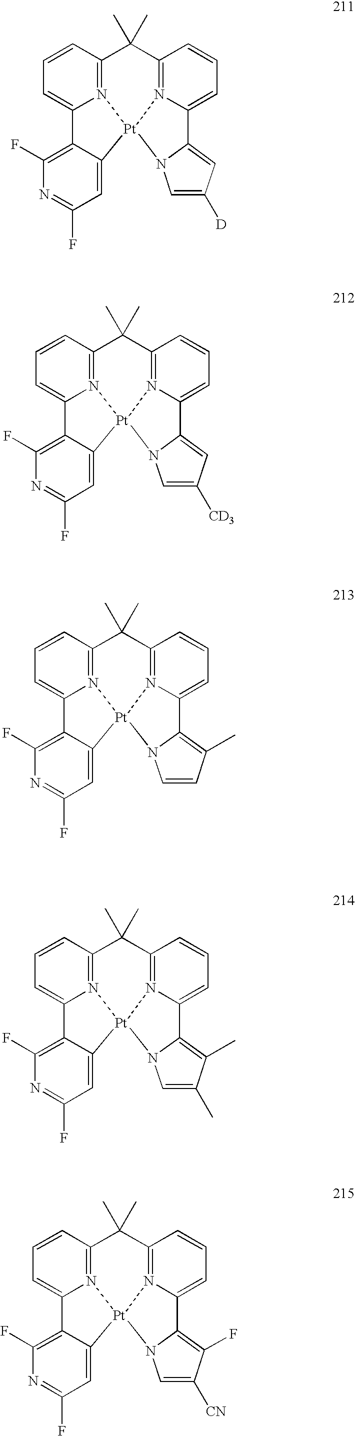

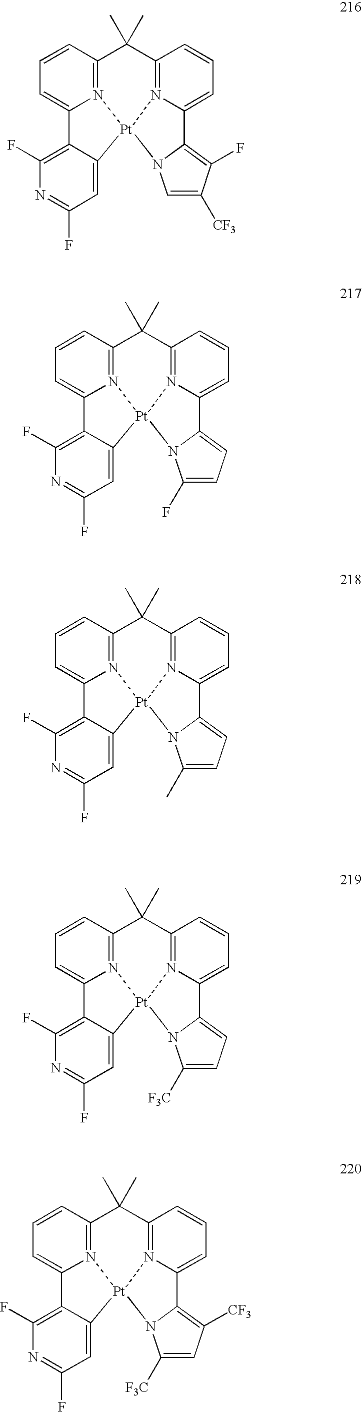

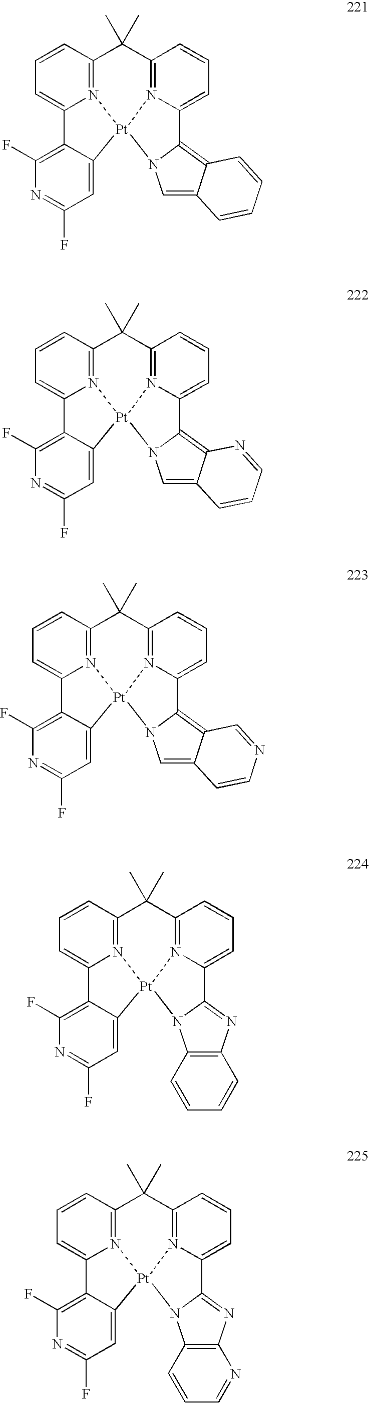

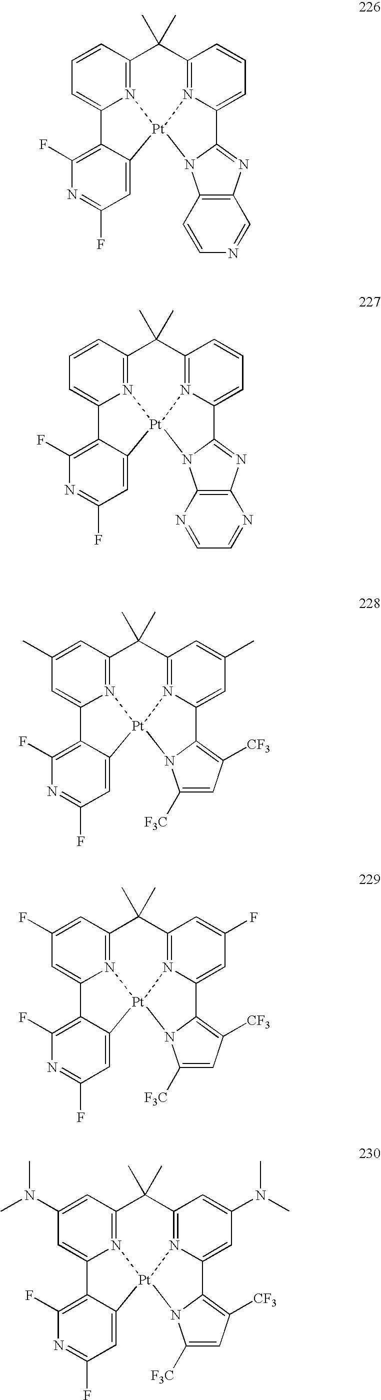

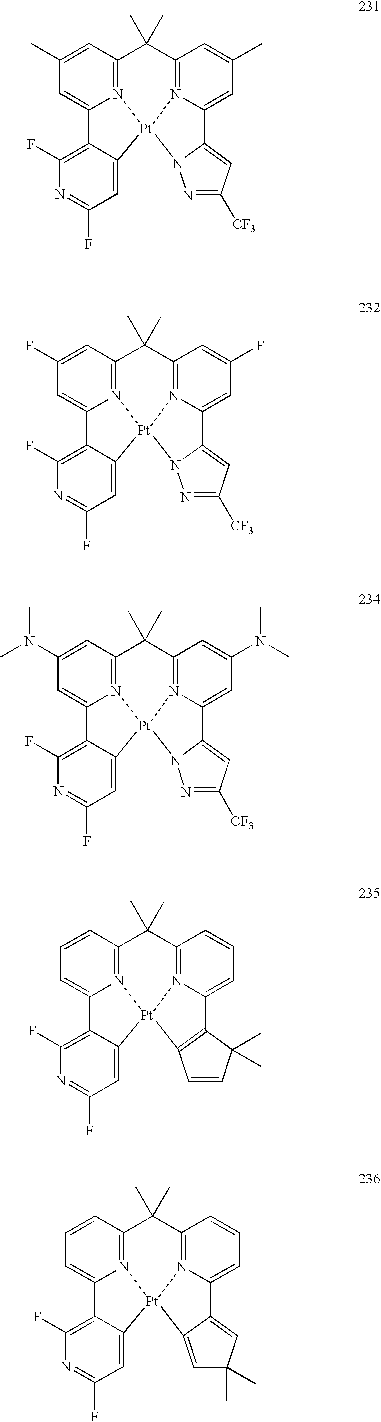

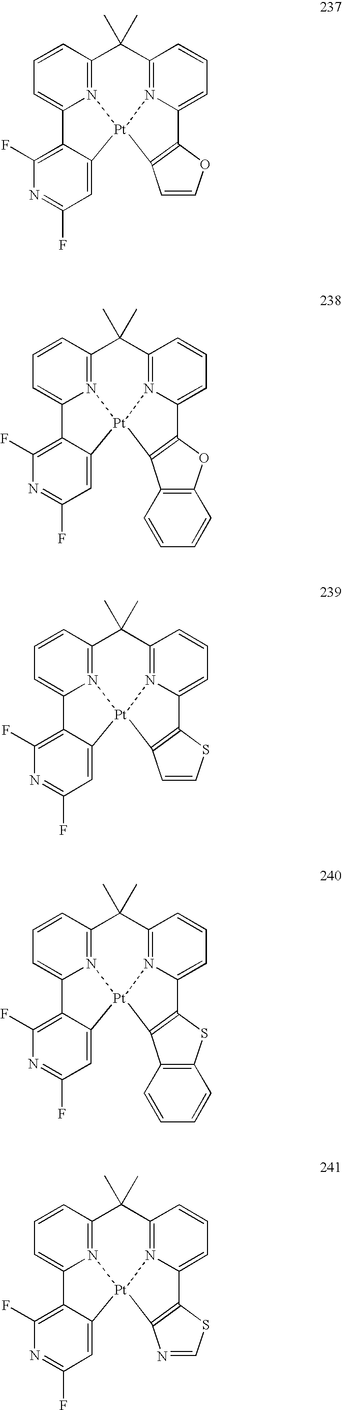

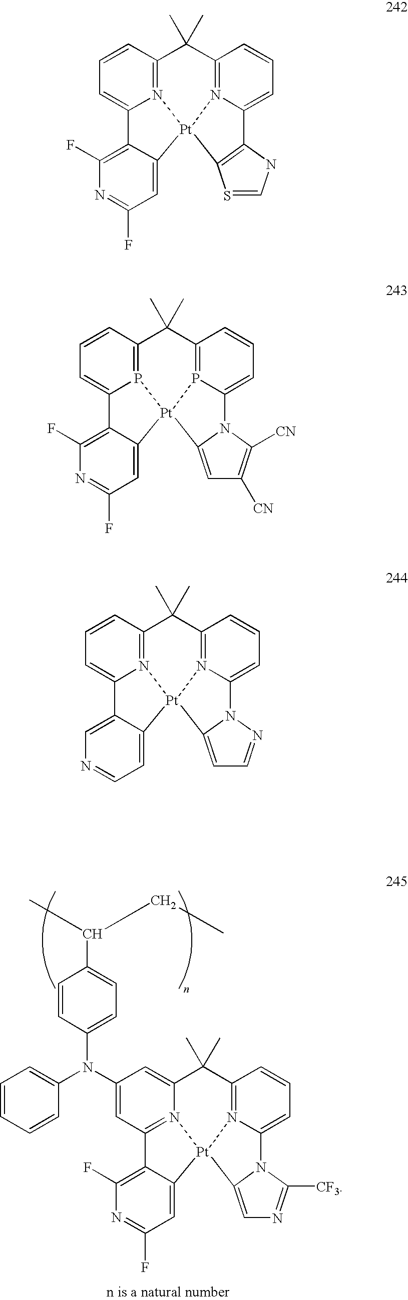

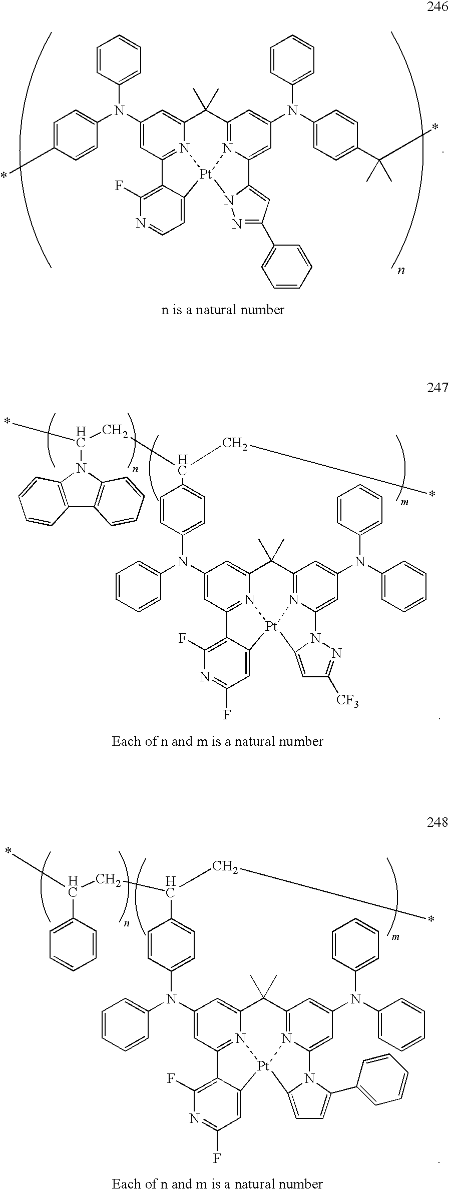

Next, a method for manufacturing the metal complex represented by the general formula (5) is described.

The metal complex represented by the general formula (5) can be obtained by allowing a compound represented by the following general formula (C-0) (hereinafter also referred to as “ligand”) to react with a platinum salt in the presence of a solvent.

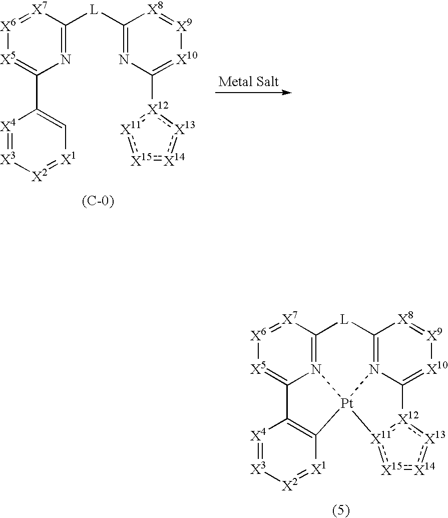

In the general formula (C-0), X¹, X², X³, X⁴, X⁵, X⁶, X⁷, X⁸, X⁹, X¹⁰, X¹¹, X¹², X¹³, X¹⁴, X¹⁵ and L are synonymous with X¹, X², X³, X⁴, X⁵, X⁶, X⁷, X⁸, X⁹, X¹⁰, X¹¹, X¹², X¹³, X¹⁴, X¹⁵ and L in the foregoing general formula (5), respectively, and preferred ranges thereof are also the same.

In the manufacture of the platinum complex, as to the platinum salt which is used at the time of a complex forming reaction with the ligand, examples of a compound containing divalent platinum include platinum chloride, platinum bromide, platinum iodide, platinum acetylacetonate, bis(benzonitrile)dichloroplatinum, bis(acetonitrile)dichloroplatinum, dichloro(1,5-cyclooctadiene)platinum, dibromobis(triphenylphosphine)platinum, dichloro(1,10-phenanthroline)platinum, dichlorobis(triphenylphosphine)platinum, ammonium tetrachloroplatinate, diamminedibromoplatinum, diamminedichloroplatinum, diamminediiodoplatinum, potassium tetrabromoplatinate, potassium tetrachloroplatinate, sodium tetrachloroplatinate, dimethyl bis(dimethyl sulfoxide)platinum, dimethyl bis(dimethyl sulfide)platinum and dimethyl(bicyclo[2.2.1]hepta-2,5-diene)platinum.

More preferred examples of the platinum salt include platinum halides such as platinum chloride, platinum bromide and platinum iodide; nitrile complexes such as bis(benzonitrile)dichloroplatinum and bis(acetonitrile)dichloroplatinum; and olefin complexes such as dichloro(1,5-cyclooctadiene)platinum. Of these, platinum halides such as platinum chloride and platinum bromide; and nitrile complexes such as bis(benzonitrile)dichloroplatinum and bis(acetonitrile)dichloroplatinum are further preferable.

The platinum salt which is used in the manufacture of the platinum complex may contain water of crystallization, a solvent of crystallization or a coordinating solvent. Though the valence of the metal is not particularly limited, the metal is preferably divalent or zero-valent, and more preferably divalent.

In the manufacture of the platinum complex, as to the use amount of the platinum salt which is used at the time of a complex forming reaction with the ligand, in the case where the platinum salt contains one metal atom capable of forming a complex, it is usually from 0.1 to 10 moles, preferably from 0.5 to 5 moles, and more preferably from 1 to 3 moles based on one mole of the ligand. In the case where the platinum salt contains the metal atom capable of forming a complex in the number of “n”, its use amount may be 1/n times.

In the manufacture of the platinum complex, examples of a solvent which is used at the time of a complex forming reaction between the platinum salt and the ligand include amides such as N,N-dimethylformamide, formamide and N,N-dimethylacetamide; nitriles such as acetonitrile, propionitrile, butyronitrile and benzonitrile; halogenated hydrocarbons such as dichloromethane, 1,2-dichloroethane, chloroform, carbon tetrachloride, chlorobenzene and o-dichlorobenzene; aliphatic hydrocarbons such as pentane, hexane, octane and decane; aromatic hydrocarbons such as benzene, toluene, xylene and mesitylene; ethers such as diethyl ether, diisopropyl ether, butyl ether, tert-butyl methyl ether, 1,2-dimethoxyethane, tetrahydrofuran and 1,4-dioxane; ketones such as acetone, methyl ethyl ketone and methyl isobutyl ketone; alcohols such as methanol, ethanol, 1-propanol, 2-propanol, tert-butyl alcohol, 2-methoxyethanol, 2-ethoxyethanol, ethylene glycol and glycerin; and water.

More preferred examples of the solvent include nitriles such as acetonitrile, propionitrile, butyronitrile and benzonitrile; aromatic hydrocarbons such as benzene, toluene, xylene and mesitylene; and alcohols such as methanol, ethanol, 1-propanol, 2-propanol, tert-butyl alcohol, 2-methoxyethanol, 2-ethoxyethanol, ethylene glycol and glycerin. Of these, nitriles such as acetonitrile, propionitrile, butyronitrile and benzonitrile; and aromatic hydrocarbons such as benzene, toluene, xylene and mesitylene are further preferable.

Such a solvent may be used singly or may be used in admixture of two or more kinds thereof.

In the manufacture of the platinum complex, the amount of the solvent which is used at the time of a complex forming reaction between the platinum salt and the ligand is not particularly limited so as far the reaction can be thoroughly advanced. It is usually from 1 to 200 times by volume, and preferably from 5 to 100 times by volume relative to the ligand to be used.

In the manufacture of the platinum complex, in the case where an acidic substance such as a hydrogen halide is formed at the time of a complex forming reaction between the platinum salt and the ligand, the reaction may be carried out in the presence of a basic substance. Examples of the basic substance include tertiary amines such as triethylamine, diisopropylethylamine, pyridine and 1,8-dimethylaminonaphthalene; metal alkoxides such as sodium methoxide and sodium ethoxide; and inorganic bases such as sodium hydroxide, potassium hydroxide, potassium carbonate and sodium hydrogencarbonate.

In the manufacture of the platinum complex, it is preferable that the complex forming reaction between the platinum salt and the ligand is carried out in an inert gas atmosphere. Examples of the inert gas include nitrogen and argon.

In the manufacture of the platinum complex, the reaction temperature, reaction time and reaction pressure at the time of a complex forming reaction between the platinum salt and the ligand vary depending upon the raw materials, solvent and the like. The reaction temperature is usually in the range of from 20° C. to 300° C., preferably from 50° C. to 250° C., and more preferably from 80° C. to 220° C. The reaction time is usually from 30 minutes to 24 hours, preferably from 1 to 12 hours, and more preferably from 2 to 10 hours. Though the reaction pressure is usually atmospheric pressure, it may be an elevated pressure or a reduced pressure, if desired.

In the manufacture of the platinum complex, a heating measure at the time of a complex forming reaction between the platinum salt and the ligand is not particularly limited. Specifically, heating by an oil bath or a mantle heater, or heating by means of irradiation with microwaves is useful.

The thus obtained platinum complex can be isolated and purified, if desired. Examples of a method for achieving the isolation and purification include column chromatography, recrystallization, reprecipitation and sublimation. These methods may be employed singly or in combinations.

Among the platinum complexes represented by the general formula (5), the platinum complex represented by the general formula (6a-1) can also be synthesized according to the following manufacturing method. However, it should not be construed that the invention is limited to the following method.

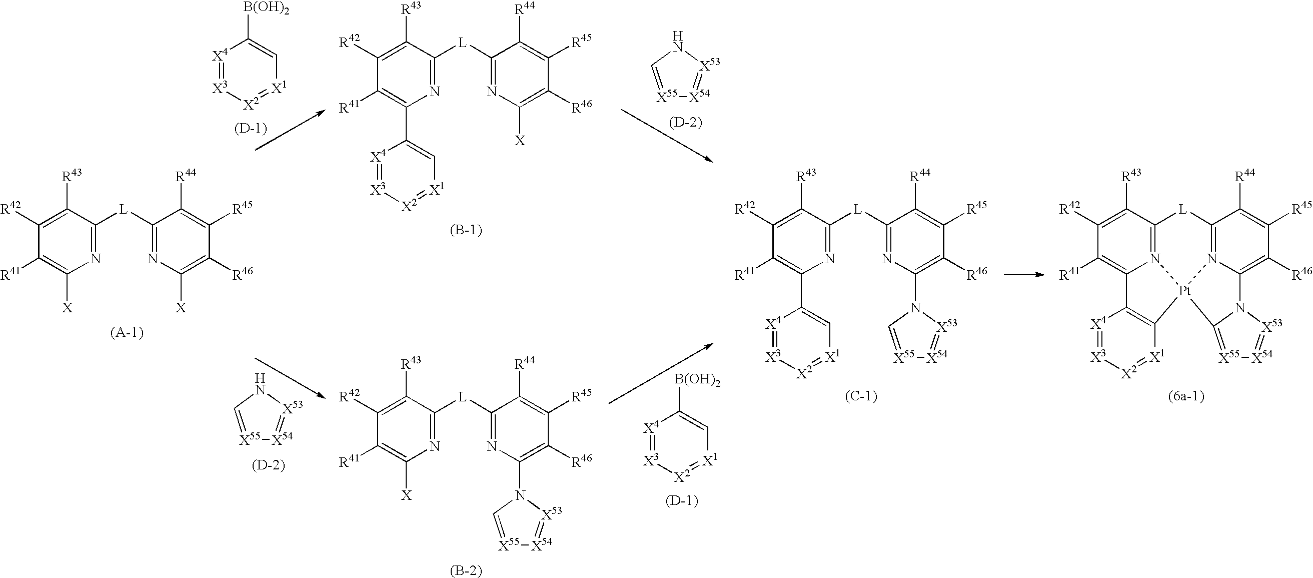

In the foregoing formulae, X¹, X², X³, X⁴, X⁵³, X⁵⁴, X⁵⁵, R⁴¹, R⁴², R⁴³, R⁴⁴, R⁴⁵, R⁴⁶ and L are synonymous with those in the foregoing general formula (6a-3).

As the step of obtaining (B-1) from (A-1) and the step of obtaining (C-1) from (B-2), each of the desired compounds can be synthesized by employing a method described in Synth. Commun., 11, 513 (1981) or other methods.

As the step of obtaining (C-1) from (B-1) and the step of obtaining (B-2) from (A-1), each of the desired compounds can be synthesized by utilizing a method described in Angew. Chem. Int. Ed., 42, 2051 to 2053 (2003) or other methods.

As to the step of obtaining the platinum complex represented by the general formula (6a-1) from (C-1), the desired compound can be synthesized by dissolving the compound (C-1) and from 1 to 1.5 equivalents of platinous chloride in benzonitrile, heating the solution at from 130° C. to the heat reflux temperature (boiling point of benzonitrile: 191° C.) and stirring it for from 30 minutes to 4 hours. The platinum complex represented by the general formula (5) can be purified by recrystallization from chloroform, dichloromethane, toluene, xylene, acetonitrile, butyronitrile, benzonitrile, ethyl acetate, etc., silica gel column chromatography, purification by sublimation, or the like.

In the foregoing manufacturing method, in the case where the defined substituents are changed under a condition of a certain synthesis method or are inadequate for carrying out the instant method, the manufacture can be easily made by measures such as protection and deprotection of a functional group (see, for example, Protective Groups in Organic Synthesis, written by T. W. Greene, John Wiley & Sons Inc. (1981), etc.). Also, the order of the reaction steps such as introduction of a substituent can be properly changed, if desired.

### [Compound Represented by the General Formula (T-1)]

A compound represented by the following general formula (T-1) is described.

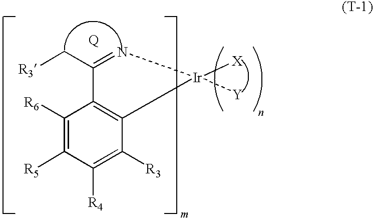

In the general formula (T-1), R₃′ represents an alkyl group, a heteroalkyl group, an aryl group or a heteroaryl group and may further have a substituent Z.

R₅ represents an aryl group or a heteroaryl group and may be further substituted with a non-aromatic group.

The ring Q represents an aromatic heterocyclic ring or a condensed aromatic heterocyclic ring each having at least one nitrogen atom, which is coordinated on Ir, and may be further substituted with a non-aromatic group.

Each of R₃, R₄ and R₆ represents a hydrogen atom, an alkyl group, an alkenyl group, an alkynyl group, —CN, a perfluoroalkyl group, a trifluorovinyl group, —CO₂R, —C(O)R, —NR₂, —NO₂, —OR, a halogen atom, an aryl group or a heteroaryl group and may further have a substituent Z.

R₃ and R₄ may be bonded to each other to form a condensed 4-membered to 7-membered ring, the condensed 4-membered to 7-membered ring is a cycloalkyl, a cycloheteroalkyl, an aryl or a heteroaryl, and the condensed 4-membered to 7-membered ring may further have a substituent Z.

R₃′ and R₆ may be connected to each other via a connecting group selected among —CR₂—CR₂—, —CR═CR—, —CR₂, —O—, —NR—, —O—CR₂—, —NR—CR₂— and —N═CR— to form a ring; and each R independently represents a hydrogen atom, an alkyl group, an alkenyl group, an alkynyl group, a heteroalkyl group, an aryl group or a heteroaryl group and may further have a substituent Z.

Each Z independently represents a halogen atom, —R′, —OR′, —N(R′)₂, —SR′, —C(O)R′, —C(O)OR′, —C(O)N(R′)₂, —CN, —NO₂, —SO₂, —SOR′, —SO₂R′ or —SO₃R′; and each R′ independently represents a hydrogen atom, an alkyl group, a perhaloalkyl group, an alkenyl group, an alkynyl group, a heteroalkyl group, an aryl group or a heteroaryl group.

(X—Y) represents an auxiliary ligand.

m represents an integer of from 1 to 3; and n represents an integer of from 0 to 2.

(m+n) is 3.

The general formula (T-1) is a complex having iridium (Ir) as a metal and is excellent from the viewpoint of high light emission quantum yield.

The alkyl group represented by each of R₃′, R₃, R₄ and R₆ may have a substituent and may be saturated or unsaturated. Examples of a group which may be substituted include the foregoing substituent Z.

The alkyl group represented by each of R₃′, R₃, R₄ and R₆ is preferably an alkyl group having a total carbon atom number of from 1 to 8, and more preferably an alkyl group having a total carbon atom number of from 1 to 6. Examples thereof include a methyl group, an ethyl group, an isopropyl group, a cyclohexyl group and a t-butyl group.

The alkenyl group represented by each of R₃, R₄ and R₆ is preferably an alkenyl group having a total carbon atom number of from 2 to 30, and more preferably an alkenyl group having a total carbon atom number of from 2 to 20, and especially preferably an alkenyl group having a total carbon atom number of from 2 to 10. Examples thereof include a vinyl group, an allyl group, a 1-propenyl group, a 1-isopropenyl group, a 1-butenyl group, a 2-butenyl and a 3-pentenyl group.

The alkynyl group represented by each of R₃, R₄ and R₆ is preferably an alkynyl group having a total carbon atom number of from 2 to 30, and more preferably an alkynyl group having a total carbon atom number of from 2 to 20, and especially preferably an alkynyl group having a total carbon atom number of from 2 to 10. Examples thereof include an ethynyl group, a propargyl group, a 1-propynyl and a 3-pentynyl group.

Examples of the heteroalkyl group represented by R₃′ include groups obtained by substituting at least one carbon of the foregoing alkyl group with O, NR or S.

The aryl group represented by each of R₃′ and R₃ to R₆ is preferably a substituted or unsubstituted aryl group having from 6 to 30 carbon atoms, and examples thereof include a phenyl group, a tolyl group and a naphthyl group.

The heteroaryl group represented by each of R₃′ and R₃ to R₆ is preferably a heteroaryl group having from 5 to 8 carbon atoms, and more preferably a 5- or 6-membered, substituted or unsubstituted heteroaryl group. Examples thereof include a pyridyl group, a pyrazinyl group, a pyridazinyl group, a pyrimidinyl group, a triazinyl group, a quinolinyl group, an isoquinolinyl group, a quinazolinyl group, a cinnolinyl group, a phthalazinyl group, a quinoxalinyl group, a pyrrolyl group, an indolyl group, a furyl group, a benzofuryl group, a thienyl group, a benzothienyl group, a pyrazolyl group, an imidazolyl group, a benzimidazolyl group, a triazolyl group, an oxazolyl group, a benzoxazolyl group, a thiazolyl group, a benzothiazolyl group, an isothiazolyl group, a benzisothiazolyl group, a thiadiazolyl group, an isoxazolyl group, a benzisoxazolyl group, a pyrrolidinyl group, a piperidinyl group, a piperazinyl group, an imidazolidinyl group, a thiazolinyl group and a sulforanyl group.

Preferred examples of the heteroaryl group represented by R₃′ include a pyridyl group, a pyrimidinyl group, an imidazolyl group and a thienyl group, with a pyridyl group and a pyrimidinyl group being more preferable.

R₃′ is preferably a methyl group, an ethyl group, a propyl group or a butyl group; more preferably a methyl group or an ethyl group; and further preferably a methyl group.

R₅ represents an aryl or a heteroaryl, and the aryl or heteroaryl may be substituted with one or more non-aromatic groups.

The non-aromatic group in R₅ is preferably an alkyl group, an alkoxy group, a fluoro group, a cyano group, an alkylamino group or a diarylamino group; more preferably an alkyl group, a fluoro group or a cyano group; and further preferably an alkyl group.

R₅ is preferably a phenyl group, a p-tolyl group or a naphthyl group, and more preferably a phenyl group.

Each of R₃, R₄ and R₆ is preferably a hydrogen atom, an alkyl group, a cyano group, a trifluoromethyl group, a perfluoroalkyl group, a dialkylamino group, a fluoro group, an aryl group or a heteroaryl group; more preferably a hydrogen atom, an alkyl group, a cyano group, a trifluoromethyl group, a fluoro group or an aryl group; and further preferably a hydrogen atom, an alkyl group or an aryl group.

The substituent Z in each of R₃, R₄ and R₆ is preferably an alkyl group, an alkoxy group, a fluoro group, a cyano group or a dialkylamino group; and more preferably a hydrogen atom.

Examples of the aromatic heterocyclic ring represented by the ring Q include a pyridine ring, a pyrazine ring, a pyrimidine ring, a pyrazole ring, an imidazole ring, a triazole ring, an oxazole ring, an oxadiazole ring, a thiazole ring and a thiadiazole ring. Of these, a pyridine ring and a pyrazine ring are preferable; and a pyridine ring is more preferable.

Examples of the condensed aromatic heterocyclic ring represented by the ring Q include a quinoline ring, an isoquinoline ring and a quinoxaline ring. Of these, a quinoline ring and an isoquinoline ring are preferable; and a quinoline ring is more preferable.

The non-aromatic group in the ring Q is preferably an alkyl group, an alkoxy group, a fluoro group, a cyano group, an alkylamino group or a diarylamino group; and more preferably an alkyl group, a fluoro group or a cyano group.

m is preferably from 1 to 3, more preferably from 2 to 3, and further preferably 2.

n is preferably from 0 to 1, and more preferably 1.

It is more preferable that m is 2, whereas n is 1.

(X—Y) represents an auxiliary ligand. Since it may be considered that such a ligand does not directly contribute to optical activity characteristics but is able to change the optical activity characteristics of a molecule, it is called “auxiliary”. The definitions of optical activity and auxiliary are aimed to mean a non-limitative theory. For example, in the case of Ir, with respect to a bidentate ligand, n can be 0, 1 or 2. The auxiliary ligand which is used in the light emitting material can be selected among those which are known in the art. Non-limitative examples of the auxiliary ligand are disclosed on pages 89 to 90 of Lamansky, et al., WO 02/15645A which is incorporated herein by reference. Preferred examples of the auxiliary ligand include an acetylacetonate (acac) and a picolinate (pic) and derivatives thereof. In the invention, from the viewpoint that stability and high luminous efficiency of the complex are obtainable, the auxiliary ligand is preferably an acetylacetonate.

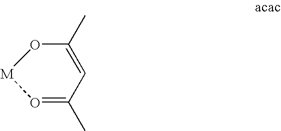

One of preferred embodiments of the compound represented by the foregoing general formula (T-1) is a compound represented by the following general formula (T-2).

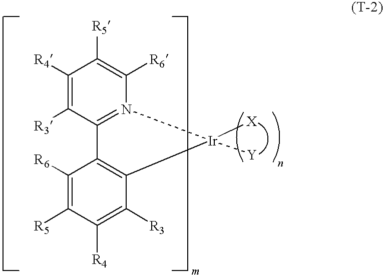

In the general formula (T-2), R₃′ represents an alkyl group, a heteroalkyl group, an aryl group or a heteroaryl group and may further have a substituent Z.

Each of R₄′ to R₆′ independently represents a hydrogen atom, an alkyl group, an alkenyl group, an alkynyl group, a heteroalkyl group, an aryl group or a heteroaryl group and may further have a substituent Z.

R₃′ and R₄′, R₄′ and R₅′, and R₅′ and R₆′ may be each independently bonded to each other to form a condensed 4-membered to 7-membered ring, the condensed 4-membered to 7-membered ring is a cycloalkyl, a cycloheteroalkyl, an aryl or a heteroaryl, and the condensed 4-membered to 7-membered ring may further have a substituent Z.

R₃′ and R₆ may be connected to each other via a connecting group selected among —CR₂—CR₂—, —CR═CR—, —CR₂, —O—, —NR—, —O—CR₂—, —NR—CR₂— and —N═CR— to form a ring; and each R independently represents a hydrogen atom, an alkyl group, an alkenyl group, an alkynyl group, a heteroalkyl group, an aryl group or a heteroaryl group and may further have a substituent Z.

R₅ represents an aryl group or a heteroaryl group and may be further substituted with a non-aromatic group.

Each of R₃, R₄ and R₆ independently represents a hydrogen atom, an alkyl group, an alkenyl group, an alkynyl group, —CN, —a perfluoroalkyl group, a trifluorovinyl group, —CO₂R, —C(O)R, —NR₂, —NO₂, —OR, a halogen atom, an aryl group or a heteroaryl group and may further have a substituent Z.

R₃ and R₄ may be bonded to each other to form a condensed 4-membered to 7-membered ring, the condensed 4-membered to 7-membered ring is a cycloalkyl, a cycloheteroalkyl, an aryl or a heteroaryl, and the condensed 4-membered to 7-membered ring may further have a substituent Z.

Each Z independently represents a halogen atom, —R′, —OR′, —N(R′)₂, —SR′, —C(O)R′, —C(O)OR′, —C(O)N(R′)₂, —CN, —NO₂, —SO₂, —SOR′, —SO₂R′ or —SO₃R′; and each R′ independently represents a hydrogen atom, an alkyl group, a perhaloalkyl group, an alkenyl group, an alkynyl group, a heteroalkyl group, an aryl group or a heteroaryl group.

(X—Y) represents an auxiliary ligand.

m represents an integer of from 1 to 3; and n represents an integer of from 0 to 2.

(m+n) is 3.

In the general formula (T-2), R₃′, R₃ to R₆, (X—Y), m and n are synonymous with R₃′, R₃ to R₆, (X—Y), m and n in the general formula (T-1), respectively, and preferred ranges thereof are also the same.

Each of R₄′ to R₆′ is synonymous with R₃′.

R₄′ is preferably a hydrogen atom, an alkyl group, an aryl group or a fluoro group, and more preferably a hydrogen atom.

Each of R₅′ and R₆′ preferably represents a hydrogen atom, or R₅′ and R₆′ are preferably bonded to each to form a condensed 4-membered to 7-membered ring. The condensed 4-membered to 7-membered ring is more preferably a cycloalkyl, a cycloheteroalkyl, an aryl or a heteroaryl, and further preferably an aryl.

The substituent Z in each of R₄′ to R₆′ is preferably an alkyl group, an alkoxy group, a fluoro group, a cyano group, an alkylamino group or a diarylamino group, and more preferably an alkyl group.

One of preferred embodiments of the compound represented by the foregoing general formula (T-1) is a compound represented by the following general formula (A9).

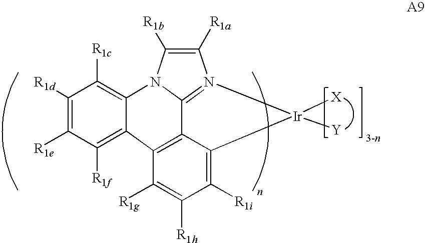

In the general formula (A9), the definitions of R₁ₐ to R₁ᵢ and preferred ranges thereof are the same as those in R₃ to R₆ in the general formula (T-1), respectively. The definitions of X and Y and preferred ranges thereof are the same as those in X and Y in the general formula (T-1), respectively. n represents an integer of from 0 to 3.

Specific examples of the compound represented by the general formula (T-1) are enumerated below, but it should not be construed that the invention is limited thereto.

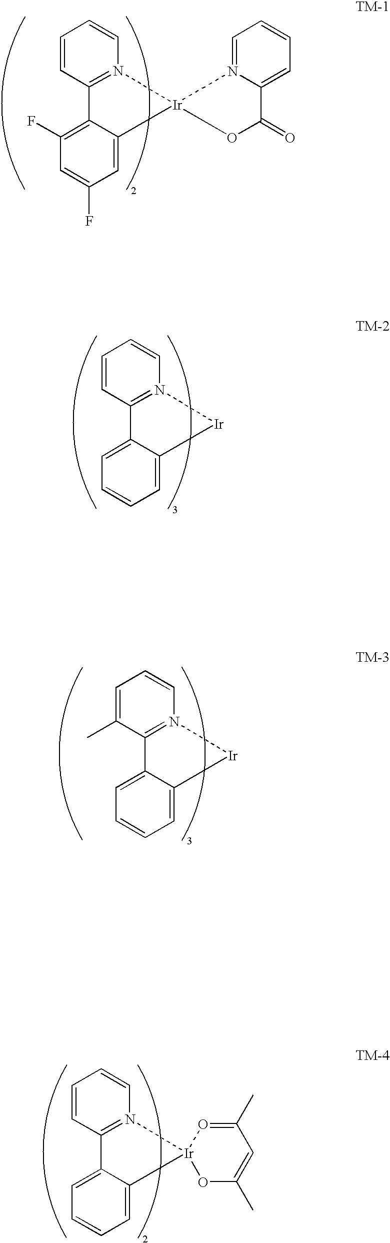

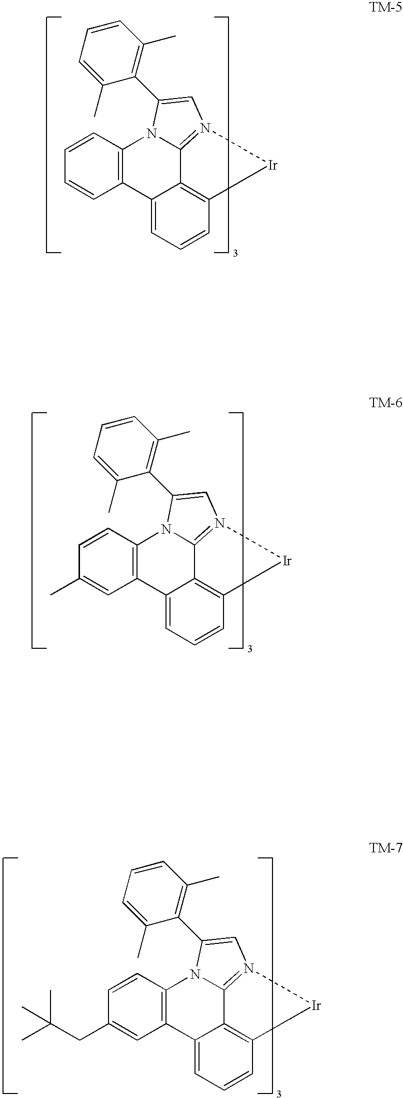

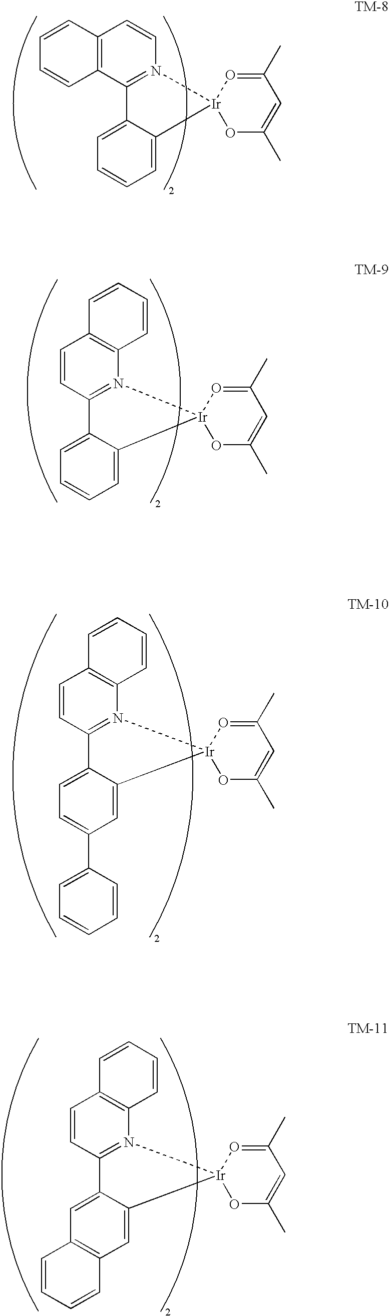

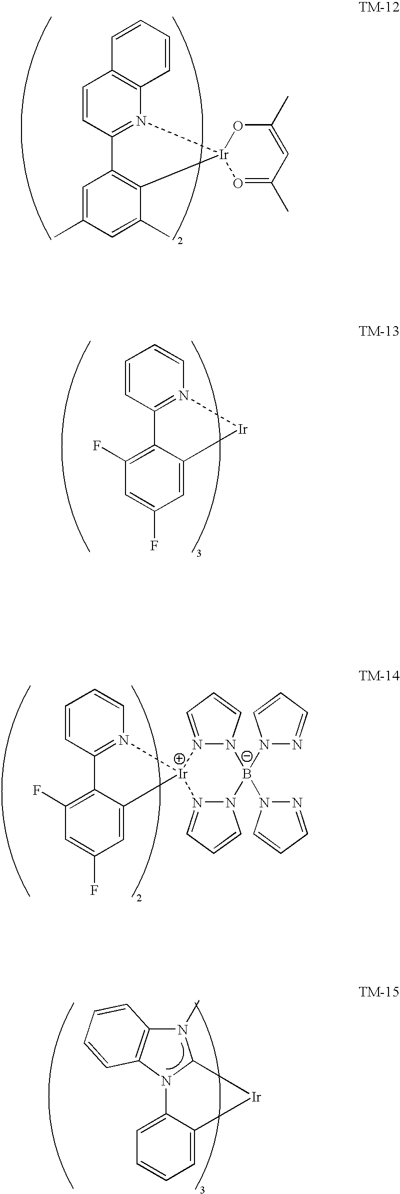

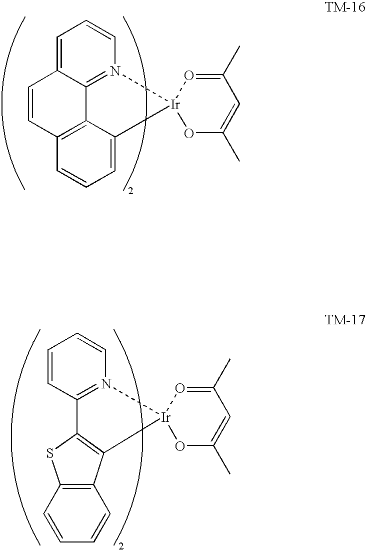

The compounds exemplified as the compound represented by the foregoing general formula (T-1) can be synthesized by a method disclosed in JP-A-2009-99783 and various methods disclosed in U.S. Pat. No. 7,279,232, etc. For example, TR-1 can be synthesized using 2-chloromethylquinoline as a starting raw material by a method disclosed at column 24, line 1 to column 27, line 33 of U.S. Pat. No. 7,279,232. Also, TG-1 can be synthesized using 2-bromo-3-methylpyridine as a starting raw material by a method disclosed at column 29, line 1 to column 31, line 29 of U.S. Pat. No. 7,279,232.

In the invention, though the compound represented by the general formula (T-1) is contained in the light emitting layer, its applications are not limited, but the compound represented by the general formula (T-1) may be further contained in any layer within the organic layer.

In the invention, in order to more suppress a change in chromaticity at the time of high-temperature driving, it is preferable that the compound represented by the general formula (1) and the compound represented by the general formula (T-1) are contained in the light emitting layer.

The light emitting material in the light emitting layer is generally contained in an amount of from 0.1% by mass to 50% by mass relative to the mass of all of the compounds capable for forming the light emitting layer in the light emitting layer. From the viewpoints of durability and external quantum efficiency, a content of the light emitting material is preferably from 1% by mass to 50% by mass, and more preferably from 2% by mass to 40% by mass.

Though a thickness of the light emitting layer is not particularly limited, in general, it is preferably from 2 nm to 500 nm. From the viewpoint of external quantum efficiency, the thickness of the light emitting layer is more preferably from 3 nm to 200 nm, and further preferably from 5 nm to 100 nm.

### <Host Material>

In addition to the compound of the invention, for example, examples of the host material which is used in the invention include the following materials.

The host material includes an electron transport material and a hole transport material. The host material may be a single kind or two or more kinds. For example, there is exemplified a constitution of a mixture of an electron transporting host material and a hole transporting host material.

Examples thereof include pyrrole, indole, carbazole, azaindole, azacarbazole, triazole, oxazole, oxadiazole, pyrazole, imidazole, thiophene, polyarylalkanes, pyrazoline, pyrazolone, phenylenediamine, arylamines, amino-substituted chalcone, styrylanthracene, fluorenone, hydrazone, stilbene, silazane, aromatic tertiary amine compounds, styrylamine compounds, porphyrin based compounds, polysilane based compounds, poly(N-vinylcarbazole), aniline based copolymers, thiophene oligomers, conductive high-molecular weight oligomers such as polythiophene, organic silanes, carbon films, pyridine, pyrimidine, triazine, imidazole, pyrazole, triazole, oxazole, oxadiazole, fluorenone, anthraquinodimethane, anthrone, diphenylquinone, thiopyrane dioxide, carbodiimide, fluorenylidenemethane, distyrylpyrazine, fluorine-substituted aromatic compounds, heterocyclic tetracarboxylic acid anhydrides such as naphthaleneperillene, phthalocyanine, metal complexes or metal phthalocyanines of an 8-quinolinol derivative, various metal complexes represented by metal complexes containing benzoxazole or benzothiazole as a ligand and derivatives thereof (may have a substituent or a condensed ring).

In the light emitting layer in the invention, from the standpoints of color purity, luminous efficiency and driving durability, it is preferable that the lowest excited triplet energy (T₁ energy) of the host material (including the compound of the invention) is higher than a T₁ energy of the phosphorescent material. T₁ of the host material is higher than T₁ of the phosphorescent material by preferably 0.1 eV or more, more preferably 0.2 eV or more, and further preferably 0.3 eV or more.

When T₁ of the host material is smaller than T₁ of the phosphorescent material, the light emission is quenched, and hence, the host material is required to have larger T₁ than the phosphorescent material. Also, even in the case where T₁ of the host material is larger than T₁ of the phosphorescent material, when a difference in T₁ between the both is small, reverse energy transport from the phosphorescent material to the host material partially occurs, and hence, a lowering in the efficiency or a lowering in the durability may be caused. Accordingly, a host material having sufficiently large T₁ and high chemical stability and carrier injection and transport properties is demanded.

Also, though a content of the host compound in the invention is not particularly limited, from the viewpoints of luminous efficiency and driving voltage, it is preferably 10% by mass or more and not more than 99% by mass, more preferably 30% by mass or more and not more than 97% by mass, and further preferably 50% by mass or more and not more than 95% by mass relative to the mass of all of the compounds capable for forming the light emitting layer. In the case where a mixture of the compound of the invention and other host compound is used as the host material, the amount of the compound of the invention is preferably 30% by mass or more and not more than 100% by mass, more preferably 50% by mass or more and not more than 100% by mass, and further preferably 70% by mass or more and not more than 100% by mass relative to the total mass of the host material.

The light emitting layer may contain additives such as a charge trapping agent, an alignment control agent and an intermolecular mutual action adjustor in addition to the light emitting material and the host material. Though any material can be used as the additive, it is preferably a hydrocarbon compound.

Also, the hydrocarbon compound is preferably a compound represented by the following general formula (VI).

By appropriately using the compound represented by the general formula (VI) together with the light emitting material, an intermolecular mutual action of the material is appropriately controlled, and an energy gap mutual action between adjacent molecules is made uniform. Thus, it becomes possible to more lower the driving voltage.

Also, the compound represented by the general formula (VI) which is used in the organic electroluminescence device is excellent in chemical stability, small in denaturation such as decomposition of the material during the device driving and capable of preventing a lowering of the efficiency of the organic electroluminescence device or a lowering of the device life due to a decomposition product of the material.

The compound represented by the general formula (VI) is described.

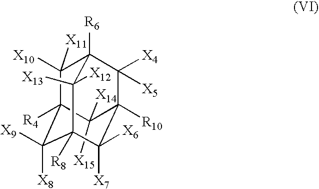

In the general formula (VI), each of R₄, R₆, R₈, R₁₀ and X₄ to X₁₅ independently represents a hydrogen atom, an alkyl group or an aryl group.

The alkyl group represented by each of R₄, R₆, R₈, R₁₀ and X₄ to X₁₅ in the general formula (VI) may be substituted with an adamantane structure or an aryl structure. The alkyl group has preferably from 1 to 70 carbon atoms, more preferably from 1 to 50 carbon atoms, further preferably from 1 to 30 carbon atoms, even further preferably from 1 to 10 carbon atoms, and especially preferably from 1 to 6 carbon atoms, with a linear alkyl group having from 2 to 6 carbon atoms being most preferable.

Examples of the alkyl group represented by each of R₄, R₆, R₈, R₁₀ and X₄ to X₁₅ in the general formula (VI) include an n-C₅₀H₁₀₁ group, an n-C₃₀H₆₁ group, a 3-(3,5,7-triphenyladamantan-1-yl)propyl group (carbon atom number: 31), a trityl group (carbon atom number: 19), a 3-(adamantan-1-yl)propyl group (carbon atom number: 13), a 9-decalyl group (carbon atom number: 10), a benzyl group (carbon atom number: 7), a cyclohexyl group (carbon atom number: 6), an n-hexyl group (carbon atom number: 6), an n-pentyl group (carbon atom number: 5), an n-butyl group (carbon atom number: 4), an n-propyl group (carbon atom number: 3), a cyclopropyl group (carbon atom number: 3), an ethyl group (carbon atom number: 2) and a methyl group (carbon atom number: 1).

The aryl group represented by each of R₄, R₆, R₈, R₁₀ and X₄ to X₁₅ in the general formula (VI) may be substituted with an adamantane structure or an alkyl structure. The aryl group has preferably from 6 to 30 carbon atoms, more preferably from 6 to 20 carbon atoms, further preferably from 6 to 15 carbon atoms, especially preferably from 6 to 10 carbon atoms, and most preferably 6 carbon atoms.

Examples of the aryl group represented by each of R₄, R₆, R₈, R₁₀ and X₄ to X₁₅ in the general formula (VI) include a 1-pyrenyl group (carbon atom number: 16), a 9-anthracenyl group (carbon atom number: 14), a 1-naphthyl group (carbon atom number: 10), a 2-naphthyl group (carbon atom number: 10), a p-t-butylphenyl group (carbon atom number: 10), a 2-m-xylyl group (carbon atom number: 8), a 5-m-xylyl group (carbon atom number: 8), an o-tolyl group (carbon atom number: 7), an m-tolyl group (carbon atom number: 7), a p-tolyl group (carbon atom number: 7) and a phenyl group (carbon atom number: 6).

Though each of R₄, R₆, R₈ and R₁₀ in the general formula (VI) may be a hydrogen atom, an alkyl group or an aryl group, from the viewpoint that a high glass transition temperature is preferable, it is preferable that at least one of them is an aryl group; it is more preferable that at least two of them are an aryl group; and it is especially preferable that three or four of them are an aryl group.

Though each of X₄ to X₁₅ in the general formula (VI) may be a hydrogen atom, an alkyl group or an aryl group, each of X₄ to X₁₅ is preferably a hydrogen atom or an aryl group, and especially preferably a hydrogen atom.

Since the organic electroluminescence device is prepared using a vacuum vapor deposition process or a solution coating process, from the viewpoints of vapor deposition adaptability and solubility, a molecular weight of the compound represented by the general formula (VI) in the invention is preferably not more than 2,000, more preferably not more than 1,200, and especially preferably not more than 1,000. Also, from the viewpoint of vapor deposition adaptability, when the molecular weight is too low, a vapor pressure is small, change from a gas phase to a solid phase does not take place, and it is difficult to form an organic layer. Therefore, the molecular weight of the compound represented by the general formula (VI) is preferably 250 or more, more preferably 350 or more, and especially preferably 400 or more.

It is preferable that the compound represented by the general formula (VI) is a solid at room temperature (25° C.); it is more preferable that the compound represented by the general formula (VI) is a solid in the range of from room temperature (25° C.) to 40° C.; and it is especially preferable that the compound represented by the general formula (VI) is a solid in the range of from room temperature (25° C.) to 60° C.

In the case where the compound represented by the general formula (VI), which does not form a solid at room temperature (25° C.), is used, it is possible to form a solid phase at the normal temperature upon being combined with other material.

The compound represented by the general formula (VI) is not limited with respect to its applications and may be contained in any layer within the organic layer. As to the layer into which the compound represented by the general formula (VI) in the invention is introduced, the compound represented by the general formula (VI) is preferably contained in any one or a plurality of a light emitting layer, a hole injection layer, a hole transport layer, an electron transport layer, an electron injection layer, an exciton blocking layer and a charge blocking layer; more preferably contained in any one or a plurality of a light emitting layer, a hole injection layer, a hole transport layer, an electron transport layer and an electron injection layer; especially preferably contained in any one or a plurality of a light emitting layer, a hole injection layer and a hole transport layer; and most preferably contained in a light emitting layer as described later.

In the case where the compound represented by the general formula (VI) is used in the organic layer, it is necessary that a content of the compound represented by the general formula (VI) is controlled to an extent that charge transport properties are not hindered. The content of the compound represented by the general formula (VI) is preferably from 0.1 to 70% by mass, more preferably from 0.1 to 30% by mass, and especially preferably from 0.1 to 25% by mass.

Also, in the case where the compound represented by the general formula (VI) is used in plural organic layers, it is preferable that the compound represented by the general formula (VI) is contained in an amount falling within the foregoing range in each of the layers.

Only one kind of the compound represented by the general formula (VI) may be contained in any one organic layer; and plural kinds of the compound represented by the general formula (VI) may be combined in an arbitrary proportion and contained.

Specific examples of the compound represented by the general formula (VI) are enumerated below, but it should not be construed that the invention is limited thereto.

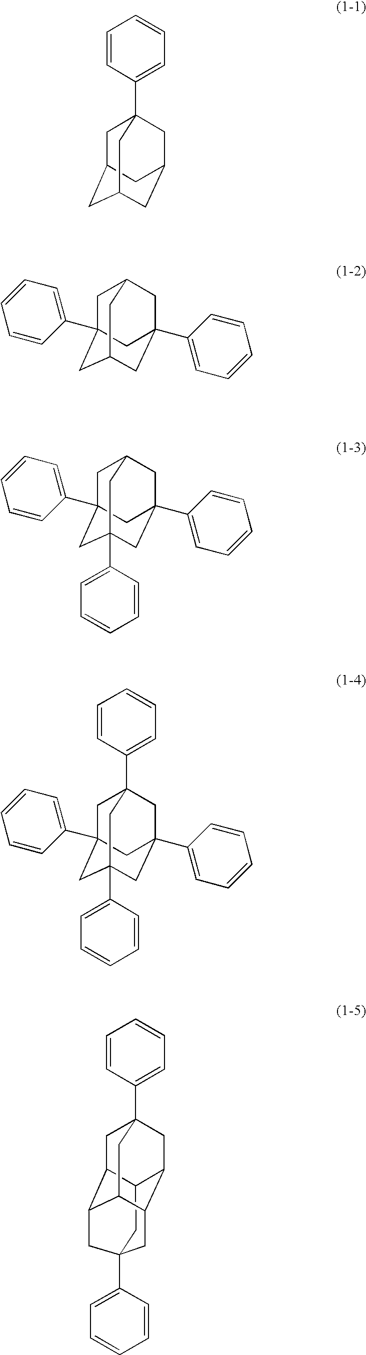

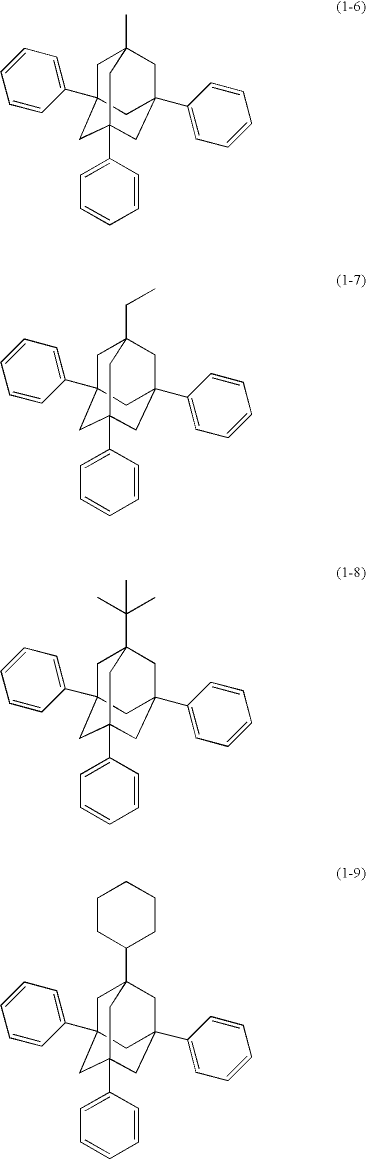

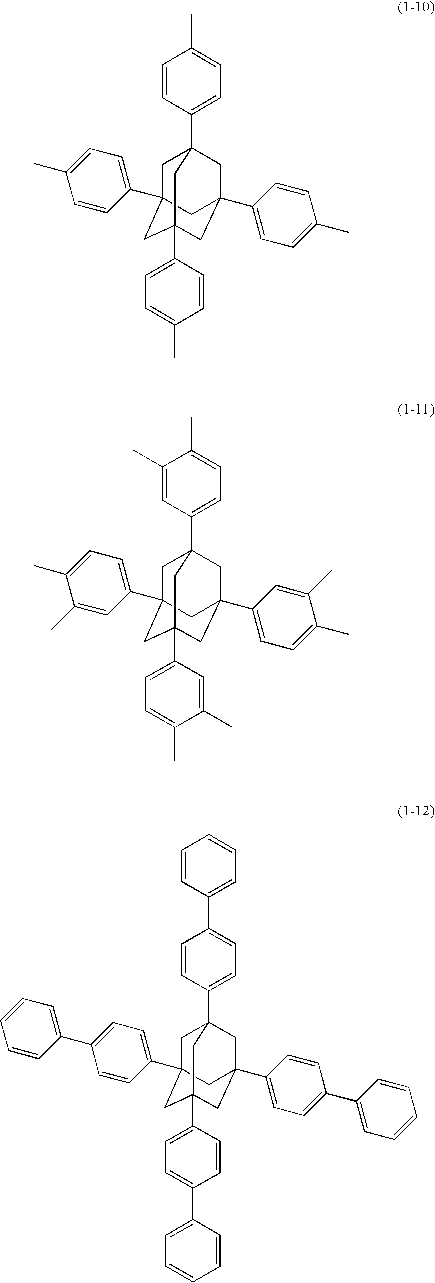

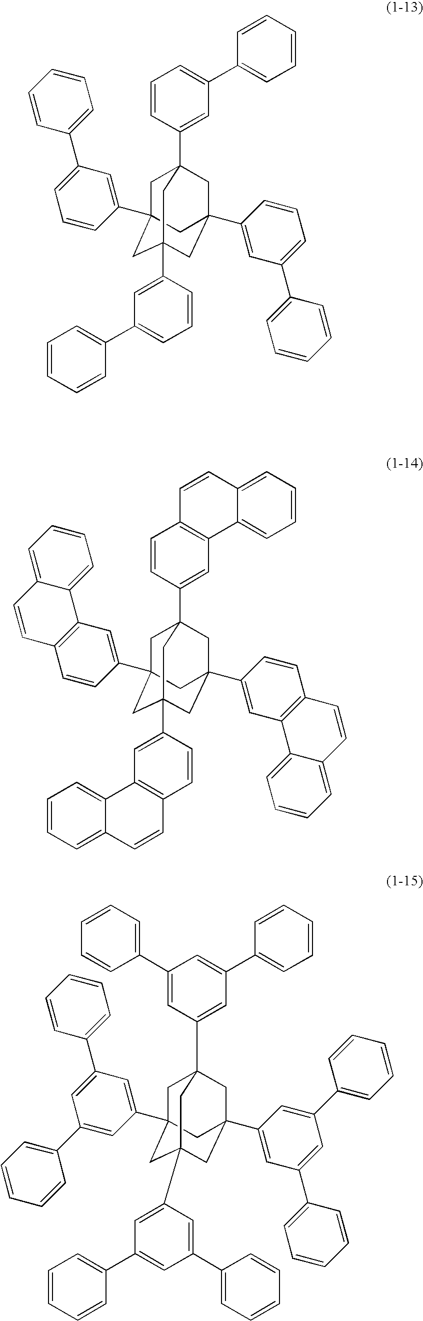

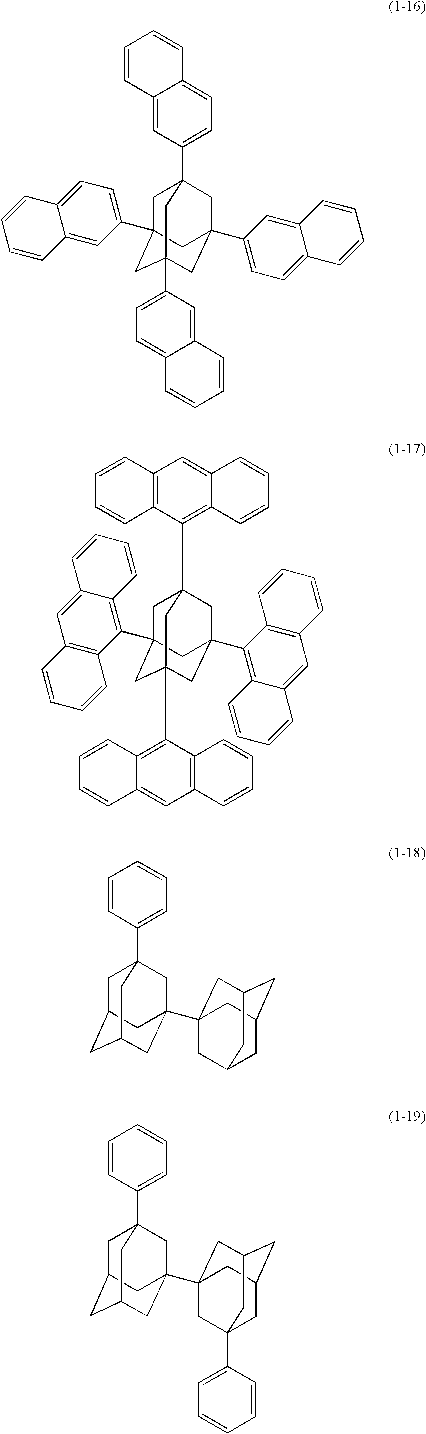

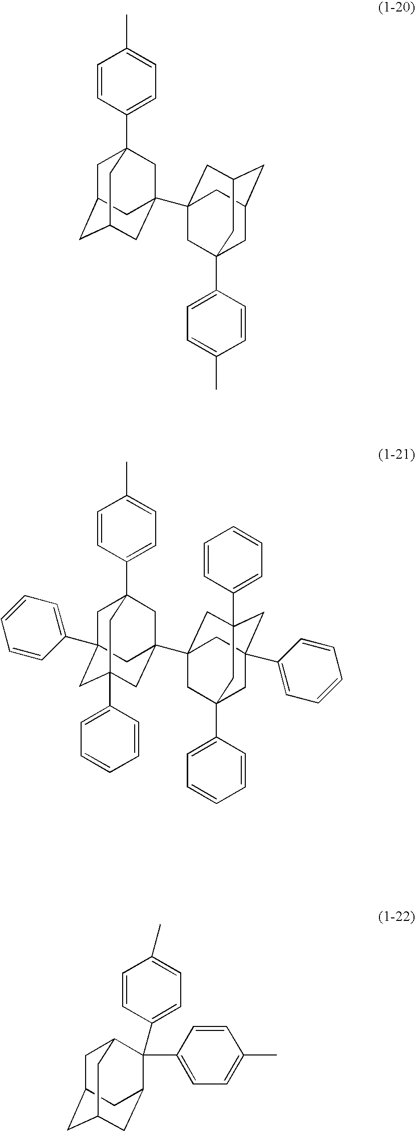

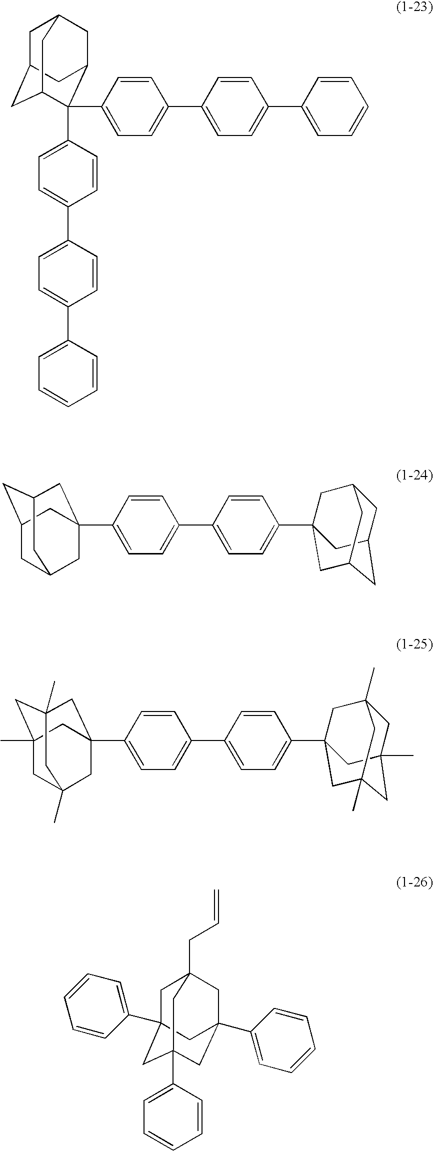

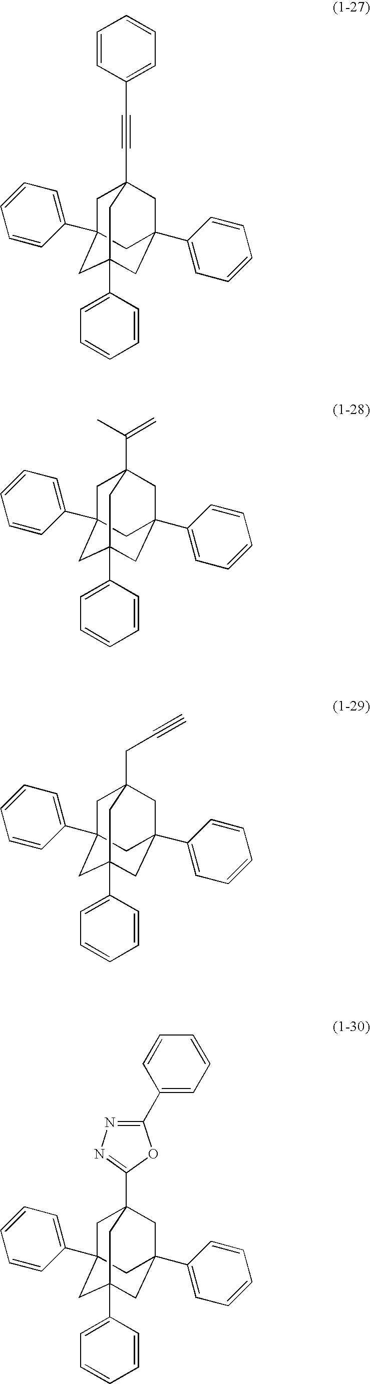

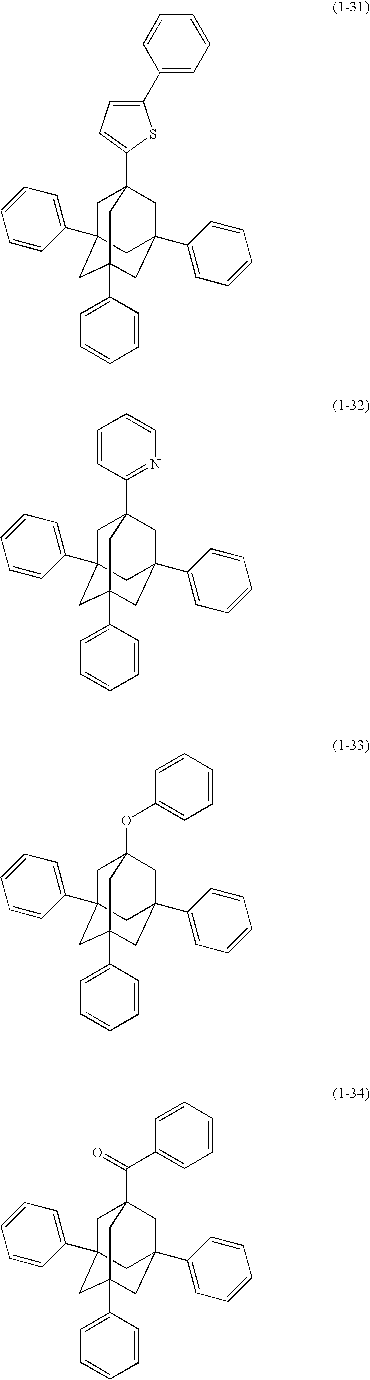

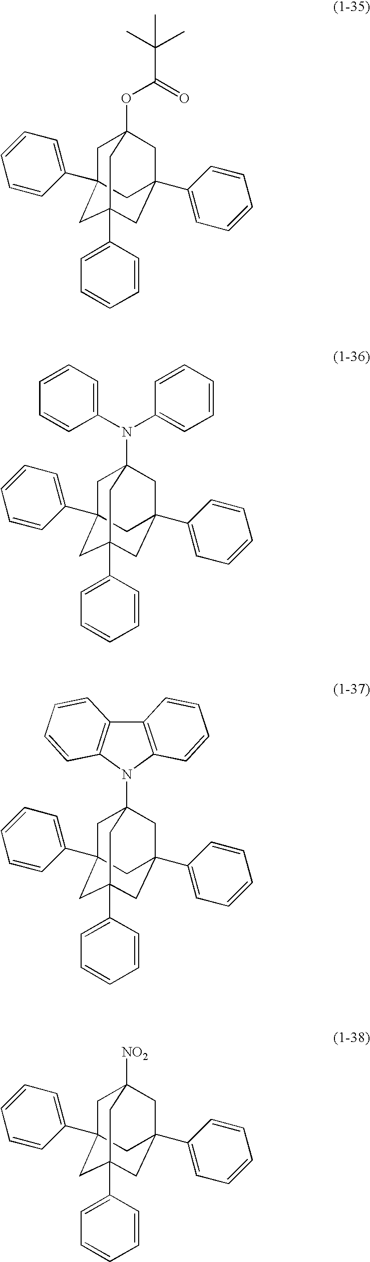

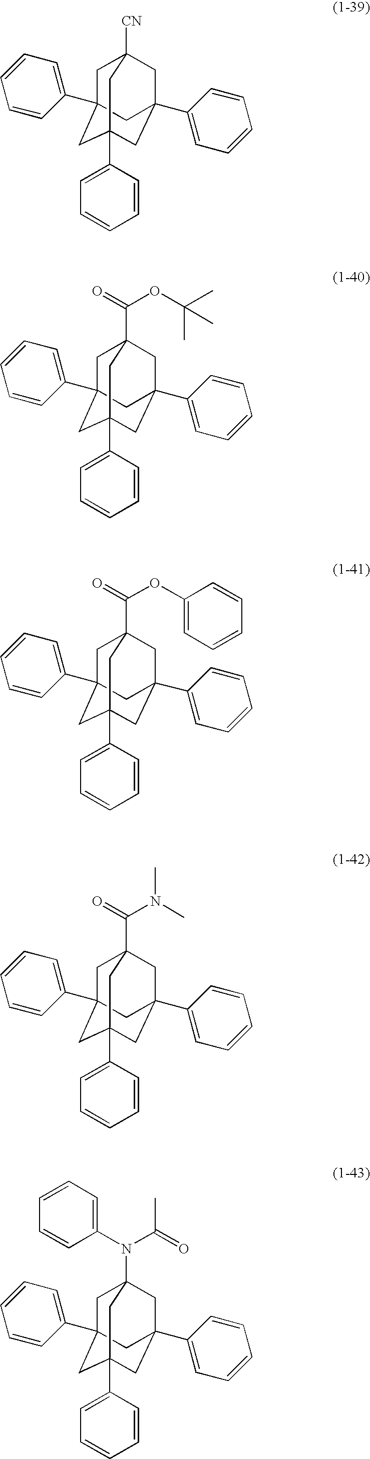

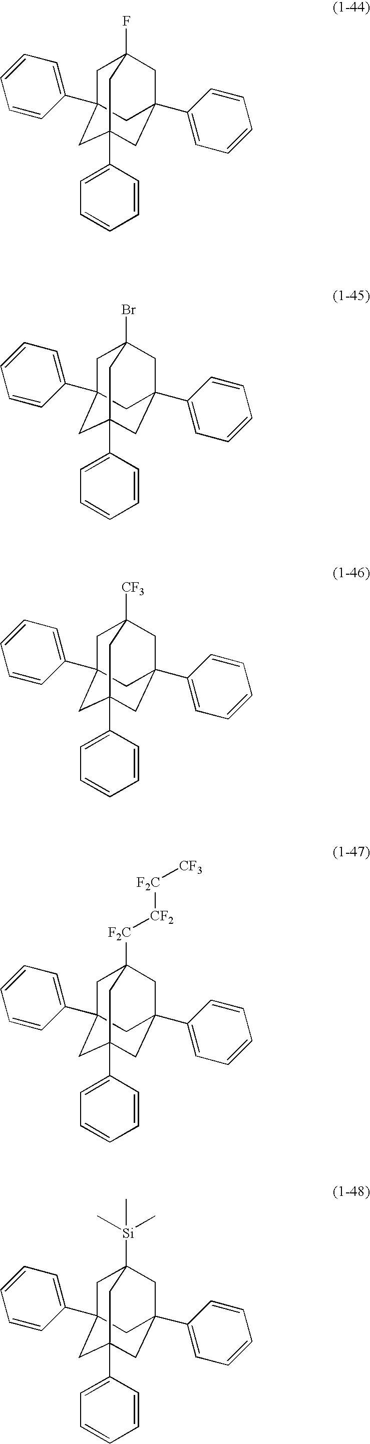

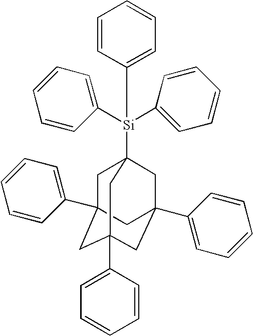

The compound represented by the general formula (VI) can be synthesized by properly combining adamantane or a halogenated adamantane with an alkyl halide or an alkyl magnesium halide (Grignard reagent). For example, a halogenated adamantane can be coupled with an alkyl halide using indium (see Document 1). Also, an alkyl halide can be converted into an alkyl copper reagent and then coupled with a Grignard reagent of an aromatic compound (see Document 2). Also, an alkyl halide can be coupled with an appropriate aryl boric acid using a palladium catalyst (see Document 3).

Document 1: Tetrahedron Lett., 39, 9557 to 9558 (1998)

Document 2: Tetrahedron Lett., 39, 2095 to 2096 (1998)

Document 3: J. Am. Chem. Soc., 124, 13662 to 13663 (2002)

The adamantane structure having an aryl group can be synthesized by properly combining adamantane or a halogenated adamantane with a corresponding arene or aryl halide.

In the foregoing manufacturing method, in the case where the defined substituents are changed under a condition of a certain synthesis method or are inadequate for carrying out the instant method, the manufacture can be easily made by means of, for example, protection, deprotection, etc. of a functional group (see, for example, Protective Groups in Organic Synthesis, written by T. W. Greene, John Wiley & Sons Inc. (1981), etc.). Also, the order of the reaction steps such as introduction of a substituent can be properly changed, if desired.

### (Charge Transport Layer)

The charge transport layer as referred to herein is a layer in which when a voltage is impressed to the organic electroluminescence device, charge transport takes place. Specific examples thereof include a hole injection layer, a hole transport layer, an electron blocking layer, a light emitting layer, a hole blocking layer, an electron transport layer and an electron injection layer. Of these, a hole injection layer, a hole transport layer, an electron blocking layer and a light emitting layer are preferable. So far as the charge transport layer formed by the coating method is a hole injection layer, a hole transport layer, an electron blocking layer or a light emitting layer, it is possible to manufacture an organic electroluminescence device at low cost and high efficiency. Also, the charge transport layer is more preferably a hole injection layer, a hole transport layer or an electron blocking layer.

### (Hole Injection Layer and Hole Transport Layer)

Each of the hole injection layer and the hole transport layer is a layer having a function of accepting a hole from the anode or the anode side to transport it into the cathode side. Each of a hole injection material and a hole transport material which are used in these layers may be a low-molecular weight compound or a polymer compound.

Specifically, each of the hole injection layer and the hole transport layer is preferably a layer containing, in addition to the compound of the invention, a pyrrole derivative, a carbazole derivative, a triazole derivative, an oxazole derivative, an oxadiazole derivative, an imidazole derivative, a polyarylalkane derivative, a pyrazoline derivative, a pyrazolone derivative, a phenylenediamine derivative, an arylamine derivative, an amino-substituted chalcone derivative, a styrylanthracene derivative, a fluorenone derivative, a hydrazone derivative, a stilbene derivative, a silazane derivative, an aromatic tertiary amine compound, a styrylamine compound, a phthalocyanine based compound, a porphyrin based compound, a thiophene derivative, an organic silane derivative, carbon, a metal complex of every sort such as an iridium complex or the like.

An electron-accepting dopant can be contained in the hole injection layer or the hole transport layer of the organic EL device of the invention. As the electron-accepting dopant which is introduced into the hole injection layer or the hole transport layer, any inorganic compound or organic compound can be used so far as it is electron-accepting and has properties of oxidizing an organic compound.

Specifically, examples of the inorganic compound include metal halides such as ferric chloride, aluminum chloride, gallium chloride, indium chloride and antimony pentachloride; and metal oxides such as vanadium pentoxide and molybdenum trioxide.

As the organic compound, compounds having, as a substituent, a nitro group, a halogen, a cyano group, a trifluoromethyl group, etc., quinone based compounds, acid anhydride based compounds, fullerenes and the like can be suitably used.

Besides, compounds disclosed in JP-A-6-212153, JP-A-11-111463, JP-A-11-251067, JP-A-2000-196140, JP-A-2000-286054, JP-A-2000-315580, JP-A-2001-102175, JP-A-2001-160493, JP-A-2002-252085, JP-A-2002-56985, JP-A-2003-157981, JP-A-2003-217862, JP-A-2003-229278, JP-A-2004-342614, JP-A-2005-72012, JP-A-2005-166637, JP-A-2005-209643, etc. can also be suitably used.

Of these, hexacyanobutadiene, hexacyanobenzene, tetracyanoethylene, tetracyanoquinodimethane, tetrafluorotetracyanoquinodimethane, p-fluoranyl, p-chloranyl, p-bromanyl, p-benzoquinone, 2,6-dichlorobenzoquinone, 2,5-dichlorobenzoquinone, 1,2,4,5-tetracyanobenzene, 1,4-dicyanotetrafluorobenzene, 2,3-dichloro-5,6-dicyanobenzoquinone, p-dinitrobenzene, m-dinitrobenzene, o-dinitrobenzene, 1,4-naphthoquinone, 2,3-dichloronaphthoquinone, 1,3-dinitronaphthalene, 1,5-dinitronaphthalene, 9,10-anthraquinone, 1,3,6,8-tetranitrocarbazole, 2,4,7-trinitro-9-fluorenone, 2,3,5,6-tetracyanopyridine and fullerene C60 are preferable; hexacyanobutadiene, hexacyanobenzene, tetracyanoethylene, tetracyanoquinodimethane, tetrafluorotetracyanoquinodimethane, p-fluoranyl, p-chloranyl, p-bromanyl, 2,6-dichlorobenzoquinone, 2,5-dichlorobenzoquinone, 2,3 -dichloronaphthoquinone, 1,2,4,5-tetracyanobenzene, 2,3-dichloro-5,6-dicyanobenzoquinone and 2,3,5,6-tetracyanopyridine are more preferable; and tetrafluorotetracyanoquinodimethane is especially preferable.

Such an electron-accepting dopant may be used singly or in combinations of two or more kinds thereof. Though the use amount of the electron-accepting dopant varies depending upon the kind of the material, it is preferably from 0.01% by mass to 50% by mass, more preferably from 0.05% by mass to 20% by mass, and especially preferably from 0.1% by mass to 10% by mass relative to the hole transport layer material.

From the viewpoint of lowering the driving voltage, a thickness of each of the hole injection layer and the hole transport layer is preferably not more than 500 nm.

The thickness of the hole transport layer is preferably from 1 nm to 500 nm, more preferably from 5 nm to 200 nm, and further preferably from 10 nm to 100 nm Also, the thickness of the hole injection layer is preferably from 0.1 nm to 200 nm, more preferably from 0.5 nm to 100 nm, and further preferably from 1 nm to 100 nm.

Each of the hole injection layer and the hole transport layer may be of a single layer structure composed of one or two or more kinds of the foregoing materials or may be of a multilayer structure composed of a plurality of layers of the same or different compositions.

### (Electron Injection Layer and Electron Transport Layer)

Each of the electron injection layer and the electron transport layer is a layer having a function of accepting an electron from the cathode or the cathode side and to transport it into the anode side. Each of an electron injection material and an electron transport material which are used in these layers may be a low-molecular weight compound or a polymer compound.

Specifically, each of the electron injection layer and the electron transport layer is preferably a layer containing, in addition to the compound of the invention, a pyridine derivative, a quinoline derivative, a pyrimidine derivative, a pyrazine derivative, a phthalazine derivative, a phenanthroline derivative, a triazine derivative, a triazole derivative, an oxazole derivative, an oxadiazole derivative, an imidazole derivative, a fluorenone derivative, an anthraquinodimethane derivative, an anthrone derivative, a diphenylquinone derivative, a thiopyrandioxide derivative, a carbodiimide derivative, a fluorenylidenemethane derivative, a distyrylpyrazine derivative, an aromatic tetracarboxylic acid anhydride of naphthalene, perylene, etc., a phthalocyanine derivative, a complex of every sort represented by complexes of an 8-quinolinol derivative and complexes containing, as a ligand, phthalocyanine, benzoxazole or benzothiazole, an organic silane derivative represented by silole, or the like.

An electron-donating dopant can be contained in the electron injection layer or the electron transport layer of the organic EL device of the invention. As the electron-donating dopant which is introduced into the electron injection layer or the electron transport layer, alkali metals such as Li, alkaline earth metals such as Mg, transition metals including rare earth metals, reducing organic compounds and the like are suitably used so far as they are electron-donating and have properties of reducing an organic compound. In particular, a metal having a work function of not more than 4.2 eV can be suitably used as the metal. Specific examples thereof include Li, Na, K, Be, Mg, Ca, Sr, Ba, Y, Cs, La, Sm, Gd and Yb. Also, examples of the reducing organic compound include nitrogen-containing compounds, sulfur-containing compounds and phosphorus-containing compounds.

Besides, materials disclosed in JP-A-6-212153, JP-A-2000-196140, JP-A-2003-68468, JP-A-2003-229278, JP-A-2004-342614, etc. can be used.

Such an electron-donating dopant may be used singly or in combinations of two or more kinds thereof. Though the use amount of the electron-donating dopant varies depending upon the kind of the material, it is preferably from 0.1% by mass to 99% by mass, more preferably from 1.0% by mass to 80% by mass, and especially preferably from 2.0% by mass to 70% by mass relative to the electron transport layer material.

From the viewpoint of lowering the driving voltage, a thickness of each of the electron injection layer and the electron transport layer is preferably not more than 500 nm.

The thickness of the electron transport layer is preferably from 1 nm to 500 nm, more preferably from 5 nm to 200 nm, and further preferably from 10 nm to 100 nm. Also, the thickness of the electron injection layer is preferably from 0.1 nm to 200 nm, more preferably from 0.2 nm to 100 nm, and further preferably from 0.5 nm to 50 nm.

Each of the electron injection layer and the electron transport layer may be of a single layer structure composed of one or two or more kinds of the foregoing materials or may be of a multilayer structure composed of a plurality of layers of the same or different compositions.

### (Hole Blocking Layer)

The hole blocking layer is a layer having a function of preventing permeation of the hole having been transported from the anode side to the light emitting layer into the cathode side. In the invention, the hole blocking layer can be provided as an organic layer adjacent to the light emitting layer on the cathode side.

Examples of the compound constituting the hole blocking layer include aluminum complexes such as aluminum(III) bis(2-methyl-8-quinolinato)4-phenylphenolate (abbreviated as “BAlq”); triazole derivatives; and phenanthroline derivatives such as 2,9-dimethyl-4,7-diphenyl-1,10-phenanthroline (abbreviated as “BCP”).

A thickness of the hole blocking layer is preferably from 1 nm to 500 nm, more preferably from 5 nm to 200 nm, and further preferably from 10 nm to 100 nm.

The hole blocking layer may be of a single layer structure composed of one or two or more kinds of the foregoing materials or may be of a multilayer structure composed of a plurality of layers of the same or different compositions.

### (Electron Blocking Layer)

The electron blocking layer is a layer having a function of preventing permeation of an electron having been transported from the cathode side to the light emitting layer into the anode side. In the invention, the electron blocking layer can be provided as an organic layer adjacent to the light emitting layer on the anode side.

Examples of the compound constituting the electron blocking layer include those exemplified above as the hole transport material.

A thickness of the electron blocking layer is preferably from 1 nm to 500 nm, more preferably from 5 nm to 200 nm, and further more preferably from 10 nm to 100 nm.

The electron blocking layer may be of a single layer structure composed of one or two or more kinds of the foregoing materials or may be of a multilayer structure composed of a plurality of layers of the same or different compositions.

### (Protective Layer)

In the invention, the whole of the organic EL device may be protected by a protective layer.

As a material to be contained in the protective layer, any material having a function of inhibiting the incorporation of a substance promoting the deterioration of the device, such as moisture and oxygen, into the device is useful.

The protective layer is described in detail in, for example, JP-A-2008-270736 and JP-A-2007-266458, and the matters disclosed in these patent documents can be applied to the invention.

### (Sealing)

Furthermore, in the device of the invention, the whole of the device may be sealed using a sealing vessel.

Also, a moisture absorber or an inert liquid may be sealed in a space between the sealing vessel and the luminescence device. Though the moisture absorber is not particularly limited, examples thereof include barium oxide, sodium oxide, potassium oxide, calcium oxide, sodium sulfate, calcium sulfate, magnesium sulfate, phosphorus pentoxide, calcium chloride, magnesium chloride, copper chloride, cesium fluoride, niobium fluoride, calcium bromide, vanadium bromide, molecular sieve, zeolite and magnesium oxide. Though the inert liquid is not particularly limited, examples thereof include paraffins, liquid paraffins, fluorine based solvents such as perfluoroalkanes, perfluoroamines and perfluoroethers, chlorine based solvents and silicon oils.

According to the device of the invention, light emission can be obtained by impressing a voltage of direct current (optionally including an alternating current component) (usually from 2 volts to 15 volts) or a current of direct current between the anode and the cathode.

As to the driving method of the device of the invention, driving methods disclosed in JP-A-2-148687, JP-A-6-301355, JP-A-5-29080, JP-A-7-134558, JP-A-8-234685, JP-A-8-241047, Japanese Patent No. 2784615 and U.S. Pat. Nos. 5,828,429 and 6,023,308 can be applied.

The device of the invention can be suitably utilized for display devices, displays, backlights, electro-photographs, light emission apparatuses, illumination light sources, recording light sources, exposure light sources, read light sources, markers, signboards, interiors, optical communications and so on.

Next, the light emission apparatus of the invention is described while referring to FIG. 2.

The light emission apparatus of the invention is one using the foregoing organic electroluminescence device.

FIG. 2 is a sectional view diagrammatically showing an example of the light emission apparatus of the invention.

A light emission apparatus 20 of FIG. 2 is configured to include a transparent substrate (supporting substrate) 2, an organic electroluminescence device 10, a sealing vessel 16 and so on.

The organic electroluminescence device 10 is configured such that an anode (first electrode) 3, an organic layer 11 and a cathode (second electrode) 9 are laminated in this order on the substrate 2. Also, a protective layer 12 is laminated on the cathode 9, and furthermore, the sealing vessel 16 is provided on the protective layer 12 via an adhesive layer 14. A part of each of the electrodes 3 and 9, a partition, an insulating layer and the like are omitted.

Here, a photocurable adhesive or a thermosetting adhesive such as an epoxy resin can be used as the adhesive layer 14, and for example, a thermosetting adhesive sheet can be used.

The application of the light emission apparatus of the invention is not particularly limited, and examples thereof include, in addition to illumination apparatuses, display apparatuses of television receiver, personal computer, mobile phone, electronic paper, etc.

Next, the illumination apparatus of the invention is described while referring to FIG. 3.

FIG. 3 is a sectional view diagrammatically showing an example of the illumination apparatus according to an embodiment of the invention.

As shown in FIG. 3, an illumination apparatus 40 according to an embodiment of the invention is provided with the foregoing organic EL device 10 and a light scattering member 30. More specifically, the illumination apparatus 40 is configured such that the substrate 2 of the organic EL device 10 and the light scattering member 30 come into contact with each other.

The light scattering member 30 is not particularly limited so far as it is able to scatter light. In FIG. 3, the light scattering member 30 is a member having a fine particle 32 dispersed in a transparent substrate 31. As the transparent substrate 31, for example, a glass substrate can be suitably exemplified. As the fine particle 32, a transparent resin fine particle can be suitably exemplified. As the glass substrate and the transparent resin fine particle, those which are known can be used. Such an illumination apparatus 40 is a unit which when light emission from the organic electroluminescence device 10 comes into a light incident surface 30A of the light scattering member 30, scatters the incident light by the light scattering member 30 and outputs the scattered light as illumination light from a light outgoing surface 30B.

### Examples

The invention is hereunder described in detail with reference to the following Examples, but it should not be construed that the invention is limited thereto.

The following Illustrative Compounds and Comparative Compounds were synthesized.

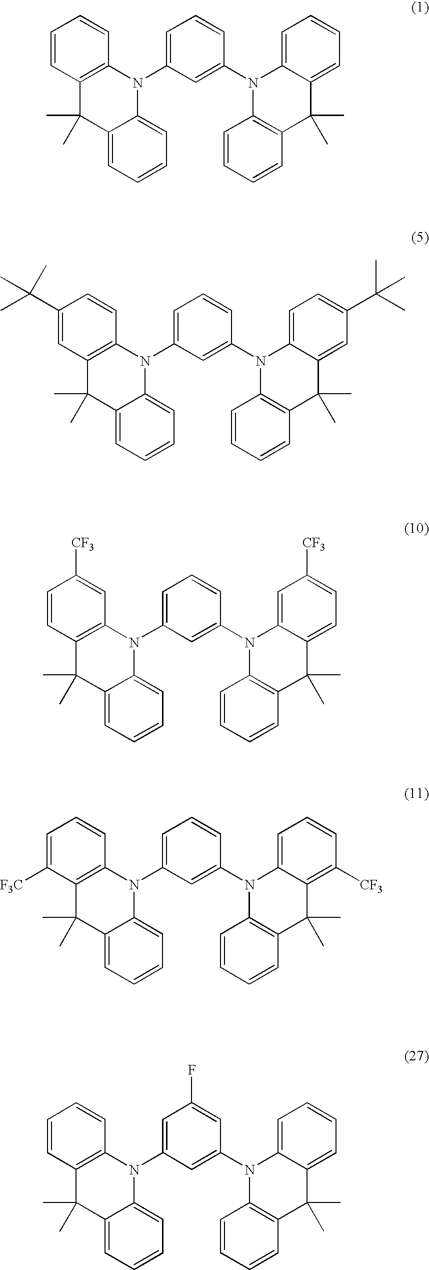

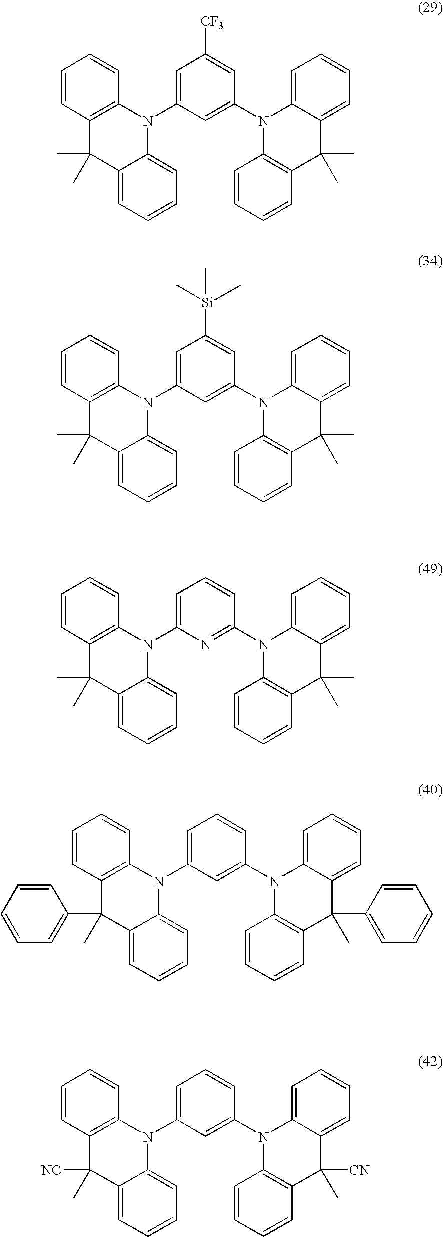

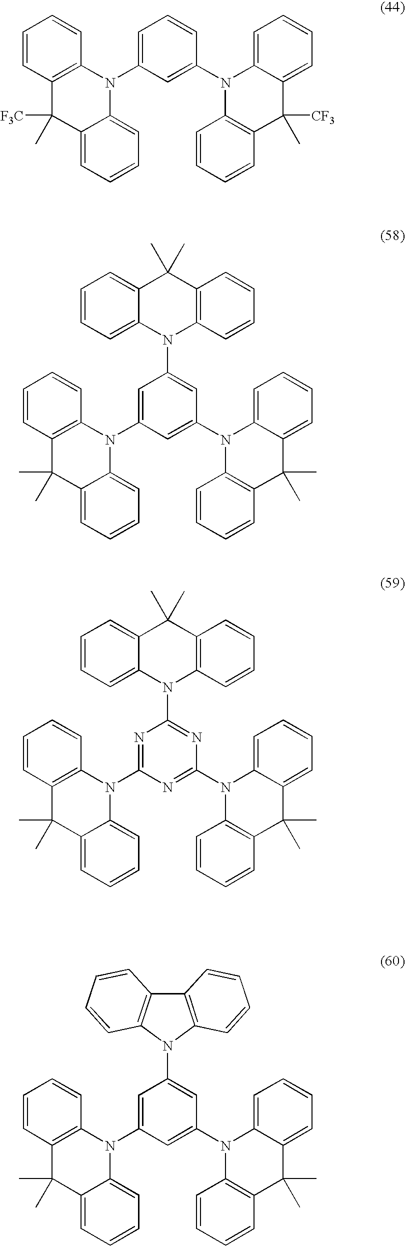

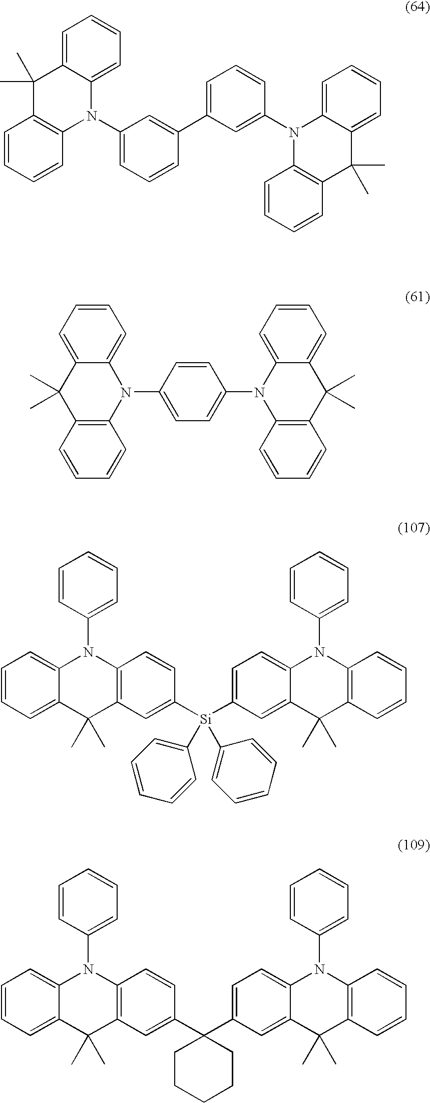

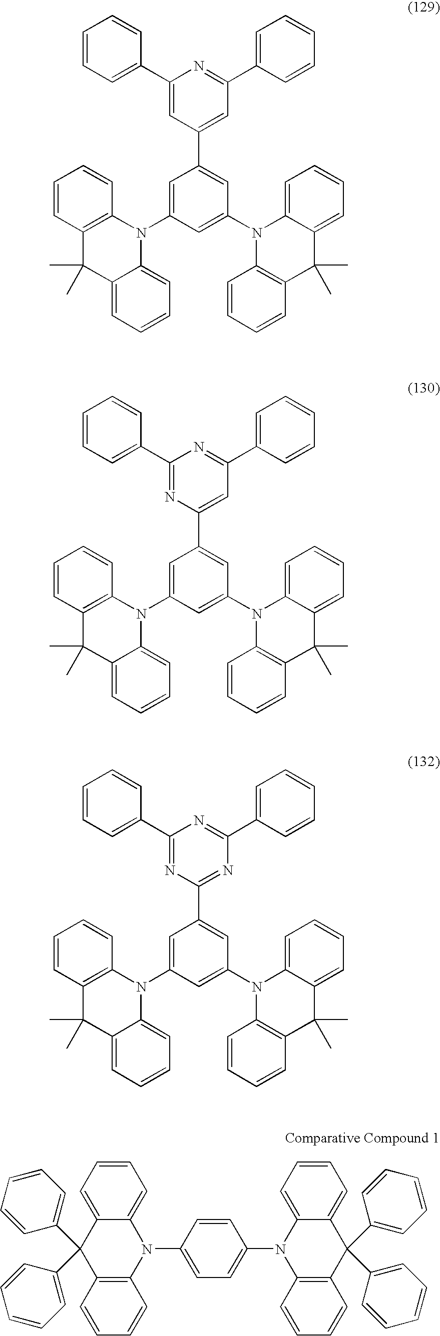

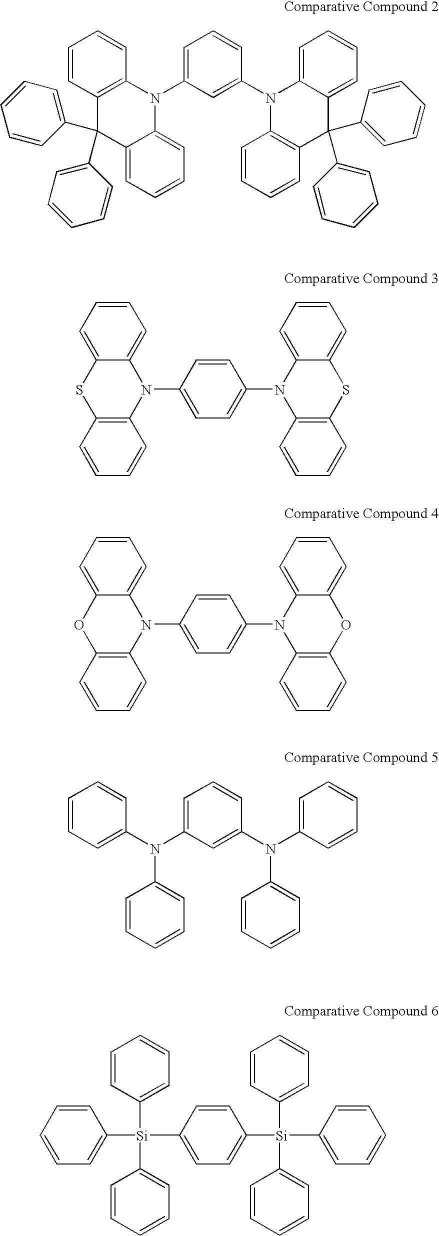

### Synthesis Examples

### Synthesis Example 1

### Synthesis of Illustrative Compound (1)

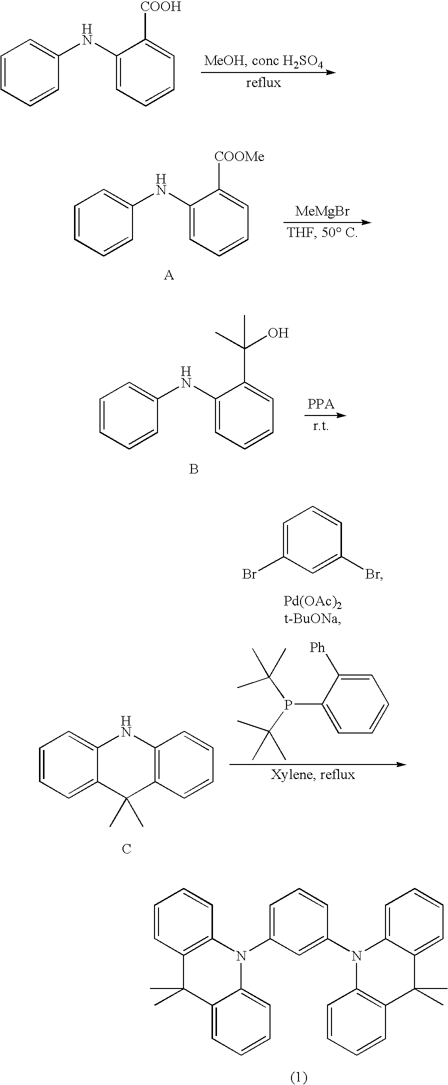

N-Phenylanthranilic acid (21.3 g, 100 mmoles), methanol (500 mL) and concentrated sulfuric acid (25 mL) were heated for refluxing under a nitrogen gas stream and stirred for 7 hours. After returning the temperature to room temperature, pure water, ethyl acetate and hexane were added, thereby extracting an organic phase. The organic phase was dried over sodium sulfate; the solvent was distilled off in vacuo; and the residue was then purified by silica gel column chromatography (hexane/ethyl acetate=4/1), thereby obtaining Synthetic Intermediate A (13.3 g, 58.5 mmoles, yield: 59%).

Synthetic Intermediate A (9.10 g, 40.0 mmoles), a 0.93 moles/L THF solution of methyl magnesium bromide (150 mL, 140 0 mmoles) and dry THF (50 mL) were mixed at 0° C. under a nitrogen gas stream, and the mixture was then stirred at 50° C. for one hour. The reaction solution was put into ice water and neutralized with an ammonium chloride aqueous solution, to which was then added ethyl acetate to extract an organic phase. The organic phase was dried over sodium sulfate, and the solvent was then distilled off in vacuo to obtain Synthetic Intermediate B (9.10 g, 100 mmoles, yield: 100%).

Synthetic Intermediate B (6.50 g, 28.5 mmoles) and polyphosphoric acid (50 mL) were stirred at room temperature for one hour under a nitrogen gas stream. Pure water was added, and the mixture was neutralized with a sodium hydrogencarbonate aqueous solution, to which was then added ethyl acetate to extract an organic phase. The organic phase was dried over sodium sulfate; the solvent was distilled off in vacuo; and the residue was then recrystallized from hexane to obtain Synthetic Intermediate C (4.60 g, 22.0 mmoles, yield: 77%).

Synthetic Intermediate C (1.50 g, 7.17 mmoles), 1,3-dibromobenzene (769 mg, 3.26 mmoles), palladium acetate (74 mg, 0.33 mmoles), 2-(di-t-butylphosphino)biphenyl (394 mg, 1.32 mmoles), t-butoxy sodium (125 g, 13.0 mmoles) and dry xylene (20 mL) were heated for refluxing under a nitrogen gas stream, and heating was continued for 6 hours. Ethyl acetate and toluene were added; a solid was filtered off; and pure water was then added to extract an organic phase. The organic phase was dried over sodium sulfate; the solvent was distilled off in vacuo; and the residue was then purified by silica gel column chromatography (toluene/hexane=1/1). The solvent was distilled off in vacuo, and the residue was then recrystallized from chloroform/methanol (1/1) to obtain Compound (1) (1.00 g, 2.03 mmoles, yield: 62%).

¹H-NMR (300 MHz, CDCl₃) δ=7.90 (t, 1H), 7.54 (d, 2H), 7.47 (d, 4H), 7.36 (s, 1H), 7.05 (t, 4H), 6.96 (t, 4H), 6.43 (d, 4H), 1.67 (s, 12H) ppm

### Synthesis Example 2

### Synthesis of Illustrative Compounds (10) and (11)

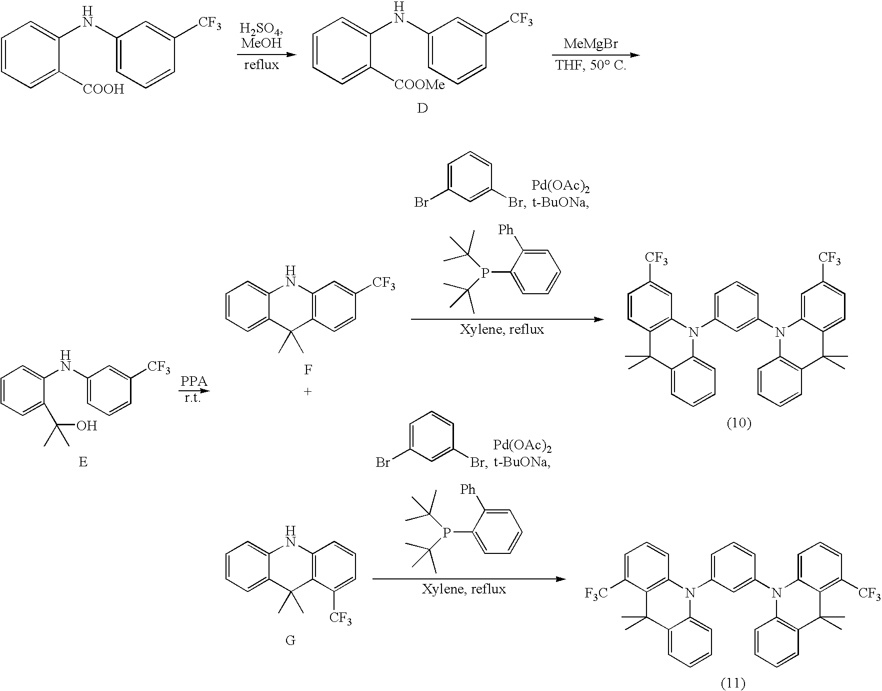

A mixture of Synthetic Intermediates F and G was obtained in the foregoing synthesis route according to the method of Synthesis Example 1. This mixture was subjected to isolation from each other by column chromatography (hexane/ethyl acetate=9/1). Compound (10) and Compound (11) were synthesized from Synthetic Intermediate F and Synthetic Intermediate G, respectively according to the method of Synthesis Example 1.

Compound (10): ¹H-NMR (300 MHz, CDCl₃) δ=8.00 (t, 1H), 7.56 to 7.52 (m, 4H), 7.46 (d, 2H), 7.35 (t, 1H), 7.17 (d, 2H), 7.07 (t, 2H), 6.99 (t, 2H), 6.60 (s, 2H), 6.39 (d, 2H), 1.68 (s, 12H) ppm

Compound (11): ¹H-NMR (300 MHz, CDCl₃) δ=7.94 (t, 1H), 7.60 to 7.54 (m, 4H), 7.43 (d, 2H), 7.36 (t, 1H), 7.15 to 7.00 (m, 6H), 6.80 (d, 2H), 6.52 (d, 12H), 1.79 (s, 12H) ppm

### Synthesis Example 3

### Synthesis of Illustrative Compounds (27), (34), (49), (58) and (64)

Compounds (27), (34), (49), (58) and (64) were synthesized according to Synthesis Examples 1 and 2.

Compound (27): ¹H-NMR (300 MHz, CDCl₃) δ=7.46 (d, 4H), 7.28 (d, 2H), 7.18 (s, 1H), 7.07 (t, 4H), 6.97 (t, 4H), 6.46 (d, 4H), 1.66 (s, 12H) ppm

Compound (34): ¹H-NMR (300 MHz, CDCl₃) δ=7.62 (d, 2H), 7.46 (d, 4H), 7.30 (t, 1H), 7.04 (t, 4H), 6.94 (t, 4H), 6.38 (d, 4H) ppm

Compound (49): ¹H-NMR (300 MHz, CDCl₃) δ=7.69 (t, 1H), 7.45 (d, 4H), 7.28 (d, 4H), 7.13 to 7.03 (m, 10H), 1.64 (s, 12H) ppm

Compound (58): ¹H-NMR (300 MHz, CDCl₃) δ=7.56 (s, 3H), 7.47 (d, 6H), 7.12 (t, 6H), 6.99 (t, 6H), 6.61 (d, 6H), 1.65 (s, 18H) ppm

Compound (64): ¹H-NMR (300 MHz, CDCl₃) δ=8.02 (d, 2H), 7.82 to 7.76 (m, 4H), 7.49 (d, 4H), 7.37 (d, 2H), 6.97 (t, 4H), 6.88 (t, 4H), 6.20 (d, 4H), 1.62 (s, 12H) ppm

### Synthesis Example 4

### Synthesis of Illustrative Compound (40)

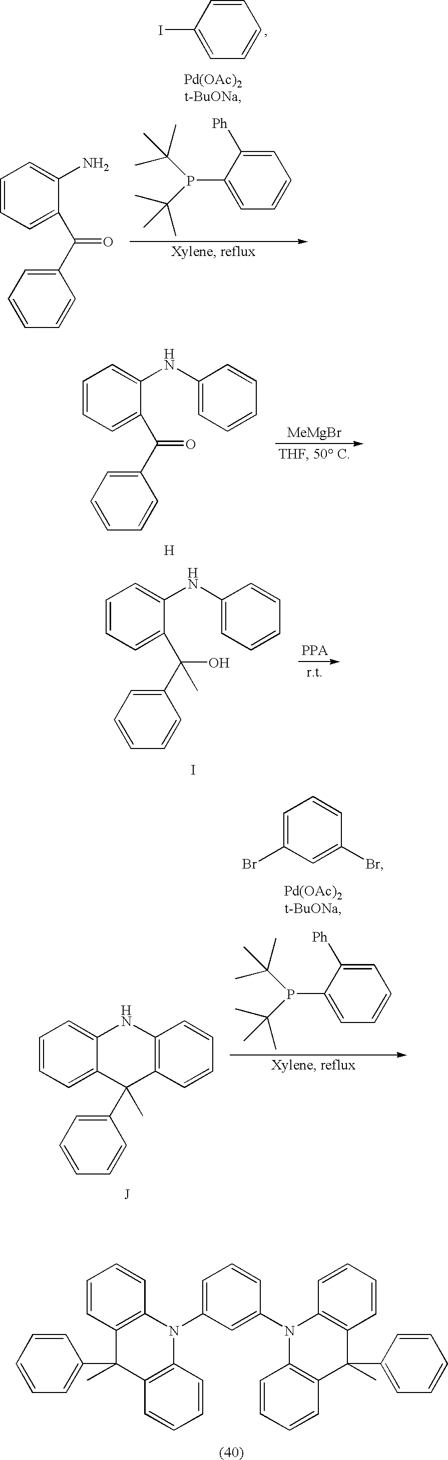

Compound (40) was synthesized in the foregoing synthesis route according to the method of Synthesis Example 1.

¹H-NMR (300 MHz, CDCl₃) δ=7.91 (t, 1H), 7.53 (d, 2H), 7.45 to 7.20 (m, 11H), 7.03 to 6.97 (m, 4H), 6.84 to 6.75 (m, 8H), 6.44 (d, 4H) ppm

### Synthesis Example 5

### Synthesis of Illustrative Compound (59)

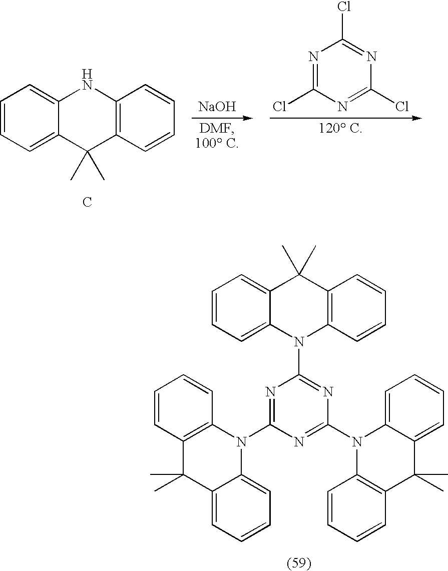

Synthetic Intermediate C (3.60 g, 17.2 mmoles), 50% by mass oily sodium hydride (1.04 g, 20.8 mmoles) and dry DMF (40 mL) were mixed under a nitrogen gas stream; the mixture was stirred at 100° C. for 5 minutes; and the temperature was then returned to room temperature. Cyanuric chloride (954 mg, 5.22 mmoles) dissolved in DMF (20 mL) was added; the mixture was further stirred at 120° C. for one hour; and the temperature was then returned to room temperature. 200 mL of pure water was added, and a deposit was collected by filtration and then washed with pure water. After drying, the resultant was purified by silica gel column chromatography (hexane/ethyl acetate=4/1) and subsequently recrystallized from isopropanol/methylene chloride (10/1) to obtain Compound (59) (500 mg. 0.711 moles, yield: 14%).

¹H-NMR (300 MHz, CDCl₃) δ=7.57 (d, 6H), 7.36 (d, 2H), 7.12 (t, 6H), 7.04 (t, 6H), 1.52 (s, 18H) ppm

### Synthesis Example 6

### Synthesis of Comparative Compound 2

Comparative Compound 2 was synthesized according to a description on pages 12 to 14 of WO 2007/110228.

Comparative Compound 2: ¹H-NMR (300 MHz, CDCl₃) δ=7.66 (t, 1H), 7.25 to 6.89 (m, 35H), 6.49 (d, 4H) ppm

A quartz substrate having a thickness of 0.5 mm and a size of 2.5 cm in square was ultrasonically washed in 2-propanol and then subjected to a UV-ozone treatment for 30 minutes. Thereafter, each of the foregoing Illustrative Compounds and Comparative Compounds 2, 5 and 6 was subjected to vacuum vapor deposition in a thickness of 50 nm on this quartz substrate and measured with respect to the following various physical properties.

### (a) Ionization Potential Energy (Ip):

Ip was measured by a photoelectron spectrophotometer (AC-1, manufactured by Riken Keiki Co., Ltd.).

### (b) Electron Affinity (Ea):

A band gap Eg was determined from a long-wavelength end of an absorption spectrum, from which was then determined Ea (Ea=Ip−Eg).

### (c) Excited Triplet Level (Tᵢ Energy):

A phosphorescent spectrum was measure at −196° C., and the T₁ energy was determined from a light emission short-wavelength end.

The results are shown in Table 1.

TABLE 1

| Compound No. | Ip (eV) | Ea (eV) | T₁ (eV) |
|---|---|---|---|
| Compound (1) | 5.6 | 1.8 | 3.0 |
| Compound (5) | 5.6 | 1.8 | 3.1 |
| Compound (10) | 6.0 | 2.2 | 3.0 |
| Compound (11) | 6.0 | 2.2 | 3.0 |
| Compound (27) | 5.8 | 1.9 | 3.0 |
| Compound (29) | 5.9 | 2.0 | 3.0 |
| Compound (34) | 5.7 | 1.9 | 3.1 |
| Compound (49) | 5.6 | 2.2 | 3.1 |
| Compound (40) | 5.7 | 2.0 | 3.0 |
| Compound (42) | 5.8 | 2.0 | 3.0 |
| Compound (44) | 5.8 | 2.0 | 3.0 |
| Compound (58) | 5.7 | 1.8 | 3.1 |
| Compound (59) | 5.8 | 2.0 | 3.1 |
| Compound (60) | 5.7 | 2.1 | 2.9 |
| Compound (64) | 5.6 | 1.9 | 2.8 |
| Compound (61) | 5.6 | 1.8 | 3.0 |
| Compound (107) | 5.6 | 1.8 | 3.1 |
| Compound (109) | 5.6 | 1.8 | 3.1 |
| Compound (129) | 5.6 | 2.1 | 2.9 |
| Compound (130) | 5.6 | 2.2 | 2.9 |
| Compound (132) | 5.6 | 2.2 | 2.9 |
| Comparative Compound 2 | 5.8 | 2.1 | 3.1 |
| Comparative Compound 5 | 5.6 | 1.9 | 2.7 |
| Comparative Compound 6 | — | — | 3.2 |

Since Ip of Comparative Compound 6 exceeded the measurement critical value (up to 6.0 eV) of AC-1, it was impossible for the measurement.

Also, an ITO film-provided glass substrate having a thickness of 0.5 mm and a size of 2.5 cm in square (manufactured by Geomatec Co., Ltd., surface resistance: 10Ω/□) was put in a washing vessel, ultrasonically washed in 2-propanol and then subjected to a UV-ozone treatment for 30 minutes. Compound (1) was subjected to thin-film deposition in a thickness of about 1 μm on this transparent anode (ITO film) by means of vacuum vapor deposition, and subsequently, an aluminum metal was vapor deposited in a thickness of 100 nm, thereby forming an electrode. A hole mobility of Compound (1) was determined by applying the time-of-flight (TOF) method to the thus obtained device. As a result, the hole mobility of Compound (1) at an electric field intensity of 1,000 V/cm was estimated to be 3.0×10⁻⁴ cm²/Vs.

### <Organic Electroluminescence Device>

### Example 1

### Preparation of Device 1-1

An ITO film-provided glass substrate having a thickness of 0.5 mm and a size of 2.5 cm in square (manufactured by Geomatec Co., Ltd., surface resistance: 10Ω/□) was put in a washing vessel, ultrasonically washed in 2-propanol and then subjected to a UV-ozone treatment for 30 minutes. The following organic compound layers were successively vapor deposited on this transparent anode (ITO film) by means of vacuum vapor deposition.

First layer: Copper phthalocyanine (CuPc), thickness: 10 nm

Second layer: NPD, thickness: 50 nm

Third layer (light emitting layer): Compound (1) of the invention and D-158 (weight ratio: 90/10; in Table 2, this is described as “Compound (1)+10% D-158”)

Fourth layer: BAlq, thickness: 30 nm

0.1 nm-thick lithium fluoride and a 100 nm-thick aluminum metal were vapor deposited in this order thereon, thereby forming a cathode.

This was placed in a nitrogen gas-purged glove box without being exposed to the air and sealed using a glass-made sealing can and a UV-curable adhesive (XNR5516HV, manufactured by Nagase-CHIBA Ltd.), thereby obtaining Organic Electroluminescence Device 1-1. Structures of the used compounds are shown below.

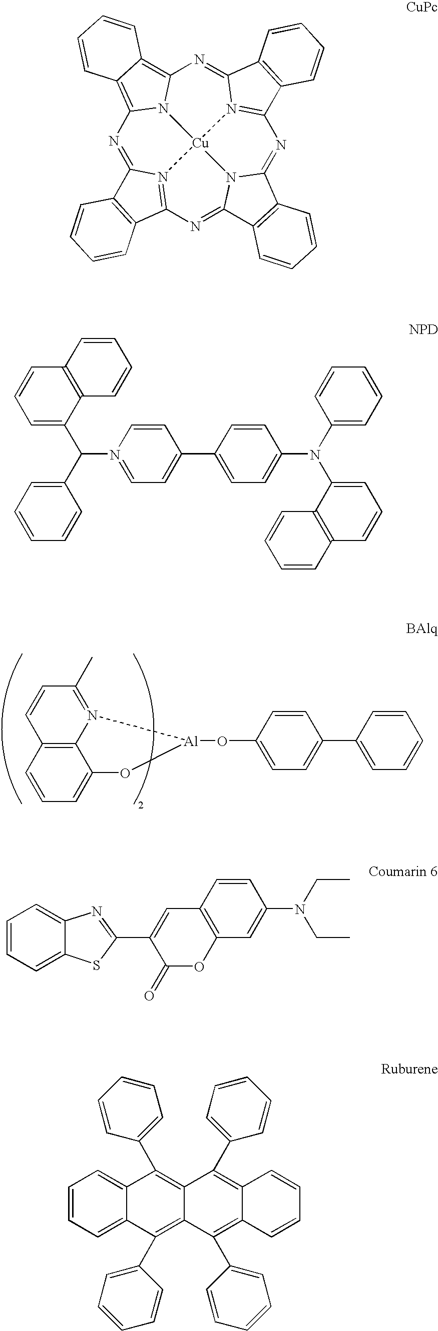

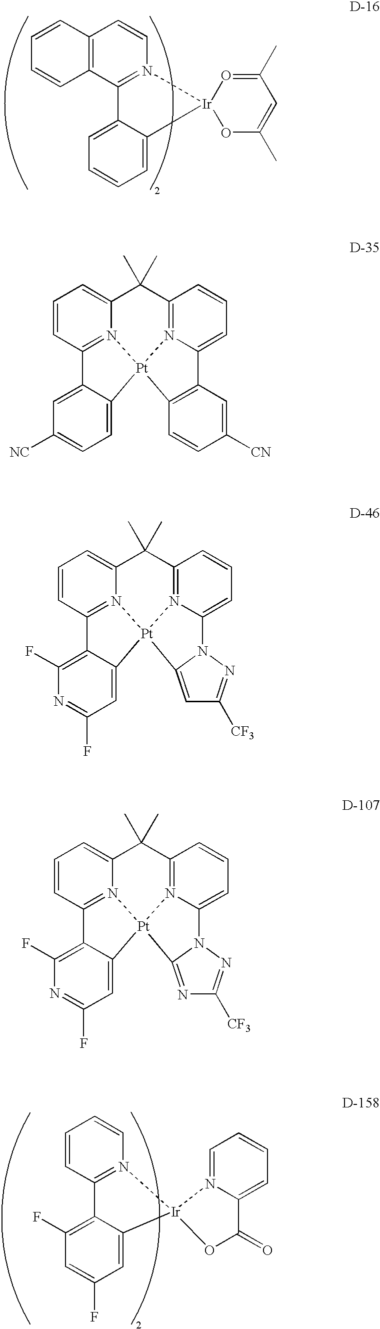

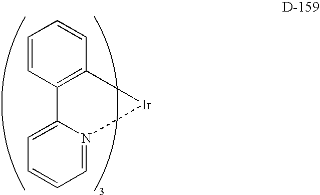

### [Preparation of Other Devices]

Each of devices was prepared in the same as in [Preparation of Device 1-1], except for employing a light emitting layer configuration shown in Table 2.

TABLE 2

| | | Maximum light | Relative | | |
|---|---|---|---|---|---|
| | | emission | external | Relative | Relative |
| | | wavelength | quantum | driving | driving |
| Device No. | Light emitting layer configuration | (nm) | efficiency | voltage | durability |
| Device 1-1 of the invention | Compound (1) + 10% D-158 | 469 | 10 | 10 | 10 |
| Device 1-10 of the invention | Compound (10) + 10% D-158 | 469 | 10 | 11 | 9 |
| Device 1-27 of the invention | Compound (27) + 10% D-158 | 470 | 10 | 11 | 7 |
| Device 1-49 of the invention | Compound (49) + 10% D-158 | 469 | 11 | 10 | 6 |
| Device 1-40 of the invention | Compound (40) + 10% D-158 | 470 | 10 | 11 | 9 |
| Device 1-42 of the invention | Compound (42) + 10% D-158 | 469 | 9 | 10 | 10 |
| Device 1-58 of the invention | Compound (58) + 10% D-158 | 469 | 10 | 10 | 6 |
| Device 1-61 of the invention | Compound (61) + 10% D-158 | 470 | 9 | 10 | 6 |
| Device 1-107 of the invention | Compound (107) + 10% D-158 | 469 | 9 | 13 | 8 |
| Comparative Device 1-1 | Comparative Compound 1 + 10% D-158 | 470 | 9 | 14 | <0.1 |
| Comparative Device 1-3 | Comparative Compound 3 + 10% D-158 | 470 | 2 | 21 | <0.1 |
| Comparative Device 1-5 | Comparative Compound 5 + 10% D-158 | 469 | 3 | 20 | <0.1 |
| Comparative Device 1-6 | Comparative Compound 6 + 10% D-158 | 469 | 5 | 16 | 1 |

### Example 2

Each of devices was prepared in the same as in [Preparation of Device 1-1], except for employing a light emitting layer configuration shown in Table 3.

TABLE 3

| | | Maximum light | Relative | | |
|---|---|---|---|---|---|
| | | emission | external | Relative | Relative |
| | | wavelength | quantum | driving | driving |
| Device No. | Light emitting layer configuration | (nm) | efficiency | voltage | durability |
| Device 2-1 of the invention | Compound (1) + 10% D-46 | 466 | 10 | 10 | 10 |
| Device 2-11 of the invention | Compound (11) + 10% D-46 | 466 | 10 | 11 | 9 |
| Device 2-27 of the invention | Compound (27) + 10% D-46 | 466 | 10 | 11 | 7 |
| Device 2-49 of the invention | Compound (49) + 10% D-46 | 464 | 11 | 11 | 5 |
| Device 2-40 of the invention | Compound (40) + 10% D-46 | 467 | 10 | 11 | 9 |
| Device 2-42 of the invention | Compound (42) + 10% D-46 | 466 | 10 | 10 | 9 |
| Device 2-44 of the invention | Compound (44) + 10% D-46 | 466 | 10 | 12 | 9 |
| Device 2-59 of the invention | Compound (59) + 10% D-46 | 467 | 10 | 10 | 6 |
| Device 2-60 of the invention | Compound (60) + 10% D-46 | 466 | 11 | 10 | 9 |
| Device 2-64 of the invention | Compound (64) + 10% D-46 | 466 | 9 | 9 | 11 |
| Device 2-107 of the invention | Compound (107) + 10% D-46 | 467 | 8 | 12 | 7 |
| Comparative Device 2-1 | Comparative Compound 1 + 10% D-46 | 466 | 9 | 14 | <0.1 |
| Comparative Device 2-2 | Comparative Compound 2 + 10% D-46 | 466 | 10 | 13 | <0.1 |
| Comparative Device 2-3 | Comparative Compound 3 + 10% D-46 | Could not be evaluated because of large sub-light | | | |
| | | emission. | | | |
| Comparative Device 2-4 | Comparative Compound 4 + 10% D-46 | Could not be evaluated because of large sub-light | | | |
| | | emission. | | | |
| Comparative Device 2-5 | Comparative Compound 5 + 10% D-46 | Could not be evaluated because of large sub-light | | | |
| | | emission. | | | |
| Comparative Device 2-6 | Comparative Compound 6 + 10% D-46 | 466 | 3 | 21 | 0.1 |

### Example 3

Each of devices was prepared in the same as in [Preparation of Device 1-1], except for employing a light emitting layer configuration shown in Table 4.

TABLE 4

| | | Maximum light | Relative | | |
|---|---|---|---|---|---|
| | | emission | external | Relative | Relative |
| | | wavelength | quantum | driving | driving |
| Device No. | Light emitting layer configuration | (nm) | efficiency | voltage | durability |
| Device 3-1 of the invention | Compound (1) + 10% D-107 | 461 | 10 | 10 | 10 |
| Device 3-5 of the invention | Compound (5) + 10% D-107 | 462 | 10 | 13 | 10 |
| Device 3-27 of the invention | Compound (27) + 10% D-107 | 461 | 10 | 11 | 6 |
| Device 3-40 of the invention | Compound (40) + 10% D-107 | 462 | 10 | 11 | 10 |
| Device 3-42 of the invention | Compound (42) + 10% D-107 | 462 | 10 | 10 | 8 |
| Device 3-58 of the invention | Compound (58) + 10% D-107 | 461 | 10 | 10 | 6 |
| Device 3-61 of the invention | Compound (61) + 10% D-107 | 461 | 8 | 10 | 6 |
| Device 3-109 of the invention | Compound (109) + 10% D-107 | 461 | 9 | 13 | 8 |
| Comparative Device 3-1 | Comparative Compound 1 + 10% D-107 | Could not be evaluated because of large sub-light | | | |
| | | emission. | | | |
| Comparative Device 3-2 | Comparative Compound 2 + 10% D-107 | Could not be evaluated because of large sub-light | | | |
| | | emission. | | | |
| Comparative Device 3-5 | Comparative Compound 5 + 10% D-107 | Could not be evaluated because of large sub-light | | | |
| | | emission. | | | |
| Comparative Device 3-6 | Comparative Compound 6 + 10% D-107 | 461 | 4 | 17 | 0.3 |

### Example 4

Each of devices was prepared in the same as in [Preparation of Device 1-1], except for employing a light emitting layer configuration shown in Table 5.

TABLE 5

| | | Maximum light | Relative | | |
|---|---|---|---|---|---|
| | | emission | external | Relative | Relative |
| | | wavelength | quantum | driving | driving |
| Device No. | Light emitting layer configuration | (nm) | efficiency | voltage | durability |
| Device 4-1 of the invention | Compound (1) + 10% D-159 | 513 | 10 | 10 | 10 |
| Device 4-10 of the invention | Compound (10) + 10% D-159 | 514 | 10 | 12 | 8 |
| Device 4-40 of the invention | Compound (40) + 10% D-159 | 513 | 10 | 12 | 9 |
| Device 4-109 of the invention | Compound (109) + 10% D-159 | 513 | 8 | 13 | 8 |
| Comparative Device 4-1 | Comparative Compound 1 + 10% D-159 | 514 | 9 | 13 | <0.1 |
| Comparative Device 4-3 | Comparative Compound 3 + 10% D-159 | 514 | 6 | 12 | <0.1 |
| Comparative Device 4-6 | Comparative Compound 6 + 10% D-159 | 513 | 6 | 15 | 2 |

### Example 5

Each of devices was prepared in the same as in [Preparation of Device 1-1], except for employing a light emitting layer configuration shown in Table 6.

TABLE 6

| | | Maximum light | Relative | | |
|---|---|---|---|---|---|
| | | emission | external | Relative | Relative |
| | | wavelength | quantum | driving | driving |
| Device No. | Light emitting layer configuration | (nm) | efficiency | voltage | durability |
| Device 5-1 of the invention | Compound (1) + 10% D-35 | 503 | 10 | 10 | 10 |
| Device 5-29 of the invention | Compound (29) + 10% D-35 | 501 | 9 | 12 | 8 |
| Device 5-34 of the invention | Compound (34) + 10% D-35 | 504 | 10 | 11 | 10 |
| Device 5-44 of the invention | Compound (44) + 10% D-35 | 503 | 9 | 11 | 9 |
| Device 5-61 of the invention | Compound (61) + 10% D-35 | 503 | 8 | 10 | 6 |
| Comparative Device 5-1 | Comparative Compound 1 + 10% D-35 | 504 | 9 | 12 | <0.1 |
| Comparative Device 5-3 | Comparative Compound 3 + 10% D-35 | 504 | 5 | 12 | <0.1 |
| Comparative Device 5-6 | Comparative Compound 6 + 10% D-35 | 503 | 5 | 15 | 1 |

### Example 6

Each of devices was prepared in the same as in [Preparation of Device 1-1], except for employing a light emitting layer configuration shown in Table 7.

TABLE 7

| | | Maximum light | Relative | | |
|---|---|---|---|---|---|
| | | emission | external | Relative | Relative |
| | | wavelength | quantum | driving | driving |
| Device No. | Light emitting layer configuration | (nm) | efficiency | voltage | durability |
| Device 6-1 of the invention | Compound (1) + 10% D-16 | 625 | 10 | 10 | 10 |
| Device 6-29 of the invention | Compound (29) + 10% D-16 | 626 | 9 | 11 | 9 |
| Device 6-40 of the invention | Compound (40) + 10% D-16 | 627 | 9 | 11 | 9 |
| Device 6-44 of the invention | Compound (44) + 10% D-16 | 626 | 9 | 11 | 8 |
| Device 6-64 of the invention | Compound (64) + 10% D-16 | 626 | 8 | 10 | 9 |
| Comparative Device 6-1 | Comparative Compound 1 + 10% D-16 | 629 | 6 | 13 | <0.1 |
| Comparative Device 6-3 | Comparative Compound 3 + 10% D-16 | 629 | 5 | 12 | <0.1 |
| Comparative Device 6-6 | Comparative Compound 6 + 10% D-16 | 626 | 7 | 14 | 2 |

### Example 7

Each of devices was prepared in the same as in [Preparation of Device 1-1], except for employing a light emitting layer configuration shown in Table 8.

TABLE 8

| | | Maximum | Relative | | |
|---|---|---|---|---|---|
| | | light emission | external | Relative | Relative |
| | | wavelength | quantum | driving | driving |
| Device No. | Light emitting layer configuration | (nm) | efficiency | voltage | durability |
| Device 7-1 of the invention | Compound (1) + 10% Coumarin 6 | 496 | 10 | 10 | 10 |
| Device 7-40 of the invention | Compound (40) + 10% Coumarin 6 | 496 | 10 | 10 | 9 |
| Device 7-107 of the invention | Compound (107) + 10% Coumarin 6 | 497 | 10 | 12 | 9 |
| Comparative Device 7-1 | Comparative Compound 1 + 10% Coumarin 6 | 496 | 7 | 12 | 1 |
| Comparative Device 7-3 | Comparative Compound 3 + 10% Coumarin 6 | 497 | 6 | 12 | 2 |
| Comparative Device 7-6 | Comparative Compound 6 + 10% Coumarin 6 | 496 | 8 | 14 | 4 |

### Example 8

Each of devices was prepared in the same as in [Preparation of Device 1-1], except for employing a light emitting layer configuration shown in Table 9.

TABLE 9

| | | Maximum light | Relative | | |
|---|---|---|---|---|---|
| | | emission | external | Relative | Relative |
| | | wavelength | quantum | driving | driving |
| Device No. | Light emitting layer configuration | (nm) | efficiency | voltage | durability |
| Device 8-1 of the invention | Compound (1) + 10% Rubrene | 555 | 10 | 10 | 10 |
| Device 8-10 of the invention | Compound (10) + 10% Rubrene | 556 | 10 | 11 | 8 |
| Device 8-109 of the invention | Compound (109) + 10% Rubrene | 555 | 8 | 12 | 7 |
| Comparative Device 8-1 | Comparative Compound 1 + 10% Rubrene | 556 | 7 | 12 | 1 |
| Comparative Device 8-3 | Comparative Compound 3 + 10% Rubrene | 555 | 7 | 11 | 1 |
| Comparative Device 8-6 | Comparative Compound 6 + 10% Rubrene | 555 | 8 | 13 | 4 |

### Example 9

Each of devices was prepared in the same as in [Preparation of Device 1-1], except for employing a hole transport layer configuration and a light emitting layer configuration shown in Table 10.

In Table 10, the terms “NPD (47 nm)/Compound (3 nm)” mean that a layer prepared by vapor depositing NPD in a thickness of 47 nm and then vapor depositing the compound of the invention in a thickness of 3 nm was used as the hole transport layer.

TABLE 10

| | | | Maximum | | | |
|---|---|---|---|---|---|---|
| | | | light | Relative | | |
| | | | emission | external | Relative | Relative |
| | | | wavelength | quantum | driving | driving |
| Device No. | Hole transport layer configuration | Light emitting layer configuration | (nm) | efficiency | voltage | durability |
| Device 9-1 of the invention | NPD (47 nm)/Compound (1) (3 mm) | Compound (1) + 10% D-46 | 466 | 10 | 10 | 10 |
| Device 9-2 of the invention | Compound (1) (50 nm) | Compound (1) + 10% D-46 | 466 | 10 | 13 | 5 |
| Device 9-3 of the invention | NPD (47 nm)/Compound (1) (3 mm) | Comparative Compound 6 + | 466 | 8 | 15 | 3 |
| | | 10% D-46 | | | | |
| Device 9-4 of the invention | NPD (47 nm)/Compound (34) | Comparative Compound 6 + | 466 | 7 | 16 | 3 |
| | (3 mm) | 10% D-46 | | | | |
| Device 9-5 of the invention | NPD (47 nm)/Compound (60) | Comparative Compound 6 + | 466 | 8 | 15 | 2 |
| | (3 mm) | 10% D-46 | | | | |
| Comparative Device 9-1 | NPD (50 nm) | Comparative Compound 6 + | 466 | 1 | 23 | <0.1 |
| | | 10% D-46 | | | | |

### Examples 10 to 13

Each of devices was prepared in the same as in [Preparation of Device 1-1], except for changing the layer configuration to the following layer configuration.

First layer: 2-TNATA+F₄-TCNQ (mass ratio: 99.7/0.3), thickness: 120 nm

Second layer: NPD, thickness: 7 nm

Third layer: Shown in Tables 11 to 13, thickness: 3 nm

Fourth layer (light emitting layer): Shown in Tables 11 to 13, thickness: 30 nm

Fifth layer: Shown in Tables 11 to 13, thickness: 29 nm

Sixth layer: BCP, thickness: 1 nm

Cathode: LiF (thickness: 0.1 nm)/Al (thickness: 100 nm)

The used materials are shown below.

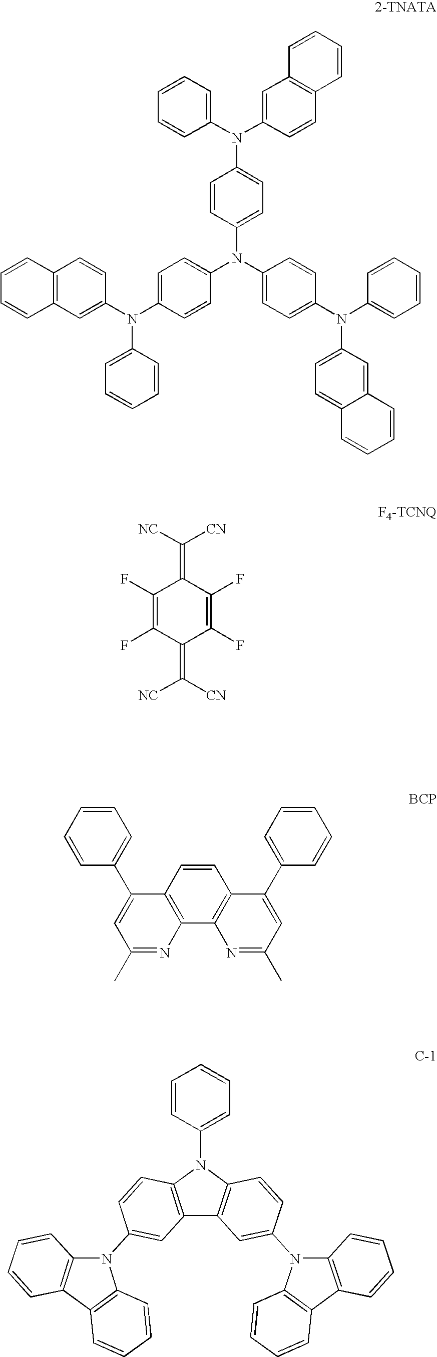

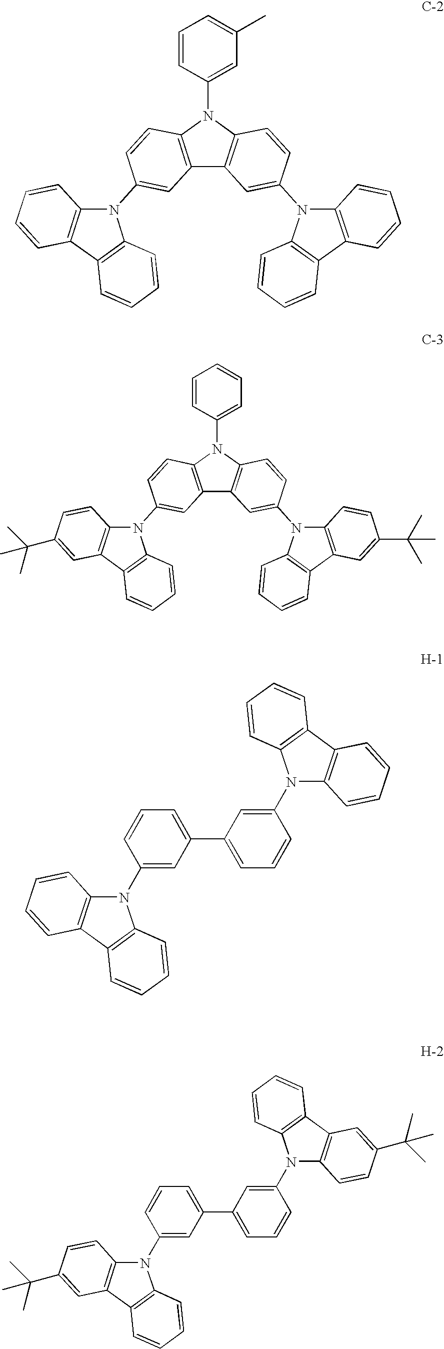

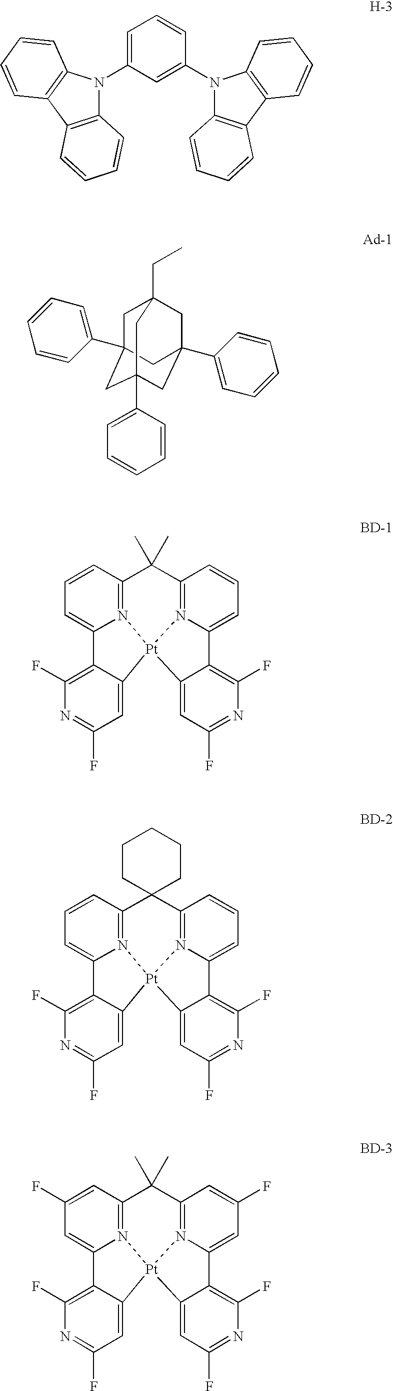

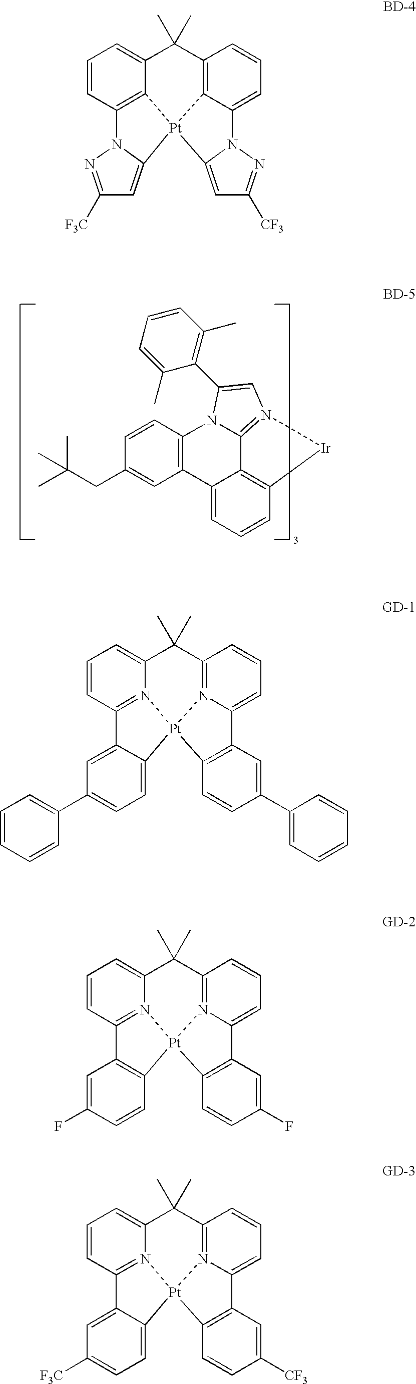

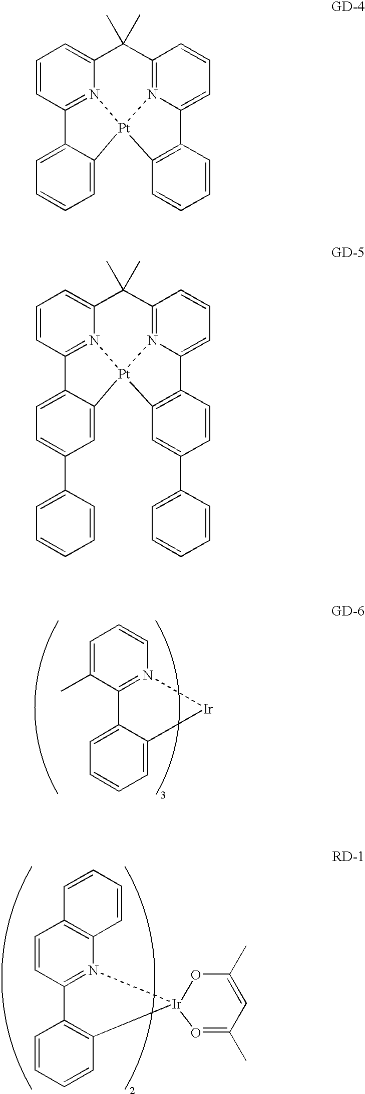

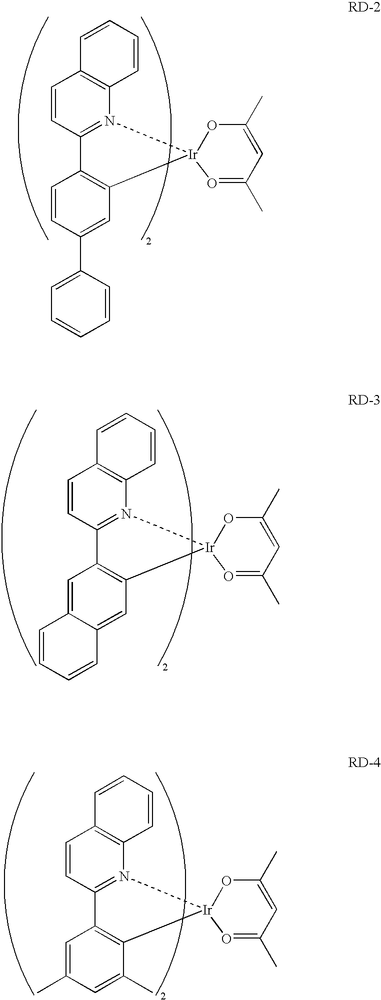

TABLE 11

| | | | | Maximum | | | |
|---|---|---|---|---|---|---|---|
| | | | | light | Relative | | |
| | | | | emission | external | Relative | Relative |
| | Third layer | | Fifth layer | wavelength | quantum | driving | driving |
| Device No. | material | Fourth layer material | material | (nm) | efficiency | voltage | durability |
| Device 10-1 | C-1 | Compound (1) + 10% BD-1 | BAlq | 468 | 10 | 10 | 10 |
| Device 10-2 | C-1 | Compound (1) + 10% Ad-1 + | BAlq | 468 | 11 | 9 | 10 |
| | | 10% BD-1 | | | | | |
| Device 10-3 | Compound | Compound (1) + 10% BD-1 | BAlq | 468 | 10 | 9 | 10 |
| | (1) | | | | | | |
| Device 10-4 | C-1 | Compound (1) + 10% BD-1 | BAlq + 20% | 468 | 11 | 10 | 11 |
| | | | Compound (1) | | | | |
| Device 10-5 | Compound | Compound (1) + 10% BD-1 | BAlq + 20% | 468 | 11 | 9 | 11 |
| | (1) | | Compound (1) | | | | |
| Device 10-6 | C-2 | Compound (64) + 10% BD-1 | BAlq | 468 | 10 | 10 | 12 |
| Device 10-7 | C-3 | Compound (107) + 10% BD-1 | BAlq | 468 | 9 | 11 | 9 |
| Comparative | C-1 | Comparative Compound 1 + 10% BD-1 | BAlq | 468 | 8 | 13 | <0.1 |
| Device 10-1 | | | | | | | |
| Device 10-8 | C-1 | Compound (5) + 10% BD-2 | BAlq | 466 | 8 | 12 | 9 |
| Device 10-9 | C-1 | Compound (5) + 10% Ad-1 + | BAlq | 466 | 9 | 11 | 9 |
| | | 10% BD-2 | | | | | |
| Device 10-10 | C-2 | Compound (27) + 10% BD-2 | BAlq + 50% | 466 | 8 | 10 | 8 |
| | | | Compound (27) | | | | |
| Device 10-11 | Compound | Compound (109) + 10% BD-2 | BAlq | 466 | 8 | 11 | 8 |
| | (109) | | | | | | |
| Comparative | C-1 | Comparative Compound 2 + 10% BD-2 | BAlq | 466 | 6 | 15 | <0.1 |
| Device 10-2 | | | | | | | |
| Device 10-12 | C-2 | Compound (1) + 45% H-1 + 10% BD-3 | BAlq | 464 | 8 | 11 | 10 |
| Device 10-13 | C-1 | Compound (40) + 45% H-1 + | BAlq | 464 | 8 | 12 | 8 |
| | | 10% BD-3 | | | | | |
| Comparative | C-1 | Comparative Compound 5 + 45% H-1 + | BAlq | Could not be evaluated because of large | | | |
| Device 10-3 | | 10% BD-3 | | sub-light emission. | | | |
| Device 10-14 | C-1 | Compound (34) + 45% H-3 + | BAlq | 456 | 7 | 10 | 7 |
| | | 10% BD-4 | | | | | |
| Device 10-15 | Compound | Compound (60) + 45% H-3 + | BAlq | 456 | 7 | 9 | 8 |
| | (60) | 10% BD-4 | | | | | |
| Comparative | C-1 | Comparative Compound 6 + 45% H-3 + | BAlq | 456 | 5 | 13 | 2 |
| Device 10-4 | | 10% BD-4 | | | | | |
| Device 10-16 | C-3 | Compound (1) + 10% BD-5 | BAlq | 462 | 8 | 10 | 8 |
| Device 10-17 | Compound | Compound (5) + 10% BD-5 | BAlq | 462 | 8 | 11 | 9 |
| | (5) | | | | | | |
| Device 10-18 | H-2 | Compound (27) + 45% H-2 + | BAlq | 462 | 8 | 10 | 8 |
| | | 10% BD-5 | | | | | |
| Device 10-19 | H-2 | Compound (49) + 45% H-2 + | BAlq | 462 | 9 | 9 | 9 |
| | | 10% BD-5 | | | | | |
| Device 10-20 | H-2 | Compound (59) + 45% H-2 + | BAlq + 30% | 462 | 9 | 9 | 9 |
| | | 10% BD-5 | Compound (59) | | | | |
| Comparative | C-3 | Comparative Compound 6 + 10% BD-5 | BAlq | 462 | 5 | 14 | 3 |
| Device 10-5 | | | | | | | |
| Comparative | H-2 | Comparative Compound 6 + 45% H-2 + | BAlq | 462 | 6 | 13 | 4 |
| Device 10-6 | | 10% BD-5 | | | | | |

TABLE 12

| | | | | Maximum light | Relative | | |
|---|---|---|---|---|---|---|---|
| | | | | emission | external | | Relative |
| | Third layer | | | wavelength | quantum | Relative | driving |
| Device No. | material | Fourth layer material | Fifth layer material | (nm) | efficiency | driving voltage | durability |
| Device 11-1 | C-2 | Compound (1) + 10% GD-1 | BAlq | 531 | 10 | 10 | 10 |
| Device 11-2 | C-2 | Compound (129) + 10% GD-1 | BAlq | 530 | 11 | 9 | 11 |
| Device 11-3 | C-2 | Compound (129) + 10% GD-1 | Compound (129) | 530 | 11 | 8 | 12 |
| Comparative | C-2 | Comparative Compound 5 + 10% GD-1 | BAlq | 531 | 8 | 12 | <0.1 |
| Device 11-1 | | | | | | | |
| Device 11-4 | C-1 | Compound (27) + 10% GD-2 | BAlq | 528 | 10 | 10 | 8 |
| Device 11-5 | C-1 | Compound (130) + 10% GD-2 | BAlq | 527 | 10 | 9 | 10 |
| Device 11-6 | Compound | Compound (130) + 10% GD-2 | Compound (130) | 527 | 12 | 8 | 11 |
| | (130) | | | | | | |
| Comparative | C-1 | Comparative Compound 6 + 10% GD-2 | BAlq | 528 | 8 | 13 | 3 |
| Device 11-2 | | | | | | | |
| Device 11-7 | C-2 | Compound (107) + 10% GD-3 | BAlq | 501 | 9 | 9 | 7 |
| Device 11-8 | C-2 | Compound (64) + 10% GD-3 | BAlq | 501 | 9 | 9 | 9 |
| Comparative | C-2 | Comparative Compound 3 + 10% GD-3 | BAlq | Could not be evaluated because of large sub-light | | | |
| Device 11-3 | | | | emission. | | | |
| Device 11-9 | C-3 | Compound (58) + 10% GD-4 | BAlq | 511 | 9 | 10 | 8 |
| Device 11-10 | C-1 | Compound (132) + 10% GD-4 | BAlq | 511 | 9 | 9 | 7 |
| Comparative | C-1 | Comparative Compound 2 + 10% GD-4 | BAlq | 511 | 7 | 12 | <0.1 |
| Device 11-4 | | | | | | | |
| Device 11-11 | C-2 | Compound (10) + 10% GD-5 | BAlq | 538 | 11 | 9 | 11 |
| Device 11-12 | C-1 | Compound (44) + 10% GD-5 | BAlq | 538 | 10 | 10 | 9 |
| Comparative | C-2 | Comparative Compound 1 + 10% GD-5 | BAlq | 538 | 8 | 12 | <0.1 |
| Device 11-5 | | | | | | | |
| Device 11-13 | C-1 | Compound (1) + 10% GD-6 | BAlq | 523 | 12 | 8 | 13 |
| Device 11-14 | Compound | Compound (1) + 10% GD-6 | BAlq | 523 | 12 | 7 | 13 |
| | (1) | | | | | | |
| Device 11-15 | C-2 | Compound (129) + 10% GD-6 | BAlq | 523 | 13 | 7 | 14 |
| Device 11-16 | Compound | Compound (129) + 10% GD-6 | Compound (129) | 523 | 13 | 7 | 15 |
| | (129) | | | | | | |
| Comparative | C-1 | Comparative Compound 2 + 10% GD-6 | BAlq | 523 | 9 | 10 | 1 |
| Device 11-6 | | | | | | | |

TABLE 13

| | | | | Maximum | Relative | | |
|---|---|---|---|---|---|---|---|
| | | | | light emission | external | Relative | Relative |
| | | | | wavelength | quantum | driving | driving |
| Device No. | Third layer material | Fourth layer material | Fifth layer material | (nm) | efficiency | voltage | durability |
| Device 12-1 | C-2 | Compound (1) + 10% RD-1 | BAlq | 603 | 10 | 10 | 10 |
| Device 12-2 | C-1 | Compound (132) + 10% RD-1 | BAlq | 605 | 10 | 10 | 9 |
| Device 12-3 | Compound (129) | Compound (129) + 10% RD-1 | Compound (129) | 602 | 11 | 9 | 11 |
| Comparative | C-1 | Comparative Compound 3 | BAlq | 603 | 7 | 13 | <0.1 |
| Device 12-1 | | | | | | | |
| Device 12-4 | C-2 | Compound (5) + 10% RD-2 | BAlq | 608 | 10 | 10 | 10 |
| Device 12-5 | C-3 | Compound (109) + 10% RD-2 | BAlq | 609 | 10 | 10 | 10 |
| Comparative | C-3 | Comparative Compound 6 + 10% RD-2 | BAlq | 610 | 8 | 13 | 5 |
| Device 12-2 | | | | | | | |
| Device 12-6 | C-3 | Compound (130) + 10% RD-3 | BAlq | 630 | 12 | 8 | 13 |
| Device 12-7 | C-2 | Compound (130) + 10% D-35 + 1% RD-3 | Balq | 630 | 13 | 8 | 15 |
| Device 12-8 | Compound (130) | Compound (130) + 10% D-35 + 1% RD-3 | Compound (130) | 630 | 13 | 7 | 15 |
| Comparative | C-2 | Comparative Compound 2 + 10% RD-3 | BAlq | 631 | 10 | 10 | <0.1 |
| Device 12-3 | | | | | | | |
| Device 13-1 | C-2 | Compound (5) + 10% RD-4 | BAlq | 617 | 11 | 10 | 14 |
| Device 13-2 | C-3 | Compound (27) + 10% RD-4 | BAlq | 617 | 11 | 10 | 13 |
| Comparative | C-2 | Comparative Compound 1 + 10% RD-4 | BAlq | 617 | 9 | 12 | 1 |
| Device 13-1 | | | | | | | |

### Example 14

An ITO film-provided glass substrate having a thickness of 0.5 mm and a size of 2.5 cm in square (manufactured by Geomatec Co., Ltd., surface resistance: 10Ω/□) was put in a washing vessel, ultrasonically washed in 2-propanol and then subjected to a UV-ozone treatment for 30 minutes. A PEDOT (poly(3,4-ethylenedioxythiophene)/PSS (polystyrenesulfonic acid) aqueous solution (Baytron P (standard product)) was spin coated (at 4,000 rpm for 60 seconds) on this transparent anode (ITO film) and then dried at 120° C. for 10 minutes, thereby forming a hole transporting buffer layer.

Subsequently, a toluene solution containing 1% by mass of Compound (1) and 0.05% by mass of D-159 was spin coated (at 2,000 rpm for 60 seconds) on the foregoing buffer layer, thereby forming a light emitting layer.

BAlq was vapor deposited in a thickness of 50 nm on this light emitting layer by means of vacuum vapor deposition, thereby forming an electron transport layer. Furthermore, 0.1 nm-thick lithium fluoride and a 100 nm-thick aluminum metal were vapor deposited in this order thereon, thereby forming a cathode.

This was placed in a nitrogen gas-purged glove box without being exposed to the air and sealed using a glass-made sealing can and a UV-curable adhesive (XNR5516HV, manufactured by Nagase-CHIBA Ltd.), thereby obtaining Organic Electroluminescence Device 14-1. Also, Devices 14-2 to 14-3 and Comparative Devices 14-1 to 14-2 were obtained in the same manner, except for changing the Compound (1) as a host material to a material shown in Table 14.

TABLE 14

| | | Maximum | Relative | | |
|---|---|---|---|---|---|
| | | light emission | external | Relative | Relative |
| | | wavelength | quantum | driving | driving |
| Device No. | Host material | (nm) | efficiency | voltage | durability |
| Device 14-1 | Compound (1) | 513 | 10 | 10 | 10 |
| Device 14-2 | Compound (5) | 513 | 9 | 11 | 9 |
| Device 14-3 | Compound (107) | 513 | 8 | 10 | 7 |
| Comparative Device 14-1 | Comparative Compound 2 | 513 | 7 | 13 | <0.1 |
| Comparative Device 14-2 | Comparative Compound 6 | 513 | 5 | 15 | 1 |

The obtained devices were evaluated in the following manners. The results are shown in Tables 2 to 14.

### Evaluation Items:

### (a) Relative External Quantum Efficiency:

Each of the devices was subjected to light emission upon being impressed with a direct current voltage using a source measure unit MODEL 2400, manufactured by Toyo Corporation. Its brightness was measured using a brightness meter BM-8, manufactured by Topcon Corporation. An emission spectrum and a light emission wavelength were measured using a spectral analyzer PMA-11, manufactured by Hamamatsu Photonics K.K. An external quantum efficiency at a brightness in the vicinity of 1,000 cd/m² was calculated based on these measured values according to the brightness conversion method. It is preferable that this value is as high as possible.

### (b) Relative Driving Voltage:

Each of the devices was subjected to light emission upon being impressed with a direct current voltage such that the brightness was 1,000 cd/m². At that time, an impressed voltage was defined as an index for the evaluation of driving voltage. It is preferable that this value is as low as possible.

### (c) Relative Driving Durability:

Each of the devices was subjected to light emission upon being impressed with a direct current voltage such that the brightness was 1,000 cd/m². At that time, an impressed voltage was defined as an index for the evaluation of driving voltage. It is preferable that this value is as high as possible.

### (d) Maximum Light Emission Wavelength:

Each of the devices was subjected to light emission upon being impressed with a direct current voltage such that the brightness was 1,000 cd/m². At that time, a maximum light emission wavelength was determined from the emission spectrum.

It is understood from the foregoing results that the organic EL devices using the compound of the invention have high efficiency, low driving voltage and high driving durability as compared with those using the comparative compounds.

According to the invention, a charge transport material having high chemical stability and large T₁ is provided. Also, according to the invention, an organic EL device having high efficiency, low driving voltage and high driving durability using the instant charge transport material is provided.

The entire disclosure of Japanese Patent Application No. 2009-001161 filed on Jan. 6, 2009, from which the benefit of foreign priority has been claimed in the present application, is incorporated herein by reference, as if fully set forth.

1. A compound represented by the following general formula (1-1) or (2-1): 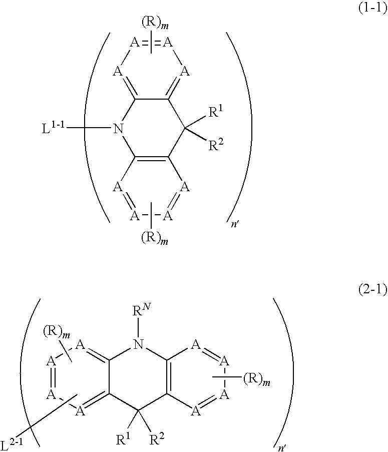
wherein
each of R and R^{N} independently represents a substituent;
each of R¹ and R² independently represents a substituent, provided that R¹ and R² do not represent an aryl group at the same time;
each A independently represents a carbon atom or a nitrogen atom;
each of L¹⁻¹ and L²⁻¹ independently represents phenylene or biphenylene;
n′ represents an integer of 2 or more and not more than 10; and
each m independently represents an integer.

2. A charge transport material represented by the following general formula (1) or (2): 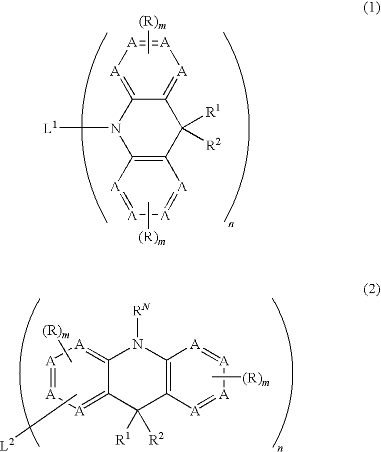
wherein
each of L¹ and L² independently represents a connecting group;
each of R and R^{N} independently represents a substituent;
each of R¹ and R² independently represents a substituent, provided that R¹ and R² do not represent an aryl group at the same time;
each A independently represents a carbon atom or a nitrogen atom;
n represents an integer of 2 or more and not more than 10; and
each m independently represents an integer.

3. The charge transport material according to claim 2, wherein
the general formula (1) or (2) is represented by the following general formula (1-1) or (2-1): 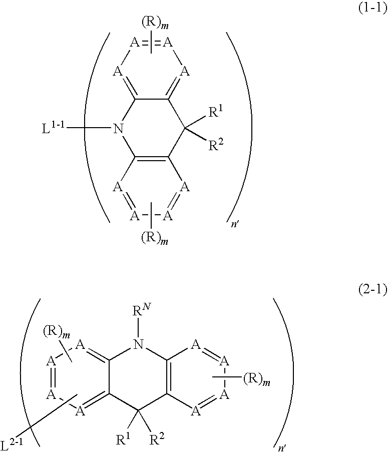
wherein
each of R and R^{N} independently represents a substituent;
each of R¹ and R² independently represents a substituent, provided that R¹ and R² do not represent an aryl group at the same time;
each A independently represents a carbon atom or a nitrogen atom;
each of L¹⁻¹ and L²⁻¹ independently represents phenylene or biphenylene;
n′ represents an integer of 2 or more and not more than 10; and
each m independently represents an integer.

4. The charge transport material according to claim 2, wherein
the general formula (1) is represented by the following general formula (3): 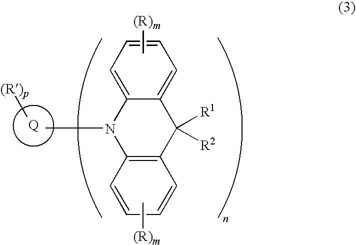
wherein
each of R and R′ independently represents a substituent;
each of R¹ and R² independently represents a substituent, provided that R¹ and R² do not represent an aryl group at the same time;
Q represents a 5-membered ring or a 6-membered ring;
n represents 2 or 3; and
each of m and p represents an integer.

5. The charge transport material according to claim 4, wherein
the general formula (3) is represented by the following general formula (4): 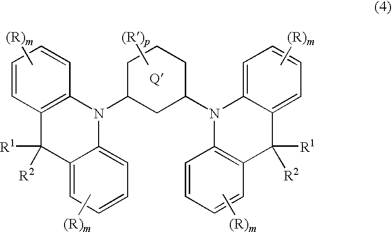
wherein
each of R and R′ independently represents a substituent;
each of R¹ and R² independently represents a substituent, provided that R¹ and R² do not represent an aryl group at the same time;
Q′ represents an aromatic 6-membered ring; and
each of m and p represents an integer.

6. The charge transport material according to claim 5, wherein
R¹ in the general formula (4) is a methyl group.

7. The charge transport material according to claim 2, which has an excited triplet level (T₁) in a thin film state of 3.0 eV or more and not more than 3.5 eV.

8. A composition comprising:
the compound according to claim 1.

9. A thin film comprising:
the compound according to claim 1.

10. An organic electroluminescence device comprising:
at least one organic layer including a light emitting layer containing a light emitting material between a cathode and an anode,
wherein
the at least one organic layer contains the compound according to claim 1.

11. The organic electroluminescence device according to claim 10, wherein
the light emitting layer contains a phosphorescent material.

12. The organic electroluminescence device according to claim 11, wherein
the phosphorescent material is an Ir complex or a Pt complex.

12. (canceled)

13. The organic electroluminescence device according to claim 11, wherein
the phosphorescent material is a Pt complex including a tridentate or more multidentate ligand.

14. The organic electroluminescence device according to claim 13, wherein
the phosphorescent material is a Pt complex represented by the following general formula (C-2):
General Formula (C-2) 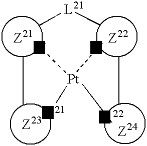
wherein
L²¹ represents a single bond or a divalent connecting group;
each of A²¹ and A²² independently represents C or N;
each of Z²¹ and Z²² independently represents a nitrogen-containing aromatic heterocyclic ring; and
each of Z²³ and Z²⁴ independently represents a benzene ring or an aromatic heterocyclic ring.

15. The organic electroluminescence device according to claim 14, wherein
the phosphorescent material is a Pt complex represented by the following general formula (5): 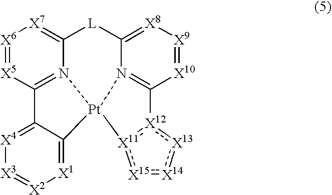
wherein
each of X¹, X², X³ and X⁴ independently represents a carbon atom or a nitrogen atom, provided that at least one of X¹, X², X³ and X⁴ represents a nitrogen atom;
each of X⁵, X⁶, X⁷, X⁸, X⁹ and X¹⁰ independently represents a carbon atom or a nitrogen atom;
each of X¹¹ and X¹² independently represents a carbon atom or a nitrogen atom;
each of X¹³, X¹⁴ and X¹⁵ independently represents a carbon atom, a nitrogen atom, an oxygen atom or a sulfur atom;
the number of nitrogen atoms contained in the 5-membered ring structure formed by X¹¹, X¹², X¹³, X¹⁴ and X¹⁵ is not more than 2; and
L represents a single bond or a divalent connecting group.

16. The organic electroluminescence device according to claim 12, wherein
the phosphorescent material is an Ir complex represented by the following general formula (T-1): 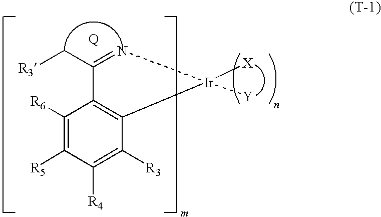
wherein
R₃′ represents an alkyl group, a heteroalkyl group, an aryl group or a heteroaryl group and may further have a substituent Z;
R₅ represents an aryl group or a heteroaryl group and may be further substituted with a non-aromatic group;
the ring Q represents an aromatic heterocyclic ring or a condensed aromatic heterocyclic ring each having at least one nitrogen atom, which is coordinated on Ir, and may be further substituted with a non-aromatic group;
each of R₃, R₄ and R₆ represents a hydrogen atom, an alkyl group, an alkenyl group, an alkynyl group, —CN, a perfluoroalkyl group, a trifluorovinyl group, —CO₂R, —C(O)R, —NR₂, —NO₂, —OR, a halogen atom, an aryl group or a heteroaryl group and may further have a substituent Z;
R₃ and R₄ may be bonded to each other to form a condensed 4-membered to 7-membered ring, the condensed 4-membered to 7-membered ring is a cycloalkyl, a cycloheteroalkyl, an aryl or a heteroaryl, and the condensed 4-membered to 7-membered ring may further have a substituent Z;
R₃′ and R₆ may be connected to each other via a connecting group selected among —CR₂—CR₂—, —CR═CR—, —CR₂—, —O—, —NR—, —O—CR₂—, —NR—CR₂— and —N═CR— to form a ring; and
each R independently represents a hydrogen atom, an alkyl group, an alkenyl group, an alkynyl group, a heteroalkyl group, an aryl group or a heteroaryl group and may further have a substituent Z;
each Z independently represents a halogen atom, —R′, —OR′, —N(R′)₂, —SR′, —C(O)R′, —C(O)OR′, —C(O)N(R′)₂, —CN, —NO₂, —SO₂, —SOR′, —SO₂R′ or —SO₃R′;
each R′ independently represents a hydrogen atom, an alkyl group, a perhaloalkyl group, an alkenyl group, an alkynyl group, a heteroalkyl group, an aryl group or a heteroaryl group;
(X—Y) represents an auxiliary ligand; and
m represents an integer of from 1 to 3 and n represents an integer of from 0 to 2, provided that m+n is 3.

17. The organic electroluminescence device according to claim 11, wherein
the phosphorescent material has a maximum light emission wavelength of not more than 500 nm.

18. A light emission apparatus comprising:
the organic electroluminescence device according to claim 10.

19. A display apparatus comprising:
the organic electroluminescence device according to claim 10.

20. An illumination apparatus comprising:
the organic electroluminescence device according to claim 10.

21. A composition comprising:
the charge transport material according to claim 2.

22. A thin film comprising:
the charge transport material according to claim 2.

23. An organic electroluminescence device comprising:
at least one organic layer including a light emitting layer containing a light emitting material between a cathode and an anode,
wherein
the at least one organic layer contains the charge transport material according to claim 2.

24. The organic electroluminescence device according to claim 23, wherein
the light emitting layer contains a phosphorescent material.

25. The organic electroluminescence device according to claim 24, wherein
the phosphorescent material is an Ir complex or a Pt complex.

26. The organic electroluminescence device according to claim 25, wherein
the phosphorescent material is a Pt complex including a tridentate or more multidentate ligand.

27. The organic electroluminescence device according to claim 26, wherein
the phosphorescent material is a Pt complex represented by the following general formula (C-2):
General Formula (C-2) 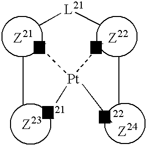
wherein
L²¹ represents a single bond or a divalent connecting group;
each of A²¹ and A²² independently represents C or N;
each of Z²¹ and Z²² independently represents a nitrogen-containing aromatic heterocyclic ring; and
each of Z²³ and Z²⁴ independently represents a benzene ring or an aromatic heterocyclic ring.

28. The organic electroluminescence device according to claim 27, wherein
the phosphorescent material is a Pt complex represented by the following general formula (5): 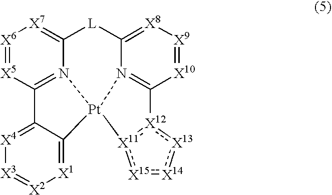
wherein
each of X¹, X², X³ and X⁴ independently represents a carbon atom or a nitrogen atom, provided that at least one of X¹, X², X³ and X⁴ represents a nitrogen atom;
each of X⁵, X⁶, X⁷, X⁸, X⁹ and X¹⁰ independently represents a carbon atom or a nitrogen atom;
each of X¹¹ and X¹² independently represents a carbon atom or a nitrogen atom;
each of X¹³, X¹⁴ and X¹⁵ independently represents a carbon atom, a nitrogen atom, an oxygen atom or a sulfur atom;
the number of nitrogen atoms contained in the 5-membered ring structure formed by X¹¹, X¹², X¹³, X¹⁴ and X¹⁵ is not more than 2; and
L represents a single bond or a divalent connecting group.

29. The organic electroluminescence device according to claim 25, wherein
the phosphorescent material is an Ir complex represented by the following general formula (T-1): 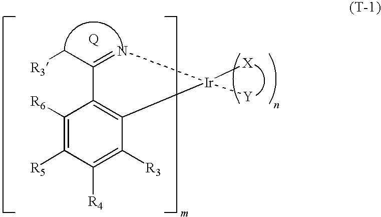
wherein
R₃′ represents an alkyl group, a heteroalkyl group, an aryl group or a heteroaryl group and may further have a substituent Z;
R₅ represents an aryl group or a heteroaryl group and may be further substituted with a non-aromatic group;
the ring Q represents an aromatic heterocyclic ring or a condensed aromatic heterocyclic ring each having at least one nitrogen atom, which is coordinated on Ir, and may be further substituted with a non-aromatic group;
each of R₃, R₄ and R₆ represents a hydrogen atom, an alkyl group, an alkenyl group, an alkynyl group, —CN, a perfluoroalkyl group, a trifluorovinyl group, —CO₂R, —C(O)R, —NR₂, —NO₂, —OR, a halogen atom, an aryl group or a heteroaryl group and may further have a substituent Z;
R₃ and R₄ may be bonded to each other to form a condensed 4-membered to 7-membered ring, the condensed 4-membered to 7-membered ring is a cycloalkyl, a cycloheteroalkyl, an aryl or a heteroaryl, and the condensed 4-membered to 7-membered ring may further have a substituent Z.
R₃′ and R₆ may be connected to each other via a connecting group selected among —CR₂—CR₂—, —CR═CR—, —CR₂—, —O—, —NR—, —O—CR₂—, —NR—CR₂— and —N═CR— to form a ring; and
each R independently represents a hydrogen atom, an alkyl group, an alkenyl group, an alkynyl group, a heteroalkyl group, an aryl group or a heteroaryl group and may further have a substituent Z;
each Z independently represents a halogen atom, —R′, —OR′, —N(R′)₂, —SR′, —C(O)R′, —C(O)OR′, —C(O)N(R′)₂, —CN, —NO₂, —SO₂, —SOR′, —SO₂R′ or —SO₃R′; and
each R′ independently represents a hydrogen atom, an alkyl group, a perhaloalkyl group, an alkenyl group, an alkynyl group, a heteroalkyl group, an aryl group or a heteroaryl group;
(X—Y) represents an auxiliary ligand; and
m represents an integer of from 1 to 3 and n represents an integer of from 0 to 2, provided that m+n is 3.

30. The organic electroluminescence device according to claim 24, wherein
the phosphorescent material has a maximum light emission wavelength of not more than 500 nm.

31. A light emission apparatus comprising:
the organic electroluminescence device according to claim 23.

32. A display apparatus comprising:
the organic electroluminescence device according to claim 23.

33. An illumination apparatus comprising:
the organic electroluminescence device according to claim 23.
